(19) United States
(12) Patent Application Publication
(10) Pub. No.: US 2003/0228301 A1
(43) Pub. Date: Dec. 11, 2003

(54) Novel human proteins, polynucleotides encoding them and methods of using the same

(76) Inventors: Li Li, Branford, CT (US); Katarzyna Furtak, Ansonia, CT (US); Amanda Perna, Hamden, CT (US); Meera Patturajan, Branford, CT (US); Richard A. Shimkets, Guilford, CT (US); Xiaojia Sasha Guo, Branford, CT (US); Stacie J. Casman, North Haven, CT (US); Catherine E. Burgess, Wethersfield, CT (US); Uriel M. Malyankar, Branford, CT (US); Velizar T. Tchernev, Branford, CT (US); Corine A. Vernet, Branford, CT (US); Kimberly A. Spytek, New Haven, CT (US); Michele Agee, Wallingford, CT (US); Luca Rastelli, Guilford, CT (US); Suresh G. Shenoy, Branford, CT (US); William M. Grosse, Branford, CT (US); John P. Alsobrook II, Madison, CT (US); Denise M. Lepley, Branford, CT (US); Valerie Gerlach, Branford, CT (US); Schlomit R. Edinger, New Haven, CT (US); John R. MacDougall, Hamden, CT (US); John A. Peyman, New Haven, CT (US); Erik Gunther, Branford, CT (US); David J. Stone, Guilford, CT (US); Karen Ellerman, Branford, CT (US); Esha A. Gangolli, Madison, CT (US)

Correspondence Address: Ivor R. Elrifi, Esq., Mintz, Levin, Cohn, Ferris,, Glovsky and Popeo, P.C., One Financial Center, Boston, MA, 02111, US

(21) Appl. No.: 10/004,378

(22) Filed: Oct. 24, 2001

Related U.S. Application Data
(60) Provisional application No. 60/242,882, filed on Oct. 24, 2000.
(60) Provisional application No. 60/242,765, filed on Oct. 24, 2000.
(60) Provisional application No. 60/300,206, filed on Jun. 22, 2001.
(60) Provisional application No. 60/242,789, filed on Oct. 24, 2000.
(60) Provisional application No. 60/242,768, filed on Oct. 24, 2000.
(60) Provisional application No. 60/242,767, filed on Oct. 24, 2000.
(60) Provisional application No. 60/243,622, filed on Oct. 26, 2000.
(60) Provisional application No. 60/273,047, filed on Mar. 2, 2001.
(60) Provisional application No. 60/243,591, filed on Oct. 26, 2000.
(60) Provisional application No. 60/243,950, filed on Oct. 27, 2000.
(60) Provisional application No. 60/316,509, filed on Aug. 31, 2001.
(60) Provisional application No. 60/243,593, filed on Oct. 26, 2000.
(60) Provisional application No. 60/243,502, filed on Oct. 26, 2000.

(51) Int. Cl. C12Q 1/68; C07H 21/04; A61K 39/395; C12P 21/02; C12N 5/06; C07K 14/47; C07K 16/40

(52) U.S. Cl. 424/130.1; 435/6; 435/183; 435/69.1; 435/320.1; 435/325; 530/350; 530/388.1; 536/23.2

(57) ABSTRACT

Disclosed are novel polypeptides and nucleic acids encoding same. Also disclosed are vectors, host cells, antibodies and recombinant methods for producing the polypeptides and polynucleotides, as well as methods for using same.

### RELATED APPLICATIONS

This application claims priority to U.S. Ser. No. 60/242,882 (Attorney Ref.: 21402-179), filed Oct. 24, 2000; U.S. Ser. No. 60/242,765 (Attorney Ref.: 21402-180), filed Oct. 24, 2000; U.S. Ser. No. 60/300,206 (Attorney Ref.: 21402-180A), filed Jun. 22, 2001; U.S. Ser. No. 60/242,789 (Attorney Ref.: 21402-181), filed Oct. 24, 2000; U.S. Ser. No. 60/242,768 (Attorney Ref.: 21402-182), filed Oct. 24, 2000; U.S. Ser. No. 60/242,767 (Attorney Ref.: 21402-183), filed Oct. 24, 2000; U.S. Ser. No. 60/243,622 (Attorney Ref.: 21402-184), filed Oct. 26, 2000; U.S. Ser. No. 60/273,047 (Attorney Ref.: 21402-184A), filed Mar. 2, 2001; U.S. Ser. No. 60/243,591 (Attorney Ref.: 21402-185), filed Oct. 26, 2000; U.S. Ser. No. 60/243,950 (Attorney Ref.: 21402-187), filed Oct. 27, 2000; U.S. Ser. No. 60/316,509 (Attorney Ref.: 21402-187A), filed Aug. 31, 2001; U.S. Ser. No. 60/243,593 (Attorney Ref.: 21402-188), filed Oct. 26, 2000; and U.S. Ser. No. 60/243,502 (Attorney Ref.: 21402-190), filed Oct. 26, 2000, each of which is incorporated by reference in its entirety.

### FIELD OF THE INVENTION

The invention relates to polynucleotides and the polypeptides encoded by such polynucleotides, as well as vectors, host cells, antibodies and recombinant methods for producing the polypeptides and polynucleotides, as well as methods for using the same.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, NOV9, and NOV10 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS:1, 3, 5, 7, 9, 11, 13, 15, 17, 19, 21, 23, 25, 27, 28, and 30. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18, 20, 22, 24, 26, 29, and 31. The nucleic acid can be, for example, a genomic DNA fragment or a cDNA molecule that includes the nucleic acid sequence of any of SEQ ID NOS: 1, 3, 5, 7, 9, 11, 13, 15, 17, 19, 21, 23, 25, 27, 28, and 30.

Also included in the invention is an oligonucleotide, e.g., an oligonucleotide which includes at least 6 contiguous nucleotides of a NOVX nucleic acid (e.g., SEQ ID NOS: 1, 3, 5, 7, 9, 11, 13, 15, 17, 19, 21, 23, 25, 27, 28, and 30) or a complement of said oligonucleotide. Also included in the invention are substantially purified NOVX polypeptides (SEQ ID NOS:2, 4, 6, 8, 10, 12, 14, 16, 18, 20, 22, 24, 26, 29, and 31). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., Cancer, Leukodystrophies, Breast cancer, Ovarian cancer, Prostate cancer, Uterine cancer, Hodgkin disease, Adenocarcinoma, Adrenoleukodystrophy, Cystitis, incontinence, Von Hippel-Lindau (VHL) syndrome, hypercalceimia, Endometriosis, Hirschsprung's disease, Crohn's Disease, Appendicitis, Cirrhosis, Liver failure, Wolfram Syndrome, Smith-Lemli-Opitz syndrome, Retinitis pigmentosa, Leigh syndrome; Congenital Adrenal Hyperplasia, Xerostomia; tooth decay and other dental problems; Inflammatory bowel disease, Diverticular disease, fertility, Infertility, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, Hemophilia, Hypercoagulation, Idiopathic thrombocytopenic purpura, obesity, Diabetes Insipidus and Mellitus with Optic Atrophy and Deafness, Pancreatitis, Metabolic Dysregulation, transplantation recovery, Autoimmune disease, Systemic lupus erythematosus, asthma, arthritis, psoriasis, Emphysema, Scleroderma, allergy, ARDS, Immunodeficiencies, Graft vesus host, Alzheimer's disease, Stroke, Parkinson's disease, Huntington's disease, Cerebral palsy, Epilepsy, Multiple sclerosis, Ataxia-telangiectasia, Behavioral disorders, Addiction, Anxiety, Pain, Neurodegeneration, Muscular dystrophy, Lesch-Nyhan syndrome, Myasthenia gravis, schizophrenia, and other dopamine-dysfunctional states, levodopa-induced dyskinesias, alcoholism, pileptic seizures and other neurological disorders, mental depression, Cerebellar ataxia, pure; Episodic ataxia, type 2; Hemiplegic migraine, Spinocerebellar ataxia-6, Tuberous sclerosis, Renal artery stenosis, Interstitial nephritis, Glomerulonephritis, Polycystic kidney disease, Renal tubular acidosis, IgA nephropathy, and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

NOVX nucleic acids and polypeptides are further useful in the generation of antibodies that bind immuno-specifically to the novel NOVX substances for use in therapeutic or diagnostic methods. These NOVX antibodies may be generated according to methods known in the art, using prediction from hydrophobicity charts, as described in the “Anti-NOVX Antibodies” section below. The disclosed NOVX proteins have multiple hydrophilic regions, each of which can be used as an immunogen. These NOVX proteins can be used in assay systems for functional analysis of various human disorders, which will help in understanding of pathology of the disease and development of new drug targets for various disorders.

The NOVX nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their encoded polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | | Nucleic | Poly- |
|---|---|---|---|---|
| | | | Acid | peptide |
| NOVX | | | SEQ ID | SEQ ID |
| No. | Internal Acc. No. | Homology | NO. | NO. |
| 1a | nh0318116_{—} | TRAF5 | 1 | 2 |
| | 20000809_da1 | | | |
| 1b | nh0318116_{—} | TRAF5 | 3 | 4 |
| | 20000809_da2 | | | |
| 2a | 246b18_20000718 | KIAA 1246 | 5 | 6 |
| 2b | CG55265-02 | Fibronectin/LLR/Ig | 7 | 8 |
| 2c | CG55265-03 | Fibronectin/LLR/Ig | 9 | 10 |
| 3 | ba342c24_{—} | Dematin | 11 | 12 |
| | 20000805_da1 | | | |
| 4a | 14578444_0_47 | Matrilin-2 | 13 | 14 |
| 4b | CG51018-03 | Matrilin-2 | 15 | 16 |
| 5 | SC85803748_A | GABA-receptor | 17 | 18 |
| 6a | ba465b22_{—} | Giant larvae | 19 | 20 |
| | 20000727 | homolog | | |
| 6b | CG55891-02 | Giant larvae | 21 | 22 |
| | | homolog | | |
| 7 | dj1182a14_da1 | Macrophage | 23 | 24 |
| | | stimulating protein | | |
| | | precursor | | |
| 8a | 138531995 | Nucleotide-sugar | 25 | 26 |
| | | transporter | | |
| 8b | CG111627-01 | Nucleotide-sugar | 27 | 26 |
| | | transporter | | |
| 9 | AC018755_da1 | OB binding | 28 | 29 |
| | | protein-2 | | |
| 10 | 30675745_0_499_{—} | Trypsin-like | 30 | 31 |
| | da1 | protein | | |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The present invention is based in part on nucleic acids encoding proteins that are new members of the following protein families: TRAF5, KIAA 1246-like Leucine rich repeat and fibronectin containing membrane protein, dematin, matrilin-2, GABA-receptor, giant larvae homolog, macrophage stimulating protein precursor, nucleotide-sugar transporter, OB binding protein-2 and trypsin-like protein. More particularly, the invention relates to nucleic acids encoding novel polypeptides, as well as vectors, host cells, antibodies, and recombinant methods for producing these nucleic acids and polypeptides.

NOV1 is homologous to a TRAF5 family of proteins. Thus, the NOV1 nucleic acids, polypeptides, antibodies and related compounds according to the invention will be useful in therapeutic and diagnostic applications implicated in, for example: Von Hippel-Lindau (VHL) syndrome, Alzheimer's disease, Stroke, Tuberous sclerosis, Hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Diabetes, Autoimmune disease, Immunodeficiencies, Renal artery stenosis, Interstitial nephritis, Glomerulonephritis, Polycystic kidney disease, Systemic lupus erythematosus, Renal tubular acidosis, IgA nephropathy, Systemic lupus erythematosus, Asthma, Emphysema, Scleroderma, Allergy, ARDS, Hemophilia, Hypercoagulation, Idiopathic thrombocytopenic purpura, Transplantation, Graft versus host disease (GVHD), Lymphaedema, Pancreatitis, Obesity, Hyperparathyroidism, Hypoparathyroidism, Endocrine dysfunctions, Growth and reproductive disorders, Fertility, Inflammatory bowel disease, Diverticular disease, Ulcers, Tonsilitis, Endometriosis, and/or other pathologies/disorders.

Tumor necrosis factor (TNF) receptor-associated factors (TRAFs) are signal transducers for members of the TNF receptor superfamily. TRAF proteins are composed of an N-terminal cysteine/histidine-rich region containing zinc RING and/or zinc finger motifs, a coiled coil (leucine zipper) motif, and a homologous region in the C terminus that defines the TRAF family, the TRAF domain. The TRAF domain is involved in self-association and receptor binding. Members of tumor necrosis factor receptor (TNFR) family signal largely through interactions with death domain proteins and TRAF proteins. Tumor necrosis factor (TNF) receptor-associated factors (TRAFs) are signal transducers for members of the TNF receptor superfamily. The previously identified murine TRAF5 (mTRAF5) has been shown to specifically interact with the lymphotoxin-beta receptor (LT-beta R) and activate the transcription factor NF-kappa B.

NOV2 is homologous to the KIAA 1246/fibronectin/leucine repeat family of proteins. Thus NOV2 nucleic acids, polypeptides, antibodies and related compounds according to the invention will be useful in therapeutic and diagnostic applications implicated in, for example: Lymphatic Diseases, Skin and Connective Tissue Diseases, Diabetes and Kidney Disease, Cancers, tumors, and Brain Disorders, disorders that can be addressed by controlling and directing cell migration, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Inflammatory bowel disease, Diverticular disease, Crohn's Disease and/or other pathologies/disorders.

The KIAA 1246-like proteins include the leucine-rich repeat and fibronectin containing membrane protein-like proteins.

The leucine rich-like proteins generally comprise leucine-rich repeats (LRRs), relatively short motifs (22-28 residues in length) found in a variety of cytoplasmic, membrane and extracellular proteins. Although theses proteins are associated with widely different functions, a common property involves protein-protein interaction. Although little is known about the 3-D structure of LRRS, it is believed that they can form amphipathic structures with hydrophilic surfaces capable of acting with membranes. In vitro studies of a synthetic LRR from Drosophila Toll protein have indicated that the peptides formm gels by adopting beta-sheet structures that form extended filaments. These results are consistent with the idea that LRRs mediate protein-protein interactions and cellular adhesion. Other functions of LRR-containing proteins include, for example, binding to enzymes and vascular repair. The 3-D structure of ribonuclease inhibitor, a protein containing 15 LRRs, hasd been determined, revealing LRRs to be a new class of alpha/beta fold. LRRs form elongated non globular structures and are often flanked by cysteine-rich domains.

Fibronectins are multi-domain glycoproteins found in a soluble form in plasma, and in an insoluble form in loose connective tissue and basement membranes. They contain multiple copies of 3 repeat regions (types I, II and III), which bind to a variety of substances including heparin, collagen, DNA, actin, fibrin and fibronectin receptors on cell surfaces. The wide variety of these substances means that fibronectins are involved in a number of important functions: e.g., wound healing; cell adhesion; blood coagulation; cell differentiation and migration; maintenance of the cellular cytoskeleton; and tumour metastasis. The role of fibronectin in cell differentiation is demonstrated by the marked reduction in the expression of its gene when neoplastic transformation occurs. Cell attachment has been found to be mediated by the binding of the tetrapeptide RGDS to integrins on the cell surface, although related sequences can also display cell adhesion activity. The fibronectin type III repeat region is an approximately 100 amino acid domain, different tandem repeats of which contain binding sites for DNA, heparin and the cell surface. The superfamily of sequences believed to contain FnIII repeats represents 45 different families, the majority of which are involved in cell surface binding in some manner, or are receptor protein tyrosine kinases, or cytokine receptors.

Based on the presence of LRR, fibronectin type III and immunoglobulin domains, it is likely that this novel protein mediates interactions between the cell and its surrounding environment.

NOV3 is homologous to a dematin protein. Thus, the NOV3 nucleic acids and polypeptides, antibodies and related compounds according to the invention will be useful in a variety of therapeutic and diagnostic applications.

Dematin is identified as a human erythroid cytoskeletal, actin-bundling protein. Dematin bundles actin filaments in a phosphorylation-dependent manner and is widely and abundantly expressed. In solution, it exists as a trimer of two 48 kDa subunits and one 52 kDa subunit. The 48 kDa subunit contains a villin-like headpiece domain. Villin is an actin-binding protein of the brush border cytoskeleton. The headpiece domain is essential for villin's actin bundling and actin modulating activity in the microvillar cytoskeleton. Unlike villin, the actin bundling activity of dematin is regulated by cAMP protein kinase-mediated phosphorylation. In comparison to the 48 kDa subunit, the 52 kDa subunit contains an additional 22 amino acid sequence in the C-terminal headpiece domain. This insertion contains a novel 11 amino acid motif that is shared with human erythrocyte protein 4.2 (palladin). The 11 amino acid stretch forms a nucleotide binding P-loop that directly and specifically binds ATP. Each monomer of dematin contains two F-actin binding sites, one in the headpiece and one in the N-terminal domain. Thus, dematin is an erythroid actin bundling protein whose function may be facilitated via its interaction with ATP.

NOV4 is homologous to a matrilin-2 family of proteins. Thus, NOV4 nucleic acids, polypeptides, antibodies and related compounds according to the invention will be useful in therapeutic and diagnostic applications implicated in, for example: diseases of the heart, blood vessels, lungs, or other smooth muscle tissue as well as diseases of the extracellular matrix tissue, and/or other pathologies/disorders.

The matrilin family at present has four members that all share a structure made up of von Willebrand factor A domains, epidermal growth factor-like domains and a coiled coil alpha-helical module. The first member of the family, matrilin-1 (previously called cartilage matrix protein or CMP), is expressed mainly in cartilage. Matrilin-3 has a similar tissue distribution, while matrilin-2 and -4 occur in a wide variety of extracellular matrices. Matrilin-1 is associated with cartilage proteoglycans as well as being a component of both collagen-dependent and collagen-independent fibrils and on the basis of the related structures other matrilins may play similar roles. The matrilin genes are strictly and differently regulated and their expression may serve as markers for cellular differentiation.

NOV5 is homologous to the GABA-receptor protein family. Thus NOV5 nucleic acids, polypeptides, antibodies and related compounds according to the invention will be useful in therapeutic and diagnostic applications implicated in, for example: cancer, trauma, regeneration (in vitro and in vivo), viral/bacterial/parasitic infection, multiple sclerosis, leukodystrophies, pain, Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, Ataxia-telangiectasia, behavioral disorders, addiction, anxiety, retinal and visual disorders, and/or other pathologies/disorders.

Neurotransmitter-gated ion channels, such as gamma-aminobutyric acid (GABA), are transmembrane receptor-ion channel complexes that open transiently upon binding of specific ligands, allowing rapid transmission of signals at chemical synapses. Of the five families known, four have been shown to form a sequence-related super-family. These are the gamma-aminobutyric acid (GABA), nicotinic acetylcholine, glycine and the serotonin receptors. The ionotropic glutamate receptors have a distinct primary structure. However, all these receptors are made up of varying subunits surrounding a central pore. Each of these subunits contains a large extracellular N-terminal ligand-binding region; 3 hydrophobic transmembrane domains; a large intracellular region; and a fourth hydrophobic domain. GABA receptors are regulated by anions. They play an important role in regulating neurotransmission. GABA receptors are heterodimers and it serves as an inhibitory neurotransmitter to block the transmission of an impulse from one cell to another in the central nervous system. Medically, GABA has been used to treat both epilepsy and hypertension where it is thought to induce tranquility in individuals who have a high activity of manic behavior and acute agitation. the expression of different subunits might play a role in neurotransmission in different organs. For example, these subunits show distinct patterns of expression in the brain and their expression is thought to be differentially regulated depending on the environment. GABA is the product of a biochemical decarboxylation reaction of glutamic acid by the vitamin pyridoxal.

NOV6 is homologous to the giant larvae homolog family of proteins. Thus NOV6 nucleic acids, polypeptides, antibodies and related compounds according to the invention will be useful in therapeutic and diagnostic applications implicated in, for example: cancer, disorders of the eyes, ovaries, digestive tract, brain, and/or other pathologies/disorders.

Lethal (2) giant larvae was the first of more than 27 recessive oncogenes identified in Drosophila, which provides an excellent model to study neoplastic mechanisms due to the fact that homologs of human oncogenes and tumor suppressors have been isolated and most of the complexes and associated pathways are conserved. The Drosophila tumor suppressor genes scribble, discs large and lethal giant larvae appear to act in a common pathway. Mutations in any of these genes lead to loss of apical-basal cell polarity and overproliferation of epithelia, revealing a close connection between cytoarchitecture and growth control. Further, loss of cell polarity and tissue architecture are characteristics of malignant cancers derived from epithelial tissues.

NOV7 is homologous to members of the macrophage stimulating protein precursor family of proteins. Thus, the NOV7 nucleic acids, polypeptides, antibodies and related compounds according to the invention will be useful in therapeutic and diagnostic applications implicated in, for example: Aicardi-Goutieres syndrome 1; Brugada syndrome; Deafness, autosomal recessive 6; Heart block, nonprogressive; Heart block, progressive, 2; Ichthyosiforme erythroderma, congenital, nonbullous; Long QT syndrome-3; Night blindness; congenital stationary; Pituitary ACTH-secreting adenoma; Small-cell cancer of lung; Ventricular fibrillation, idiopathic; entricular tachycardia, idiopathic; HIV infection, susceptibility/resistance to; Von Hippel-Lindau (VHL) syndrome; Cirrhosis; Transplantation, and/or other pathologies/disorders.

Macrophage-stimulating protein (MSP) is an 80-kD serum protein with homology to hepatocyte growth factor (HGF). Its receptor, RON tyrosine kinase, is a new member of the HGF receptor family. The MSP-RON signaling pathway has been implicated in the functional regulation of mononuclear phagocytes. However, the function of this pathway in other types of cells has not been elucidated. In contrast to the HGF receptor, which was expressed at the basolateral surface, RON was localized at the apical surface of ciliated epithelia in the airways and oviduct. In addition, MSP was found in the bronchoalveolar space at biologically significant concentrations. MSP bound to RON on normal human bronchial epithelial cells with a high affinity (Kd=0.5 nM) and induced autophosphorylation of RON. Activation of RON by MSP led to a significant increase in ciliary beatfrequency of human nasal cilia. These findings indicate that the ciliated epithelium of the mucociliary transport apparatus is a novel target of MSP. As ciliary motility is critical for mucociliary transport, such findings suggest that the MSP-RON signaling pathway is a novel regulatory system of mucociliary function and might be involved in the host defense and fertilization.

NOV8 is homologous to the nucleotide-sugar transporter family of proteins. Thus, NOV8 nucleic acids and polypeptides, antibodies and related compounds according to the invention will be useful in therapeutic and diagnostic applications implicated in, for example; cancer (preferably ovarian and pancreatic tumors). Furthermore, they could have efficacy for treatment of patients suffering from metabolic diseases, (preferably diabetes), and/or other pathologies/disorders.

Nucleotide sugar transporters are mainly located in the Golgi membranes and carry nucleotide sugars, that are produced outside the Golgi apparatus, into the organelle, where they serve as substrates for the elongation of carbohydrate chains by glycosyltransferases. They are thus indispensable for cellular glycoconjugate synthesis. Moreover, they may have regulatory roles in producing the structural variety of cellular glycoconjugates.

NOV9 is homologous to a OB binding protein-2 protein. Thus, the NOV9 nucleic acids, polypeptides, antibodies and related compounds according to the invention will be useful in therapeutic and diagnostic applications implicated in, for example: 3-methylglutaconicaciduria, type III; Charcot-Marie-Tooth disease, type 4F; Colorectal cancer; Cone-rod retinal dystrophy-2; DNA ligase I deficiency; Glutaricaciduria, type IIB; Heart block, progressive familial, type I; Hydatidiform mole; Hyperferritinemia-cataract syndrome; Leber congenital amaurosis due to defect in CRX; Liposarcoma; Myotonic dystrophy; Retinitis pigmentosa, late-onset dominant; Spinocereballar ataxia-13; T-cell acute lymphoblastic leukemia; Trichothiodystrophy; Xeroderma pigmentosum, group D; Diabetes mellitus, noninsulin-dependent; Polio, susceptibility to; Hemophilia; Hypercoagulation; Idiopathic thrombocytopenic purpura; Immunodeficiencies; Graft vesus host; Von Hippel-Lindau (VHL) syndrome; Cirrhosis; Fertility; Systemic lupus erythematosus, Autoimmune disease, Asthma, Emphysema, Scleroderma, allergy, ARDS, and/or other pathologies/disorders.

A novel leptin-binding protein of the immunoglobulin superfamily (OB-BP1) and a cross-hybridizing clone (OB-BP2) are identical to Siglec-5. Siglec-5 (sialic acid-binding Ig-like lectin-5), is a novel transmembrane member of the immunoglobulin superfamily, highly related to the myeloid antigen, CD33. The sialic acid-binding immunoglobulin-like lectins (SIGLECs), such as CD33, are a subgroup of the immunoglobulin (Ig) superfamily that mediate protein-carbohydrate interactions. Thus, OB-BP1, OB-BP2/Siglec-5, and CD33/Siglec-3 constitute a unique related subgroup with a high level of overall amino acid identity and may mediate cell-cell recognition events by interacting with sialylated glycoprotein ligands expressed on specific cell populations. Further, OB-BP1 may have a role in leptin physiology, as a molecular sink to regulate leptin serum levels.

NOV10 is homologous to members of the trypsin-like family of proteins. Thus, the NOV10 nucleic acids, polypeptides, antibodies and related compounds according to the invention will be useful in therapeutic and diagnostic applications implicated in, for example: digestion, blood clotting, immune reactions, fertilization of the ovum, various cancers (prostate and/or breast), infectious disease, cystic fibrosis, and/or other pathologies/disorders.

Trypsin is a proteolytic enzyme, or proteinase that acts to degrade protein. Trypsin is one of the three principal digestive proteinases, the other two being pepsin and chymotrypsin. In the digestive process, trypsin acts with the other proteinases to break down dietary protein molecules to their component peptides and amino acids. Trypsin continues the process of digestion (begun in the stomach) in the small intestine where a slightly alkaline environment (about pH 8) promotes its maximal enzymatic activity. Trypsin, produced in an inactive form by the pancreas (pancreatic protease), is remarkably similar in chemical composition and in structure to the other chief pancreatic proteinase, chymotrypsin. Both enzymes also appear to have similar mechanisms of action; residues of histidine and serine are found in the active sites of both. The chief difference between the two molecules seems to be in their specificity. Trypsin is the most discriminating of all the proteolytic enzymes in terms of the restricted number of chemical bonds that it will attack. For this reason, trypsin is widely employed as a reagent for the orderly and unambiguous cleavage of the amino acid sequence of proteins. Trypsin is a pancreatic serine protease with substrate specificity based upon positively charged lysine and arginine side chains. Serine proteases are one of a group of endoproteases from both animal and bacterial sources that share a common reaction mechanism based on formation of an acyl enzyme intermediate on a specific active serine residue. Serine proteases are all irreversibly inactivated by a series of organophosphorus esters, such as diisopropylfluorophosphate (DFP) and by naturally occurring inhibitors (serpins). This group includes enzymes active in digestion, blood clotting, immune reactios, and fertilization of the ovum. Proteolytic enzymes that exploit serine in their catalytic activity are ubiquitous, being found in viruses, bacteria and eukaryotes. They include a wide range of peptidase activity, including exopeptidase, endopeptidase, oligopeptidase and omega-peptidase activity.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

NOV1 includes two novel TNF Receptor Associated Factor 5-like (TRAF5-like) proteins. The disclosed sequences have been named NOV1a and NOV1b.

NOV1a

A disclosed NOV1a nucleic acid of 3675 nucleotides (also referred to as wugc_draft_h_nh0318116_{—}20000809_da1) (SEQ ID NO:1) encoding a novel TRAF5-like protein is shown in Table 1A. An open reading frame was identified beginning with an ATG initiation codon at nucleotides 55-57 and ending with a TAG codon at nucleotides 1408-1410. Putative untranslated regions upstream from the initiation codon and downstream from the termination codon are underlined in Table 1A. The start and stop codons are in bold letters.

TABLE 1A
NOV1a nucleotide sequence.

(SEQ ID NO:1)

GCAGCAGCCGCGCCTGCAGACCGGCCTCGCGGAGCCCGCGCGCCGAGCCCCACAATGGCTTATTCAGAAGAG
CATAAAGGTATGCCCTGTGGTTTCATCCGCCAGAATTCCGGCAACTCCATTTCCTTGGACTTTGAGCCCAGT
ATAGAGTACCAGTTTGTGGAGCGGTTGGAAGAGCGCTACAAATGTGCCTTCTGCCACTCGGTGCTTCACAAC
CCCCACCAGACAGGATGTGGGCACCGCTTCTGCCAGCACTGCATCCTGTCCCTGAGAGAATTAAACACAGTG
CCAATCTGCCCTGTAGATAAAGAGGTCATCAAATCTCAGGAGGTTTTTAAAGACAATTGTTGCAAAAGAGAA
GTCCTCAACTTATATGTATATTGCAGCAATGCTCCTGGATGTAATGCCAAGGTTATTCTGGGCCGGTACCAG
GATAAACGGAGGAACCTGCAGCAACATGAGCATTCAGCCTTACGGGAGCACATGCGTTTGGTTTTAGAAAAG
AATGTCCAATTAGAAGAACAGATTTCTGACTTACACAAGAGCCTAGAACAGAAAGAAAGTAAAATCCAGCAG
CTAGCAGAAACTATAAAGAAACTTGAAAACGAGTTCAAGCAGTTTGCACAGTTGTTTGGCAAAAATGGAAGC
TTCCTCCCAAACATCCAGGTTTTTGCCAGTCACATTGACAAGTCAGCTTGGCTAGAAGCTCAAGTGCATCAA
TTATTACAAATGGTTAACCAGCAACAAAATAAATTTGACCTGAGACCTTTGATGGAAGCAGTTGATACAGTG
AAACAGAAAATTACCCTGCTAGAAAACAATGATCAAAGATTAGCCGTTTTAGAAGAGGAAACTAACAAACAT
GATACCCACATTAATATTCATAAAGCACAGCTGAGTAAAAATGAAGAGCGATTTAAACTGCTGGAGGGTACT
TGCTATAATGGAAAGCTCATTTGGAAGGTGACAGATTACAAGATGAAGAAGAGAGAGGCGGTGGATGGGCAC
ACAGTGTCCATCTTCAGCCAGTCCTTCTACACCAGCCGCTGTGGCTACCGGCTCTGTGCTAGAGCATACCTG
AATGGGGATGGGTCAGGGAGGGGGTCACACCTGTCCCTATACTTTGTGGTCATGCGAGGAGAGTTTGACTCA
CTGTTGCAGTGGCCATTCAGGCAGAGGGTGACCCTGATGCTTCTGGACCAGAGTGGCAAAAAGAACATTATG
GAGACCTTCAAACCTGACCCCAATAGCAGCAGCTTTAAAAGACCTGATGGGGAGATGAACATTGCATCTGGC
TGTCCCCGCTTTGTGGCTCATTCTGTTTTGGAGAATGCCAAGAACGCCTACATTAAAGATGACACTCTCTTC
TTGAAAGTGGCCGTGGACTTAACTGACCTGGAGGATCTCTAGTCACTGTTATGGGGTGATAAGAGGACTTCT
TGGGGCCAGAACTGTGGAGGAGAGCACATTTGATTATCATATTGACCTGGATTTAGACTCAAAGCACATTTG
TATTTGCCTTTTTCCTTAACGTTTGAAGTCAGTTTAAAACTTCTCAAGTGCTGTCTTTTTACATTTTACTCT
GTCCCAGTTTGAAACTTAAAACTCTTAGAATATTCTCTTATTATTTATATTTTTATATTTCTTGAAAGATGG
TAAGTTTCTTGAAGTTTTTGGGGCGTTTCTCTTTTACTGGTGCTTAGCGCAGTGTCTCGGGCACTCTAAATA
TTGAGTGTTATGGAGGACACAGAGGTAGCAGAATCCCAGTTGAAAATGTTTTGATATTTTATTGTTTGGCCT
ATTGATTCTAGACCTGGCCTTAAGTCTGCAAAAGCCATCTTTATAAGGTAGGCTGTTCCAGTTAAGAAGTGG
GTGATGTAGTTACAAAGATAATATGCTCAGTTTGGACCTTTTTTTCAGTTAAATGCTAAATATATGAAAATT
ACTATACCTCTAAGTATTTTCATGAAATTCACCAGCAGTTTGCAAGCACAGTTTTGCAAGGCTCCATAAGAA
CTGGTGAATGGGGTAAGCATTTTCATTCTTCCTGCTGAAGTAAAGCAGAAAGTACTGCATAGTATATGAGAT
ATAGCCAGCTAGCTAAAGTTCAGATTTTGTTAGGTTCAACCCTATGAAAAAAACTATTTTCATAGGTCAAAA
ATGGTAAAAAATTAGCAGTTTCATAAGATTCAACCAAATAAATATATATATACACACACACATACATATACA
CCTATATATGTGTGTATACAAACAGTTCGAATGTATTTTGGTGACAGTAATAAATCAATGTGAGGATGGATA
GAATTTAGTATATGATAGAGAAAATGTCATAAATGGATAAAAGGAATTTACAACTTGAGGAGAAAACCTTTA
CAATTTCCTATGGGTGTCAGAAGTACTCTCAGCGAAAACTGATGGCTAAAACAGTATCTACTATTCTCTGAT
AACTTTTTTTTTGAGACAGAGTTTCATTGTCACCCAGGCTGGAGTACAGTGGCATGATCTCAGCTCACTGCA
AACTCTGCCTCCCGAATTCAAGTGATTCTCCTGCCTCAGCCTCCTGAGTAGCTGGGATTACAGGCGCCCGTC
ACCACACCCAGGTAATTTTTGTATTTTTAGTAGAGACGGAGTTTTGCCATGTTGGCCAAGCTGATCTCAAAC
TCCTGACCTCAAGTGATCTGCCCGCCTCGGCCTCCCAAAGTGCTGAGATTACAGGCATGACCCACCGCGTCA
AGCCTCTGACAACTATTGAATTTGTAAGCTGCTATGCAAATGGGCATTTATATAAACTTGTGATGTTTCTTG
TCAGAATTCTGAGTACTCTGTGAAGAACAGAAATGATCATATTCTTATGCATCTATCTGTATGGCTCTGAAG
GTGTATATACAAACTGAGATGAGTCCTTATGACTCTTGATAAGCCTGAGTTTAACAACAACAAAAATGCCAA
GTTGTCCTGAGCCCTTCTGCGTTGTTATGCCACTTCCCTACTGCTCATATGCACGCTGGCTCCCCTGGGCAC
GCAAGGATGAGTATGGGCCATGGGCCCCTGTAGAGCTGCTTACCTGGTGATGACCATGCACCTTACAATTTC
TGAACAGTTAACCCTATAGAAGCATGCTTTATATGAGTGTCTTCTGGGAAGAGGAACCTTCTTAATCTCTTC
TGTGGGATTTTCAAAATGCTAAAGACTCACACTGCAGCAATCATCCCAGATGATTAAATTCAAAGAAATAGG
TTCACAACAGGAATATACTGAAGAACTAGAGTGTCACTGCTGGTGAACTGTGGCACGGTTGCTCAACACATC
ACCTCGGACAAATTCAGGAAGCATTTCTTTAGCCCACAAGTCCAGACCCAGGTGCTCTGTATGTTTGTTTTT
AATATTCATCATATCCAAGTTCACTCTGTCTTCCTGAGCAGTGGAAGATCATATTGCTGTAACTTCTTTTAA
GTAGTTGATGTGGAAAACATTTTAAAGTGAATTTGTCAAAATGCTGGTTTTGTGTTTTATCCAACTTTTGTG
CATATATATAAAGTATGTCATGGCATGGTTTGCTTAGGAGTTCAGAGTTCCTTCATCATCGAAATAGTGATT
AAGTGATCCCAGAACAAGGAATACTAGAGTAAAAAGCACCTCTTTTTCAGAAAAAAAAAAAAAAAAAAAAAA
AAA

The TRAF5 disclosed in this invention maps to chromosome 1.

The sequence of the invention was derived by laboratory cloning of cDNA fragments covering the full length and/or part of the DNA sequence of the invention, and/or by in silico prediction of the full length and/or part of the DNA sequence of the invention from public human sequence databases. The laboratory cloning was performed by the following methods-SeqCalling™ or Exon Linking. These methods are described in detail in Example 2.

RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. The following human samples from different donors were used adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus for the RACE reaction. The sequences derived from these procedures were included in the SeqCalling Assembly process described in the preceding paragraph.

In a search of sequence databases, it was found, for example, that the nucleic acid sequence of the disclosed NOV1a has 3675 of 3993 bases (92%) identical to a 3993 bp TNF Receptor Associated Factor 5 mRNA from Homo sapiens (GENBANK-ID: AB000509|acc:AB000509). Public nucleotide databases include all GenBank databases and the GeneSeq patent database.

In all BLAST alignments herein, the “E-value” or “Expect” value is a numeric indication of the probability that the aligned sequences could have achieved their similarity to the BLAST query sequence by chance alone, within the database that was searched. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNN”) or the letter “X” in protein sequences (e.g., “XXX”). Low-complexity regions can result in high scores that reflect compositional bias rather than significant position-by-position alignment. Wootton and Federhen, Methods Enzymol 266:554-571, 1996.

The encoded protein of NOV1a having 451 amino acid residues (SEQ ID NO:2) is presented using the one-letter code in Table 1B. The full amino acid sequence of the protein of the invention was found to have 451 of 557 amino acid residues (81%) identical to, and 451 of 557 amino acid residues (81%) similar to, the 557 amino acid residue TNF Receptor Associated Factor 5 protein from Homo sapiens (Human) (SPTREMBL-ACC:O00463 TRAF5).

TABLE 1B
Encoded NOV1a protein sequence.

(SEQ ID NO:2)

MAYSEEHKGMPCGFIRQNSGNSISLDFEPSIEYQFVERLEERYKCAFCHSVLHNPHQTGCGHRFCQHCILSL
RELNTVPICPVDKEVIKSQEVFKDNCCKREVLNLYVYCSNAPGCNAKVILGRYQDKRRNLQQHEHSALREHM
RLVLEKNVQLEEQISDLHKSLEQKESKIQQLAETIKKLEKEFKQFAQLFGKNGSFLPNIQVFASHIDKSAWL
EAQVHQLLQMVNQQQNKFDLRPLMEAVDTVKQKITLLENNDQRLAVLEEETNKHDTHINIHKAQLSKNEERF
KLLEGTCYNGKLIWKVTDYKMKKREAVDGHTVSIFSQSPYTSRCGYRLCARAYLNGDGSGRGSHLSLYFVVM
RGEFDSLLQWPFRQRVTLMLLDQSGKKNIMETFKPDPNSSSFKRPDGEMNIASGCPRFVAHSVLENAKNAYI
KDDTLFLKVAVDLTDLEDL

NOV1b

In an alternative embodiment, a NOV1 variant is NOV1b of 3480 nucleotides (also referred to as wugc_draft_h_nh0318116_{—}20000809_da2) (SEQ ID NO:3) is shown in Table 1C. An open reading frame was identified beginning with an ATG initiation codon at nucleotides 55-57 and ending with a TAG codon at nucleotides 1213-1215. Putative untranslated regions upstream from the initiation codon and downstream from the termination codon are underlined in Table 1C. The start and stop codons are in bold letters.

TABLE 1C
NOV1b nucleotide sequence.

(SEQ ID NO:3)

GCAGCAGCCGCGCCTGCAGACCGGCCTCGCGGAGCCCGCGCGCCGAGCCCCACAATGGCTTATTCACAAGAG
CATAAAGGTATGCCCTGTGGTTTCATCCGCCAGAATTCCGGCAACTCCATTTCCTTGGACTTTGAGCCCAGT
ATAGAGTACCAGTTTGTGGAGCGGTTGGAAGAGCGCTACAAATGTGCCTTCTGCCACTCGGTGCTTCACAAC
CCCCACCAGACAGGATGTGGGCACCGCTTCTGCCAGCACTGCATCCTGTCCCTGAGAGAATTAAACACAGTG
CCAATCTGCCCTGTAGATAAAGAGGTCATCAAATCTCAGGAGATTTCTGACTTACACAAGAGCCTAGAACAG
AAAGAAAGTAAAATCCAGCAGCTAGCAGAAACTATAAAGAAACTTGAAAAGGAGTTCAAGCAGTTTGCACAG
TTGTTTGGCAAAAATGGAAGCTTCCTCCCAAACATCCAGGTTTTTGCCAGTCACATTGACAAGTCAGCTTGG
CTAGAAGCTCAAGTGCATCAATTATTACAAATGGTTAACCAGCAACAAAATAAATTTGACCTGAGACCTTTG
ATGGAAGCAGTTGATACAGTGAAACAGAAAATTACCCTGCTAGAAAACAATGATCAAAGATTAGCCGTTTTA
GAAGAGGAAACTAACAAACATGATACCCACATTAATATTCATAAAGCACAGCTGAGTAAAAATGAAGAGCGA
TTTAAACTGCTGGAGGGTACTTGCTATAATGGAAAGCTCATTTGGAAGGTGACAGATTACAAGATGAAGAAG
AGAGAGGCGGTGGATGGGCACACAGTGTCCATCTTCAGCCAGTCCTTCTACACCAGCCGCTGTGGCTACCGG
CTCTGTGCTAGAGCATACCTGAATGGGGATGGGTCAGGGAGGGGGTCACACCTGTCCCTATACTTTGTGGTC
ATGCGAGGAGAGTTTGACTCACTGTTGCAGTGGCCATTCAGGCAGAGGGTGACCCTGATGCTTCTGGACCAG
AGTGGCAAAAAGAACATTATGGAGACCTTCAAACCTGACCCCAATAGCAGCAGCTTTAAAAGACCTGATGGG
GAGATGAACATTGCATCTGGCTGTCCCCGCTTTGTGGCTCATTCTGTTTTGGAGAATGCCAAGAACGCCTAC
ATTAAAGATGACACTCTGTTCTTGAAAGTGGCCGTGGACTTAACTGACCTGGAGGATCTCTAGTCACTGTTA
TGGGGTGATAAGAGGACTTCTTGGGGCCAGAACTGTGGAGGAGAGCACATTTGATTATCATATTGACCTGGA
TTTAGACTCAAAGCACATTTGTATTTGCCTTTTTCCTTAACGTTTGAAGTCAGTTTAAAACTTCTGAAGTGC
TGTCTTTTTACATTTTACTCTGTCCCAGTTTGAAACTTAAAACTCTTAGAATATTCTCTTATTATTTATATT
TTTATATTTCTTGAAAGATGGTAAGTTTCTTGAAGTTTTTGGGGCGTTTCTCTTTTACTGGTGCTTAGCGCA
GTGTCTCGGGCACTCTAAATATTGAGTGTTATGGAGGACACAGAGGTAGCAGAATCCCAGTTGAAAATGTTT
TGATATTTTATTGTTTGGCCTATTGATTCTAGACCTGGCCTTAAGTCTGCAAAAGCCATCTTTATAAGGTAG
GCTGTTCCAGTTAAGAAGTGGGTGATGTAGTTACAAAGATAATATGCTCAGTTTGGACCTTTTTTTCAGTTA
AATGCTAAATATATGAAAATTACTATACCTCTAAGTATTTTCATGAAATTCACCAGCAGTTTGCAAGCACAG
TTTTGCAAGGCTGCATAAGAACTGGTGAATGGGGTAAGCATTTTCATTCTTCCTGCTGAAGTAAACCAGAAA
GTACTGCATAGTATATGAGATATAGCCAGCTAGCTAAAGTTCAGATTTTGTTAGGTTCAACCCTATGAAAAA
AACTATTTTCATAGGTCAAAAATGGTAAAAAATTAGCAGTTTCATAAGATTCAACCAAATAAATATATATAT
ACACACACACATACATATACACCTATATATGTGTGTATACAAACAGTTCGAATGTATTTTGGTGACAGTAAT
AAATCAATGTGAGGATGGATAGAATTTAGTATATGATAGAGAAAATGTCATAAATGGATAAAAGGAATTTAC
AACTTGAGGAGAAAACCTTTACAATTTCCTATGGGTGTCAGAAGTACTCTCAGCGAAAACTGATGGCTAAAA
CAGTATCTACTATTCTCTGATAACTTTTTTTTTGAGACAGAGTTTCATTGTCACCCAGGCTGGAGTACAGTG
GCATGATCTCAGCTCACTGCAAACTCTGCCTCCCGAATTCAAGTGATTCTCCTGCCTCAGCCTCCTGAGTAG
CTGGGATTACAGGCGCCCGTCACCACACCCAGGTAATTTTTGTATTTTTAGTAGAGACGGAGTTTTGCCATG
TTGGCCAAGCTGATCTCAAACTCCTGACCTCAAGTGATCTGCCCGCCTCGGCCTCCCAAAGTGCTGAGATTA
CAGGCATGACCCACCGCGTCAAGCCTCTGACAACTATTGAATTTGTAAGCTGCTATGCAAATGGGCATTTAT
ATAAACTTGTGATGTTTCTTGTCAGAATTCTGAGTACTCTGTGAAGAACAGAAATGATCATATTCTTATGCA
TCTATCTGTATGGGTCTGAAGGTGTATATACAAACTGAGATGAGTCCTTATGACTCTTGATAAGCCTGAGTT
TAACAACAACAAAAATGCCAAGTTGTCCTGAGCCCTTCTGCGTTGTTATGCCACTTCCCTACTGCTCATATG
CACGCTGGCTCCCCTGGGCACGCAAGGATGAGTATGGGCCATGGGCCCCTGTAGAGCTGCTTACCTGGTGAT
GACCATGCACCTTACAATTTCTGAACAGTTAACCCTATAGAAGCATGCTTTATATGAGTGTCTTCTGGGAAG
AGGAACCTTCTTAATCTCTTCTGTGGGATTTTCAAAATGCTAAAGACTCACACTGCAGCAATCATCCCAGAT
GATTAAATTCAAAGAAATAGGTTCACAACAGGAATATACTGAAGAACTAGAGTGTCACTGCTGGTGAACTGT
GGCACGGTTGCTCAACACATCACCTCGGACAAATTCAGGAAGCATTTCTTTAGCCCACAAGTCCAGACCCAG
GTGCTCTGTATGTTTGTTTTTAATATTCATCATATCCAAGTTCACTCTGTCTTCCTGAGCAGTGGAAGATCA
TATTGCTGTAACTTCTTTTAAGTAGTTGATGTGGAAAACATTTTAAAGTGAATTTGTCAAAATGCTGGTTTT
GTGTTTTATCCAACTTTTGTGCATATATATAAAGTATGTCATGGCATGGTTTGCTTAGGAGTTCAGAGTTCC
TTCATCATCGAAATAGTGATTAAGTGATCCCAGAACAAGGAATACTAGAGTAAAAAGCACCTCTTTTTCAGA
AAAAAAAAAAAAAAAAAAAAAAAA

In a search of sequence databases, it was found, for example, that the nucleic acid sequence of NOV1b has 3480 of 3993 bases (87%) identical to a 3993 bp TNF Receptor Associated Factor 5 mRNA from Homo sapiens (GENBANK-ID: AB000509|acc:AB000509).

The encoded protein of NOV1b (SEQ ID NO:4) having 386 amino acid residues is presented using the one-letter code in Table 1D.

TABLE 1D
Encoded NOV1b protein sequence.

(SEQ ID NO:4)

MAYSEEHKGMPCGFIRQNSGNSISLDFEPSIEYQFVERLEERYKCAFCHSVLHNPHQTGCGHRFCQECILSL
RELNTVPICPVDKEVIKSQEISDLHKSLEQKESKIQQLAETIKKLEKEFKQFAQLFGKNGSFLPNIQVFASH
IDKSAWLEAQVHQLLQMVNQQQNKFDLRPLMEAVDTVKQKITLLENNDQRLAVLEEETNKHDTHINIHKAQL
SKNEERFKLLEGTCYNGKLIWKVTDYKMKKREAVDGHTVSIFSQSFYTSRCGYRLCARAYLNGDGSGRGSHL
SLYFVVMRGEFDSLLQWPFRQRVTLMLLDQSGKKNIMETFKPDPNSSSFKRPDGEMNIASGCPRFVAHSVLE
NAKNAYIKDDTLFLKVAVDLTDLEDL

The full amino acid sequence of the protein of NOV1b was found to have 386 of 557 amino acid residues (69%) identical to, and 386 of 557 amino acid residues (69%) similar to, the 557 amino acid residue TNF Receptor Associated Factor 5 protein from Homo sapiens (Human) (SPTREMBL-ACC:O00463 TRAF5).

The TRAF5-like protein, NOV1, disclosed in this invention is expressed in at least the following tissues: Aorta, Ascending Colon, Brain, Chorionic villus, Colon, Dermis, Epidermis, Foreskin, Kidney, Lung Lymphoid tissue, Pancreas, Parathyroid Gland, Peripheral Blood, Pituitary, Placenta, Prostate, Small Intestine, Spleen, Stomach, Tonsils, Uterus, Whole organism.

The SignalP, Psort and/or Hydropathy results predict that NOV1a and NOV1b have no known signal peptide and are likely to be localized to the cytoplasm with a certainty of 0.4500. In alternative embodiments, NOV1 polypeptide is located to the microbody (peroxisome) with a certainty of 0.3000, the mitochondrial matrix space with a certainty of 0.1000, or the lysosome (lumen) with a certainty of 0.1000.

Homologies to any of the above NOV1 proteins will be shared by the other NOV1 protein insofar as they are homologous to each other. Any reference to NOV1 is assumed to refer to both of the NOV1 proteins in general, unless otherwise noted.

Additional SNP variants of NOV1 are disclosed in Example 3 and TaqMan data can be found in Example 1. The amino acid sequence of NOV1 has high homology to other proteins as shown in Table 1E.

TABLE IE
BLASTX results for NOV1

| | | Smallest |
|---|---|---|
| | | Sum |
| | High | Prob |
| Sequences producing High-scoring Segment Pairs: | Score | P(N) |
| patp: AAW27609 Murine TRAF5 - Mus sp, 558 aa | 1386 | 1.9e−205 |
| patp: AAW29257 Murine TRAF5, a TNF receptor | 1381 | 6.5e−205 |
| associated factor family protein - Murine | | |
| sp, 558 aa | | |
| patp: AAW27610 Human TRAF5 - Homo sapiens, | 1689 | 1.0e−173 |
| 557 aa | | |
| patp: AAW29258 Human TRAF5, a TNF receptor | 1689 | 1.0e−173 |
| associated factor family protein - Homo | | |
| sapiens, 557 aa | | |
| patp: AAY98168 Human TRAF5 protein - Homo | 1689 | 1.0e−173 |
| sapiens, 557 aa | | |

NOV1 also has homology to the proteins shown in the BLASTP data in Table 1F.

TABLE IF
BLASTP results for NOV1

| Gene Index/ | Protein/ | Length | | | |
|---|---|---|---|---|---|
| Identifier | Organism | (aa) | Identity (%) | Positives (%) | Expect |
| ptnr: SPTREMBL- | TRAF5 - Mus | 558 | 252/325 | 296/325 | 2.8e−205 |
| ACC: P70191 | musculus (Mouse) | | (80%) | (91%) | |
| ptnr: SPTREMBL- | TRAF5 - Mus | 558 | 261/325 | 295/325 | 9.5e−205 |
| ACC: Q61480 | musculus (Mouse) | | (80%) | (90%) | |
| ptnr: SPTREMBL- | TRAF5 (TNF | 557 | 336/392 | 348/392 | 1.5e−173 |
| ACC: O00463 | RECEPTOR | | (85%) | (88%) | |
| | ASSOCIATED | | | | |
| | FACTOR 5) - Homo | | | | |
| | sapiens (Human) | | | | |
| ptnr: pir-id: S68467 | CD40 receptor- | 543 | 148/335 | 228/335 | 1.5e−94 |
| | associated protein | | (44%) | (68%) | |
| | CAP-1 - human | | | | |
| ptnr: SWISSNEW- | TNF receptor | 568 | 163/401 | 252/401 | 1.0e−71 |
| ACC: Q13114 | associated factor 3 | | (40%) | (62%) | |
| | (CD40 receptor | | | | |
| | associated factor 1) | | | | |
| | (CRAF1) (CD40 | | | | |
| | binding protein) | | | | |
| | (CD40BP) (LMP1 | | | | |
| | associated protein) | | | | |
| | (LAP1) (CAP-1) - | | | | |
| | Homo sapiens | | | | |
| | (Human) | | | | |

The homology between the sequences in Table 1F as well as between the NOV1 variants is shown graphically in the ClustalW analysis shown in Table 1G. In the ClustalW alignment of the NOV1 proteins, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results for NOV1 as disclosed in Table 1H, were collected from the Conserved Domain Database (CDD) with Reverse Position Specific BLAST analyses. This BLAST analysis software samples domains found in the Smart and Pfam collections. For Table 1H and all successive DOMAIN sequence alignments, fully conserved single residues are indicated by black shading or by the sign (|) and “strong” semi-conserved residues are indicated by grey shading or by the sign (+). The “strong” group of conserved amino acid residues may be any one of the following groups of amino acids: STA, NEQK, NHQK, NDEQ, QHRK, MILV, MILF, HY, FYW.

Table 1H lists the domain description from DOMAIN analysis results against NOV1. This indicates that the NOV1 sequence has properties similar to those of other proteins known to contain this domain.

TABLE 1H
Domain Analysis of NOV1

IPR003007; MATH domain Score=138.8, Expect = 9.6e−38
Secreted forms of the alpha subunit of recombinant mouse meprin A
include an NH2-terminal prosequence, a catalytic domain, and three
COOH-terminal domains designated as MAM (meprin, A-5 protein, receptor
protein-tyrosine phosphatase mu), MATH (meprin and TRAF homology), and
AM (after MATH). The MAM domain may be necessary for correct folding
and transport through the secretory pathway, the MATH domain may be
required for folding of an activable zymogen, and the AM domain may be
important for activity against proteins and efficient secretion of the
protein [PUB00006426]

| MATH | | TIKNFSKIKEEAKEGREGEEYYTSPVEERFNIPWRLNVLRIYRNGGG | | (SEQ ID NO:37) |
|---|---|---|---|---|
| | | +++++ + \|+\|\|++\|++++++++\|++++\| ++ + \| \|+\|\|+\| | | |
| NOV1 | 303 | KVTDYKMKKREAVDGHTVSIFSQSFYTSRCGYRLC---ARAYLNGDG | 346 | (SEQ ID NO:38) |
| MATH | | EGRSGKFLGLYLHCLKEEKDSPTIENLKWSIETEFTLKLVSDNGKSIRRM | | |
| | | \|\| \| +\|+\|\|++++ +\|+\|\| \|+\|+++ \|\|+\|++++\|\|+ | | |
| NOV1 | 347 | SGR-GSHLSLYFVVMRGEFDS----LLQWPFRQRVTLMLLDQSGKK---- | 387 | |
| MATH | | SSTTLTKKTKDAKNNSHVFEKPTGEGWGKSGFKKFISWDDLEDDYNGYLV | | |
| | | ++++\|+\|++ +\|\| +\| +\|+\|\|++++\|\|+++\|+ ++ \|\|++ \|+\|++ | | |
| NOV1 | 388 | -NIMETFKPD---PNSSSFKRPDGEMNIASGCPRFVAHSVLENAKNAYIK | 433 | |
| MATH | | DDSIIIEAEVKI | | |
| | | \|\|+++ + \|+ | | |
| NOV1 | 434 | DDTLFLKVAVDL 445 | | |

The NOV1 nucleic acids encoding the TRAF5-like proteins of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below.

The disclosed NOV1a protein has multiple hydrophilic regions, each of which can be used as an immunogen. In one embodiment, a contemplated NOV1a epitope is from about amino acids 10 to 65. In another embodiment, a contemplated NOV1a epitope is from about amino acids 92 to 110. In other specific embodiments, contemplated NOV1a epitopes are from about amino acids 120 to 195, 218 to 245, 250 to 325, 330 to 350, 375 to 430 and 440 to 455.

The disclosed NOV1b protein has multiple hydrophilic regions, each of which can be used as an immunogen. In one embodiment, a contemplated NOV1b epitope is from about amino acids 10 to 70. In another embodiment, a contemplated NOV1b epitope is from about amino acids 82 to 135. In other specific embodiment, contemplated NOV1b epitopes are from about amino acids 146 to 177, 180 to 265, 270 to 287, 302 to 348, and 354 to 368.

NOV2

NOV2 includes three novel KIAA 1246-like proteins. The disclosed sequences have been named NOV2a, NOV2b and NOV2c.

NOV2a

The disclosed NOV2a nucleic acid of 3386 nucleotides (designated CuraGen Acc. No. JGIGC_ORDERED_CIT-HSPC_{—}246B18_{—}20000718_DA1 and also referred to as CG55265-01) (SEQ ID NO:5) encoding a novel human KIAA1246-like protein is shown in Table 2A.

TABLE 2A
NOV2a nucleotide sequence.

(SEQ ID NO:5)

CATGGCTCCAGGACCCTTCTCCTCGGCCCTCCTCTCGCCGCCGCCCGCTGCCCTGCCCTT
TCTGCTGCTGCTCTGGGCGGGGGCATCTCGTGGCCAGCCCTGCCCCGGCCGCTGCATCTG
CCAGAACGTGGCGCCCACACTGACAATGCTGTGCGCCAAGACCGGCTTGCTCTTTGTGCC
GCCCGCCATCGACCGGCGCGTGGTGGAGCTGCGGCTCACCGACAACTTCATCGCCGCCGT
GCGCCGCCGAGACTTCGCCAACATGACCAGCCTGGTGCACCTCACTCTCTCCCGGAACAC
CATCGGCCAGGTGGCAGCTGGCGCCTTCGCCGACCTGCGTGCCCTCCGGGCCCTGCACCT
GGACAGCAACCGCCTGGCGGAGGTGCGCGGCGACCAGCTCCGCGGCCTGGGCAACCTCCG
CCACCTGATCCTTGGAAACAACCAGATCCGCCGGGTGGAGTCGGCGGCCTTTGACGCCTT
CCTGTCCACCGTGGAGGACCTGGATCTGTCCTACAACAACCTGGAGGCCCTGCCGTGGGA
GGCGGTGGGCCAGATGGTGAACCTAAACACCCTCACGCTGGACCACAACCTCATCGACCA
CATCGCGGAGGGGACCTTCGTGCAGCTTCACAAGCTGGTCCGTCTGGACATGACCTCCAA
CCGCCTGCATAAACTCCCGCCCGACGGGCTCTTCCTGAGGTCGCAGGGCACCGGGCCCAA
GCCGCCCACCCCGCTGACCGTCAGCTTCGGCGGCAACCCCCTGCACTGCAACTGCGAGCT
GCTCTGGCTGCGGCGGCTGACCCGCGAGGACGACTTAGAGACCTGCGCCACGCCCGAACA
CCTCACCGACCGCTACTTCTGGTCCATCCCCGAGGAGGAGTTCCTGTGTGAGCCCCCGCT
GATCACACGGCAGGCGGGGGGCCGGGCCCTGGTGGTGGAAGGCCAGGCGGTGAGCCTGCG
CTGCCGAGCGGTGGGTGACCCCGAGCCGGTGGTGCACTGGGTGGCACCTGATGGGCGGCT
GCTGGGGAACTCCAGCCGGACCCGGGTCCGGGGGGACGGGACGCTGGATGTGACCATCAC
CACCTTGAGGGACAGTGGCACCTTCACTTGTATCGCCTCCAATGCTGCTGGGGAAGCGAC
GGCGCCCGTGGAGGTGTGCGTGGTACCTCTGCCTCTGATGGCACCCCCGCCGGCTGCCCC
GCCGCCTCTCACCGAGCCCGGCTCCTCTGACATCGCCACGCCGGGCAGACCAGGTGCCAA
CGATTCTGCGGCTGAGCGTCGGCTCGTGGCAGCCGAGCTCACCTCGAACTCCGTGCTCAT
CCGCTGGCCAGCCCAGAGGCCTGTGCCCGGAATACGCATGTACCAGGTTCAGTACAACAG
TTCCGTTGATGACTCCCTCGTCTACAGGATGATCCCGTCCACCAGTCAGACCTTCCTGGT
GAATGACCTGGCGGCGGGCCGTGCCTACGACTTGTGCGTGCTGGCGGTCTACGACGACGG
GGCCACAGCGCTGCCGGCAACGCGAGTGGTGGGCTGTGTACAGTTCACCACCGCTGGGGA
TCCGGCGCCCTGCCGCCCGCTGAGGGCCCATTTCTTGGGCGGCACCATGATCATCGCCAT
CGGGGGCGTCATCGTCGCCTCGGTCCTCGTCTTCATCGTTCTGCTCATGATCCGCTATAA
GGTGTATGGCGACGGGGACAGCCGCCGCGTCAAGGGCTCCAGGTCGCTCCCGCGGGTCAG
CCACGTGTGCTCGCAGACCAACGGCGCAGGCACAGGCGCGGCACAGGCCCCGGCCCTGCC
GGCCCAGGACCACTACGAGGCGCTGCGCGAGGTGGAGTCCCAGGCTGCCCCCGCCGTCGC
CGTCGAGGCCAAGGCCATGGAGGCCGAGACGGCATCCGCGGAGCCGGAGGTGGTCCTTGG
ACGTTCTCTGGGCGGCTCGGCCACCTCGCTGTGCCTGCTGCCATCCGAGGAAACTTCCGG
GGAGGAGTCTCGGGCCGCGGTGGGCCCTCGAAGGAGCCGATCCGGCGCCCTGGAGCCACC
AACCTCGGCGCCCCCTACTCTAGCTCTAGTTCCTGGGGGAGCCGCGGCCCGGCCGAGGCC
GCAGCAGCGCTATTCGTTCGACGGGGACTACGGGGCACTATTCCAGAGCCACAGTTACCC
GCGCCGCGCCCGGCGGACAAAGCGCCACCGGTCCACGCCGCACCTGGACGGGGCTGGAGG
GGGCGCGGCCGGGGAGGATGGAGACCTGGGGCTGGGCTCCGCCAGGGCGTGCCTGGCTTT
CACCAGCACCGAGTGGATGCTGGAGAGTACCGTGTGAGCGGCGGGCGGGCGCCGGGACGC
CTGGGTGCCGCAGACCAAACGCCCAGCCGCACGGACGCTGGGGCGGGACTGGGAGAAAGC
GCAGCGCCAAGACATTGGACCAGAGTGGAGACGCGCCCTTGTCCCCGGGAGGGGGCGGGG
CAGCCTCGGGCTGCGGCTCGAGGCCACGCCCCCGTGCCCAGGGCGGGGTTCGGGGACCGG
CTGCCGGCCTCCCTTCCCCTATGGACTCCTCGACCCCCCTCCTACCCCTCCCCTCGCGCG
CTCGCGGACCTCGCTGGAGCCGGTGCCTTACACAGCGAAGCGCGGGGAGGGGCAGGGCCC
CCTGACACTGCAGCACTGAGACACGAGCCCCCTCCCCCAGCCCGTCACCCGGGGCCGGGG
CGAGGGGCCCATTTCTTGTATCTGGCTGGACTAGATCCTATTCTGTCCCGCGGCGGCCTC
CAAAGCCTCCCACCCCACCCCACGCACATTCCTGGTCCGGTCGGGTCTGGCTTGGGGTCC
CCCTTTCTCTGTTTCCCTCGTTTGTCTCTATCCCGCCCTCTTGTCGTCTCTCTGTAGTGC
CTGTCTTTCCCTATTTGCCTCTCCTTTCTCTCTGTCCTGTCGTCTCTTGTCCCTCGGCCC
TCCCTGGTTTTGTCTAGTCTCCCTGTCTCTCCTGATTTCTTCTCTTTACTCATTCTCCCG
GGCAGGTCCCACTGGAAGGACCAGACTCTCCCAAATAAATCCCCACACGAACAAAATCCA
AAACCAAATCCCCCTCCCTACCGGAGCCGGGACCCTCCGCCGCAGCAGAATTAAACTTTT
TTCTGTGTCTGAGGCCCTGCTGACCTGTGTGTGTGTCTGTATGTGTGTCCGCGTGTAGTG
TGTGTGTGTGTGTGTGTGTGTGTGTGTGTGTGTGTGTGTGTGTTGGGGGAGGGTGACCTA
GATTGCAGCATAAGGACTCTAAGTGAGACTGAAGGAAGATGGGAAGATGACTAACTGGGG
CCGGAGGAGACTGGCAGACAGGCTTTTATCCTCTGAGAGACTTAGAGGTGGGGAATAATC
ACAAAAATAAAATGATCATAATAGCT

A NOV2a polypeptide (SEQ ID NO:6) encoded by SEQ ID NO:5 has 771 amino acid residues and is presented using the one-letter code in Table 2B.

TABLE 2B
Encoded NOV2a protein sequence.

(SEQ ID NO:6)

MAPGPFSSALLSPPPAALPFLLLLWAGASRGQPCPGRCICQNVAPTLTML
CAKTGLLFVPPAIDRRVVELRLTDNFIAAVRRRDFANMTSLVHLTLSRNT
IGQVAAGAFADLRALRALHLDSNRLAEVRGDQLRGLGNLRHLILGNNQIR
RVESAAFDAFLSTVEDLDLSYNNLEALPWEAVGQMVNLNTLTLDHNLIDH
IAEGTFVQLHKLVRLDMTSNRLHKLPPDGLFLRSQGTGPKPPTPLTVSFG
GNPLHCNCELLWLRRLTREDDLETCATPEHLTDRYFWSIPEEEFLCEPPL
ITRQAGGRALVVEGQAVSLRCRAVGDPEPVVHWVAPDGRLLGNSSRTRVR
GDGTLDVTITTLRDSGTFTCIASNAAGEATAPVEVCVVPLPLMAPPPAAP
PPLTEPGSSDIATPGRPGANDSAAERRLVAAELTSNSVLIRWPAQRPVPG
IRMYQVQYNSSVDDSLVYRMIPSTSQTFLVNDLAAGRAYDLCVLAVYDDG
ATALPATRVVGCVQFTTAGDPAPCRPLRAHFLGGTMIIAIGGVIVASVLV
FIVLLMIRYKVYGDGDSRRVKGSRSLPRVSHVCSQTNGAGTGAAQAPALP
AQDHYEALREVESQAAPAVAVEAKAMEAETASAEPEVVLGRSLGGSATSL
CLLPSEETSGEESRAAVGPRRSRSGALEPPTSAPPTLALVPGGAAARPRP
QQRYSFDGDYGALFQSHSYPRRARRTKRHRSTPHLDGAGGGAAGEDGDLG
LGSARACLAFTSTEWMLESTV

NOV2b

The DNA sequence and protein sequence for a novel Leucine rich repeat and fibronectin containing membrane protein-like gene were obtained by exon linking and extended by RACE and are reported here as NOV2b (also referred to as CuraGen Acc. No. CG55265-02). The NOV2b nucleic acid of 2451 nucleotides (SEQ ID NO:7) is shown in FIG. 2c. An open reading frame was identified beginning at nucleotides 52-54 and ending at nucleotides 2377-2379. The start (ATG) and stop (TGA) codons of the open reading frame are highlighted in bold type. Putative untranslated regions (underlined), if any, are found upstream from the initiation codon and downstream from the termination codon.

TABLE 2C
NOV2b nucleotide sequence.

(SEQ ID NO:7)

TCCTTCCCCGGCAGGTCTCACCTCCCACCCCTCCTGCCTTCCCACTGCACCATGGCTCCAGGACC
CTTCTCCTCGGCCCTCCTCTCGCCGCCGCCCGCTGCCCTGCCCTTTCTGCTGCTGCTCTGGGCGG
GGGCATCTCGTGGCCAGCCCTGCCCCGGCCGCTGCATCTGCCAGAACGTGGCGCCCACACTGACA
ATGCTGTGCGCCAAGACCGGCTTGCTCTTTGTGCCGCCCGCCATCGACCGGCGCGTGGTGGAGCT
GCGGCTCACCGACAACTTCATCGCCGCCGTGCGCCGCCGAGACTTCGCCAACATGACCAGCCTGG
TGCACCTCACTCTCTCCCGGAACACCATCGGCCAGGTGGCAGCTGGCGCCTTCGCCGACCTGCGT
GCCCTCCGGGCCCTGCACCTGGACAGCAACCGCCTGGCGGAGGTGCGCGGCGACCAGCTCCGCGG
CCTGGGCAACCTCCGCCACCTGATCCTTGGAAACAACCAGATCCGCCGGGTGGAGTCGGCGGCCT
TTGACGCCTTCCTGTCCACCGTGGAGGACCTGGATCTGTCCTACAACAACCTGGAGGCCCTGCCG
TGGGAGGCGGTGGGCCAGATGGTGAACCTAAACACCCTCACGCTGGACCACAACCTCATCGACCA
CATCGCGGAGGGGACCTTCGTGCAGCTTCACAAGCTGGTCCGTCTGGACATGACCTCCAACCGCC
TGCATAAACTCCCGCCCGACGGGCTCTTCCTGAGGTCGCAGGGCACCGGGCCCAAGCCGCCCACC
CCGCTGACCGTCAGCTTCGGCGGCAACCCCCTGCACTGCAACTGCGAGCTGCTCTGGCTGCGGCG
GCTGACCCGCGAGGACGACTTAGAGACCTGCGCCACGCCCGAACACCTCACCGACCGCTACTTCT
GGTCCATCCCCGAGGAGGAGTTCCTGTGTGAGCCCCCGCTGATCACACGCAGGCGGGGGGCCGGG
CCCTGGTGGTGGAAGGCCAGGCGGTGAGCCTGCGCTGCCGAGCGGTGGGTGACCCCGAGCCGGTG
GTGCACTGGGTGGCACCTGATGGGCGGCTGCTGGGGAACTCCAGCCGGACCCGGGTCCGGGGGGA
CGGGACGCTGGATGTGACCATCACCACCTTGAGGGACAGTGGCACCTTCACTTGTATCGCCTCCA
ATGCTGCTGGGGAAGCGACGGCGCCCGTGGAGGTGTGCGTGGTACCTCTGCCTCTGATGGCACCC
CCGCCGGCTGCCCCGCCGCCTCTCACCGAGCCCGGCTCCTCTGACATCGCCACGCCGGGCAGACC
AGGTGCCAACGATTCTGCGGCTGAGCGTCGGCTCGTGGCAGCCGAGCTCACCTCGAACTCCGTGC
TCATCCGCTGGCCAGCCCAGAGGCCTGTGCCCGGAATACGCATGTACCAGGTTCAGTACAACAGT
TCCGTTGATGACTCCCTCGTCTACAGGTGGGTGTACAGGATGATCCCGTCCACCAGTCAGACCTT
CCTGGTGAATGACCTGGCGGCGGGCCGTGCCTACGACTTGTCCGTGCTGGCGGTCTACGACGAC
GGGGCCACAGCGCTGCCGGCAACGCGAGTGGTGGGCTGTGTACAGTTCACCACCGCTGGGGATCC
GGCGCCCTGCCGCCCGCTGAGGGCCCATTTCTTGGGCGGCACCATGATCATCGCCATCGGGGGCG
TCATCGTCGCCTCGGTCCTCGTCTTCATCGTTCTGCTCATGATCCGCTATAAGGTGTATGGCGAC
GGGGACAGCCGCCGCGTCAAGGGCTCCAGGTCGCTCCCGCGGGTCAGCCACGTGTGCTCGCAGAC
CAACGGCGCAGGCACAGGCGCGGCACAGGCCCCGGCCCTGCCGGCCCAGGACCACTACGAGGCGC
TGCGCGAGGTGGAGTCCCAGGCTGCCCCCGCCGTCGCCGTCGAGGCCAAGGCCATGGAGGCCGAG
ACGGCATCCGCGGAGCCGGAGGTGGTCCTTGGACGTTCTCTGGGCGGCTCGGCCACCTCGCTGTG
CCTGCTGCCATCCGAGGAAACTTCCGGGGAGGAGTCTCGGGCCGCGGTGGGCCCTCGAAGGAGCC
GATCCGGCGCCCTGGAGCCACCAACCTCGGCGCCCCCTACTCTAGCTCTAGTTCCTGGGGGAGCC
GCGGCCCGGCCGAGGCCGCAGCAGCGCTATTCGTTCGACGQGGACTACGGGGCACTATTCCAGAG
CCACAGTTACCCGCGCCGCGCCCGGCGGACAAAGCGCCACCGGTCCACGCCGCACCTGGACGGGG
CTGGAGGGGGCGCGGCCGGGGAGGATGGAGACCTGGGGCTGGGCTCCGCCAGGGCGTGCCTGGCT
TTCACCAGCACCGAGTGGATGCTGGAGAGTACCGTGTGAGCGGCGGGCGGGCGCCGGGACGCCTG
GGTGCCGCAGACCAAACGCCCAGCCGCACGGACGCTGGGGCGGGAC

The sequence of NOV2b was derived by laboratory cloning of cDNA fragments, by in silico prediction of the sequence. cDNA fragments covering either the full length of the DNA sequence, or part of the sequence, or both, were cloned. In silico prediction was based on sequences available in Curagen's proprietary sequence databases or in the public human sequence databases, and provided either the full length DNA sequence, or some portion thereof. The DNA sequence and protein sequence for a kiaa 1246-like gene were obtained by exon linking and are reported here as NOV2. These primers and methods used to amplify NOV2 cDNA are described in the Examples.

The NOV2b encoded protein having 775 amino acid residues is presented using the one-letter code in Table 2D.

TABLE 2D
NOV2b polypeptide.

(SEQ ID NO:8)

MAPGPFSSALLSPPPAALPFLLLLWAGASRGQPCPGRCICQNVAPTLTML
CAKTGLLFVPPAIDRRVVELRLTDNFIAAVRRRDFANMTSLVHLTLSRNT
IGQVAAGAFADLRALRALHLDSNRLAEVRGDQLRGLGNLRHLILGNNQIR
RVESAAFDAFLSTVEDLDLSYNNLEALPWEAVGQMVNLNTLTLDHNLIDH
IAEGTFVQLHKLVRLDMTSNRLHKLPPDGLFLRSQGTGPKPPTPLTVSFG
GNPLHCNCELLWLRRLTREDDLETCATPEHLTDRYFWSIPEEEFLCEPPL
ITRQAGGRALVVEGQAVSLRCRAVGDPEPVVHWVAPDGRLLGNSSRTRVR
GDGTLDVTITTLRDSGTFTCIASNAAGEATAPVEVCVVPLPLMAPPPAAP
PPLTEPGSSDIATPGRPGANDSAAERRLVAAELTSNSVLIRWPAQRPVPG
IRMYQVQYNSSVDDSLVYRWVYRMIPSTSQTFLVNDLAAGRAYDLCVLAV
YDDGATALPATRVVGCVQFTTAGDPAPCRPLRAHFLGGTMIIAIGGVIVA
SVLVFIVLLMIRYKVYGDGDSRRVKGSRSLPRVSHVCSQTNGAGTGAAQA
PALPAQDHYEALREVESQAAPAVAVEAKAMEAETASAEPEVVLGRSLGGS
ATSLCLLPSEETSGEESRAAVGPRRSRSGALEPPTSAPPTLALVPGGAAA
RPRPQQRYSFDGDYGALFQSHSYPRRARRTKRHRSTPHLDGAGGGAAGED
GDLGLGSARACLAFTSTEWMLESTV

In a search of sequence databases, it was found, for example, that the nucleic acid sequence of NOV2 has 1742 of 2177 bases (80%) identical to a gb:GENBANK-ID:AB040917|acc:AB040917.1 mRNA from Homo sapiens (Homo sapiens mRNA for KIAA1484 protein, partial cds). The full amino acid sequence of the protein of NOV2 was found to have 460 of 468 amino acid residues (98%) identical to, and 462 of 468 amino acid residues (98%) similar to, the 492 amino acid residue ptnr:TREMBLNEW-ACC:AAH04018 protein from Mus musculus (Mouse) (UNKNOWN (PROTEIN FOR MGC:7599)).

The PSORT, SignalP and hydropathy profile for NOV2b predict that this sequence has a signal peptide and is likely to be localized at the plasma membrane with a certainty of 0.7000. In alternative embodiments, NOV2b is located to the endoplasmic reticulum (membrane) with a certainty of 0.2000 or the mitochondrial inner membrane with a certainty of 0.1000. The signal peptide is predicted by SignalP to be cleaved between amino acid 31 and 32: SRG-QP.

NOV2c

The NOV2c nucleic acid of 3568 nucleotides (also referred to as CG55265-03) (SEQ ID NO:9) is shown in FIG. 2E. An open reading frame was identified beginning at nucleotides 100-102 and ending at nucleotides 2497-2499. The start and stop codons of the open reading frame are highlighted in bold type. Putative untranslated regions (underlined), if any, are found upstream from the initiation codon and downstream from the termination codon.

TABLE 2E
NOV2c nucleotide sequence

(SEQ ID NO:9)

GTTGTTAATAGCTGATAGTGGCTTCCTCTCACCATCCTATGGAACCCAGCTGTAGGCGGT
AGGGGAGGGATCCAGGCCTGACTCGTAGCAGGACATCCCATGGCGGCCAAGTCCCACGCC
TGGGTAACCGGCTCCTTCCCCGGCAGGTCTCACCTCCCACCCCTCCTGCCTTCCCACTGC
ACCATGGCTCCAGGACCCTTCTCCTCGGCCCTCCTCTCGCCGCCGCCCGCTGCCCTGCCC
TTTCTGCTGCTGCTCTGGGCGGGGGCATCTCGTGGCCAGCCCTGCCCCGGCCGCTGCATC
TGCCAGAACGTGGCGCCCACACTGACAATGCTGTGCGCCAAGACCGGCTTGCTCTTTGTG
CCGCCCGCCATCGACCGGCGCGTGGTGGAGCTGCGGCTCACCGACAACTTCATCGCCGCC
GTGCGCCGCCGAGACTTCGCCAACATGACCAGCCTGGTGCACCTCACTCTCTCCCGGAAC
ACCATCGGCCAGGTGGCAGCTGGCGCCTTCGCCGACCTGCGTGCCCTCCGGGCCCTGCAC
CTGGACAGCAACCGCCTGGCGGAGGTGCGCGGCGACCAGCTCCGCGGCCTGGGCAACCTC
CGCCACCTGATCCTTGGAAACAACCAGATCCGCCGGGTGGAGTCGGCGGCCTTTGACGCC
TTCCTGTCCACCGTGGAGGACCTGGATCTGTCCTACAACAACCTGGAGGCCCTGCCGTGG
GAGGCGGTGGGCCAGATGGTGAACCTAAACACCCTCACGCTGGACCACAACCTCATCGAC
CACATCGCGGAGGGGACCTTCGTGCAGCTTCACAAGCTGGTCCGTCTGGACATGACCTCC
AACCGCCTGCATAAACTCCCGCCCGACGGGCTCTTCCTGAGGTCGCAGGGCACCGGGCCC
AAGCCGCCCACCCCGCTGACCGTCAGCTTCGGCGGCAACCCCCTGCACTGCAACTGCGAG
CTGCTCTGGCTGCGGCGGCTGACCCGCGAGGACGACTTAGAGACCTGCGCCACGCCCGAA
CACCTCACCGACCGCTACTTCTGGTCCATCCCCGAGGAGGAGTTCCTGTGTGAGCCCCCG
CTGATCACACGGCAGGCGGGGGGCCGGGCCCTGGTGGTGGAAGGCCAGGCGGTGAGCCTG
CGCTGCCGAGCGGTGGGTGACCCCGAGCCGGTGGTGCACTGGGTGGCACCTGATGGGCGG
CTGCTGGGGAACTCCAGCCGGACCCGGGTCCGGGGGGACGGGACGCTGGATGTGACCATC
ACCACCTTGAGGGACAGTGGCACCTTCACTTGTATCGCCTCCAATGCTGCTGGGGAAGCG
ACGGCGCCCGTGGAGGTGTGCGTGGTACCTCTGCCTCTGATGGCACCCCCGCCGGCTGCC
CCGCCGCCTCTCACCGAGCCCGGCTCCTCTGACATCGCCACGCCGGGCAGACCAGGTGCC
AACGATTCTGCCGCTGAGCGTCGGCTCGTGGCAGCCGAGCTCACCTCGAACTCCGTGCTC
ATCCGCTGGCCAGCCCAGAGGCCTGTGCCCGGAATACGCATGTACCAGGTTCAGTACAAC
AGTTCCGTTGATGACTCCCTCGTCTACAGGATGATCCCGTCCACCAGTCAGACCTTCCTG
GTGAATGACCTGGCGGCGGGCCGTGCCTACGACTTGTGCGTGCTGGCGGTCTACGACGAC
GGGGCCACAGCGCTGCCGGCAACGCGAGTGGTGGGCTGTGTACAGTTCACCACCGCTGGG
GATCCGGCGCCCTGCCGCCCGCTGAGGGCCCATTTCTTGGGCGGCACCATGATCATCGCC
ATCGGGGGCGTCATCGTCGCCTCGGTCCTCGTCTTCATCGTTCTGCTCATGATCCGCTAT
AAGGTGTATGGCGACCGGGACAGCCGCCGCGTCAAGGGCTCCAGGTCGCTCCCGCGGGTC
AGCCACGTGTCCTCCCAGACCAACGGCGCAGGCACAGGCGCGGCACAGGCCCCGGCCCTG
CCGGCCCAGGACCACTACGAGGCGCTGCGCGAGGTGGAGTCCCAGGCTGCCCCCGCCGTC
GCCGTCGAGGCCAAGGCCATGGAGGCCGAGACGGCATCCGCGGAGCCGGAGGTGGTCCTT
GGACGTTCTCTGGGCGGCTCGGCCACCTCGCTGTGCCTGCTGCCATCCGAGGAAACTTCC
GGGGAGGAGTCTCGGGCCGCGGTGGGCCCTCGAAGGAGCCGATCCGGCGCCCTGGAGCCA
CCAACCTCGGCGCCCCCTACTCTAGCTCTAGTTCCTGGGGGAGCCGCGGCCCGGCCGAGG
CCGCAGCAGCGCTATTCGTTCGACGGGGACTACGGGGCACTATTCCAGAGCCACAGTTAC
CCGCGCCGCGCCCGGCGGACAAAGCGCCACCGGTCCACGCCGCACCTGGACGGGGCTGGA
GGGGGCGCGGCCGGGGAGGATGGAGACCTGGGGCTGGGCTCCGCCAGGGCGTGCCTGGCT
TTCACCAGCACCGAGTGGATGCTGGAGAGTACCGTGTGAGCGGCGGGCGGGCGCCGGGAC
GCCTGGGTGCCGCAGACCAAACGCCCAGCCGCACGGACGCTGGGGCGGGACTGGGAGAAA
GCGCAGCGCCAAGACATTGGACCAGAGTGGAGACGCGCCCTTGTCCCCGGGAGGGGGCGG
GGCAGCCTCGGGCTGCGGCTCGAGGCCACGCCCCCGTGCCCAGGGCGGGGTTCGGGGACC
GGCTGCCGGCCTCCCTTCCCCTATGGACTCCTCGACCCCCCTCCTACCCCTCCCCTCGCG
CGCTCGCGGACCTCGCTGGAGCCGGTGCCTTACACAGCGAAGCGCGGGGAGGGGCAGGGC
CCCCTGACACTGCAGCACTGAGACACGAGCCCCCTCCCCCAGCCCGTCACCCGGGGCCGG
GGCGAGGGGCCCATTTCTTGTATCTGGCTGGACTAGATCCTATTCTGTCCCGCGGCGGCC
TCCAAAGCCTCCCACCCCACCCCACGCACATTCCTGGTCCGGTCGGGTCTGGCTTGGGGT
CCCCCTTTCTCTGTTTCCCTCGTTTGTCTCTATCCCGCCCTCTTGTCGTCTCTCTGTAGT
GCCTGTCTTTCCCTATTTGCCTCTCCTTTCTCTCTGTCCTGTCGTCTCTTGTCCCTCGGC
CCTCCCTGGTTTTGTCTAGTCTCCCTGTCTCTCCTGATTTCTTCTCTTTACTCATTCTCC
CGGGCAGGTCCCACTGGAAGGACCAGACTCTCCCAAATAAATCCCCACACGAACAAAATC
CAAAACCAAATCCCCCTCCCTACCGGAGCCGGGACCCTCCGCCGCAGCAGAATTAAACTT
TTTTCTGTGTCTGAGGCCCTGCTGACCTGTGTGTGTGTCTGTATGTGTGTCCGCGTGTAG
TGTGTGTGTGTGTGTGTGTGTGTGTGTGTGTGTGTGTGTGTGTGTTGGGGGAGGGTGACC
TAGATTGCAGCATAAGGACTCTAAGTGAGACTGAAGGAAGATGGGAAGATGACTAACTGG
GGCCGGAGGAGACTGGCAGACAGGCTTTTATCCTCTGAGAGACTTAGAGGTGGGGAATAA
TCACAAAAATAAAATGATCATAATAGCT

The NOV2c encoded protein having 799 amino acid residues (SEQ ID NO:10) is presented using the one-letter code in Table 2F.

TABLE 2F
NOV2c polypeptide

(SEQ ID NO:10)

MAAKSHAWVTGSFPGRSHLPPLLPSHCTMAPGPFSSALLSPPPAALPFLL
LLWAGASRGQPCPGRCICQNVAPTLTMLCAKTGLLFVPPAIDRRVVELRL
TDNFIAAVRRRDFANMTSLVHLTLSRNTIGQVAAGAFADLRALRALHLDS
NRLAEVRGDQLRGLGNLRHLILGNNQIRRVESAAFDAFLSTVEDLDLSYN
NLEALPWEAVGQMVNLNTLTLDHNLIDHIAEGTFVQLHKLVRLDMTSNRL
HKLPPDGLFLRSQGTGPKPPTPLTVSFGGNPLHCNCELLWLRRLTREDDL
ETCATPEHLTDRYFWSIPEEEFLCEPPLITRQAGGRALVVEGQAVSLRCR
AVGDPEPVVHWVAPDGRLLGNSSRTRVRGDGTLDVTITTLRDSGTFTCIA
SNAAGEATAPVEVCVVPLPLMAPPPAAPPPLTEPGSSDIATPGRPGANDS
AAERRLVAAELTSNSVLIRWPAQRPVPGIRMYQVQYNSSVDDSLVYRMIP
STSQTFLVNDLAAGRAYDLCVLAVYDDGATALPATRVVGCVQFTTAGDPA
PCRPLRAHFLGGTMIIAIGGVIVASVLVFIVLLMIRYKVYGDGDSRRVKG
SRSLPRVSHVCSQTNGAGTGAAQAPALPAQDHYEALREVESQAAPAVAVE
AKAMEAETASAEPEVVLGRSLGGSATSLCLLPSEETSGEESRAAVGPRRS
RSGALEPPTSAPPTLALVPGGAAARPRPQQRYSFDGDYGALFQSHSYPRR
ARRTKRHRSTPHLDGAGGGAAGEDGDLGLGSARACLAFTSTEWMLESTV

In a search of sequence databases, it was found, for example, that the nucleic acid sequence of NOV2c has 1353 of 1641 bases (82%) identical to a gb:GENBANK-ID:BC004018|acc:BC004018.1 mRNA from Mus musculus (Mus musculus, clone MGC:7599, mRNA, complete cds). The full amino acid sequence of the protein of NOV2c was found to have 700 of 700 amino acid residues (100%) identical to, and 700 of 700 amino acid residues (100%) similar to, the 700 amino acid residue ptnr:SPTREMBL-ACC:Q9P244 protein from Homo sapiens (Human) (KIAA1484 PROTEIN).

The PSORT, SignalP and hydropathy profile for NOV2c predict that this sequence has a signal peptide and is likely to be localized at the endoplasmic reticulum (membrane) with a certainty of 0.8500. In alternative embodiments, NOV2c is located to the plasma membrane with a certainty of 0.4400, the microbody (peroxisome) with a certainty of 0.3000, or the mitochondrial inner membrane with a certainty of 0.1000. The signal peptide is predicted by SignalP to be cleaved at amino acid 59-60: SRG-QP

Homologies to any of the above NOV2 proteins will be shared by the other NOV2 proteins insofar as they are homologous to each other. Any reference to NOV2 is assumed to refer to all three of the NOV2 proteins in general, unless otherwise noted.

The amino acid sequence of NOV2 has high homology to other proteins as shown in Table 2G.

TABLE 2G
BLASTX results for NOV2

| | | Smallest |
|---|---|---|
| | | Sum |
| | High | Prob |
| Sequences producing High-scoring Segment Pairs: | Score | P(N) |
| patp: AAB09968 Human brain-specific transmembrane | 1637 | 1.3e−173 |
| glycoprotein - Homo sapiens, 789 aa. | | |
| patp: AAB12448 Human hh00149 protein-Homo sapiens, 785 aa | 1637 | 1.3e−173 |
| patp: AAB84469 Amino acid sequence of an interferon | 1668 | 1.7e−171 |
| omega-1 like protein - Homo sapiens, 628 aa | | |
| patp: AAB70072 Human secreted protein #11 - Homo sapiens, 468 aa | 981 | 4.3e−102 |
| patp: AAB42689 Human ORFX ORF2453 polypeptide sequence | 408 | 3.7e−37 |
| Homo sapiens, 144 aa | | |

NOV2 also has homology to the amino acid sequences shown in the BLASTP data listed in Table 2H.

TABLE 2H
BLASTP results for NOV2

| Gene Index/ | Protein/ | Length | Identity | Positives | |
|---|---|---|---|---|---|
| Identifier | Organism | (aa) | (%) | (%) | Expect |
| ptnr: SPTREMBL- | KIAA1484 | 700 (frag- | 700/700 | 700/700 | 0.0 |
| ACC: Q9P244 | PROTEIN - | ment) | (100%) | (100%) | |
| | Homo sapiens | | | | |
| | (Human) | | | | |
| ptnr: SPTREMBL- | UNKNOWN | 492 | 460/468 | 462/468 | 7.7e−250 |
| ACC: Q99KT6 | (PROTEIN FOR | | (98%) | (98%) | |
| | MGC: 7599) - | | | | |
| | Mus musculus | | | | |
| | (Mouse) | | | | |
| ptnr: SPTREMBL- | KIAA1246 | 832 (frag- | 334/606 | 432/606 | 1.9e−173 |
| ACC: Q9ULH4 | PROTEIN - | ment) | (55%) | (71%) | |
| | Homo sapiens | | | | |
| | (Human) | | | | |
| ptnr: SPTREMBL- | HYPOTHETICAL | 789 | 334/606 | 431/606 | 1.9e−173 |
| ACC: Q9BE71 | 84.7 KDA | | (55%) | (71%) | |
| | PROTEIN - | | | | |
| | Macaca | | | | |
| | fascicularis (Crab | | | | |
| | eating macaque) | | | | |
| | (Cynomolgus | | | | |
| | monkey) | | | | |
| ptnr: SPTREMBL- | 5730420O05RIK | 788 | 341/617 | 432/617 | 8.1e−173 |
| ACC: Q9CYK3 | PROTEIN - Mus | | (55%) | (70%) | |
| | musculus | | | | |
| | (Mouse) | | | | |

The homology of the sequences shown in Table 2H as well as between the NOV2 variants is shown graphically in the ClustalW analysis shown in Table 2I.

[image]

[image]

[image]

[image]

The presence of identifiable domains in the protein disclosed herein was determined by searches versus domain databases such as Pfam, PROSITE, ProDom, Blocks or Prints and then identified by the Interpro domain accession number. Significant domains are summarized in Table 2J.

TABLE 2J
Domain Analysis of NOV2

| | | Score | Expect |
|---|---|---|---|
| IPR001611; LRR | Leucine Rich Repeat | 91.3 | 1.9e−23 |
| IPR000483; LRRCT | Leucine rich repeat C-terminal | 29.4 | 8.5e−05 |
| | domain | | |
| IPR003600; ig | Immunoglobulin domain | 34.4 | 4.1e−09 |
| IPR003961; fn3 | Fibronectin type III domain | 34.5 | 2.4e−06 |

Leucine-rich repeats (LRRs) are relatively short motifs (22-28 residues in length) found in a variety of cytoplasmic, membrane and extracellular proteins. Although these proteins are associated with widely different functions, a common property involves protein-protein interaction. Little is known about the 3D structure of LRRs, although it is believed that they can form amphipathic structures with hydrophobic surfaces capable of interacting with membranes. In vitro studies of a synthetic LRR from Drosophila Toll protein have indicated that the peptides form gels by adopting beta-sheet structures that form extended filaments. These results are consistent with the idea that LRRs mediate protein-protein interactions and cellular adhesion. Other functions of LRR-containing proteins include, for example, binding to enzymes and vascular repair. The 3-D structure of ribonuclease inhibitor, a protein containing 15 LRRs, has been determined, revealing LRRs to be a new class of alpha/beta fold. LRRs form elongated non-globular structures and are often flanked by cysteine rich domains.

| LRR: domain 1 of 7, from 66 to 89: score 5.1, E = 1.3e + 02 | | | | |
|---|---|---|---|---|
| | | NLEELDLSNNNLSGSLPPESFGNLP | | (SEQ ID NO:44) |
| | | + \|\| \| +\| + + \|+\|++ | | |
| NOV2 | 66 | RVVELRLTDNFIA-AVRRRDFANMT | 89 | (SEQ ID NO:45) |
| LRR: domain 2 of 7, from 90 to 113: score 15.7, E = 1.1 | | | | |
| | | NLEELDLSNNNLSGSLPPESFGNLP | | (SEQ ID NO:46) |
| | | +\| +\| \|\|+\| + ++ +++\|++\|+ | | |
| NOV2 | 90 | SLVHLTLSRNTIG-QVAAGAFADLR | 113 | (SEQ ID NO:47) |
| LER: domain 3 of 7, from 114 to 137: score 9.3, E = 45 | | | | |
| | | NLEELDLSNNNLSGSLPPESFGNLP | | (SEQ ID NO:48) |
| | | \|+ +\|++\|+\| ++ + +++\| | | |
| NOV2 | 114 | ALRALHLDSNRLA-EVRGDQLRGLG | 137 | (SEQ ID NO:49) |
| LRR: domain 4 of 7, from 138 to 161: score 15.5, E = 1.3 | | | | |
| | | NLEELDLSNNNLSGSLPPESFGNLP | | (SEQ ID NO:50) |
| | | \|\|++\| \| \|\|+++ +++ +\| | | |
| NOV2 | 138 | NLRHLILGNNQIR-RVESAAFDAFL | 161 | (SEQ ID NO:51) |
| LRR: domain 5 of 7, from 163 to 186: score 17.8, E = 0.25 | | | | |
| | | NLEELDLSNNNLSGSLPPESFGNLP | | (SEQ ID NO:52) |
| | | \| \|\|\|\|+\|\|\|+ \|\| \|+ \|++ | | |
| NOV2 | 163 | TVEDLDLSYNNLE-ALPWEAVGQMV | 186 | (SEQ ID NO:53) |
| LRR: domain 6 of 7, from 187 to 210: score 16.0, E = 0.9 | | | | |
| | | NLEELDLSNNNLSGSLPPESFGNLP | | (SEQ ID NO:54) |
| | | \|\| +\| \|++\| + ++ +++\| +\|+ | | |
| NOV2 | 187 | NLNTLTLDHNLID-HIAEGTFVQLH | 210 | (SEQ ID NO:55) |
| LRR: domain 7 of 7, from 211 to 233: score 11.5, E = 20 | | | | |
| | | NLEELDLSNNNLSGSLPPESFGNLP | | (SEQ ID NO:56) |
| | | +\| +\|\|+ +\|+\| +\|\|\|+ \|+ | | |
| NOV2 | 211 | KLVRLDMTSNRLH-KLPPD-GLFLR | 233 | (SEQ ID NO:57) |

Leucine Rich Repeats are short sequence motifs present in a number of proteins with diverse functions and cellular locations. Leucine Rich Repeats are often flanked by cysteine rich domains. This domain is often found at the C-terminus of tandem leucine rich repeats.

CD molecules are leucocyte antigens on cell surfaces. CD antigens nomenclature is updated at http://www.ncbi.nlm.nih.gov/prow/cd/index_molecule.htm

Some platelet glycoproteins belong to this group. The CD42a-d-complex serves as receptor for von Willebrand factor (vWf) and thrombin. The actual binding site for vWf and thrombin lies on CD42b (GPIb alpha). The complex mediates adhesion of platelets to subendothelial matrices (exposed upon damage to the endothelium) at high shear rates and amplifies the platelet response to thrombin during platelet activation where thrombin is involved. CD42a is the platelet glycoprotein IX (GPIX), CD42b is the platelet glycoprotein Ib alpha chain (GPIba) also called GPIbalpha or glycocalicin, CD42c is the platelet glycoprotein Ib beta chain (GPIBb or GPIb-beta) and CD42d is the platelet glycoprotein V (GPV). These proteins contain the leucine-rich repeat and the two cysteine-rich flanking regions.

| LRRCT: domain 1 of 1, from 252 to 297: score 29.4, E = 8.5e−0 | | | | |
|---|---|---|---|---|
| | | NPFNCDCELRWLLRWLRETNPRRLEDQEDLRCASPESLRGQPLLELL | | (SEQ ID NO:58) |
| | | \|\|++\|+\|\|\|+\|\|+\|+ \|\| \|\| +\|\| \|\|+\| ++ ++++ | | |
| NOV2 | 252 | NPLHCNCELLWLRRLTRE---DDLE-----TCATPEHLTDRYFWSIP | 290 | (SEQ ID NO:59) |
| | | PSDFSCP | | |
| | | ++\| \|+ | | |
| NOV2 | 291 | EEEFLCE | 297 | |

The basic structure of immunoglobulin (Ig) molecules is a tetramer of two light chains and two heavy chains linked by disulfide bonds. There are two types of light chains: kappa and lambda, each composed of a constant domain (CL) and a variable domain (VL). There are five types of heavy chains: alpha, delta, epsilon, gamma and mu, all consisting of a variable domain (VH) and three (in alpha, delta and gamma) or four (in epsilon and mu) constant domains (CH1 to CH4). Members of the immunoglobulin superfamily are found in hundreds of proteins of different functions. Examples include antibodies, the giant muscle kinase titin and receptor tyrosine kinases. Immunoglobulin-like domains may be involved in protein-protein and protein-ligand interactions. This entry includes IG domains that cannot be classified into one of IGv1, IGc1, IGc2 and IG.

| ig: domain 1 of 1, from 314 to 372: score 34.4, E = 4.1e-09 | | | | |
|---|---|---|---|---|
| | | GESVTLTCSVSGFGPP.P.VTWLRNGK..............LSLTI. | | (SEQ ID NO:60) |
| | | \|+ \|+\|+\|++ \|+\|+\| \|+\| ++++ +++++++ +++ +\| ++ | | |
| NOV2 | 314 | GQAVSLRCRAV--GDPEPVVHWVAPDGRLLGNSSRTRVRGDGTLDVT | 358 | (SEQ ID NO:61) |
| | | SVTPEDSGGTYTCVV | | |
| | | +\| \|\| \|\|+\|\| + | | |
| NOV2 | 359 | ITTLRDS-GTFTCIA | 372 | |

Fibronectins are multi-domain glycoproteins found in a soluble form in plasma, and in an insoluble form in loose connective tissue and basement membranes. They contain multiple copies of 3 repeat regions (types I, II and III), which bind to a variety of substances including heparin, collagen, DNA, actin, fibrin and fibronectin receptors on cell surfaces. The wide variety of these substances means that fibronectins are involved in a number of important functions: e.g., wound healing; cell adhesion; blood coagulation; cell differentiation and migration; maintenance of the cellular cytoskeleton; and tumour metastasis. The role of fibronectin in cell differentiation is demonstrated by the marked reduction in the expression of its gene when neoplastic transformation occurs. Cell attachment has been found to be mediated by the binding of the tetrapeptide RGDS to integrins on the cell surface, although related sequences can also display cell adhesion activity.

Plasma fibronectin occurs as a dimer of 2 different subunits, linked together by 2 disulphide bonds near the C-terminus. The difference in the 2 chains occurs in the type III repeat region and is caused by alternative splicing of the mRNA from one gene. The observation that, in a given protein, an individual repeat of one of the 3 types (e.g., the first FnIII repeat) shows much less similarity to its subsequent tandem repeats within that protein than to its equivalent repeat between fibronectins from other species, has suggested that the repeating structure of fibronectin arose at an early stage of evolution. It also seems to suggest that the structure is subject to high selective pressure.

The fibronectin type III repeat region is an approximately 100 amino acid domain, different tandem repeats of which contain binding sites for DNA, heparin and the cell surface. The superfamily of sequences believed to contain FnIII repeats represents 45 different families, the majority of which are involved in cell surface binding in some manner, or are receptor protein tyrosine kinases, or cytokine receptors.

| fn3: domain 1 of 1, from 422 to 502: score 34.5, E = 2.4e−06 | | | | |
|---|---|---|---|---|
| | | PSAPTNLTVTDVTSTSLTLSWSPPTGNGPITGYEVTYRQPKNGGEWN | | (SEQ ID NO:62) |
| | | \| ++\|++ ++\|\|+\|+ ++\| + ++ \| \|+\|+\| ++ + + + | | |
| NOV2 | 422 | SAAERRLVAAELTSNSVLIRWPAQRPVPGIRMYQVQY-NSSVDDSLV | 467 | (SEQ ID NO:63) |
| | | ELTVPGTTTSYTLTGLKPGTEYEVRVQAVNGGGGPES | | |
| | | +++\|+\|+ ++ +++\| +\|+ \| ++\| \|\| ++ ++ | | |
| NOV2 | 468 | YRMIPSTSQTFLVNDLAAGRAYDLCVLAVYDD--GAT | 502 | |

The NOV2 nucleic acids encoding the KIAA 1246-like proteins of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below.

The disclosed NOV2b protein has multiple hydrophilic regions, each of which can be used as an immunogen. In one embodiment, a contemplated NOV2b epitope is from about amino acids 42 to 50. In another embodiment, a contemplated NOV2b epitope is from about amino acids 70 to 90. In other specific embodiments, contemplated NOV2b epitopes are from about amino acids 110 to 160, 172 to 180, 210 to 250, 255 to 300, 325 to 360, 400 to 427, 450 to 480, 522 to 530, 560 to 590, 598 to 622, 668 to 685, and 695 to 750.

The disclosed NOV2c protein has multiple hydrophilic regions, each of which can be used as an immunogen. In one embodiment, a contemplated NOV2c epitope is from about amino acids 8 to 26. In another embodiment, a contemplated NOV2c epitope is from about amino acids 67 to 74. In other specific embodiment, contemplated NOV2c epitopes are from about amino acids 100 to 120, 150 to 185, 198 to 205, 242 to 275, 280 to 330, 355 to 385, 425 to 454, 470 to 498, 550 to 558, 580 to 650, 680 to 710, and 727 to 780.

NOV3

A disclosed NOV3 nucleic acid of 2660 nucleotides (also referred to as SGGC_DRAFT_BA342E24_{—}20000805_DA1) encoding a novel human DEMATIN-like protein is shown in Table 3A. An open reading frame was identified beginning with a ATG initiation codon at nucleotides 451-453 and ending with a CTG codon at nucleotides 1599-1661. Putative untranslated regions upstream from the initiation codon and downstream from the termination codon are underlined in Table 3A. The start and stop codons are in bold letters.

TABLE 3A
NOV3 Nucleotide Sequence

(SEQ ID NO:11)

CACGAGAAGACAGGAGGAAGAAAGGGAGAGAGGGCCAGGCAGTCGCACTGTGAACAGAACAGGAGAAGGCG
AAGCGGGGCAAAGTTCCCTGCCCACCGACGCCAGCCTGCTTGGATGACTTGCCTCGTTTCATAATTCACTT
ACTGTCTGCACCAGCCGGCCTCAGCCTGGCTGGACCCTGCTGCCTGTGTGGCCCGGAGCCAGAGGCCCCCA
CACTCCCAGCTGCTCTTCTACAGATGCCATCAACGAGCAGGACTCTGGGTGGCTCCACTGTCTAAGCCTGG
AGAGTCACCGCCCAGGGATGAGGACGCGCCAGCCCGGGGGAACGCGCCAGCTGCTTTCGCGGCCCCAAGCG
CGCAGTGCCCAGCAGCCGCGCCGAGCCTGACACGCTGTCCTCTCCCCTCGCGCACAGGGCTCTGCGAGTGA
CCCGGCGGGCGAGCTCCGTGCTGCATGGAACGGCTGCAGAAGCAACCACTTACCTCCCCCGGGAGCGTGAG
CCCCTCCCGAGATTCCAGTGTGCCTGGCTCTCCCTCCAGCATCGTGGCCAAGATGGACAATCAGGTGCTGG
GCTACAAGGACCTGGCTGCCATCCCCAAGGACAAGGCCATCCTGGACATCGAGCGGCCCGACCTCATGATC
TACGAGCCTCACTTCACTTATTCCCTCCTGGAACACGTGGAGCTGCCTCGCAGCCGCGAGCGCTCGCTGTC
ACCCAAATCCACATCCCCCCCACCATCCCCAGAGGTGTGGGCGGACAGCCGGTCGCCTGGAATCATCTCTC
AGGCCTCGGCCCCCAGAACCACTGGAACCCCCCGGACCAGACTGCCCCATTTCCACCACCCTGAGACCTCC
CGCCCAGATTCCAACATCTACAACAAGCCTCCCATCTATAAGCAGAGAGAGTCCGTGGGAGGCAGCCCTCA
GACCAAGCACCTCATCGAGGATCTCATCATCGAGTCATCCAAGTTTCCTGCAGCCCAGCCCCCAGACCCCA
ACCAGCCAGCCAAAATCGAAACCGACTACTGGCCATGCCCCCCGTCTCTGGCTGTTGTGGAGACAGAATGG
ACGAAGCGGAAGGCGTCTCGGAGGGGAGCAGAGGAAGAGGAGGAGGAGGAAGATGACGACTCTGGAGAGGA
GATGAAGGCTCTCAGGGAGCGTCAGAGAGAGGAACTCAGTAAGGTTACTTCCAACTTGGGAAAGATGATCT
TGAAAGAAGAGATGGAAAAGTCATTGCCGATCCGAAGGAAAACCCGCTCTCTGCCTGACCGGACACCCTTC
CATACCTCCTTGCACCAGGGAACGTCTAAATCTTCCTCTCTCCCCGCCTATGGCAGGACCACCCTGAGCCG
GCTACAGTCCACAGAGTTCAGCCCATCAGGGAGTGAGACTGGAAGCCCAGGCCTGCAGATCTATCCCTATC
AAATGCTAGTGGTGACCAACAAGGGGCGAACCAAGCTGCCACCGGGGGTGGATCGGATGCGGCTTCAGAGC
CATCTGTCTGCCGAGGACTTCTCAAGGGTATTTGCCATGTCCCCTGAAGAGTTTGGCAAGCTGGCTCTGTG
GAAGCGGAATGAGCTCAAGAAGAAGGCCTCTCTCTTCTGATGGCCCCCACCTGCTCCGGGACGGCCCCCTT
ACCCCTGCTGCTTCAGGGTTTTTCCCCGGCGGGTTGGGAGGGGCACGAGGTGGGGTGGAAATAGGGTGGGC
TCCTTTCCTCAGGTAGAGTGGGGGGCCAAAACCTCTGCAGTCCCCCGCAGTGAGCTATGGACTTTCTTCCC
CCTCACGAGGCTGGGGGCCTCCTGCTCTCGTCCCTGGCCCTCCCTGTACAGGGCAAAGCCAGTCTGGGCTC
TGGCACACAGAGTTCATGTTTGCCGCCCTCTCCCTGCCCCTCACCCCAGAGGTGAGAGGAATGAGGGGCAT
TGGTGGTTAGGCCGGTTGGCTGTCTTGAACAGCTGGAGGGAAGATGCAGGGGTGGGAAGCGGCCAGGCAGA
AAGAGCTCCAGGCTCTTGTGTCGCCCACCCAGCCCTCCCATACTCACTCCTGACAGCTTTCCTGCACTGCA
GCTTCCTGCTCCTCTGACTCTAGTGGGAACAGGCCCCAGCTCAGCCTCCGCGAGGGAGGTCACCCCTCCAC
TTCAGCTTGCCCTGACCTCCGCTCGCAAACCCCGAGCTTCCAAGCCTTTTGCTCCAGCCCTGCGGCTTCCC
CAGAAGCCTGGGCTTAGGGTGGAGATGCCGCCTACCCGATCCTGGCCCTCCACCTGCCTCCAGGCCACGAA
ATGGGAATTCCAGCACTAAGCCAGGCACCGGGCAGAAGCTGGGCCTTCCGCCTCCCTTGGATGGGCTCAAG
AGGCCAGGCCTGGCACATTTTGGAGTGTCCTGGCTACCAGCTCTCACACCTACACCCACGCACCCCCTCAC
ACACTATGCTCTCTCAAGAATGTAATTTATTGGGGCCCCCCCAGCTGCTTTCCTCACCTGCCCCTGCCCTA
CCTTACACCCCCAGCTTGACTTCTTTCCAGTCCACGTGGATATAATGATATCTATATTTTTGCCCAGGTCT
GGGTATTGCTCCTGCCCAGACCCTGACATCCCTTTCCACTGTGTGTGTGACCATGCTGGGGGAGGGGGACT
CTGCTTCGAATTAAAAGGTTGCTTTGGGTCCCT

The disclosed NOV3 nucleic acid sequence has 1504 of 1516 bases (99%) identical to human dematin (GENBANK-ID: L19713) (E=0).

A disclosed NOV3 protein (SEQ ID NO:12) encoded by SEQ ID NO:11 has 383 amino acid residues, and is presented using the one-letter code in Table 3B. Signal P, Psort and/or Hydropathy results predict that NOV3 contains a signal peptide, and is likely to be localized to the microbody (peroxisome) with a certainty of 0.3000. In other embodiments, it is likely to be localized to nucleus with a certainty of 0.3000 and to the mitochondrial matrix space with a certainty of 0.1000.

TABLE 3B
Encoded NOV3 protein sequence.

(SEQ ID NO:12)

MERLQKQPLTSPGSVSPSRDSSVPGSPSSIVAKMDNQVLGYKDLAAIPKDKAILDIERPDLMIYEPHFTYS
LLEHVELPRSRERSLSPKSTSPPPSPEVWADSRSPGIISQASAPRTTGTPRTRLPHFHHPETSRPDSNIYK
KPPIYKQRESVGGSPQTKHLIEDLIIESSKFPAAQPPDPNQPAKIETDYWPCPPSLAVVETEWRKRKASRR
GAEEEEEEEDDDSGEEMKALRERQREELSKVTSNLGKMILKEEMEKSLPIRRKTRSLPDRTPFHTSLHQGT
SKSSSLPAYGRTTLSRLQSTEFSPSGSETGSPGLQIYPYEMLVVTNKGRTKLPPGVDRMRLERHLSAEDFS
RVFAMSPEEFGKLALWKRNELKKKASLF

The amino acid sequence of NOV3 had high homology to other proteins as shown in Table 3C.

TABLE 3C
BLASTX results from PatP database for NOV3

patp: AAG03676 Human secreted protein
Score = 305 (107.4 bits), Expect = 4.0e−25, P = 4.0e−25
IDENTITIES = 59/61 (96%), POSITIVES = 59/61 (96%), FRAME = +1

In addition, the NOV3 amino acid sequence has 382 of 405 amino acid residues (94%) identical to, and 382 of 405 amino acid residues (94%) similar to, the human erythrocyte membrane protein band 4.9 (dematin) (GENBANK ID: AAH06318) (E=1.0e⁻¹⁶⁸). The global sequence homology is 62.396% amino acid homology and 54.576% amino acid identity.

NOV3 also has homology to the amino acid sequences shown in the BLASTP data listed in Table 3D.

TABLE 3D
BLAST results for NOV3

| Gene Index/ | Protein/ | Length | | | |
|---|---|---|---|---|---|
| Identifier | Organism | (aa) | Identity (%) | Positives (%) | Expect |
| gi\|13623437\|gb\| | erythrocyte | 405 | 382/405 | 382/405 | 1e−168 |
| AAH06318.1\|AAH06318 | membrane | | (94%), | (94%) | |
| | protein | | | | |
| | band 4.9 | | | | |
| | (dematin) | | | | |
| | [Homo | | | | |
| | sapiens] | | | | |
| gi\|14751945\|ref\| | erythrocyte | 383 | 380/383 | 380/383 | 1e−168 |
| XP_048027.1\| | membrane | | (99%), | (99%) | |
| | protein | | | | |
| | band 4.9 | | | | |
| | (dematin) | | | | |
| | [Homo | | | | |
| | sapiens] | | | | |
| gi\|585044\|sp\| | DEMA_HUMAN | 383 | 378/383 | 379/383 | 1e−168 |
| Q08495\| | DEMATIN | | (98%), | (98%) | |
| | (ERYTHROCYTE | | | | |
| | MEMBRANE | | | | |
| | PROTEIN | | | | |
| | BAND 4.9) | | | | |
| gi\|4503581\|ref\| | Erythrocyte | 405 | 380/405 | 380/405 | 1e−167 |
| NP_001969.1\| | membrane | | (93%), | (93%) | |
| | protein | | | | |
| | band 49 | | | | |
| | (dematin) | | | | |
| | [Homo | | | | |
| | sapiens] | | | | |
| gi\|14751939\|ref\| | erythrocyte | 405 | 380/405 | 380/405 | 1e−166 |
| XP_048026.1\| | membrane | | (93%), | (93%) | |
| | protein | | | | |
| | band 4.9 | | | | |
| | (dematin) | | | | |
| | [Homo | | | | |
| | sapiens] | | | | |

The homology of these sequences is shown graphically in the ClustalW analysis shown in Table 3E.

[image]

[image]

Tables 3F-3G list the domain description from DOMAIN analysis results against NOV3. This indicates that the NOV3 sequence likely has properties similar to those of other proteins known to contain these domains.

TABLE 3F
Domain Analysis of NOV3

gnh|Smart|smart00153, VHP, Villin headpiece domain (SEQ ID NO:69)
Length = 36 residues, 100.0% aligned
Score = 58.5 bits (140), Expect = 7e−10

| | | | | |
|---|---|---|---|---|
| NOV3: | 348 | HLSAEDFSRVFAMSPSEEFGKLALWKRNELKKKASLF | 383 | (SEQ ID NO:70) |
| | | +\|\| \|\|\| \|\| \|+ \|\|\| \|\| \|\|\|+\|+\|\|\|\| \|\| | | |
| smart00153: | 1 | YLSDEDFEEVFGMTKEEFYKLPLWKQNQLKKKLGLF | 36 | |

TABLE 3F
Domain Analysis of NOV3

gnl|Pfam|pfam02209, VHP, Villin headpiece domain (SEQ ID NO:71)
Length = 36 residues, 100.0% aligned
Score = 57.4 bits (137), Expect = 1e−09

| | | | | |
|---|---|---|---|---|
| NOV3: | 348 | HLSAEDFSRVFAMSPEEFGKLALWKRNELKKKASLF | 383 | (SEQ ID NO:72) |
| | | +\|\| \|\|\| \|\| \|+ \|\|\| \|\| \|\|+\|+\|\|\|\| \|\| | | |
| pfam02209: | 1 | YLSDEDFEEVFGMTKEEFYKLPAWKQNQLKKKLSLF | 36 | |

As shown in the domain analyses above, a disclosed NOV3 polypeptide is a member of the erythroid actin-bundling protein family. One of the conserved regions found in this protein family is a villin-like headpiece domain. This headpiece domain is essential for actin bundling and actin modulating activity.

NOV3 is expressed in at least the following tissues: Brain, anaplastic oligodendroglioma, and Colon. In addition, the NOV3 sequence is predicted to be expressed in the Liver because of the expression pattern of a closely related Papio insulin-like growth factor binding protein-3 complex acid-labile subunit homolog (GENBANK-ID: S83462).

The expression pattern, and protein similarity information for the invention suggest that the novel human DEMATIN protein described in this invention may be useful in potential therapeutic applications implicated in various diseases and disorders. The homology to antigenic secreted and membrane proteins suggests that antibodies directed against the novel genes may be useful in treatment and prevention of certain diseases and disorders.

NOV4

NOV4 includes two novel matrilin-2-like proteins disclosed below. The disclosed sequences have been named NOV4a and NOV4b.

NOV4a

The disclosed NOV4a nucleic acid of 3447 nucleotides (also designated CuraGen Acc. No. 14578444_{—}0_{—}47) encoding a novel matrilin-2-like protein is shown in Table 4A. An open reading frame was identified beginning with an ATG initiation codon at nucleotides 64-66 and ending with a TGA codon at nucleotides 2932-2934. Putative untranslated regions upstream from the initiation codon and downstream from the termination codon are underlined in Table 4A. The start and stop codons are in bold letters.

TABLE 4A
NOV4a nucleotide sequence.

(SEQ ID NO:13)

GGTAGCCGACGCGCCGGCCGGCGCGTGACCTTGCCCCTCTTGCTCGCCTTGAAAATGGAAAAGATGCTCG
CAGGCTGCTTTCTGCTGATCCTCGGACAGATCGTCCTCCTCCCTGCCGAGGCCAGGCAGCGGTCACGTGG
GAGGTCCATCTCTAGGGGCAGACACGCTCGGACCCACCCGCAGACGGCCCTTCTGGAGAGTTCCTGTGAG
AACAAGCGGCCAGACCTGGTTTTCATCATTGACAGCTCTCGCAGTGTCAACACCCATGACTATGCAAAGG
TCAAGGAGTTCATCGTGGACATCTTGCAATTCTTGGACATTGGTCCTGATGTCACCCGAGTGGGCCTGCT
CCAATATGGCAGCACTGTCAAGAATGAGTTCTCCCTCAAGACCTTCAAGAGGAAGTCCGACGTGGAGCGT
GCTGTCAAGAGGATGCGGCATCTGTCCACGGGCACCATGACTGGGCTGGCCATCCAGTATGCCCTGAACA
TCGCATTCTCAGAAGCAGAGGGGGCCCGGCCCCTGAGGGAGAATGTGCCACGGGTCATAATGATCGTGAC
AGATGGGAGACCTCAGGACTCCGTGGCCGAGGTGGCTGCTAAGGCACGGGACACGGGCATCCTAATCTTT
GCCATTGGTGTGGGCCAGGTAGACTTCAACACCTTGAAGTCCATTGGGAGTGAGCCCCATGAGGACCATG
TCTTCCTTGTGGCCAATTTCAGCCAGATTGAGACGCTGACCTCCGTCTTCCAGAAGAAGTTGTGCACGGC
CCACATGTGCAGCACCCTGGAGCATAACTGTGCCCACTTCTGCATCAACATCCCTGGCTCATACGTCTGC
AGGTGCAAACAAGGCTACATTCTCAACTCGGATCAGACGACTTGCAGAATCCAGGATCTGTGTGCCATGG
AGGACCACAACTGTGAGCAGCTCTGTGTGAATGTGCCGGGCTCCTTCGTCTGCCAGTGCTACAGTGGCTA
CGCCCTGGCTGAGGATGGGAAGAGGTGTGTGGCTGTGGACTACTGTGCCTCAGAAAACCACGGATGTGAA
CATGAGTGTGTAAATGCTGATGGCTCCTACCTTTGCCAGTGCCATGAAGGATTTGCTCTTAACCCAGATG
AAAAAACGTGCACAAAGATAGACTACTGTGCCTCATCTAATCATGGATGTCAGTACGAGTGTGTTAACAC
AGATGATTCCTATTCCTGCCACTGCCTGAAAGGCTTTACCCTGAATCCAGATAAGAAAACCTGCAGAAGG
ATCAACTACTGTGCACTGAACAAACCGGGCTGTGAGCATGAGTGCGTCAACATGGAGGAGAGCTACTACT
GCCGCTGCCACCGTGGCTACACTCTGCACCCCAATGGCAAACCCTGCAGCCGAGTGGACCACTGTGCACA
GCAGGACCATGGCTGTGAGCAGCTGTGTCTGAACACGGAGGATTCCTTCGTCTGCCAGTGCTCAGAAGGC
TTCCTCATCAACGAGGACCTCAAGACCTGCTCCCGGGTGGATTACTGCCTCCTGAGTGACCATGGTTGTG
AATACTCCTGTGTCAACATGGACAGATCCTTTGCCTGTCACTGTCCTGAGGGACACGTGCTCCGCAGCGA
TGGGAAGACGTGTGCAAAATTGGACTCTTGTGCTCTGGGGGACCACGCTTGTGAACATTCGTGTGTAAGC
AGTGAAGATTCGTTTGTGTGCCAGTGCTTTGAAGGTTATATACTCCGTGAAGATGGAAAAACCTGCACAA
GCAAAGATGTCTCCCAAGCTATAGACCATGGCTGTGAACACATTTGTCTCAACACTGACGACTCATACAC
GTGCGAGTGCTTGGAGGGATTCCGGCTCACTGAGGATGGGAAACGCTGCCGAATTTCCTCAGGGAAGGAT
GTCTGCAAATCAACCCACCATGGCTGCGAACACATTTGTGTTAATAATGGGAATTCCTACATCTGCAAAT
GCTCAGAGGGATTTGTTCTAGCTGAGGACGGAAGACGGTGCAAGAAATGCACTGAAGGCCCAATTGACCT
CGTCTTTGTGATCGATGGATCCAAGAGTCTTGGAGAAGAGAATTTTGAGGTCGTGAAGCAGTTTGTCACT
GGAATTATAGATTCCTTGACAATTTCCCCCAAACCCGCTCGAGTGGGGCTGCTCCAGTATTCCACACAGG
TCCACACAGAGTTCACTCTGAGAAACTTCAACTCAGCCAAAGACATGAAAAAAGCCGTCCCCCACATGAA
ATACATGGGAAAGGGCTCTATGACTGGGCTGGCCCTGAAACACATGTTTGAGAGAAGTTTTACCCAAGGA
GAAGGCCCCAGGCCCTTTTCCACAAGGGTGCCCAGAGCAGCCATTCTGTTCACCGACGGACCCGCTCAGG
ATGACGTCTCCGAGTGGGCCAGTAAAGCCAAGGCCAATGGTATCACTATGTATGCTGTTGGGGTAGGAAA
AGCCATTGAGGAGGAACTACAAGACATTGCCTCTGAGCCCACAAACAAGCATCTCTTCTATGCCGAAGAC
TTCAGCACAATGGATGAGATAAGTGAAAAACTCAAGAAAGGCATCTGTGAAGCTCTAGAAGACTCCGATG
GAACACAGGACTCTCCAGCAGGGGAACTCCCAAAAACGGTCCAACAGCCAACAGAATCTGAGCCAGTCAC
CATAAATATCCAAGACCTACTTTCCTGTTCTAATTTTGCAGTGCAACACAGATATCTGTTTGAAGAAGAC
AATCTTTTACGGTCTACACAAAAGCTTTCCCATTCAACAAAACCTTCAGGAAGCCCTTTGGAAGAAAAAC
ACGATCAATGCAAATGTGAAAACCTTATAATGTTCCAGAACCTTGCAAACGAAGAAGTAAGAAAATTAAC
ACAGCGCTTAGAAGAAATGACACAGAGAATGGAAGCCCTGGAAAATCGCCTCAGATACAGATGAAGATTA
GAAATCGCGACACATTTGTAGTCATTGTATCACCGATTACAATGAACGCAGTGCAGAGCCCCAAAGCTCA
GGCTATTGTTAAATCAATAATGTTGTGAAGTAAAACAATCAGTACTGAGAAACCTGGTTTGCCACAGAAC
AAAGACAAGAAGTATACACTAACTTGTATAAATTTATCTAGGAAAAAAATCCTTCAGAATTCTAAGATGA
ATTTACCAGGTGAGAATGAATAAGCTATGCAAGGTATTTTGTAATATACTGTGGACACAACTTGCTTCTG
CCTCATCCTGCCTTAGTGTGCAATCTCATTTGACTATACGATAAAGTTTGCACAGTCTTACTTCTGTAGA
ACACTGGCCATAGGAAATGCTGTTTTTTTGTACTGGACTTTACCTTGATATATGTATATGGATGTATGCA
TAAAATCATAGGACATATGTACTTGTGGAACAAGTTGGATTTTTTATACAATATTAAAATTCACCACTTC
AGAGAAAAGTAAAAAAA

The NOV4a encoded protein having 977 amino acid residues (SEQ ID NO:13) is presented using the one-letter code in Table 4B.

TABLE 4B

Encoded NOV4a protein sequence (SEQ ID NO:14).

GSRRAGRRVTLPLLLALKMEKMLAGCFLLILGQIVLLPAEARQRSRGRSISRGRHARTHPQTALLESSCENK
RADLVFIIDSSRSVNTHDYAKVKEFIVDILQFLDIGPDVTRVGLLQYGSTVKNEFSLKTFKRKSEVERAVKR
MRHLSTGTMTGLAIQYALNIAFSEAEGARPLRENVPRVIMIVTDGRPQDSVAEVAAKARDTGILIFAIGVGQ
VDFNTLKSIGSEPHEDHVFLVANFSQIETLTSVFQKKLCTAHMCSTLEHNCAHFCINIPGSYVCRCKQGYIL
NSDQTTCRIQDLCAMEDHNCEQLCVNVPGSFVCQCYSGYALAEDGKRCVAVDYCASENHGCEHECVNADGSY
LCQCHEGFALNPDEKTCTKIDYCASSNHGCQYECVNTDDSYSCHCLKGFTLNPDKKTCRRINYCALNKPGCE
HECVNMEESYYCRCHRGYTLDPNGKPCSRVDHCAQQDHGCEQLCLNTEDSFVCQCSEGFLINEDLKTCSRVD
YCLLSDHGCEYSCVNMDRSFACQCPEGHVLRSDGKTCAKLDSCALGDHGCEHSCVSSEDSFVCQCFEGYILR
EDGKTCRRKDVCQAIDHGCEHICVNSDDSYTCECLEGFRLTEDGKRCRISSGKDVCKSTHHGCEHICVNNGN
SYICKCSEGFVLAEDGRRCKKCTEGPIDLVFVIDGSKSLGEENFEVVKQFVTGIIDSLTISPKAARVGLLQY
STQVHTEFTLRNFNSAKDMKKAVAHMKYMGKGSMTGLALKHMFERSFTQGEGARPFSTRVPRAAIVFTDGRA
QDDVSEWASKAKANGITMYAVGVGKAIEEELQEIASEPTNKHLFYAEDFSTMDEISEKLKKGICEALEDSDG
RQDSPAGELPKTVQQPTESEPVTINIQDLLSCSNFAVQHRYLFEEDNLLRSTQKLSHSTKPSGSPLEEKHDQ
CKCENLIMFQNLANEEVRKLTQRLEEMTQRMEALENRLRYR

In a search of sequence databases, it was found, for example, that the NOV4a nucleic acid sequence has 1571 of 1862 bases (84%) identical to a Mus musculus matrilin-2 precursor mRNA (GENBANK-ID: MMU69262). The full amino acid sequence of the NOV4a protein of the invention was found to have 829 of 959 amino acid residues (86% ) identical to, and 887 of 959 residues (92%) positive with, the 956 amino acid residue MATRILIN-2 PRECURSOR protein from Mus musculus (ptnr: SWISSPROT-ACC:O08746).

The NOV4a matrilin-2-like protein disclosed in this invention is expressed in at least the following tissues: dense and loose connective tissue structures; subepithelial connective tissue of the skin and digestive tract; specialized cartilages; and blood vessel walls; fibroblasts of the dermis, tendon, ligaments, perichondrium and periosteum; connective tissue elements of the heart; smooth muscle cells; and epithelia and loose connective tissue cells of the alimentary canal and respiratory tract.

NOV4b

The NOV4b nucleic acid of 2756 nucleotides (also designated CuraGen Acc. No. CG51015-03) encoding a novel MATRILIN-2-like protein is shown in Table 4C. An open reading frame was identified beginning at nucleotides 30-32 and ending at nucleotides 2718-2720. This open reading from begins with an ATG initiation codon and ends with a TGA stop codon. The start and stop codons of the open reading frame are highlighted in bold type. Putative untranslated regions (underlined) are found upstream from the initiation codon and downstream from the termination codon.

TABLE 4C

NOV4b nucleotide sequence (SEQ ID NO:15).

TGACCTTGCCCCTCTTGCTCGCCTTGAAAATGGAAAAGATGCTCGCACGCTGCTTTCTGCTGATC
CTCGGACAGATCGTCCTCCTCCCTGCCGAGGCCAGGGAGCGGTCACGTGGGAGGTCCATCTCTAG
GGGCAGACACGCTCGGACCCACCCGCAGACGGCCCTTCTGGAGAGTTCCTGTGAGAACATGCGGG
CAGACCTGGTTTTCATCATTGACAGCTCTCGCAGTGTCAACACCCATGACTATGCAAAGGTCAAG
GAGTTCATCGTGGACATCTTGCAATTCTTGGACATTGGTCCTGATGTCACCCGAGTGGGCCTGCT
CCAATATGGCAGCACTGTCAAGAATGAGTTCTCCCTCAAGACCTTCAAGAGGAAGTCCGAGGTGG
AGCGTGCTGTCAAGAGGATGCGGCATCTGTCCACGGGCACCATGACCGGGCTGGCCATCCAGTAT
GCCCTGAACATCGCATTCTCAGAAGCAGAGGGGGCCCGGCCCCTGAGGGAGAATGTGCCACGGGT
CATAATGATCGTGACAGATGGGAGACCTCAGGACTCCGTGGCCGAGGTGGCTGCTAAGGCACGGG
ACACGGGCATCCTAATCTTTGCCATTGGTGTGGGCCAGGTAGACTTCAACACCTTGAAGTCCATT
GGGAGTGAGCCCCATGAGGACCATGTCTTCCTTGTGGCCAATTTCAGCCAGATTGAGACGCTGAC
CTCCGTGTTCCAGAAGAAGTTGTGCACGGCCCACATGTGCAGCACCCTGGAGCATAACTGTGCCC
ACTTCTGCATCAACATCCCTGGCTCATACGTCTCCAGGTGCAAACAAGGCTACATTCTCAACTCG
GATCAGACGACTTGCAGAATCCAGGATCTGTGTGCCATGGAGGACCACAACTGTGAGCAGCTCTG
TGTGAATGTGCCGGGCTCCTTCGTCTGCCAGTGCTACAGTGGCTACGCCCTGGCTGAGGATGGGA
AGAGGTGTGTGGCTGTGGACTACTGTGCCTCATCTAATCACGGATGTCAGCACGAGTGTGTTAAC
ACAGATGATTCCTATTCCTGCCACTGCCTGAAAGGCTTTACCCTGAATCCAGATAAGAAAACCTG
CAGAAGGATCAACTACTGTGCACTGAACAAACCGGGCTGTGAGCATGAGTGCGTCAACATGGAGG
AGAGCTACTACTGCCGCTGCCACCGTGGCTACACTCTGGACCCCAATGGCAAAACCTGCAGCCGA
GTGGACCACTGTGCACAGCAGGACCATGGCTGTGAGCAGCTGTGTCTGAACACGGAGGATTCCTT
CGTCTGCCAGTGCTCAGAAGGCTTCCTCATCAACGAGGACCTCAAGACCTGCTCCCGGGTGGATT
ACTGCCTGCTGAGTGACCATGGTTGTGAATACTCCTGTGTCAACATGGACAGATCCTTTGCCTGT
CAGTGTCCTGAGGGACACGTGCTCCGCAGCGATGGGAAGACGTGTGCAAAATTGGACTCTTGTGC
TCTGGGGGACCACGGTTGTGAACATTCGTGTGTAAGCAGTGAAGATTCGTTTGTGTGCCAGTGCT
TTGAAGGTTATATACTCCGTGAAGATGGAAAAACCTGCAGAAGGAAAGATGTCTGCCAAGCTATA
GACCATGGCTGTGAACACATTTGTGTGAACAGTGATGACTCATACACGTGCGAGTGCTTGGAGGG
ATTCCGGCTCGCTGAGGATGGGAAACGCTGCCGAAGGAAGGATGTCTGCAAATCAACCCACCATG
GCTGCGAACACATTTGTGTTAATAATGGGAATTCCTACATCTGCAAATGCTCAGAGGGATTTGTT
CTAGCTGAGGACGGAAGACGGTGCAAGAAATGCACTGAAGGCCCAATTGACCTGGTCTTTGTGAT
CGATGGATCCAAGAGTCTTGGAGAAGAGAATTTTGAGGTCGTGAAGCAGTTTGTCACTGGAATTA
TAGATTCCTTGACAATTTCCCCCAAAGCCGCTCGAGTGGGGCTGCTCCAGTATTCCACACAGGTC
CACACAGAGTTCACTCTGAGAAACTTCAACTCAGCCAAAGACATGAAAAAAGCCGTGGCCCACAT
GAAATACATGGGAAAGGGCTCTATGACTGGGCTGGCCCTGAAACACATGTTTGAGAGAAGTTTTA
CCCAAGGAGAAGGGGCCAGGCCCCTTTCCACAAGGGTGCCCAGAGCAGCCATTGTGTTCACCGAC
GGACGGGCTCAGGATGACGTCTCCGAGTGGGCCAGTAAAGCCAAGGCCAATGGTATCACTATGTA
TGCTGTTGGGGTAGGAAAAGCCATTGAGGACGAACTACAAGAGATTGCCTCTGAGCCCACAAACA
AGCATCTCTTCTATGCCGAAGACTTCAGCACAATGGATGAGATAAGTGAAAAACTCAAGAAAGGC
ATCTGTGAAGCTCTAGAAGACTCCGATGGAAGACAGGACTCTCCAGCACGGGAACTGCCAAAAAC
GGTCCAACAGCCAACAGTGCAACACAGATATCTGTTTGAAGAAGACAATCTTTTACGGTCTACAC
AAAAGCTTTCCCATTCAACAAAACCTTCAGGAAGCCCTTTGGAAGAAAAACACGATCAATGCAAA
TGTGAAAACCTTATAATGTTCCAGAACCTTGCAAACGAAGAAGTAAGAAAATTAACACAGCGCTT
AGAAGAAATGACACAGAGAATGGAAGCCCTGGAAAATCGCCTGAGATACAGATGAAGATTAGAAA
TCGCGACACATTTGTAAAGGGCGAAT

The NOV4 disclosed in this invention maps to chromosome 8. This assignment was made using mapping information associated with genomic clones, public genes and ESTs sharing sequence identity with the disclosed sequence and CuraGen Corporation's Electronic Northern bioinformatic tool.

The NOV4b encoded protein having 896 amino acid residues (SEQ ID NO:16) is presented using the one-letter code in Table 4D.

TABLE 4D

Encoded NOV4b protein sequence (SEQ ID NO:16).

MEKMLAGCFLLILGQIVLLPAEARERSRGRSISRGRHARTHPQTALLESS
CENMRADLVFIIDSSRSVNTHDYAKVKEFIVDILQFLDIGPDVTRVGLLQ
YGSTVKNEFSLKTFKRKSEVERAVKRMRHLSTGTMTGLAIQYALNIAFSE
AEGARPLRENVPRVIMIVTDGRPQDSVAEVAAKARDTGILIFAIGVGQVD
FNTLKSIGSEPHEDHVFLVANFSQIETLTSVFQKKLCTAHMCSTLEHNCA
HFCINIPGSYVCRCKQGYILNSDQTTCRIQDLCAMEDHNCEQLCVNVPGS
FVCQCYSGYALAEDGKRCVAVDYCASSNHGCQHECVNTDDSYSCHCLKGF
TLNPDKKTCRRINYCALNKPGCEHECVNMEESYYCRCHRGYTLDPNGKTC
SRVDHCAQQDHGCEQLCLNTEDSFVCQCSEGFLINEDLKTCSRVDYCLLS
DHGCEYSCVNMDRSFACQCPEGHVLRSDGKTCAKLDSCALGDHGCEHSCV
SSEDSFVCQCFEGYILREDGKTCRRKDVGQAIDHGCEHICVNSDDSYTCE
CLEGFRLAEDGKRCRRKDVCKSTHHGCEHICVNNGNSYICKCSEGFVLAE
DGRRCKKCTEGPIDLVFVIDGSKSLGEENFEVVKQFVTGIIDSLTISPKA
ARVGLLQYSTQVHTEFTLRNFNSAKDMKKAVAHMKYMGKGSMTGLALKHM
FERSFTQGEGARPLSTRVPRAAIVFTDGRAQDDVSEWASKAKANGITMYA
VGVGKAIEEELQEIASEPTNKHLFYAEDFSTMDEISEKLKKGICEALEDS
DGRQDSPAGELPKTVQQPTVQHRYLFEEDNLLRSTQKLSHSTKPSGSPLE
EKHDQCKCENLIMFQNLANEEVRKLTQRLEEMTQRMEALENRLRYR

In a search of sequence databases, it was found, for example, that the NOV4b nucleic acid sequence of this invention has 1897 of 1996 bases (95%) identical to a gb:GENBANK-ID:BC010444|acc:BC010444.1 mRNA from Homo sapiens (Homo sapiens, matrilin 2, clone MGC:17281 IMAGE:4215380, mRNA, complete cds) (FIG. 3A). The full amino acid sequence of the NOV4b protein of the invention was found to have 613 of 656 amino acid residues (93%) identical to, and 626 of 656 amino acid residues (95%) similar to, the 937 amino acid residue ptnr:TREMBLNEW-ACC:AAH10444 protein from Homo sapiens (Human) (MATRILIN 2).

The NOV4b MATRILIN-2-like gene disclosed in this invention is expressed in at least the following tissues: Mammalian Tissue, Adipose, Heart, Aorta, Vein, Adrenal Gland/Suprarenal gland, Pancreas, Parathyroid Gland, Thyroid, Pineal Gland, Parotid Salivary glands, Stomach, Liver, Small Intestine, Appendix, Colon, Ascending Colon, Lymph node, Cartilage, Smooth Muscle, Brain, Cerebellum, Pituitary Gland, Amygdala, Cerebral Medulla/Cerebral white matter, Basal Ganglia/Cerebral nuclei, Substantia Nigra, Hippocampus, Spinal Chord, Cervix, Mammary gland/Breast, Ovary, Placenta, Uterus, Vulva, Prostate, Testis, Lung, Urinary Bladder, Kidney, Retina, Skin. Expression information was derived from the tissue sources of the sequences that were included in the derivation of the sequence of NOV4b (CuraGen Acc. No. CG51015-03). The NOV4b sequence is predicted to be expressed in the following tissues because of the expression pattern of (GENBANK-ID: gb:GENBANK-ID:BC010444|acc:BC010444.1) a closely related Homo sapiens, matrilin 2, clone MGC:17281 IMAGE:4215380, mRNA, complete cds homolog in species Homo sapiens:brain.

The PSORT, SignalP and hydropathy profile for NOV4 predict that this sequence has a signal peptide and is likely to be localized to the endoplasmic reticulum (membrane) with a certainty of 0.8200. In alternative embodiments, NOV4 is located to the plasma membrane with a certainty of 0.2710, the endoplasmic reticulum (lumen) with a certainty of 0.1000, or extracellularly with a certainty of 0.1000. The signal peptide of NOV4a is predicted by SignalP to be cleaved at amino acid position 41 and 42: AEA-RQ. The signal peptide of NOV4b is predicted by SignalP to be cleaved at amino acid 23 and 24: AEA-RE.

Homologies to any of the above NOV4 proteins will be shared by the other NOV4 protein insofar as they are homologous to each other as shown above. Any reference to NOV4 is assumed to refer to both of the NOV4 proteins in general, unless otherwise noted.

Additional SNP variants of NOV4 are disclosed in Example 3. The amino acid sequence of NOV1 has high homology to other proteins as shown in Table 4E.

TABLE 4E
BLASTX results for NOV4

| | | Smallest |
|---|---|---|
| | | Sum |
| | High | Prob |
| Sequences producing High-scoring Segment Pairs: | Score | P(N) |
| patp: AAB20159 Human protein SECP5 - Homo | 5165 | 0.0 |
| sapiens, 959 aa | | |
| patp: AAE03877 Human gene 3 encoded secreted | 4647 | 0.0 |
| protein fragment - Homo sapiens, 983 aa | | |
| patp: AAE03843 Human gene 3 encoded secreted | 4595 | 0.0 |
| protein HOGDP46 - Homo sapiens, 934 aa | | |
| patp: AAE03820 Human gene 3 encoded secreted | 4208 | 0.0 |
| protein HOGDP46 - Homo sapiens, 794 aa | | |
| patp: AAB20158 Human protein SECP4 - Homo | 4133 | 0.0 |
| sapiens, 776 aa | | |

NOV4 also has homology to the proteins shown in the BLASTP data in Table 4F.

TABLE 4F
BLASTP results for NOV4

| Gene Index/ | | Length | Identity | Positives | |
|---|---|---|---|---|---|
| Identifier | Protein/Organism | (aa) | (%) | (%) | Expect |
| ptnr: REMTREMBL- | Sequence 9 from | 959 | 956/959 | 958/959 | 0.0 |
| ACC: CAC32426 | Patent WO0105971 | | (99%) | (99%) | |
| | Homo sapiens | | | | |
| | (Human) | | | | |
| ptnr: SWISSNEW- | Matrilin-2 | 956 | 949/959 | 952/959 | 0.0 |
| ACC: O00339 | precursor - Homo | | (98%) | (99%) | |
| | sapiens (Human) | | | | |
| ptnr: TREMBLNEW- | MATRILIN 2 - Homo | 937 | 854/863 | 856/863 | 0.0 |
| ACC: AAH10444 | sapiens (Human) | | (98%) | (99%) | |
| ptnr: SPTREMBL- | MATRILIN 2 - Mus | 956 | 832/959 | 888/959 | 0.0 |
| ACC: Q99K64 | musculus (Mouse) | | (86%) | (92%) | |
| ptnr: SWISSPROT- | MATRILIN-2 | 956 | 829/959 | 887/959 | 0.0 |
| ACC: O08746 | PRECURSOR - Mus | | (86%) | (92%) | |
| | musculus (Mouse) | | | | |

The homology between the sequences shown in Table 4F and between the NOV4 sequences is shown graphically in the ClustalW analysis shown in Table 4G.

[image]

[image]

[image]

[image]

Table 4H lists the domain description from pfam analysis results for NOV4. This indicates that the NOV4 sequence has properties similar to those of other proteins known to contain this domain.

TABLE 4H
Domain Analysis of NOV4

| | | Score | Expect |
|---|---|---|---|
| IPR002035; vwa | von Willebrand factor type A | 429.1 | 4.1e−125 |
| | domain | | |
| IPR000561; EGF | EGF-like domain | 274.8 | 1.1e−78 |

The von Willebrand factor is a large multimeric glycoprotein found in blood plasma. Mutant forms are involved in the aetiology of bleeding disorders. In von Willebrand factor, the type A domain (vWF) is the prototype for a protein superfamily. The vWF domain is found in various plasma proteins: complement factors B, C2, CR3 and CR4; the integrins (I-domains); collagen types VI, VII, XII and XIV; and other extracellular proteins. Proteins that incorporate vWF domains participate in numerous biological events (e.g., cell adhesion, migration, homing, pattern formation, and signal transduction), involving interaction with a large array of ligands. Secondary structure prediction from 75 aligned vWF sequences has revealed a largely alternating sequence of alpha-helices and beta-strands. Fold recognition algorithms were used to score sequence compatibility with a library of known structures: the vWF domain fold was predicted to be a doubly-wound, open, twisted beta-sheet flanked by alpha-helices. 3D structures have been determined for the I-domains of integrins CD11b (with bound magnesium) and CD11a (with bound manganese). The domain adopts a classic alpha/beta Rossmann fold and contains an unusual metal ion coordination site at its surface. It has been suggested that this site represents a general metal ion-dependent adhesion site (MIDAS) for binding protein ligands. The residues constituting the MIDAS motif in the CD11b and CD11a I-domains are completely conserved, but the manner in which the metal ion is coordinated differs slightly.

| Alignments of top-scoring domains: | | | | |
|---|---|---|---|---|
| vwa: domain 1 of 2, from 75 to 250: score 211.3, E = 1.4e − 59 | | | | |
| vwa | | DIVFLLDGSGSIGSQNFERVKDFVERVVERLDVGPRDKKEEDAVRVG | | (SEQ ID NO:78) |
| | | \|+\|\|++\|+\|+\|+ ++ ++\|\|+\|++++ + \|\|+\|\| \| +\|\|\| | | |
| NOV4 | 75 | DLVFIIDSSRSVNTHDYAKVKEFIVDILQFLDIGP-DV-----TRVG | 115 | (SEQ ID NO:79) |
| +0 | | | | |
| vwa | | LVQYSDNVRTEIKFKLNDYQNKDEVLQALQKIRYEDYYGGGGTNTGAALQ | | |
| | | \|+\|\|++ \|+ \| \|+\|+++++\|+\|\| +\|++++\| + +\|\|+\|\| \|+\| | | |
| NOV4 | 116 | LLQYGSTVKNE--FSLKTFKRKSEVERAVKRMR----HLSTGTMTGLAIQ | 159 | |
| +0 | | | | |
| vwa | | YVVRNLFTEASGSRIEPVAEEGAPKVLVVLTDGRSQDDPSPTIDIRDVLN | | |
| | | \|+ + +\|+\|\| \|+\|+ +\|++\|+\|+ ++\|\|\|\|+\|\|+ + +\|++ | | |
| NOV4 | 160 | YALNIAFSEAEGARP---LRENVPRVIMIVTDGRPQDS------VAEVAA | 200 | |
| +0 | | | | |
| vwa | | ELKKEAGVEVFAIGVGNADNNNLEELREIASKPD.DHVFKVSDFEALDTL | | |
| | | ++++ +\|+ +\|\|\|\|\|\|++\| + +\|++\|+\|+\| +\|\|\|\| \| +\|++++\|\| | | |
| NOV4 | 201 | KARD-TGILIFAIGVGQVD---FNTLKSIGSEPHeDHVFLVANFSQIETL | 246 | |
| +0 | | | | |
| vwa | | QELL | | |
| | | + + | | |
| NOV4 | 247 | TSVF | 250 | |
| +0 | | | | |
| vwa: domain 2 of 2, from 676 to 851: score 219.6, E +32 4.5e − 62 | | | | |
| vwa | | DIVFLLDGSGSIGSQNFERVKDFVERVVERLDVGPRDKKEEDAVRVG | | (SEQ ID NO:80) |
| | | \|+\|\|++\|\|\| \|+\|+ \|\|\| \|\|+\|\| ++++\|+++\| + +\|\|\| | | |
| NOV4 | 676 | DLVFVIDGSKSLGEENFEVVKQFVTGIIDSLTISP-KA-----ARVG | 716 | (SEQ ID NO:81) |
| +0 | | | | |
| vwa | | LVQYSDNVRTEIKFKLNDYQNKDEVLQALQKIRYEDYYGGGGTNTGAALQ | | |
| | | \|+\|\|\|++\|+\|\| \|+\|+ + + +++ +\|+ +++ \|+\|+\| +\|\| \|\|+ | | |
| NOV4 | 717 | LLQYSTQVHTE--FTLRNFNSAKDMKKAVAHMK----YMGKGSMTGLALK | 760 | |
| +0 | | | | |
| vwa | | YVVRNLFTEASGSRIEPVAEEGAPKVLVVLTDGRSQDDPSPTIDIRDVLN | | |
| | | ++ ++ \|\|+ \|+\|+ + ++\|+ ++\|+\|\|\|\| \|\|\| + + + | | |
| NOV4 | 761 | HMFERSFTQGEGARP---FSTRVPRAAIVFTDGRAQDD------VSEWAS | 801 | |
| +0 | | | | |
| vwa | | ELKKEAGVEVFAIGVGNADNNNLEELREIASKPD.DHVFKVSDFEALDTL | | |
| | | ++\|+ +\|++++\|+\|\|\|+\| +\|\|\|+\|\|\|\|+\|+++\| \|+ +\|\|++ \|++ | | |
| NOV4 | 802 | KAKA-NGITMYAVGVGKAI---EEELQEIASEPTNKHLFYAEDFSTMDEI | 847 | |
| +0 | | | | |
| vwa | | QELL | | |
| | | \|+\| | | |
| NOV4 | 848 | SEKL | 851 | |
| +PS | | | | |

NOV5

A disclosed NOV5 nucleic acid (SEQ ID NO:17) of 1779 nucleotides (also referred to as SC85803748_A) encoding a GABA receptor-like protein is shown in Table 5A. An open reading frame was identified beginning with an ATG initiation codon at nucleotides 71-73 and ending with a TGA codon at nucleotides 1445-1447. Putative untranslated regions upstream from the initiation codon and downstream from the termination codon are underlined in Table 5A, and the start and stop codons are in bold letters.

TABLE 5A

NOV5 Nucleotide Sequence

| | |
|---|---|
| AAGAAGAAACTGTGATCACAGTATTGGTTGCGTTCACCTGCATCCTTTCTGTTTTTTTGTTTTGGAAGAGATGGTC | (SEQ ID NO:17) |
| CTGGCTTTCCAGTTAGTCTCCTTCACCTACATCTGGATCATATTGGTTTGTGCTGCTTCTAACATCAAGATGACAC | |
| ACCAGCGGTGCTCCTCTTCAATGAAACAAACCGTAAGATGCTCAATGAAGAAAGATGACAGTACCAAAGCGCGGCC | |
| TCAGAAATATGAGCAACTTCTCCATATAGAGGACAACGATTTCGCAATGAGACCTGGATTTGGAGGTTCTCCAGTG | |
| CCAGTAGGTATAGATGTCCATGTTGAAAGCATTGACAGCATTTCAGAGACTAACATGGACTTTACAATGACTTTTT | |
| ATCTCAGGCATTACTGGAAAGACGAGAGGCTCTCCTTTCCTAGCACAGCAAACAAAAGCATGACATTTGATCATAG | |
| AAAGAGTATCCCCCGCCCTGAACACTTGCGGTATTCGTTATTCATCAGAAGGCTGTATCTGTTATACTGCCAGAGG | |
| TCTTTCTTCTCACCCTCATCCATACTTCCCTCATCTCCAGACATCCATGCACCTGGTACATCTAAAAGCAGTTTGT | |
| CTGATAGCCTTGTATGTATATCTGAAAAAAACTTGCCAGGACACAGTAAAAACACACCTCTTGCAATGGCCTACAA | |
| TGAGGATGACCTAATGCTATACTGGAAACACGGAAACAAGTCCTTAAATACTGAAGAACATATGTCCCTTTCTCAG | |
| TTCTTCATTGAAGACTTCAGTGCATCTAGTGGATTAGCTTTCTATAGCAGCACAGGTACAGCATTTTACATGGGTG | |
| ATTCATCAGCATTTATTGGACATCTACTGTTTTTAAATAGACATTTACATTTCTTCATCATAAATTTTGAAATTAC | |
| TCAAATATTGATGATTGGAATCACCACAGTGCTGACCATGTCCACAATCATCACTGCTGTGAGCGCCTCCATGCCC | |
| CAGGTGTCCTACCTCAAGGCTGTGGATGTGTACCTGTGGGTCAGCTCCCTCTTTGTGTTCCTGTCAGTCATTGAGT | |
| ATGCAGCTGTGAACTACCTCACCACAGTGGAAGAGCGGAAACAATTCAAGAAGACAGGAAAGGTACAGATTTCTAG | |
| GATGTACAATATTGATGCAGTTCAAGCTATGGCCTTTGATGGTTGTTACCATGACAGCGAGATTGACATGGACCAG | |
| ACTTCCCTCTCTCTAAACTCAGAAGACTTCATGAGAAGAAAATCGATATGCAGCCCCAGCACCGATTCATCTCGGA | |
| TAAAGAGAAGAAAATCCCTAGGAGGACATGTTGGTAGAATCATTCTGGAAAACAACCATGTCATTGACACCTATTC | |
| TAGGATTTTATTCCCCATTGTGTATATCCCATTGTGTATATCTTTATTTAATTTGTTTTACTGGGGTGTATATGTA | |
| TGAAGGGGAATTTCAAATGTATACAACTTTAAAGCCAGATGATGTTTAAAAACAAAACTCTTGAATATGAGTTGGA | |
| ATTGAAGACTTCAGTGCATCTAGTGGATTAGCTTTCTATAGCAGCACAGGTACAGCATTTTACATGGGTGATTCAT | |
| CAGCATTTATTGGACATCTACTGTTTTACTTTTGGTCTTTGATGATGGTGATGTACAGATGGGTTGGAATCACCAC | |
| AGTGCTGACCATGTCCACAATCATCACTGCTGTGAGCGCCTCCATGCCCCAGGTGTCCTACCTCAAGGCTGTGGAT | |
| GTGTACCTGTGGGTCAGCTCCCTCTTTGTGTTCCTGTCAGTCATTGAGTAT | |

Genomic clone AC026100 on chromosome 3 was identified by TBLASTN using a proprietary sequence file for members of GABA receptor and/or Ion channel family, run against the genomic daily files made available by GenBank or obtained from Human Genome Project Sequencing centers. These genomic clones were analyzed by Genscan and Grail and other programs to identify regions that were putative exons i.e. putative coding sequences. These clones were also analyzed by BLASTN, TBLASTN, TFASTN, TFASTA, BLASTX and/or other programs to identify genomic regions with DNA similarity or translating to proteins with similarity to the original protein or protein family of interest.

A disclosed NOV5 polypeptide (SEQ ID NO:18) encoded by SEQ ID NO:17 is 458 amino acid residues and is presented using the one-letter code in Table 5B. Signal P, Psort and/or Hydropathy results predict that NOV5 is likely to be localized in the endoplasmic reticulum (membrane) with a certainty of 0.6850. In alternative embodiments, NOV5 is localized in the plasma membrane with a certainty of 0.6400, Golgi body with a certainty of 0.4600, and endoplasmic reticulum (lumen) with a certainty of 0.1000. NOV5 has a signal peptide and is likely cleaved between amino acid residues 20 and 21, i.e., at the dash in the sequence VCA-AS.

TABLE 5B

Encoded NOV5 protein sequence

| | |
|---|---|
| MVLAFQLVSFTYIWIILVCAASNIKMTHQRCSSSMKQTVRCSMKKDDSTKARPQKYEQLLHIEDNDFAMRPG | (SEQ ID NO:18) |
| FGGSPVPVGIDVHVESIDSISETNMDFTMTFYLRHYWKDERLSFPSTANKSMTFDHRKSIPRPEHLRYSLFI | |
| RRLYLLYCQRSFFSPSSILPSSPDIHAPGTSKSSLSDSLVCISEKNLPGHSKNTPLAMAYNEDDLMLYWKHG | |
| NKSLNTEEHMSLSQFFIEDFSASSGLAFYSSTGTAFYMGDSSAFIGHLLFLNRHLHFFIINFEITQILMIGI | |
| TTVLTMSTIITAVSASMPQVSYLKAVDVYLWVSSLFVFLSVIEYAAVNYLTTVEERKQFKKTGKVQISRMYN | |
| IDAVQAMAFDGCYHDSEIDMDQTSLSLNSEDFMRRKSICSPSTDSSRIKRRKSLGGHVGRIILENNHVIDTY | |
| SRILFPIVYIPLCISLFNLFYWGVYV | |

NOV5 is expressed in at least the following tissues: kidney, retina. In addition, the NOV5 is predicted to be expressed in retinal tissues because of the expression pattern of a closely related Rat mRNA for GABA receptor rho-3 subunit, complete cds homolog in species Rattus norvegicus (GENBANK-ID: D50671). Additional data for NOV5 variants and NOV5 expression is provided in the Examples.

In a search of sequence databases, it was found, for example, that the nucleic acid sequence of this invention has 563 of 765 bases (73%) identical to a gb:GENBANK-ID: RATGABA|acc:D50671 mRNA from Rattus norvegicus (Rat mRNA for GABA receptor rho-3 subunit, complete cds). The full amino acid sequence of the protein of the invention was found to have 299 of 464 amino acid residues (64%) identical to, and 347 of 464 amino acid residues (74%) similar to, the 464 amino acid residue ptnr:SWISSPROT-ACC:P50573 Gamma-Aminobutyric-Acid Receptor Rho-3 Subunit Precursor (“GABA(A)”) Receptor protein from Rattus norvegicus (Rat).

In a search of the public sequence data bases, it was shown that NOV5 also has homology to the amino acid sequences shown in the BLASTP data listed in Table 5C.

TABLE 5C
BLASTP results for NOV5

| Gene Index/ | | Length | Identity | Positives | |
|---|---|---|---|---|---|
| Identifier | Protein/Organism | (aa) | (%) | (%) | Expect |
| SWISSPROT | GAMMA-AMINOBUTYRIC-ACID | 464 | 299/464 | 347/464 | 6.1e−138 |
| P50573 | RECEPTOR RHO-3 SUBUNIT | | (64%) | (74%) | |
| | PRECURSOR (GABA(A) | | | | |
| | RECEPTOR) - Rattus | | | | |
| | norvegicus | | | | |
| SPTREMBL | GAMMA-AMINOBUTYRIC-ACID | 470 | 216/466 | 302/466 | 6.4e−88 |
| Q9YGQ2 | RECEPTOR RHO-3 SUBUNIT | | (46%) | (64%) | |
| | PRECURSOR - Morone | | | | |
| | americana (White perch) | | | | |
| SWISSNEW | Gamma-aminobutyric-acid | 473 | 198/424 | 266/424 | 1.8e−81 |
| P24046 | receptor rho-1 subunit | | (46%) | (62%) | |
| | precursor (GABA(A) | | | | |
| | receptor) - Homo sapiens | | | | |
| SWISSPROT | GAMMA-AMINOBUTYRIC-ACID | 474 | 196/416 | 262/416 | 1.5e−79 |
| P50572 | RECEPTOR RHO-1 SUBUNIT | | (47%) | (62%) | |
| | PRECURSOR (GABA(A) | | | | |
| | RECEPTOR) - Rattus | | | | |
| | norvegicus | | | | |
| SWISSPROT | GAMMA-AMINOBUTYRIC-ACID | 474 | 196/416 | 262/416 | 4.0e−79 |
| P56475 | RECEPTOR RHO-1 SUBUNIT | | (47%) | (62%) | |
| | PRECURSOR (GABA(A) | | | | |
| | RECEPTOR) - Mus musculus | | | | |

The homology of these sequences is shown graphically in the ClustalW analysis shown in Table 5D.

[image]

[image]

In a search of the proprietary PatP sequence data base, it was shown that NOV5 also has homology to the amino acid sequences shown in the BLASTP data listed in Table 5E.

TABLE 5E
PatP BLASTP results for NOV5

| Gene Index/ | | Length | | | |
|---|---|---|---|---|---|
| Identifier | Protein/Organism | (aa) | Identity (%) | Positives (%) | Expect |
| AAR31188 | GABA-A receptor beta-2 | 474 | 119/382 | 199/382 | 8.6e−37 |
| | subunit - Homo sapiens | | (31%) | (52%) | |
| AAR59866 | GABA receptor beta2 | 474 | 119/382 | 199/382 | 8.6e−37 |
| | subunit - Homo sapiens | | (31%) | (52%) | |
| AAR93118 | GABA-A receptor epsilon | 440 | 115/394 | 201/394 | 1.1e−32 |
| | subunit - Homo sapiens | | (29%) | (51%) | |
| AAW26464 | GABA-A receptor epsilon | 440 | 115/394 | 201/394 | 1.1e−32 |
| | subunit - Homo sapiens | | (29%) | (51%) | |
| AAB00174 | Breast cancer protein | 440 | 115/394 | 201/394 | 1.1e−32 |
| | BCR3 - Homo sapiens | | (29%) | (51%) | |

In addition, the NOV5 protein is predicted to contain the following protein domains (as defined by Interpro) at the indicated nucleotide positions: domain name IPR001175 at amino acid positions 53 to 129, domain name IPR001175 at amino acid positions 287 to 346, etc. This indicates that the sequence of the invention has properties similar to those of other proteins known to contain this/these domain(s) and similar to the properties of these domains. Table 5F list the domain description from DOMAIN analysis results against NOV5. This homology indicates that the NOV5 sequence has properties similar to those of other proteins known to contain this domain.

TABLE 5F

Domain Analysis of NOV5

Scores for sequence family classification (score includes all domains)

| Model | InterPro | Score | E-value |
|---|---|---|---|
| Neur_chan_LBD | Neurotransmitter-gated ion-channel lig | 70.6 | 1.7e − 18 |
| Neur_chan_memb | Neurotransmitter-gated ion-channel tra | 23.4 | 2e − 10 |

Parsed for domains:

| | | | | | | | |
|---|---|---|---|---|---|---|---|
| Model | Domain | seq-f | seq-t | hmm-f | hmm-t | score | E-value |

| | | | | | | | |
|---|---|---|---|---|---|---|---|
| Neur_chan_LBD | 1/2 | 66 | 132 .. | 17 | 102 .. | 63.4 | 1.7e − 16 |

| | |
|---|---|
| Neur_chan_LBD: | domain 1 of 2, from 66 to 132 |

| | | | | | | | |
|---|---|---|---|---|---|---|---|
| Neur_chan_LBD | 2/2 | 203 | 249 .. | 175 | 238 .. | 7.2 | 0.69 |

| | |
|---|---|
| Neur_chan_LBD: | domain 2 of 2, from 203 to 249 |

| | | | | | | | |
|---|---|---|---|---|---|---|---|
| Neur_chan_memb | 1/1 | 274 | 454 .. | 1 | 291 [] | 23.4 | 2e − 10 |

| | |
|---|---|
| Neur_chan_memb: | domain 1 of 1, from 274 to 454 |

ClustalW alignment of NOV5 with Neur_chain_LED

| | | | | |
|---|---|---|---|---|
| Neur_chan_LBD | | *->dkrvRPvnggdvPpvtVsvgltlqqiisVdEknqdlttnvwlrqgqW | | (SEQ ID NO:107) |
| | | \| +\|\|++\|\|+ \| \| \|++++++ +\|+\|++\| \|+\|+\|++ +\|\|+ +\| | | |
| C441_NOV5 | 66 | DFAMRPGFGGS-P-VPVGIDVHVESIDSISETNMDFTMTFYLRH-YW | 109 | (SEQ ID NO:108) |
| Neur_chan_LBD | | tDpRLaWnpsdplddegdyggikslrlpsddnhdmldkI | | |
| | | +\|+\|\|+++ + \|\|++++++ ++\| | | |
| C441_NOV5 | 110 | KDERLSFPSTA----------NKSMTFDHR------KSI | 132 | |

Neurotransmitter-gated ion-channels {PDOC00209, PS00236; Neurotr_Ion_Channel} provide the molecular basis for rapid signal transmission at chemical synapses. They are post-synaptic oligomeric transmembrane complexes that transiently form a ionic channel upon the binding of a specific neurotransmitter. The neurotransmitter-gated ion-channel domain consensus pattern is as follows:

C-x-[LIVMFQ]-x-[LIVMF]-x(2)-[FY]-P-x-D-x(3)-C (SEQ ID NO:109),

wherein the two C's are linked by a disulfide bond.

The above defined information for NOV5 suggests that this NOV5 protein may function as a member of a GABA receptor protein family. GABA receptor activity in mammalian cells correlate with known diseases and disorders (See, e.g., OMIM 600232, 137192, 137142 and 600233). Therefore, the NOV5 nucleic acids and proteins of the invention are useful in potential therapeutic applications implicated in various diseases and disorders described below and/or other pathologies. For example, the NOV5 compositions of the present invention will have efficacy for treatment of patients suffering from: cancer, trauma, regeneration (in vitro and in vivo), viral/bacterial/parasitic infection, multiple sclerosis, leukodystrophies, pain, Von Hippel-Lindau (VHL) syndrome, Alzheimer's disease, stroke, tuberous sclerosis, hypercalceimia, Parkinson's disease, Huntington's disease, Angelman syndrome (AS; 105830), Prader-Willi syndrome (PWS; 176270), bipolar affective disorder, cerebral palsy, epilepsy, Lesch-Nyhan syndrome, Ataxia-telangiectasia, behavioral disorders, addiction, anxiety, retinal and visual disorders. The NOV5 nucleic acid encoding fatty acid-binding protein, and the fatty acid-binding protein of the invention, or fragments thereof, may further be useful in diagnostic applications, wherein the presence or amount of the nucleic acid or the protein are to be assessed.

NOV5 nucleic acids and polypeptides are further useful in the generation of antibodies that bind immuno-specifically to the novel NOV5 substances for use in therapeutic or diagnostic methods. These antibodies may be generated according to methods known in the art, using prediction from hydrophobicity charts, as described in the “Anti-NOVX Antibodies” section below. The disclosed NOV5 protein has multiple hydrophilic regions, each of which can be used as an immunogen. In one embodiment, a contemplated NOV5 epitope is from about amino acids 1 to 10. In another embodiment, a NOV5 epitope is from about amino acids 20 to 80. In additional embodiments, NOV5 epitopes are from about amino acids 85 to 150, from about amino acids 155 to 240, from about amino acids 325 to 430, and from about amino acids 445 to 458. These novel proteins can be used in assay systems for functional analysis of various human disorders, which will help in understanding of pathology of the disease and development of new drug targets for various disorders.

NOV6

Yet another NOVX protein of the invention, referred to herein as NOV6, is a giant larvae-like protein.

Although some members of the giant larvae-like protein family may be localized in the nucleus, the protein predicted here is similar to the Drosophila giant larvae protein, which has membrane and cytoskeletal localization and may be released extracellularly. Therefore it is likely that this novel giant larvae-like protein is available at the same sub-cellular localization and hence accessible to a diagnostic probe and for the various therapeutic applications described herein.

The giant larvae homolog-like gene disclosed in this invention maps to chromosome 17. This assignment was made using mapping information associated with genomic clones, public genes and ESTs sharing sequence identity with the disclosed sequence and CuraGen Corporation's Electronic Northern bioinformatic tool.

Two alternative NOV6 nucleic acids and encoded polypeptides are provided, namely NOV6a and NOV6b.

NOV6a

In one embodiment, a disclosed NOV6 variant is NOV6a (alternatively referred to herein as sggc_draft_ba465b22_{—}20000727), which encodes a novel giant larvae-like protein and includes the 3147 nucleotide sequence (SEQ ID NO:19) shown in Table 6A. An open reading frame for the mature protein was identified beginning with an ATG codon at nucleotides 131-133 and ending with a TGA codon at nucleotides 2843-2845. Putative untranslated regions upstream from the start codon are underlined in Table 6A, and the start codon is in bold letters.

TABLE 6A

NOV6a Nucleotide Sequence (SEQ ID NO:19).

CGCCCAGCAGCCCGTGGGCAGGCGCGGCGGAGCGAGCGGGGCCGGCGGCGGGCGCCGAGGGACGCCGAGGCCTCGG
GCGGGGGCTGGCCCGGGGTTCCAGGTCTCCAGTGGGGGCTGCAGACTAAGCAAAATGAGGCGGTTCCTGAGGCCAG
GGCATGACCCTGTGCGGGAGAGGCTCAAGCGGCACCTGTTCCAGTTTAACAAGACGGTCGAGCATGGCTTCCCGCA
CCAGCCCAGCGCCCTCGGCTACAGCCCGTCCCTGCACATCCTGGCCATCGGCACCCGTTCTGGAGCCATCAAGCTC
TACGGAGCCCCAGGCGTGGAGTTCATGGGGCTGCACCAGGAGAACAACGCTGTGACGCAGATCCACCTCCTGCCCG
GCCAGTGCCAGCTGGTCACCCTGCTGGATGACAACAGCCTGCACCTTTGGAGCCTGAAGGTCAAGGGCGGGGCATC
GGAGCTGCAGGAGGATGAGAGCTTCACACTGCGTGGACCCCCAGGGGCTGCCCCCAGTGCCACACAGATCACCGTG
GTCCTGCCACATTCCTCCTCCGAGCTGCTCTACCTGGGCACCCACAGTGGCAACGTGTTTGTCCTGCAGCTGCCAG
CTTTTCGTGCGCTGGAGGACCGGACCATCAGCTCGGACGCGGTGCTGCAGCCGTTGCCAGAGCAGGCCCGCCACCG
GCGTGTGTTCGAGATGGTGGAGGCACTGCAGGAGCACCCTCGAGACCCCAACCAGATCCTGATCGGCTACAGCCGA
GGCCTCGTTGTCATCTGGGACCTACACGGCAGCCGCGTGCTCTACCACTTCCTCACCAGCCAGCAACTGGAGAACA
TCTGGTGGCAGCGGGACGGCCGCCTGCTCGTCAGCTGTCACTCTGACGGCAGCTACTGCCAGTGGCCCGTGTCCAG
CGAAGCCCAGCAACCAGAGCCCCTCCGCAGCCTCGTGCCTTACGGTCCCTTTCCTTGCAAAGCGATTACCAGAATC
CTCTGGCTGACCACTAGGCAGGGGTTGCCCTTCACCATCTTCCAGGGTGGCATGCCACGGGCCAGCTACGGGGACC
GCCACTGCATCTCAGTGATCCACGATGGCCAGCAGACGGCCTTCGACTTCACCTCCCGTGTCATCGGCACGGTGCG
GTTCTGGGATGCCTCGGGTGTCTGCCTGCGCCTGCTCTACAAACTCAGCACTGTGCGCGTGTTCCTCACCCACACG
GACCCCAACGAGAACTTCAGTGCCCAGGGCGAGGACGAGTGGCCCCCACTCCGCAAGGTGGGCTCCTTTGACCCCT
ACAGTGATGACCCCCGGCTGGGCATCCAGAAGATCTTCCTCTGCAACTACAGCGGCTACCTGGCTGTGGCAGGCAC
GGCAGGGCAGGTGCTGGTACTGGAACTGAATGACGAGGCAGCGGAGCAGGCTGTGGAGCAGGTGGAGGCCGACCTG
CTGCAGGACCAAGAGGGCTACCGCTGGAAGGGGCACGAGCGCCTGGCAGCCCGCTCAGGGCCCGTGCGCTTTGAGC
CTGGCTTTCAGCCCTTCGTGTTGGTGCAGTGTCAGCCCCCGGCTGTGGTCACCTCCTTGGCCCTGCACTCTGAGTG
GCGGCTCGTGGCCTTCCGCACCAGCCATGGCTTTGGCCTCTTTGACCACCAGCAGCGGCGGCACGTCTTTGTTAAG
TGCACACTGCACCCCAGTGACCAGCTGGCCTTGGAGGGCCCACTCTCCCGCGTCAAGTCCCTCAAGAAGTCCTTGC
GTCAGTCATTCCGCCGGATGCGTCGGAGCCGGGTGTCCAGCCGGAAGCGGCACCCGGCTGGCCCCCCAGGAGAGGC
ACACCAGGGGAGTGCCAAGGCTGAGCGGCCAGGCCTCCAGAACATGGAGCTGGCGCCTGTGCAGCGCAAGATCGAG
GCTCGCTCGGCAGAGGACTCCTTCACAGGCTTCGTCCGGACCCTGTACTTTGCTGACACCTACCTGAAGCACAGCT
CCCGGCACTGCCCCTCGCTGTGGGCTGGCACCAATGGGGGCACCATCTATGCCTTCTCCCTGCGTGTGCCTCCCGC
CGAGCGGAGAATGGATGAGCCTGTGCGGGCAGAGCAGGCCAAGGAGATCCAGCTGATGCACCGGGCGCCGGTGGTG
GGCATCCTGGTGCTCGACGGACACAGCGTACCCCTTCCCGAGCCCCTCGAAGTGGCCCATCATCTGTCGAAGAGCC
CTGACATGCAGGGAAGCCACCAGCTGCTCGTCGTATCAGAGGAGCAGTTCAAGGTGTTCACGCTGCCCAAGGTGAG
TGCCAAGCTGAAGTTGAAGCTGACGGCCCTGGAGGGCTCAAGAGTGCGGCGGGTCAGCGTGGCCCACTTCGGCAGT
CGTCGAGCCGAGGACTACGGGGAGCACCACCTGGCAGTCCTTACCAACCTGGGCGACATCCAGGTGGTCTCGCTGC
CCCTGCTCAAGCCCCAGGTGCGCTACAGCTGCATCCGCCCGGAGGACGTCAGTGGCATCGCCTCCTGCGTCTTCAC
CAAATATGGCCAAGGCTTCTACCTGATCTCACCCTCGGAGTTTGAGCGCTTCTCTCTCTCCACCAAGTGGCTCGTG
GAGCCCCGGTGTCTGGTGGATTCAGCAGAAACCAAGAACCACCGCCCTGGTAACGGTGCGGGCCCCAAGAAGGCCC
CGAGCCGAGCCAGCAACTCAGGGACTCAGAGTGATGGCGAGGAGAAGCAGCCCGGCCTGGTGATGGAGCGCGCTCT
GCTCAGTGATGAGAGAGCGGCAACTGGCGTTCACATCGAGCCGCCGTGGGGTGCAGCCTCAGCAATCGCGGAGCAG
AGTGAGTGGCTCAGCGTCCAGGCTGCGCGATGAGCACACACTACTACTGATGGCCTTTCGGGGGTCCCTGCCCCAA
CCGGAGAGGCCGGTGCACAGGGCCCCGCCAGGGGCTGGGGGCATCCCGGCTTCCACAATGCAGCTGCTCTGGGCCT
CGGGAGAGGAGAGACCCCAGTCCCCTGGGCTGCCCTTCCCGGGCCTCGTCTGTCTGGGTCCTTTGGTCAATGTTGC
ACAGTTTTTATTGCTCCCATCCCTTTTTGTAGTGGGCTGGGTTTTAAGTTATAAATGTTAACTGCCTCTGGGTGAA
AAAGTTTTTAATAAACACCTATTACCTCTTG

The sequence of NOV6a was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The cDNA coding for the NOV6a sequence was cloned by the polymerase chain reaction (PCR). Primers were designed based on in silico predictions of the full length or some portion (one or more exons) of the cDNA/protein sequence of the invention, or by translated homology of the predicted exons to closely related human sequences or to sequences from other species. The DNA sequence and protein sequence for a novel larvae-like gene were obtained by exon linking and are reported here as NOV6a. These primers and methods used to amplify NOV6a cDNA are described in the Examples.

The NOV6a polypeptide (SEQ ID NO:20) encoded by SEQ ID NO:19 is 904 amino acid residues in length and is presented using the one-letter amino acid code in Table 6B. The SignalP, Psort and/or Hydropathy results predict that NOV6a has no known signal peptide and is likely to be localized in the nucleus with a certainty of 0.9600. In alternative embodiments, a NOV6a polypeptide is located to the microbody (peroxisome) with a certainty of 0.5072, the mitochondrial matrix space with a certainty of 0.3600, or the lysosome (lumen) with a certainty of 0.1695.

TABLE 6B

Encoded NOV6a protein sequence (SEQ ID NO:29).

MRRFLRPGHDPVRERLKRDLFQFNKTVEHGFPHQPSALGYSPSLHILAIGTRSGAIKLYGAPGVEFMGLHQ
ENNAVTQIHLLPGQCQLVTLLDDNSLHLWSLKVKGGASELQEDESFTLRGPPGAAPSATQITVVLPHSSCE
LLYLGTESGNVFVVQLPAFRALEDRTISSDAVLQRLPEEARHRRVFEMVEALQEHPRDPNQILIGYSRGLV
VIWDLQGSRVLYHFLSSQQLENIWWQRDGRLLVSCHSDGSYCQWPVSSEAQQPEPLRSLVPYGPFPCKAIT
RILWLTTRQGLPFTIFQGGMPRASYGDRHCISVIHDGQQTAFDFTSRVIGTVRFWDASGVCLRLLYKLSTV
RVFLTDTDPNENFSAQGEDEWPPLRKVGSFDPYSDDPRLGIQKIFLCKYSGYLAVAGTAGQVLVLELNDEA
AEQAVEQVEADLLQDQEGYRWKGHERLAARSGPVRFEPGFQPFVLVQCQPPAVVTSLALHSEWRLVAPGTS
HGFGLFDHQQRRQVFVKCTLHPSDQLALEGPLSRVKSLKKSLRQSFRRMRRSRVSSRKRHPAGPPGEAQEG
SAKAERPGLQNMELAPVQRKIEARSAEDSFTGFVRTLYFADTYLKDSSRHCPSLWAGTNGGTIYAFSLRVP
PAERRMDEPVRAEQAKEIQLMHRAPVVGILVLDGHSVPLPEPLEVAHDLSKSPDMQGSHQLLVVSEEQFKV
FTLPKVSAKLKLKLTALEGSRVRRVSVAHFGSRRAEDYGEHHLAVLTNLGDIQVVSLPLLKPQVRYSCIRR
EDVSGIASCVFTKYGQGFYLISPSEFERFSLSTKWLVEPRCLVDSAETKNHRPGNGAGPKKAPSRARNSGT
QSDGEEKQPGLVMAERALLSDERAATGVHIEPPWGAASAMAEQSEWLSVQAAR

NOV6b

In an alternative embodiment, a NOV6 variant is NOV6b (alternatively referred to herein as CG55891-02), which includes the 3492 nucleotide sequence (SEQ ID NO:21) shown in Table 6C. An open reading frame for the mature protein was identified beginning at nucleotides 131-133 and ending at nucleotides 3188-3190. The start and stop codons of the open reading frame are highlighted in bold type. Putative untranslated regions are underlined, and found upstream from the initiation codon and downstream from the termination codon.

TABLE 6C
NOV6b Nucleotide Sequence.

(SEQ ID NO:21)

CGCCCAGCAGCCCGTGGGCAGGCGCGGCGGAGCGAGCGGGGCCGGCGGCGGGCGCCGAGGGACGCCGAGGCC
TCGGGCGGGGGCTGGCCCGGGGTTCCAGGTCTCCAGTGGGGGCTGCAGACTAAGCAAAATGAGGCGGTTCCT
GAGGCCAGGGCATGACCCTGTGCGGGAGAGGCTCAAGCGGCACCTGTTCCAGTTTAACAAGACGGTGGAGCA
TGGCTTCCCGCACCAGCCCAGCGCCCTCGGCTACAGCCCGTCCCTGCACATCCTGGCCATCGGCACCCGTTC
TGGAGCCATCAAGCTCTACGGAGCCCCAGGCGTGGAGTTCATGGGGCTGCACCAGGAGAACAACGCTGTGAC
GCAGATCCACCTCCTGCCCGGCCAGTGCCAGCTGGTCACCCTGCTGGATGACAACAGCCTGCACCTTTGGAG
CCTGAAGGTCAAGGGCGGGGCATCGGAGCTGCAGGAGGATGAGAGCTTCACACTGCGTGGACCCCCAGGGGC
TGCCCCCAGTGCCACACAGATCACCGTGGTCCTGCCACATTCCTCCTGCGAGCTGCTCTACCTGGGCACCGA
GAGTGGCAACGTGTTTGTGGTGCAGCTGCCAGCTTTTCGTGCGCTGGAGGACCGGACCATCAGCTCGGACGC
GGTGCTGCAGCGGTTGCCAGAGGAGGCCCGCCACCGGCGTGTGTTCGAGATGGTGGAGGCACTGCAGGAGCA
CCCTCGAGACCCCAACCAGATCCTGATCGGCTACAGCCGAGGCCTCGTTGTCATCTGGGACCTACAGGGCAG
CCGCGTGCTCTACCACTTCCTCAGCAGCCAGCAACTGGAGAACATCTGGTGGCAGCGGGACGGCCGCCTGCT
CGTCAGCTGTCACTCTGACGGCAGCTACTGCCAGTGGCCCGTGTCCAGCGAAGCCCAGCAACCAGAGCCCCT
CCGCAGCCTCGTGCCTTACGGTCCCTTTCCTTGCAAAGCGATTACCAGAATCCTCTGGCTGACCACTAGGCA
GGGGTTGCCCTTCACCATCTTCCAGGGTGGCATGCCACGGGCCAGCTACGGGGACCGCCACTGCATCTCAGT
GATCCACGATGGCCAGCAGACGGCCTTCGACTTCACCTCCCGTGTCATCGGCTTCACTGTCCTCACAGAGGC
AGACCCTGCAGCCACCTTTGACGACCCCTATGCCCTGGTGGTGCTGGCTGAGGAGGAGCTGGTGGTGATTGA
CCTGCAGACAGCAGGCTGGCCACCGGTCCAGCTGCCCTACCTGGCTTCTCTGCACTGTTCCGCCATCACCTG
CTCTCACCACGTCTCCAACATCCCGCTGAAGCTGTGGGAGCGGATCATTGCCGCCGGCAGCCGGCAGAACGC
ACACTTCTCCACCATGGAGTGGCCAATTGATGGTGGCACCAGCCTGACCCCAGCCCCACCCCAGAGGGACCT
GCTGCTCACAGGGCACGAGGACGGCACGGTGCGGTTCTGGGATGCCTCGGGTGTCTGCCTGCGGCTGCTCTA
CAAACTCAGCACTGTGCGCGTGTTCCTCACCGACACGGACCCCAACGAGAACTTCAGTGCCCAGGGCGAGGA
CGAGTGGCCCCCACTCCGCAAGGTGGGCTCCTTTGACCCCTACAGTGATGACCCCCGGCTGGGCATCCAGAA
GATCTTCCTCTGCAAGTACAGCGGCTACCTGGCTGTGGCAGGCACGGCAGGGCAGGTGCTGGTACTGGAACT
GAATGACGAGGCAGCGGAGCAGGCTGTGGAGCAGGTGGAGGCCGACCTGCTGCAGGACCAAGAGGGCTACCG
CTGGAAGGGGCACGAGCGCCTGGCAGCCCGCTCAGGGCCCGTGCGCTTTGAGCCTGGCTTTCAGCCCTTCGT
GTTGGTGCAGTGTCAGCCCCCGGCTGTGGTCACCTCCTTGGCCCTGCACTCTGAGTGGCGGCTCGTGGCCTT
CGGCACCAGCCATGGCTTTGGCCTCTTTGACCACCAGCAGCGGCGGCAGGTCTTTGTTAAGTGCACACTGCA
CCCCAGTGACCAGCTGGCCTTGGAGGGCCCACTCTCCCGCGTCAAGTCCCTCAAGAAGTCCTTGCGTCAGTC
ATTCCGCCGGATGCGTCGGAGCCGGGTGTCCAGCCGGAAGCGGCACCCGGCTGGCCCCCCAGGAGAGGTGAG
GCCTGAGGCACAGGAGGGGAGTGCCAAGGCTGAGCGGCCAGGCCTCCAGAACATGGAGCTGGCGCCTGTGCA
GCGCAAGATCGAGGCTCGCTCGGCAGAGGACTCCTTCACAGGCTTCGTCCGGACCCTGTACTTTGCTGACAC
CTACCTGAAGGACAGCTCCCGGCACTGCCCCTCGCTGTGGGCTGGCACCAATGGGGGCACCATCTATGCCTT
CTCCCTGCGTGTGCCTCCCGCCGAGCGGAGAATGGATGAGCCTGTGCGGGCAGAGCAGGCCAAGGAGATCCA
GCTGATGCACCGGGCGCCGGTGGTGGGCATCCTGGTGCTCGACGGACACAGCGTACCCCTTCCCGAGCCCCT
CGAAGTGGCCCATGATCTGTCGAAGAGCCCTGACATGCAGGGAAGCCACCAGCTGCTCGTCGTATCAGAGGA
GCAGTTCAAGGTGTTCACGCTGCCCAAGGTGAGTGCCAAGCTGAAGTTGAAGCTGACGGCCCTGGAGGGCTC
AAGAGTGCGGCGGGTCAGCGTGGCCCACTTCGGCAGTCGTCGAGCCGAGGACTACGGGGAGCACCACCTGGC
AGTCCTTACCAACCTGGGCGACATCCAGGTGGTCTCGCTGCCCCTGCTCAAGCCCCAGGTGCGCTACAGCTG
CATCCGCCGGGAGGACGTCAGTGGCATCGCCTCCTGCGTCTTCACCAAATATGGCCAAGGCTTCTACCTGAT
CTCACCCTCGGAGTTTGAGCGCTTCTCTCTCTCCACCAAGTGGCTGGTGGAGCCCCGGTGTCTGGTGGATTC
AGCAGAAACCAAGAACCACCGCCCTGGTAACGGTGCGGGCCCCAAGAAGGCCCCGAGCCGAGCCAGGAACTC
AGGGACTCAGAGTGATGGCGAGGAGAAGCAGCCCGGCCTGGTGATGGAGCGCGCTCTGCTCAGTGATGAGAG
AGCGGCAACTGGCGTTCACATCGAGCCGCCGTGGGGTGCAGCCTCAGCAATGGCGGAGCAGAGTGAGTGGCT
GAGCGTCCAGGCTGCGCGATGAGCACACACTACTACTGATGGCCTTTCGGGGGTCCCTGCCCCAACCGGAGA
GGCCGGTGCACAGGGCCCCGCCAGGGGCTGGGGGCATCCCGGCTTCCACAATGCAGCTGCTCTGGGCCTCGG
GAGAGGAGAGACCCCAGTCCCCTGGGCTGCCCTTCCCGGGCCTCGTCTGTCTGGGTCCTTTGGTCAATGTTG
CACAGTTTTTATTGCTCCCATCCCTTTTTGTAGTGGGCTGGGTTTTAAGTTATAAATGTTAACTGCCTCTGG
GTGAAAAAGTTTTTAATAAACACCTATTACCTCTTG

The sequence of NOV6b was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The cDNA coding for the NOV6b sequence was cloned by the polymerase chain reaction (PCR). PCR primers were designed based on in silico predictions of the full length or some portion (one or more exons) of the cDNA/protein sequence of the invention. The DNA sequence and protein sequence for a novel larvae-like gene were obtained by exon linking, or SeqCalling™ Technology and are reported here as NOV6b. These primers and methods used to amplify NOV6b cDNA are described in the Examples.

The NOV6b polypeptide (SEQ ID NO:22) encoded by SEQ ID NO:21 is 1019 amino acid residues in length and is presented using the one-letter amino acid code in Table 6D. The SignalP, Psort and/or Hydropathy results predict that NOV6b has no known signal peptide and is likely to be localized in the nucleus with a certainty of 0.9600. In alternative embodiments, a NOV6a polypeptide is located to the microbody (peroxisome) with a certainty of 0.5028, the mitochondrial matrix space with a certainty of 0.3600, or the lysosome (lumen) with a certainty of 0.1782.

TABLE 6D
Encoded NOV6b protein sequence.

(SEQ ID NO:22)

MRRFLRPGHDPVRERLKRDLFQFNKTVEHGFPHQPSALGYSPSLHILAIGTRSGAIKLYGAPGVEFMGLHQ
ENNAVTQIHLLPGQCQLVTLLDDNSLHLWSLKVKGGASELQEDESFTLRGPPGAAPSATQITVVLPHSSCE
LLYLGTESGNVFVVQLPAFRALEDRTISSDAVLQRLPEEARHRRVFEMVEALQEHPRDPNQILIGYSRGLV
VIWDLQGSRVLYHFLSSQQLENIWWQRDGRLLVSCHSDGSYCQWPVSSEAQQPEPLRSLVPYGPFPCKAIT
RILWLTTRQGLPFTIFQGGMPRASYGDRHCISVIHDGQQTAFDFTSRVIGFTVLTEADPAATFDDPYALVV
LAEEELVVIDLQTAGWPPVQLPYLASLHCSAITCSHHVSNIPLKLWERIIAAGSRQNAHFSTMEWPIDGGT
SLTPAPPQRDLLLTGHEDGTVRFWDASGVCLRLLYKLSTVRVFLTDTDPNENFSAQGEDEWPPLRKVGSFD
PYSDDPRLGIQKIFLCKYSGYLAVAGTAGQVLVLELNDEAAEQAVEQVEADLLQDQEGYRWKGHERLAARS
GPVRFEPGFQPFVLVQCQPPAVVTSLALHSEWRLVAFGTSHGFGLFDHQQRRQVFVKCTLHPSDQLALEGP
LSRVKSLKKSLRQSFRRMRRSRVSSRKRHPAGPPGEVRPEAQEGSAKAERPGLQNMELAPVQRKIEARSAE
DSFTGFVRTLYFADTYLKDSSRHCPSLWAGTNGGTIYAFSLRVPPAERRMDEPVRAEQAKEIQLMHRAPVV
GILVLDGHSVPLPEPLEVAHDLSKSPDMQGSHQLLVVSEEQFKVFTLPKVSAKLKLKLTALEGSRVRRVSV
AHFGSRRAEDYGEHHLAVLTNLGDIQVVSLPLLKPQVRYSCIRREDVSGIASCVFTKYGQGFYLISPSEFE
RFSLSTKWLVEPRCLVDSAETKNHRPGNGAGPKKAPSRARNSGTQSDGEEKQPGLVMERALLSDERAATGV
HIEPPWGAASAMAEQSEWLSVQAAR

NOV6 Clones

Unless specifically addressed as NOV6a or NOV6b, any reference to NOV6 is assumed to encompass all variants. Residue differences between any NOVX variant sequences herein are written to show the residue in the “a” variant, the residue position with respect to the “a” variant, and the residue in the “b” variant.

The amino acid sequence of NOV6 has high homology to other proteins as shown in Table 6E.

TABLE 6E
BLASTX results from Patp database for NOV6

| | | | Smallest |
|---|---|---|---|
| | | | Sum |
| | | High | Prob |
| Sequences producing High-scoring Segment Pairs: | | Score | P(N) |
| patp: AAG75075 | Human colon cancer antigen protein | 1207 | 1.2e−122 |
| patp: AAB53381 | Human colon cancer antigen protein sequence | 154 | 9.9e−10 |
| patp: AAB68519 | Human GTP-binding associated protein | 75 | 0.026 |
| patp: AAG01592 | Human secreted protein | 84 | 0.11 |
| patp: AAU00325 | Fertilisation-independent endosperm protein | 92 | 0.64 |

In a search of sequence databases, it was found, for example, that the NOV6a nucleic acid sequence has 3147 of 3480 bases (90%) identical to a giant larvae homolog mRNA from Homo sapiens (GENBANK-ID: HSHGLHOMO|acc:X87342). Further, the full amino acid sequence of the disclosed NOV6a protein of the invention has 904 of 1015 amino acid residues (89%) identical to, and 904 of 1015 amino acid residues (89%) similar to GIANT LARVAE HOMOLOGUE protein from Homo sapiens (ACC:Q14521). NOV6a is a novel variant of the 1015 amino acid residue GIANT LARVAE HOMOLOGUE protein from Homo sapiens (ACC:Q14521). The disclosed NOV6a protein lacks 111 amino acids compared to the GIANT LARVAE HOMOLOGUE protein.

In a similar search of sequence databases, it was found, for example, that the NOV6b nucleic acid sequence has 2464 of 2723 bases (90%) identical to a gb:GENBANK-ID:HSHGLHOMO|acc:X87342.1 mRNA from Homo sapiens (H.sapiens mRNA for human giant larvae homolog). Further, the full amino acid sequence of the protein of the invention was found to have 1015 of 1019 amino acid residues (99%) identical to, and 1015 of 1019 amino acid residues (99%) similar to, the 1015 amino acid residue ptnr:SPTREMBL-ACC:Q14521 protein from Homo sapiens (Human) (GIANT LARVAE HOMOLOGUE). The amino acid sequence of the disclosed NOV6b protein of the invention has an insertion of 4 internal amino acids compared to ptnr:SPTREMBL-ACC:Q14521 protein from Homo sapiens (Human) (GIANT LARVAE HOMOLOGUE).

Additional BLASTP results are shown in Table 6F.

TABLE 6F
NOV6 BLASTP results

| Gene Index/ | | Length | Identity | Positives | |
|---|---|---|---|---|---|
| Identifier | Protein/Organism | (aa) | (%) | (%) | Expect |
| Q14521 | GIANT LARVAE HOMOLOGUE - | 1015 | 571/571 | 571/571 | 0.0 |
| | Homo sapiens (Human) | | (100%) | (100%) | |
| AAK52346 | LETHAL GIANT LARVAE-LIKE | 1027 | 491/545 | 508/545 | 0.0 |
| | PROTEIN 2 - Mus musculus | | (90%) | (93%) | |
| | (Mouse) | | | | |
| Q61856 | ORF - Mus musculus (Mouse) | 1034 | 320/538 | 403/538 | 4.2e−257 |
| | | | (59%) | (74%) | |
| Q15334 | TUMOUR SUPPRESSOR PROTEIN, | 1057 | 310/510 | 384/510 | 6.9e−246 |
| | HUGL - Homo sapiens | | (50%) | (75%) | |
| | (Human) | | | | |
| O00188 | LLGL - Homo sapiens | 1032 | 309/541 | 390/541 | 1.2e−241 |
| | (Human) | | (57%) | (72%) | |

A multiple sequence alignment is given in Table 6G, with the NOV6 protein of the invention being shown in line 1 and 2, in a ClustalW analysis comparing NOV6 with related protien sequences of Table 4F.

[image]

[image]

[image]

[image]

[image]

DOMAIN results for NOV6 were collected from the Pfam database, and then identified by the Interpro domain accession number. The results are listed in Table 6H with the statistics and domain description. This indicates that the NOV6 polypeptides have properties similar to those of other proteins known to contain these domains.

TABLE 6H
Domain Analysis of NOV6

| | | |
|---|---|---|
| | Score | E |
| PSSMs producing significant alignments: | (bits) | value |

| | | | |
|---|---|---|---|
| WD domain, | G-protein beta repeat | 3.5 | 3.3e+02 |

| | | |
|---|---|---|
| WD | 1lrtl.ghsssvtslafdpdggllatgsaDgtvriwd | (SEQ ID NO:115) |
| | ++ ++ + + +++ ++++ + ++ + + + ++++ | |
| NOV6a | NKTVEhGFPHQPSALGYSPSLHILAIGTRSGAIKLYG | (SEQ ID NO:116) |

The NOV6 disclosed in this invention is expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. This information was derived by determining the tissue sources of the sequences that were included in the invention including but not limited to SeqCalling sources, Public EST sources, Literature sources, and/or RACE sources. In addition, NOV6 is predicted to be expressed in the following tissues because of the expression pattern of (GENBANK-ID: X05426) the lethal(2) giant larvae protein in species Drosophila melanogaster: eyes, ovaries, digestive tract, brain. Further tissue expression analysis is provided in the Examples.

The protein similarity information, expression pattern, and map location for the giant larvae-like protein and nucleic acid disclosed herein suggest that this protein may have important structural and/or physiological functions. Therefore, the nucleic acids and proteins of the invention are useful in potential diagnostic and therapeutic applications. For example, the compositions of the present invention will have efficacy for treatment of patients suffering from cancer, disorders of the eyes, ovaries, digestive tract, and brain, as well as other diseases, disorders and conditions.

The novel nucleic acid encoding the Drosophila larvae-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV6 protein has multiple hydrophilic regions, each of which can be used as an immunogen. In one embodiment, a contemplated NOV6 epitope is from about amino acids 5 to 45. In another embodiment, a contemplated NOV6 epitope is from about amino acids 60 to 90. In other specific embodiments, contemplated NOV6 epitopes are from about amino acids 100 to 125, 160 to 200, 220 to 260, 290 to 320, 350 to 400, 420 to 460, 490 to 510, 520 to 600, 610 to 630, 640 to 670, 675 to 710, 725 to 740, 740 to 760, 770 to 790, and 795 to 900.

NOV7

A disclosed NOV7 nucleic acid of 2011 nucleotides (also referred to dj1182a14_da1) encoding a novel Macrophage Stimulating Protein Precursor-like receptor protein is shown in Table 7A. An open reading frame was identified beginning with an ATG initiation codon at nucleotides 1-3 and ending with a TAG codon at nucleotides 1999-2001. A putative untranslated region is found downstream from the termination codon, and is underlined in Table 7A. The start and stop codons are in bold letters.

TABLE 7A
NOV7 Nucleotide Sequence

(SEQ ID NO:23)

ATGGGGTGGCTCCCACTCCTGCTGCTTCTGACTCAATGCTTAGGGGTCCCTGGTCAGCGCTCGCCATTGAA
TGACTTCCAAGTGCTCCGGGGCACAGAGCTACAGCACCTGCTACATGCGGTGGTGCCCGGGCCTTGGCAGG
AGGATGTGGCAGATGCTGAAGAGTGTGCTGGTCGCTGTGGGCCCTTAATGGACTGCCGGGCCTTCCACTAC
AACGTGAGCAGCCATGGTTGCCAACTGCTGCCATGGACTCAACACTCGCCCCACACGAGGCTGCGGCGTTC
TGGGCGCTGTGACCTCTTCCAGAAGAAAGACTACGTACGGACCTGCATCATGAACAATGGGGTTGGGTACC
GGGGCACCATGGCCACGACCGTGGGTGGCCTGCCCTGCCAGGCTTGGAGCCACAAGTTCCCAAATGATCAC
AAGTACACGCCCACTCTCCGGAATGGCCTGGAAGAGAACTTCTGCCGTAACCCTGATGGCGACCCCGGAGG
TCCCTGGTGCTACACAACAGACCCTGCTGTGCGCTTCCAGAGCTGCGGCATCAAATCCTGCCGGGAGGCCG
CGTGTGTCTGGTGCAATGGCGAGGAATACCGCGGCGCGGTAGACCGCACGGAGTCAGGGCGCGAGTGCCAG
CGCTGGGATCTTCAGCACCCGCACCAGCACCCCTTCGAGCCGGGCAAGTTCCTCGACCAAGGTCTGGACGA
CAACTATTGCCGGAATCCTGACGGCTCCGAGCGGCCATGGTGCTACACTACGGATCCGCAGATCGAGCGAG
AGTTCTGTGACCTCCCCCGCTGCGGTTCCGAGGCACAGCCCCGCCAAGAGGCCACAACTGTCAGCTGCTTC
CGCGGGAAGGGTGAGGGCTACCGGGGCACAGCCAATACCACCACCGCGGGCGTACCTTGCCAGCGTTGGGA
CGCGCAAATCCCGCATCAGCACCGATTTACGCCAGAAAAATACGCGTGCAAGGACCTTCGGGAGAACTTCT
GCCGGAACCCCGACGGCTCAGAGGCGCCCTGGTGCTTCACACTGCGGCCCGGCATGCGCGCGGCCTTTTGC
TACCAGATCCGGCGTTGTACAGACGACGTGCGGCCCCAGACTGCTACCACGGCGCAGGGGAGCAGTACCGC
GGCACGGTCAGCAAGACCCGCAAGGGTGTCCAGTGCCAGCGCTGGTCCGCTGAGACGCCGCACAAGCCGCA
GTTCACGTTTACCTCCGAACCGCATGCACAACTGGAGGAGAACTTCTGCCGGAACCCAGATGGGGATAGCC
ATGGGCCCTGGTGCTACACGATGGACCCAAGGACCCCATTCGACTACTGTGCCCTGCGACGCTGCGCTGAT
GACCAGCCGCCATCAATCCTGGACCCCCCAGACCAGGTGCAGTTTGAGAAGTGTGGCAAGAGGGTGGATCG
GCTGGATCAGCGGCGTTCCAAGCTGCGCGTGGTTGGGGGCCATCCGGGCAACTCACCCTGGACAGTCAGCT
TGCGGAATCGGTATGCTGCCTCTCACGGGCTATGAGGTATGGTTGGGCACCCTGTTCCAGAACCCACAGCA
TGGAGAGCCAAGCCTACAGCGGGTCCCAGTAGCCAAGATGGTGTGTGGGCCCTCAGGCTCCCAGCTTGTCC
TGCTCAAGCTGGAGAGATCTGTGACCCTGAACCAGCGTGTGGCCCTGATCTGCCTGCCCCCTGAATGGTAT
GTGGTGCCTCCAGGGACCAAGTGTGAGATTGCAGGCTGGGGTGAGACCAAAGGTACGGGTAATGACACAGT
CCTAAATGTGGCCTTGCTGAATGTCATCTCCAACCAGGAGTGTAACATCAAGCACCGAGGACGTGGTGACT
ACGGGGGCCCACTTGCCTGCTTTACCCACAACTGCTGGGTCCTGGAAGGAATTATAATCCCCAACCGAGTA
TGCGCAAGGTCCTGCTGGCCAGCTGTCTTCACGCGTGTCTCTGTGTTTGTGGACTGGATTCACAAGGTCAT
GAGACTGGGTTAGGCCCAGCCTT

The disclosed NOV7 nucleic acid sequence, localized to the q21 region of chromosome 3, has 1508 of 1524 bases (98%)identical to a gb:GENBANK-ID:HUMMST1A|acc:L11924 mRNA from Homo sapiens(Homo sapiens macrophage-stimulating protein (MST1) mRNA, complete cds (E=0.0).

A disclosed NOV7 polypeptide (SEQ ID NO:24) encoded by SEQ ID NO:23 is 666 amino acid residues long and is presented using the one-letter amino acid code in Table 7B. Signal P, Psort and/or Hydropathy results predict that NOV7 contains a signal peptide and is likely to be localized in the lysosome (lumen) with a certainty of 0.5493. In other embodiments, NOV7 is also likely to be localized extracellularly with a certainty of 0.3700, to the microbody (peroxisome) with a certainty of 0.1588, and the endoplasmic reticulum (membrane) with a certainty of 0.1000. The most likely cleavage site for a NOV7 peptide is between amino acids 18 and 19, at: VPG-QR.

TABLE 7B
Encoded NOV7 protein sequence.

(SEQ ID NO:24)

MGWLPLLLLLTQCLGVPGQRSPLNDFQVLRGTELQHLLHAVVPGPWQEDVADAEECAGRCGPLMDCRAFHY
NVSSHGCQLLPWTQHSPHTRLRRSGRCDLFQKKDYVRTCIMNNGVGYRGTMATTVGGLPCQAWSHKFPNDH
KYTPTLRNGLEENFCRNPDGDPGGPWCYTTDPAVRFQSCGIKSCREAACVWCNGEEYRGAVDRTESGRECQ
RWDLQHPHQHPFEPGKFLDQGLDDNYCRNPDGSERPWCYTTDPQIEREFCDLPRCGSEAQPRQEATTVSCF
RGKGEGYRGTANTTTAGVPCQRWDAQIPHQHRFTPEKYACKDLRENFCRNPDGSEAPWCFTLRPGMRAAFC
YQIRRCTDDVRPQTATTAQGSSTAARSARPARVSSASAGPLRRRTSRSSRLPPNRMHNWRRTSAGTQMGIA
MGPGATRWTQGPHSTTVPCDAALMTSRHQSWTPQTRCSLRSVARGWIGWISGVPSCAWLGAIRATHPGQSA
CGIGMLPLTGYEVWLGTLFQNPQHGEPSLQRVPVAKMVCGPSGSQLVLLKLERSVTLNQRVALICLPPEWY
VVPPGTKCEIAGWGETKGTGNDTVLNVALLNVISNQECNIKHRGRGDYGGPLACFTHNCWVLEGIIIPNRV
CARSCWPAVFTRVSVFVDWIHKVMRLG

The NOV7 amino acid sequence has 368 of 368 amino acid residues (100%) identical to, and 368 of 368 amino acid residues (100%) similar to, the 711 amino acid residue ptnr:SPTREMBL-ACC:Q14870 protein from Homo sapiens (Human) Macrophage-Stimulating Protein Precursor (E=9.9e⁻³¹⁰).

NOV7 is expressed in at least HepG2 (liver cell line). SNP data for NOV7 can be found below in Example 3.

NOV7 also has homology to the amino acid sequence shown in the BLASTP data listed in Table 7C.

TABLE 1C
BLAST results for NOV7

| Gene Index/ | | Length | Identity | Positives | |
|---|---|---|---|---|---|
| Identifier | Protein/Organism | (aa) | (%) | (%) | Expect |
| gi\|11544434\|emb\| | dJ1182A14.3 | 648 | 454/717 | 474/717 | 0.0 |
| CAC17639.1\|(AL137798) | (similar to MST1 | | (63%) | (65%) | |
| | (macrophage | | | | |
| | stimulating 1 | | | | |
| | (hepatocyte | | | | |
| | growth factor- | | | | |
| | like))) [Homo | | | | |
| | sapiens] | | | | |
| gi\|10337615\|ref\| | macrophage | 711 | 360/367 | 360/367 | 0.0 |
| NP_066278.1\| | stimulating 1 | | (98%) | (98%) | |
| | (hepatocyte | | | | |
| | growth factor- | | | | |
| | like) [Homo | | | | |
| | sapiens] | | | | |
| gi\|15294659\|ref\| | macrophage | 711 | 360/367 | 360/367 | 0.0 |
| XP_054070.1\| | stimulating 1 | | (98%) | (98%) | |
| | (hepatocyte | | | | |
| | growth factor- | | | | |
| | like) [Homo | | | | |
| | sapiens] | | | | |
| gi\|123114\|sp\|P26927\| | HEPATOCYTE GROWTH | 711 | 359/367 | 359/367 | 0.0 |
| HGFL_HUMAN | FACTOR-LIKE | | (97%) | (97%) | |
| | PROTEIN PRECURSOR | | | | |
| | (MACROPHAGE | | | | |
| | STIMULATORY | | | | |
| | PROTEIN) (MSP) | | | | |
| | (MACROPHAGE | | | | |
| | STIMULATING | | | | |
| | PROTEIN) | | | | |
| gi\|1141775\|gb\| | hepatocyte growth | 567 | 313/332 | 321/332 | 0.0 |
| AAC63092.1\|(U28054) | factor-like | | (94%) | (96%) | |
| | protein homolog | | | | |
| | [Homo sapiens] | | | | |

The homology of these sequences is shown graphically in the ClustalW analysis shown in Table 7D.

[image]

[image]

[image]

Tables 7E-K list the domain descriptions from DOMAIN analysis results against NOV7. This indicates that the NOV7 sequence has properties similar to those of other proteins known to contain these domains.

TABLE 7E
Domain Analysis of NOV7

gnl|Pfam|pfam00051, kringle, Kringle domain. Kringle domains have
been found in plasminogen, hepatocyte growth factors, prothrombin, and
apolipoprotein A. Structure is disulfide-rich, nearly all-beta.
(SEQ ID NO:122) CD-Length = 79 residues, 100.0% aligned
Score = 117 bits (292), Expect = 3e−27

| | | | |
|---|---|---|---|
| Query: | 191 | CVWCNGEEYRGAVDRTESGRECQRWDLQHPHQHPF-EPGKFLDQGLDDNYCRNPDGSERP | 249 |
| | | \| \|\|\| \|\|\| \|\|\|\| \|\|\|\|\| \| \|\|+\| \| ++ +\|\| +\|\|\|\|\|\|\|\| \|\|\| | |
| Sbjct: | 1 | CYHGNGENYRGTASTTESGAPCQRWDSQTPHRHSKYTPERYPAKGLGENYCRNPDGDERP | 60 |
| Query: | 250 | WCYTTDPQIEREFCDLPRC 268 | |
| | | \|\|\|\|\|\|\|++ \|+\|\|+\|\|\| | |
| Sbjct: | 61 | WCYTTDPRVRWEYCDIPRC 79 | |

TABLE 7F
Domain Analysis of NOV7

gnl|Pfam|pfam00051, kringle, Kringle domain. Kringle domains have been
found in plasminogen, hepatocyte growth factors, prothrombin, and
apolipoprotein A. Structure is disulfide-rich, nearly all-beta.
(SEQ ID NO:123) CD-Length = 79 residues, 100.0% aligned
Score = 112 bits (279), Expect = 9e−26

| | | | |
|---|---|---|---|
| Query: | 283 | CFRGKGEGYRGTANTTTAGVPCQRWDAQIPHQHRF-TPEKYACKDLRENFCRNPDGSEAP | 341 |
| | | \|+ \| \|\| \|\|\|\|\|+\|\| +\| \|\|\|\|\|\|+\| \|\|+\| \|\|\|+\| \| \| \|\|+\|\|\|\|\|\| \| \| | |
| Sbjct: | 1 | CYHGNGENYRGTASTTESGAPCQRWDSQTPHRHSKYTPERYPAKGLGENYCRNPDGDERP | 60 |
| Query: | 342 | WCFTLRPGMRAAFCYQIRRC 361 | |
| | | \|\|+\| \| +\| +\| \| \|\| | |
| Sbjct: | 61 | WCYTTDPRVRWEYC-DIPRC 79 | |

TABLE 7G
Domain Analysis of NOV7

gnl|Pfam|pfam00051, kringle, Kringle domain. Kringle domains have been
found in plasminogen, hepatocyte growth factors, prothrombin, and
apolipoprotein A. Structure is disulfide-rich, nearly all-beta.
(SEQ ID NO:124) CD-Length = 79 residues, 100.0% aligned
Score = 104 bits (259) , Expect = 2e−23

| | | | |
|---|---|---|---|
| Query: | 110 | CIMNNGVGYRGTMATTVGGLPCQAWSHKFPNDH-KYTPT--LRNGLEENFCRNPDGDPGG | 166 |
| | | \| \|\| \|\|\|\| +\|\| \| \|\|\| \| + \|+ \| \|\|\|\| \|\| \|\|+\|\|\|\|\|\|\| | |
| Sbjct: | 1 | CYHGNGENYRGTASTTESGAPCQRWDSQTPHRHSKYTPERYPAKGLGENYCRNPDGDE-R | 59 |
| Query: | 167 | PWCYTTDPAVRFQSCGIKSC 186 | |
| | | \|\|\|\|\|\|\|\| \|\|++ \| \| \| | |
| Sbjct: | 60 | PWCYTTDPRVRWEYCDIPRC 79 | |

TABLE 7H
Domain Analysis of NOV7

gnl|Smart|smart00130, KR, Kringle domain; Named after a Danish pastry.
Found in several serine proteases and in ROR-like receptors. Can occur
in up to 38 copies (in apolipoprotein (a)). Plasminogen-like kringles
possess affinity for free lysine and lysine- containing peptides.
(SEQ ID NO:125) CD-Length = 83 residues, 97.6% aligned
Score = 111 bits (278), Expect = 1e−25

| | | | |
|---|---|---|---|
| Query: | 191 | CVWCNGEEYRGAVDRTESGRECQRWDLQHPHQHPFEPGKFLDQGLDDNYCRNPDG-SERP | 249 |
| | | \| \|\|\| \|\|\| \|+\|\|+ \|\|\|\|\| \| \|\| \| \| \| +\| + \|\|+ \|\|\|\|\|\|\|\| \|\| \| | |
| Sbjct: | 3 | CYAGNGESYRGTASTTKSGKPCQRWDSQTPHLHRFTPERFPELGLEHNYCRNPDGDSEGP | 62 |
| Query: | 250 | WCYTTDPQIEREFCDLPRCGS 270 | |
| | | \|\|\|\|\|\|\| + \|+\|\|+\|+\| \| | |
| Sbjct: | 63 | WCYTTDPNVRWEYCDIPQCES 83 | |

TABLE 7I
Domain Analysis of NOV7

gnl|Smart|smart00130, KR, Kringle domain; Named after a Danish pastry.
Found in several serine proteases and in ROR-like receptors. Can occur
in up to 38 copies (in apolipoprotein(a)). Plasminogen-like kringles
possess affinity for free lysine and lysine- containing peptides.
(SEQ ID NO:126) CD-Length = 83 residues, 97.6% aligned
Score = 106 bits (265), Expect = 4e−24

| | | | |
|---|---|---|---|
| Query: | 108 | RTCIMNNGVGYRGTMATTVGGLPCQAWSHKFPNDHKYTPTLRN--GLEENFCRNPDGDPG | 165 |
| | | \| \| \|\| \|\|\|\| +\|\| \| \|\|\| \| + \|+ \|++\|\| \|\|\| \|+\|\|\|\|\|\|\| | |
| Sbjct: | 1 | RDCYAGNGESYRGTASTTKSGKPCQRWDSQTPHLHRFTPERFPELGLEHNYCRNPDGDSE | 60 |
| Query: | 166 | GPWCYTTDPAVRFQSCGIKSC | 186 |
| | | \|\|\|\|\|\|\|\|\| \|\|++ \| \| \| | |
| Sbjct: | 61 | GPWCYTTDPNVRWEYCDIPQC | 81 |

TABLE 7J
Domain Analysis of NOV7

gnl|Smart|smart00130, KR, Kringle domain; Named after a Danish pastry.
Found in several serine proteases and in ROR-like receptors. Can occur
in up to 38 copies (in apolipoprotein (a)). Plasminogen-like kringles
possess affinity for free lysine and lysine- containing peptides.
(SEQ ID NO:127)
CD-Length = 83 residues, 97.6% aligned
Score = 104 bits (260), Expect = 1e−23

| | | | |
|---|---|---|---|
| Query: | 283 | CFRGKGEGYRGTANTTTAGVPCQRWDAQIPHQHRFTPEKYACKDLRENFCRNPDG-SEAP | 341 |
| | | \|+ \| \|\| \|\|\|\|\|+\|\| +\| \|\|\|\|\|\|+\| \|\| \|\|\|\|\|\|++ \| \|+\|\|\|\|\|\| \|\| \| | |
| Sbjct: | 3 | CYAGNGESYRGTASTTKSGKPCQRWDSQTPHLHRFTPERFPELGLEHNYCRNPDGDSEGP | 62 |
| Query: | 342 | WCFTLRPGMRAAFCYQIRRCTD | 363 |
| | | \|\|+\| \| +\| +\| \| +\| | |
| Sbjct: | 63 | WCYTTDPNVRWEYCD-IPQCES | 83 |

TABLE 7K
Domain Analysis of NOV7

gnl|Smart|smart00473, PAN_AP, divergent subfamily of APPLE domains;
Apple-like domains present in Plasminogen, C. elegans hypothetical
ORFs and the extracellular portion of plant receptor-like protein
kinases. Predicted to possess protein- and or carbohydrate-binding
functions (SEQ ID NO:128)
CD-Length = 79 residues, 94.9% aligned
Score = 52.0 bits (123), Expect = 1e−07

| | | | |
|---|---|---|---|
| Query: | 25 | DFQVLRGTELQHLLHAVVPGPWQEDVADAEECAGRC-GPLMDCRAFHYNVSSHGCQLLPW | 83 |
| | | \| \| \|+\| \| \|\| \|\|\|\| +\| \|\|+\| \|\| + \|\| \| | |
| Sbjct: | 5 | CFVRLPNTKL------PDFSPIVISVASLEECAQKCLNSNCSCRSFTYNNDTKGCLLWSE | 58 |
| Query: | 84 | TQHSPHTRLRRSGRCDLFQKK | 104 |
| | | + +\| \|\| \| ++\| | |
| Sbjct: | 59 | SSLGDARQLLPSGGVDYYEKI | 79 |

NOV7 also had homology to proteins in the PATP database as shown by the BLAST data in Table 7L below.

TABLE 7L
BLAST PATP results for NOV7

| Gene Index/ | | Length | Identity | Positives | Ex- |
|---|---|---|---|---|---|
| Identifier | Protein/Organism | (aa) | (%) | (%) | pect |
| patp: | Human L5/3 tumour | 711 | 368/368 | 368/368 | 0.0 |
| AAR66602 | suppressor | | (100%) | (100%) | |
| | protein | | | | |
| patp: | Amino acid | 710 | 368/368 | 368/368 | 0.0 |
| AAB84520 | sequence of human | | (100%) | (100%) | |
| | macrophage | | | | |
| | stimulating | | | | |
| | protein (MSP) | | | | |

Macrophage-stimulating protein (MSP) is an 80-kD serum protein with homology to hepatocyte growth factor (HGF) (Sakamoto O, et.al.; J ClinInvest Feb. 15, 1997;99(4):701-9). Its receptor, RON tyrosine kinase, is anew member of the HGF receptor family. The MSP-RON signaling pathway has been implicated in the functional regulation of mononuclear phagocytes. However, the function of this pathway in other types of cells has not been elucidated. Here we show that in contrast to the HGF receptor, which was expressed at the basolateral surface, RON was localized at the apical surface of ciliated epithelia in the airways and oviduct. In addition, MSP was found in the bronchoalveolar space at biologically significant concentrations. MSP bound to RON on normal human bronchial epithelial cells with a high affinity (Kd=0.5 nM) and induced autophosphorylation of RON. Activation of RON by MSP led to a significant increase in ciliary beat frequency of human nasal cilia. These findings indicate that the ciliated epithelium of the mucociliary transport apparatus is a novel target of MSP.

The above defined information for NOV7 suggests that this NOV7 protein may function as a member of a Macrophage Stimulating Protein Precursor family. Therefore, the NOV7 nucleic acids and proteins of the invention are useful in potential therapeutic applications implicated in various diseases and disorders described below and/or other pathologies. For example, the NOV7 compositions of the present invention will have efficacy for treatment of patients suffering from Aicardi-Goutieres syndrome 1; Brugada syndrome; Deafness, autosomal recessive 6; Heart block, nonprogressive; Heart block, progressive, 2; Ichthyosiforme erythroderma, congenital, nonbullous; Long QT syndrome-3; Night blindness; congenital stationary; Pituitary ACTH-secreting adenoma; Small-cell cancer of lung; Ventricular fibrillation, idiopathic; entricular tachycardia, idiopathic; HIV infection, susceptibility/resistance to; Von Hippel-Lindau (VHL) syndrome; Cirrhosis; Transplantation. The NOV7 nucleic acid encoding Macrophage Stimulating Protein Precursor receptor-like protein, and the Macrophage Stimulating Protein Precursor receptor-like protein of the invention, or fragments thereof, may further be useful in diagnostic applications, wherein the presence or amount of the nucleic acid or the protein are to be assessed.

NOV7 nucleic acids and polypeptides are further useful in the generation of antibodies that bind immuno-specifically to the novel NOV7 substances for use in therapeutic or diagnostic methods. These antibodies may be generated according to methods known in the art, using prediction from hydrophobicity charts, as described in the “Anti-NOVX Antibodies” section below. The disclosed NOV7 protein has multiple hydrophilic regions, each of which can be used as an immunogen. In one embodiment, a contemplated NOV7 epitope is from about amino acid 30 to 40. In another embodiment, a NOV7 epitope is from about amino acid 60 to 80. In additional embodiments, NOV7 epitopes are from about amino acid 85 to 140, from about amino acid 150 to 190, from about amino acid 205 to 350, from about amino acid 360 to 470, from about amino acid 480 to 620, from about amino acid 700 to 750, from about amino acid 500 to 530, from about amino acid 570 to 600, and from about amino acid 605 to 660. This novel protein can be used in assay systems for functional analysis of various human disorders, which will help in understanding of pathology of the disease and development of new drug targets for various disorders.

NOV8

Yet another NOVX protein of the invention, referred to herein as NOV8, is a nucleotide-sugar transporter-like protein.

Nucleotide sugar transporters are mainly located in the Golgi membranes and carry nucleotide sugars, that are produced outside the Golgi apparatus, into the organelle, where they serve as substrates for the elongation of carbohydrate chains by glycosyltransferases. Thus, such transporters are indispensable for cellular glycoconjugate synthesis. Moreover, the disclosed NOV8 protein of the invention may have regulatory roles in producing the structural variety of cellular glycoconjugates.

The novel nucleotide-sugar transporter gene disclosed in this invention maps to chromosome 6. This information was assigned using OMIM and the electronic northern tool from Curatools to derive the the chromosomal mapping of the SeqCalling assemblies, Genomic clones, and/or EST sequences that were included in the invention.

Two alternative NOV8 nucleic acids and encoded polypeptides are provided, namely NOV8a and NOV8b.

NOV8a

In one embodiment, a NOV8 variant is NOV8a (alternatively referred to herein as 138531995), which encodes a novel nucleotide-sugar transporter-like protein and includes the 1463 nucleotide sequence (SEQ ID NO:25) shown in Table 8A. An open reading frame for the mature protein was identified beginning with an ATG initiation codon at nucleotides 165-167 and ending with a TGA codon at nucleotides 1461-1463. Putative untranslated regions upstream from the start codon is underlined in Table 8A, and the start and stop codons are in bold letters.

TABLE 8A
NOV8a Nucleotide Sequence.

| | |
|---|---|
| GCAGGGGCGGAGGGGGCCGCGGGGAGGGAGGCGGGAAGAGCGCGGCACTTCCGCTGGCCGCTG | (SEQ ID NO:25) |
| GCTCGCTGGCCGCTCCTGGAGGCGGCGGCGGGAGCGCAGGGGGCGCGCGGCCCGGGGACTCGC | |
| ATTCCCCGGTTCCCCCTCCACCCCACGCGGCCTGGACCATGGACGCCAGATGGTGGGCAGTGG | |
| TGGTGCTGGCTGCGTTCCCCTCCCTAGGGGCAGGTGGGGAGACTCCCGAAGCCCCTCCGGAGT | |
| CATGGACCCAGCTATGGTTCTTCCGATTTGTGGTGAATGCTGCTGGCTATGCCAGCTTTATGG | |
| TACCTGGCTACCTCATGGTGCAGTACTTCAGGCGGAAGAACTACCTGGAGACCGGTAGGGGCC | |
| TCTGCTTTCCCCTGGTGAAAGCTTGTGTGTTTGGCAATGAGCCCAAGGCCTCTGATGAGGTTC | |
| CCCTGGCGCCCCGAACAGAGGCGGCAGAGACCACCCCGATGTGGCAGGCCCTGAAGCTGCTCT | |
| TCTGTGCCACAGGGCTCCAGGTGTCTTATCTGACTTGGGGTGTGCTGCAGGAAAGAGTGATGA | |
| CCCGCAGCTATGGGGCCACAGCCACATCACCGGGTGAGCGCTTTACGGACTCGCAGTTCCTGG | |
| TGCTAATGAACCGAGTGCTGGCACTGATTGTGGCTGGCCTCTCCTGTGTTCTCTGCAAGCAGC | |
| CCCGGCATGGGGCACCCATGTACCGGTACTCCTTTGCCAGCCTGTCCAATGTGCTTAGCAGCT | |
| GGTGCCAATACGAAGCTCTTAAGTTCGTCAGCTTCCCCACCCAGGTGCTGGCCAAGGCCTCTA | |
| AGGTGATCCCTGTCATGCTGATGGGAAAGCTTGTGTCTCGGCGCAGCTACGAACACTGGGAGT | |
| ACCTGACAGCCACCCTCATCTCCATTGGGGTCAGCATGTTTCTGCTATCCAGCGGACCAGAGC | |
| CCCGCAGCTCCCCAGCCACCACACTCTCAGGCCTCATCTTACTGGCAGGTTATATTGCTTTTG | |
| ACAGCTTCACCTCAAACTGGCAGGATGCCCTGTTTGCCTATAAGATGTCATCGGTGCAGATGA | |
| TGTTTGGGGTCAATTTCTTCTCCTGCCTCTTCACAGTGGGCTCACTGCTAGAACAGGGGGCCC | |
| TACTGGAGGGAACCCGCTTCATGGGGCGACACAGTGAGTTTGCTGCCCATGCCCTGCTACTCT | |
| CCATCTGCTCCGCATGTGGCCAGCTCTTCATCTTTTACACCATTGGGCAGTTTGGGGCTGCCG | |
| TCTTCACCATCATCATGACCCTCCGCCAGGCCTTTGCCATCCTTCTTTCCTGCCTTCTCTATG | |
| GCCACACTGTCACTGTGGTGGGAGGGCTGGGGGTGGCTGTGGTCTTTGCTGCCCTCCTGCTCA | |
| GAGTCTACGCGCGGGGCCGTCTAAAGCAACGGGGAAAGAAGGCTGTGCCTGTTGAGTCTCCTG | |
| TGCAGAAGGTTTGA | |

The sequence of NOV8a was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The DNA sequence and protein sequence for a novel nucleotide-sugar transporter-like gene were obtained by SeqCalling™ Technology and are reported here as NOV8a. These primers and methods used to amplify NOV8a cDNA are described in the Examples.

The NOV8a polypeptide (SEQ ID NO:26) encoded by SEQ ID NO:25 is 432 amino acid residues in length and is presented using the one-letter amino acid code in Table 8B. The SignalP, Psort and/or Hydropathy results predict that NOV8a has a signal peptide and is likely to be localized in the plasma membrane with a certainty of 0.6400. In alternative embodiments, a NOV8a polypeptide is located to the Golgi body with a certainty of 0.4600, the endoplasmic reticulum (membrane) with a certainty of 0.3700, or the endoplasmic reticulum (lumen) with a certainty of 0.1000. The SignalP predicts a likely cleavage site for a NOV8a peptide is between amino acid positions 20 and 21, i.e. at the dash in the sequence GAG-GE.

TABLE 8B
Encoded NOV8a protein sequence.

(SEQ ID NO:26)

MDARWWAVVVLAAFPSLGAGGETPEAPPESWTQLWFFRFVVNAAGYASFMVPGYLMVQYFRRKNYLETGR
GLCFPLVKACVFGNEPKASDEVPLAPRTEAAETTPMWQALKLLFCATGLQVSYLTWGVLQERVMTRSYGA
TATSPGERFTDSQFLVLMNRVLALIVAGLSCVLCKQPRHGAPMYRYSFASLSNVLSSWCQYEALKFVSFP
TQVLAKASKVIPVMLMGKLVSRRSYEHWEYLTATLISIGVSMFLLSSGPEPRSSPATTLSGLILLAGYIA
FDSFTSNWQDALFAYKMSSVQMMFGVNFFSCLFTVGSLLEQGALLEGTRFMGRHSEFAAHALLLSICSAC
GQLFIFYTIGQFGAAVFTIIMTLRQAFAILLSCLLYGHTVTVVGGLGVAVVFAALLLRVYARGRLKQRGK
KAVPVESPVQKV

Additional SNP variants of NOV8a are disclosed in Example 3.

NOV8b

In an alternative embodiment, a NOV8 variant is NOV8b (alternatively referred to herein as CG111627-01), which includes the 1742 nucleotide sequence (SEQ ID NO:27) shown in Table 8C. An open reading frame for the mature protein was identified beginning at nucleotides 111-113 and ending at nucleotides 1407-1409. The start and stop codons of the open reading frame are highlighted in bold type. Putative untranslated regions (underlined), if any, are found upstream from the initiation codon and downstream from the termination codon.

TABLE 8C
NOV8b Nucleotide Sequence.

| | |
|---|---|
| CGGCCGCGTCGACGCTGGCCGCTCCTGGAGGCGGCGGCGGGAGCGCAGGGGGCGCGCGGCCCG | (SEQ ID NO:27) |
| GGGACTCGCATTCCCCGGTTCCCCCTCCACCCCACGCGGCCTGGACCATGGACGCCAGATGGT | |
| GGGCAGTGGTGGTGCTGGCTGCGTTCCCCTCCCTAGGGGCAGGTGGGGAGACTCCCGAAGCCC | |
| CTCCGGAGTCATGGACCCAGCTATGGTTCTTCCGATTTGTGGTGAATGCTGCTGGCTATGCCA | |
| GCTTTATGGTACCTGGCTACCTCCTGGTGCAGTACTTCAGGCGGAAGAACTACCTGGAGACCG | |
| GTAGGGGCCTCTGCTTTCCCCTGGTGAAAGCTTGTGTGTTTGGCAATGAGCCCAAGGCCTCTG | |
| ATGAGGTTCCCCTGGCGCCCCGAACAGAGGCGGCAGAGACCACCCCGATGTGGCAGGCCCTGA | |
| AGCTGCTCTTCTGTGCCACAGGGCTCCAGGTGTCTTATCTGACTTGGGGTGTGCTGCAGGAAA | |
| GAGTGATGACCCGCAGCTATGGGGCCACAGCCACATCACCGGGTGAGCGCTTTACGGACTCGC | |
| AGTTCCTGGTGCTAATGAACCGAGTGCTGGCACTGATTGTGGCTGGCCTCTCCTGTGTTCTCT | |
| GCAAGCAGCCCCGGCATGGGGCACCCATGTACCGGTACTCCTTTGCCAGCCTGTCCAATGTGC | |
| TTAGCAGCTGGTGCCAATACGAAGCTCTTAAGTTCGTCAGCTTCCCCACCCAGGTGCTGGCCA | |
| AGGCCTCTAAGGTGATCCCTGTCATGCTGATGGGAAAGCTTGTGTCTCGGCGCAGCTACGAAC | |
| ACTGGGAGTACCTGACAGCCACACTCATCTCCATTGGGGTCAGCATGTTTCTGCTATCCAGCG | |
| GACCAGAGCCCCGCAGCTCCCCAGCCACCACACTCTCAGGCCTCATCTTACTGGCAGGTTATA | |
| TTGCTTTTGACAGCTTCACCTCAAACTGGCAGGATGCCCTGTTTGCCTATAAGATGTCATCGG | |
| TGCAGATGATGTTTGGGGTCAATTTCTTCTCCTGCCTCTTCACAGTGGGCTCACTGCTAGAAC | |
| AGGGGGCCCTACTGGAGGGAACCCGCTTCATGGGGCGACACAGTGAGTTTGCTGCCCATGCCC | |
| TGCTACTCTCCATCTGCTCCGCATGTGGCCAGCTCTTCATCTTTTACACCATTGGGCAGTTTG | |
| GGGCTGCCGTCTTCACCATCATCATGACCCTCCGCCAGGCCTTTGCCATCCTTCTTTCCTGCC | |
| TTCTCTATGGCCACACTGTCACTGTGGTGGGAGGGCTGGGGGTGGCTGTGGTCTTTGCTGCCC | |
| TCCTGCTCAGAGTCTACGCGCGGGGCCGTCTAAAGCAACGGGGAAAGAAGGCTGTGCCTGTTG | |
| AGTCTCCTGTGCAGAAGGTTTGAGGGTGGAAAGGGCCTGAGGGGTGAAGTGAAATAGGACCCT | |
| CCCACCATCCCCTTCTGCTGTAACCTCTGAGGGAGCTGGCTGAAAGGGCAAAATGCAGGTGTT | |
| TTCTCAGTATCACAGACCAGCTCTGCAGCAGGGGATTGGGGAGCCCAGGAGGCAGCCTTCCCT | |
| TTTGCCTTAAGTCNACCCATCTTCCANGTAAGCAGTTTATTCTGAGCCCCGGGGGTAGACAGT | |
| CCTCAGTGAGGGGTTTTGGGGAGTTTGGGGTCAAGAGAGCATAGGTAGGTTCCACAGTTACTC | |
| TTCCCACAAGTTCCCTTAAGTCTTGCCCTAGCTGTGCTCTG | |

The sequence of NOV8b was derived by laboratory cloning of cDNA fragments, by in silico prediction of the sequence. The cDNA fragments covering either the full length of the DNA sequence, or part of the sequence, or both, were cloned. In silico prediction was based on sequences available in CuraGen's proprietary sequence databases or in the public human sequence databases, and provided either the full length DNA sequence, or some portion thereof.

The cDNA coding for the NOV8b sequence was cloned by the polymerase chain reaction (PCR). PCR primers were designed based on in silico predictions of the full length or some portion (one or more exons) of the cDNA/protein sequence of the invention. The DNA sequence and protein sequence for a novel nucleotide-sugar transporter-like gene were obtained by exon linking, or SeqCalling™ Technology and are reported here as NOV8b. These primers and methods used to amplify NOV8b cDNA are described in the Examples.

The NOV8b polypeptide (SEQ ID NO:26) encoded by SEQ ID NO:27 is identical to the NOV8a polypeptide (SEQ ID NO:26).

NOV8 Clones

Unless specifically addressed as NOV8a or NOV8b, any reference to NOV8 is assumed to encompass all variants. Residue differences between any NOVX variant sequences herein are written to show the residue in the “a” variant, the residue position with respect to the “a” variant, and the residue in the “b” variant.

The amino acid sequence of NOV8 has high homology to other proteins as shown in Table 8D.

TABLE 8D.
BLASTX results from Patp database for NOV8

| | | | Smallest |
|---|---|---|---|
| | | | Sum |
| | | High | Prob |
| Sequences producing High-scoring Segment Pairs: | | Score | P(N) |
| patp: AAB12162 | Hydrophobic domain protein | 2227 | 1.0e−230 |
| patp: AAB34721 | Human secreted protein | 2227 | 1.0e−230 |
| patp: AAE03771 | Human gene 8 encoded secreted | 2227 | 1.0e−230 |
| | protein | | |
| patp: AAE03784 | Human gene 8 encoded secreted | 2227 | 1.0e−230 |
| | protein | | |
| patp: AAE03789 | Human gene 8 encoded secreted | 2227 | 1.0e−230 |
| | protein | | |

In a search of sequence databases, it was found, for example, that the NOV8a nucleic acid sequence has 220 of 376 bases (58%) identical to a gb:GENBANK-ID:AB013805|acc:AB013805 mRNA from Homo sapiens (Homo sapiens mRNA for neural plakophilin-related arm-repeat protein (NPRAP). The full amino acid sequence of the NOV8a protein of the invention was found to have 121 of 325 amino acid residues (37%) identical to, and 187 of 325 amino acid residues (57%) similar to, the 345 amino acid residue ptnr:TREMBLNEW-ACC:BAB09511 protein from Arabidopsis thaliana (Mouse-ear cress) (UDP-GALACTOSE TRANSPORTER RELATED PROTEIN-LIKE).

Similarly, in a search of sequence databases, it was found, for example, that the NOV8b nucleic acid sequence has 1716 of 1722 bases (99%) identical to a gb:GENBANK-ID:AX136243/acc:AX136243.1 mRNA from Homo sapiens (Sequence 165 from Patent EP1067182). The full amino acid sequence of the protein of the invention was found to have 197 of 397 amino acid residues (49%) identical to, and 269 of 397 amino acid residues (67%) similar to the 465 amino acid residue ptnr:SPTREMBL-AC:Q9VEI3 protein from Drosophila melanogaster (Fruit fly) (CG7623 PROTEIN).

Additional BLASTP results are shown in Table 8E.

TABLE 8E
NOV8 BLASTP results

| Gene Index/ | | Length | Identity | Positives | |
|---|---|---|---|---|---|
| Identifier | Protein/Organism | (aa) | (%) | (%) | Expect |
| AAL07487 | EMBRYONIC SEVEN-SPAN | 431 | 396/432 | 408/432 | 1.8e−211 |
| | TRANSMEMBRANE PROTEIN- | | (91%) | (94%) | |
| | LIKE PROTEIN - Mus musculus | | | | |
| | (Mouse) | | | | |
| Q9VEI3 | CG7623 PROTEIN - Drosophila | 465 | 197/397 | 269/397 | 8.3e−95 |
| | melanogaster (Fruit fly) | | (49%) | (67%) | |
| AAK93522 | SD04658P - Drosophila | 465 | 197/397 | 269/397 | 8.3e−95 |
| | melanogaster (Fruit fly) | | (49%) | (67%) | |
| O16377 | M03F8.2 PROTEIN - | 417 | 153/365 | 217/365 | 6.3e−65 |
| | Caenorhabditis elegans | | (41%) | (59%) | |
| Q9D1L5 | 1110003M08RIK PROTEIN - | 124 | 103/124 | 112/124 | 1.3e−55 |
| | Mus musculus (Mouse) | | (83%) | (90%) | |

A multiple sequence alignment is given in Table 8F, with the NOV8 protein of the invention being shown in line 1 and 2, in a ClustalW analysis comparing NOV8 with related protien sequences of Table 8E.

[image]

[image]

Domain results for NOV8 were collected from BLAST sample domains found in the Smart and Pfam collections. The results are listed in Table 8G with the statistics and domain description. This indicates that the NOV8 polypeptides have properties similar to those of other proteins known to contain these domains.

TABLE 8G
Domain Analysis of NOV8

| | | Smallest |
|---|---|---|
| | | Sum |
| | High | Prob |
| Sequences producing High-scoring Segment Pairs: | Score | P(N) |
| | Score | E |
| PSSMs producing significant alignments: | (bits) | value |
| DUF6, Integral membrane protein | −141.5 | 8.4 |

| | | | | |
|---|---|---|---|---|
| DUF6 | | sSakNAfkkcfkSiFswHNETvNIWtykkekflerlvklsHLlGfil | | (SEQ ID NO:134) |
| | | + + +++\| +++ +\| + + | | |
| NOV8a | 226 | MG----KLVSRRSYEHWE----------------------YLTATLI | 246 | (SEQ ID NO:26) |
| DUF6 | | FffllildflfllvpilasvtshLyilqdrvvfgfftdlcvhdlagWpfy | | |
| | | ++ + +++++ +++++++ ++ +++ +++ + | | |
| NOV8a | 247 | SIGVSMFLLSSGPEPRSSPATT--------LSGLIL-------LAG---Y | 278 | |
| DUF6 | | .fl.gaflCLllSsiyHtfschSlekvsefflklDYlGIsllIvaSfipi | | |
| | | ++++ +\|+ +++ ++ +++ | | |
| NOV8a | 279 | iAFdSF---------------TSNWQDALFAYKMS--------------- | 298 | |
| DUF6 | | iYyaFychpffrtlYisiilvLGliaiyvslsdkFsspkfRkrRvplRag | | |
| | | ++++++ + + + \|++++ + ++ ++ ++ ++ + | | |
| NOV8a | 299 | -------SVQMMFG-VNFFSCLFTVGSLLEQGALLEGTRFMGRHSEFAAH | 340 | |
| DUF6 | | fFvllglsGviPllHalilfgghenlkvrialpwvllmallYivGavfYg | | |
| | | +++++++ + + +++ + +++ ++ +++++++++++ ++\|+ | | |
| NOV8a | 341 | ALLLSICSA-CGQLFIFYTIGQFGA--AVFTIIMTLRQAFAILLSCLLYG | 387 | |
| DUF6 | | tRIPERffrCPHaGKFDivGhSHQlFHvlVV..laafcHyravl | | |
| | | + + \|\|+++++++ + +++ | | |
| NOV8a | 388 | -------H--T----------------VTVVggLGVAVVFAALL | 406 | |

The NOV8 disclosed in this invention is expressed in at least the following tissues: Adrenal gland, Aorta, Blood, Brain, Breast, CNS, Colon, Esophagus, Foreskin, Germ Cell, Lung, Lymph, Skeletal Muscle, Ovary, Pancreas, Parathyroid, Placenta, Pooled, Prostate, Salivary Glands (including parotid), Spleen, Stomach, Synovial membrane, Testis, Tonsil, Uterus, Whole embryo, brain, breast, breast_normal, cervix, colon, eye, kidney, leiomios, lung, lung cell line, ovary, pancreas, placenta, skin, uterus. This information was derived by determining the tissue sources of the sequences that were included in the invention including but not limited to SeqCalling sources, Public EST sources, Literature sources, and/or RACE sources. This list of tissue sources is by no way limiting. Further tissue expression analysis is provided in the Examples.

The protein similarity information, expression pattern, and map location for the novel nucleotide-sugar transporter-like protein and nucleic acid disclosed herein suggest that this novel nucleotide-sugar transporter may have important structural and/or physiological functions characteristic of the nucleotide-sugar transporter family. Therefore, the NOV8 nucleic acids and proteins of the invention are useful in potential diagnostic and therapeutic applications and as a research tool. For example, the compositions of the present invention will have efficacy for treatment of patients suffering from muscular dystrophy, Lesch-Nyhan syndrome, myasthenia gravis, Von Hippel-Lindau (VHL) syndrome, pancreatitis, xerostomia, arthritis, tendinitis, fertility, and cancer (preferably ovarian and pancreatic tumors). Furthermore, the NOV8 nucleic acids and polypeptides of the invention could have efficacy for treatment of patients suffering from metabolic diseases, preferably obesity and diabetes, as well as other diseases, disorders and conditions.

The novel nucleic acid encoding the nucleotide-sugar transporter-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV8 protein has multiple hydrophilic regions, each of which can be used as an immunogen. In one embodiment, a contemplated NOV8 epitope is from about amino acids 10 to 40. In another embodiment, a contemplated NOV8 epitope is from about amino acids 55 to 75. In other specific embodiments, contemplated NOV8 epitopes are from about amino acids 80 to 110, 125 to 155, 175 to 190, 220 to 240, 250 to 260, 275 to 290, 310 to 340, and 410 to 475.

NOV9

Still another NOVX protein of the invention, referred to herein as NOV9 (alternatively referred to as AC018755_da1), is an OB binding protein-2-like protein. OB binding proteins are located at the plasma membrane and associate with transmembrane subgroup members of the immunoglobulin superfamily, such as sialic acid-binding Ig-like lectin 5 (siglec-5). Such associations constitute a unique related subgroup with a high level of overall amino acid identity. These associations serve to mediate sialic acid-dependent binding to human erythrocytes and soluble glycoconjugates, suggesting involvement in cell-cell interactions and recognition events.

The OB Binding Protein-2 disclosed in this invention maps to chromosome 19q13.3. This information was assigned using publicly available reference material from OMIM and Pubmed.

The NOV9 nucleic acid (SEQ ID NO:28) of 1368 nucleotides encoding a novel OB binding protein-2-like protein is shown in Table 9A. An open reading frame for the mature protein was identified beginning with a ATG initiation codon at nucleotides 1-3 and ending with a TGA codon at nucleotides 1366-68. The start and stop codons are in bold letters.

TABLE 9A
NOV9 Nucleotide Sequence.

| | |
|---|---|
| ATGCTGCCCCTGCTGCTGCTGCCCCTGCTGTGGGGGGGTTCCCTGCAGGAGAAGCCAGTGTAC | (SEQ ID NO:28) |
| GAGCTGCAAGTGCAGAAGTCGGTGACGGTGCAGGAGGGCCTGTGCGTCCTTGTGCCCTGCTCC | |
| TTCTCTTACCCCTGGAGATCCTGGTATTCCTCTCCCCCACTCTACGTCTACTGGTTCCGGGAC | |
| GGGGAGATCCCATACTACGCTGAGGTTGTGGCCACAAACAACCCAGACAGAAGAGTGAAGCCA | |
| GAGACCCAGGGCCGATTCCGCCTCCTTGGGGATGTCCAGAAGAAGAACTGCTCCCTGAGCATC | |
| GGAGATGCCAGAATGGAGGACACGGGAAGCTATTTCTTCCGCGTGGAGAGAGGAAGGGATGTA | |
| AAATATAGCTACCAACAGAATAAGCTGAACTTGGAGGTGACAGCCCTGATAGAGAAACCCGAC | |
| ATCCACTTTCTGGAGCCTCTGGAGTCCGGCCGCCCCACAAGGCTGAGCTGCAGCCTTCCAGGA | |
| TCCTGTGAAGCGGGACCACCTCTCACATTCTCCTGGACGGGGAATGCCCTCAGCCCCCTGGAC | |
| CCCGAGACCACCCGCTCCTCGGAGCTCACCCTCACCCCCAGGCCCGAGGACCATGGCACCAAC | |
| CTCACCTGTCAGATGAAACGCCAAGGAGCTCAGGTGACCACGGAGAGAACTGTCCAGCTCAAT | |
| GTCTCCGATGCTCCACAGACCATCACCATCTTCAGGAACGGCATAGCCCTAGAGATCCTGCAA | |
| AACACCTCATACCTTCCGGTCCTGGAGGGCCAGGCTCTGCGGCTGCTCTGTGATGCTCCCAGC | |
| AACCCCCCTGCACACAGCTGGTTCCAGGGCTCCCCTGCCCTGAACGCCACCCCCATCTCCAAT | |
| ACCGGGATCTTGGAGCTTCGTCGAGTAAGGTCTGCAGAAGAAGGAGGCTTCACCTGCCGCGCT | |
| CAGCACCCGCTGGGCTTCCTGCAAATTTTTCTGAATCTCTCAGTTTACTGGAGATCGAACCTC | |
| GGGACAGGAGTGGTTCCTGCAGCCCTTGGTGGTGCTGGTGTCATGGCCCTGCTCTGTATCTGT | |
| CTGTGCCTCATCTTCTTTTTAATAGTGAAAGCCCGCAGGAAGCAAGCAGCTGGGAGACCAGAG | |
| AAAATGGATGATGAAGACCCCATTATGGGTACCATCACCTCGGGTTCCAGGAAGAAGCCCTGG | |
| CCAGACAGCCCCGGAGATCAAGCATCTCCTCCTGGGGATGCCCCTCCCTTGGAAGAACAAAAG | |
| GAGCTCCATTATGCCTCCCTTAGTTTTTCTGAGATGAAGTCGAGGGAGCCTAAGGACCAGGAG | |
| GCCCCAAGCACCACGGAGTACTCGGAGATCAAGACAAGCAAGTGA | |

The sequence of NOV9 was derived by laboratory cloning of cDNA fragments covering the full length and/or part of the DNA sequence of the invention, and/or by in silico prediction of the full length and/or part of the DNA sequence of the invention from public human sequence databases.

The cDNA coding for the NOV9 sequence was cloned by the polymerase chain reaction (PCR). Primers were designed based on in silico predictions of the full length or some portion (one or more exons) of the cDNA/protein sequence of the invention, or by translated homology of the predicted exons to closely related human sequences or to sequences from other species. The DNA sequence and protein sequence for an OB binding protein-2-like gene were obtained by exon linking and are reported here as NOV9. These primers and methods used to amplify NOV9 cDNA are described in the Examples.

The NOV9 polypeptide (SEQ ID NO:29) encoded by SEQ ID NO:28 is 455 amino acid residues in length and is presented using the one-letter amino acid code in Table 9B. The SignalP, Psort and/or Hydropathy results predict that NOV9 has a signal peptide and is likely to be localized in the plasma membrane with a certainty of 0.4600. In alternative embodiments, a NOV9 polypeptide is located to the endoplasmic reticulum (membrane) with a certainty of 0.1000, the endoplasmic reticulum (lumen) with a certainty of 0.1000, or outside the cell with a certainty of 0.1000. The SignalP predicts a likely cleavage site for a NOV9 peptide is between amino acid positions 16 and 17, i.e. at the dash in the sequence SLQ-EK.

TABLE 9B
Encoded NOV9 protein sequence.

(SEQ ID NO:29)

MLPLLLLPLLWGGSLQEKPVYELQVQKSVTVQEGLCVLVPCSFSYPWRSWYSSPPLYVYWFRDGEIPYYA
EVVATNNPDRRVKPETQGRFRLLGDVQKKNCSLSIGDARMEDTGSYFFRVERGRDVKYSYQQNKLNLEVT
ALIEKPDIHFLEPLESGRPTRLSCSLPGSCEAGPPLTFSWTGNALSPLDPETTRSSELTLTPRPEDHGTN
LTCQMKRQGAQVTTERTVQLNVSDAPQTITIFRNGIALEILQNTSYLPVLEGQALRLLCDAPSNPPAHSW
FQGSPALNATPISNTGILELRRVRSAEEGGFTCRAQHPLGFLQIFLNLSVYWRSNLGTGVVPAALGGAGV
MALLCICLCLIFFLIVKARRKQAAGRPEKMDDEDPIMGTITSGSRKKPWPDSPGDQASPPGDAPPLEEQK
ELHYASLSFSEMKSREPKDQEAPSTTEYSEIKTSK

SNP variants are disclosed in Example 3. The amino acid sequence of NOV has high homology to other proteins as shown in Table 9C.

TABLE 9C.
BLASTX results from Patp database for NOV9

| | | | Smallest |
|---|---|---|---|
| | | | Sum |
| | | High | Prob |
| Sequences producing High-scoring Segment Pairs: | | Score | P(N) |
| patp: AAW55884 | Human CD33-like protein - Homo sapiens | 1735 | 1.4e−178 |
| patp: AAM00948 | Human bone marrow protein | 1731 | 3.7e−178 |
| patp: AAB29191 | CD33 protein - Homo sapiens | 1724 | 2.0e−177 |
| patp: AAW25945 | Ob binding protein - Synthetic | 1632 | 1.1e−167 |
| patp: AAU02894 | Ob binding protein sequence #2 - Homo sapiens | 1632 | 1.1e−167 |

In a search of sequence databases, it was found, for example, that the nucleic acid sequence of this invention has 992 of 997 bases (99%) identical to a gb:GENBANK-ID:HSU71383|acc:U71383 mRNA from Homo sapiens (Human OB binding protein-2 (OB-BP2) mRNA). The full amino acid sequence of the protein of the invention was found to have 330 of 332 amino acid residues (99%) identical to, and 330 of 332 amino acid residues (99%) similar to, the 551 amino acid residue ptnr:SPTREMBL-ACC:O15389 protein from Homo sapiens (Human) (OB BINDING PROTEIN-2).

Additional BLASTP results are shown in Table 9D.

TABLE 9D
NOV9 BLASTP results

| Gene Index/ | | Length | Identity | Positives | |
|---|---|---|---|---|---|
| Identifier | Protein/Organism | (aa) | (%) | (%) | Expect |
| O15389 | OB BINDING PROTEIN-2 | 551 | 330/332 | 330/332 | 7.7e−179 |
| | (SIGLEC5) - Homo sapiens | | (99%) | (99%) | |
| | (Human) | | | | |
| AAD50978 | SIALIC ACID BINDING IG-LIKE | 551 | 329/332 | 330/332 | 2.0e−178 |
| | LECTIN-5 - Homo sapiens | | (99%) | (99%) | |
| | (Human) | | | | |
| O43699 | CD33L1 Protein - Homo sapiens | 442 | 249/455 | 314/455 | 2.1e−114 |
| | (Human) | | (54%) | (69%) | |
| O15388 | OB BINDING PROTEIN-1 - | 440 | 242/455 | 306/455 | 2.6e−107 |
| | Homo sapiens (Human) | | (53%) | (67%) | |
| Q9BYI9 | FOAP-9 - Homo sapiens | 463 | 227/458 | 296/458 | 7.6e−101 |
| | (Human) | | (49%) | (64%) | |

A multiple sequence alignment is given in Table 9E in a ClustalW analysis comparing NOV9 with related protein sequences disclosed in Table 9D.

[image]

[image]

[image]

Domain results for NOV9 were collected from BLAST sample domains found in the Smart and Pfam collections. The disclosed NOV9 polypeptide contains domain IPR003006 at amino acids 50 to 117, and at amino acid positions 262 to 315. This indicates that the NOV9 sequence of the invention has properties similar to those of other proteins known to contain this domain, as well as to the immunoglobulin domain itself. Table 9F lists the domain description.

TABLE 9F
Domain Analysis of NOV9

| | Score | E |
|---|---|---|
| PSSMs producing significant alignments: | (bits) | value |
| Ig, Immunoglobulin domain | 13.3 | 0.014 |

| | | | | |
|---|---|---|---|---|
| Ig | | svsgfgpp.p.vtWlrngk............................ | | (SEQ ID NO:140) |
| | | +++ +++ ++\|+++++ + + +++++++ +++++++ + | | |
| NOV9 | 50 | WYS---SPpLyVYWFRDGEipyyaevvatnnpdrrvkpetqgrfrll | 93 | (SEQ ID NO:29) |
| Ig | | .......lslti.svtpeDsgGtYt | | |
| | | ++ ++++ ++ +++++ +\|+ \| \|+ | | |
| NOV9 | 94 | gdvqkknCSLSIgDARMEDT-GSYF | 117 | |

The OB Binding Protein-2 disclosed in this invention is expressed in at least the following tissues: bone marrow, liver, spleen, lung, and peripheral blood leukocytes. This information was derived by determining the tissue sources of the sequences that were included in the invention. SeqCalling sources, PublicEST sources and publicly available reference material from OMIM and Pubmed.

In addition, the sequence is predicted to be expressed in the following tissues because of the expression pattern of (GENBANK-ID: gb:GENBANK-ID:HSU71383|acc:U71383) a closely related Human OB binding protein-2 (OB-BP2) mRNA, complete cds homolog in species Homo sapiens activated monocyte.

The protein similarity information, expression pattern, and map location for the OB Binding Protein-2-like protein and nucleic acid disclosed herein suggest that this OB Binding Protein-2 may have important structural and/or physiological functions characteristic of the sialic acid-binding immunoglobulin-like lectins (SIGLECs) family. Therefore, the NOV9 nucleic acids and proteins of the invention are useful in potential diagnostic and therapeutic applications and as a research tool. For example, the compositions of the present invention will have efficacy for treatment of patients suffering from: 3-methylglutaconicaciduria, type III; Charcot-Marie-Tooth disease, type 4F; Colorectal cancer; Cone-rod retinal dystrophy-2; DNA ligase I deficiency; Glutaricaciduria, type IIB; Heart block, progressive familial, type I; Hydatidiform mole; Hyperferritinemia-cataract syndrome; Leber congenital amaurosis due to defect in CRX; Liposarcoma; Myotonic dystrophy; Retinitis pigmentosa, late-onset dominant; Spinocereballar ataxia-13; T-cell acute lymphoblastic leukemia; Trichothiodystrophy; Xeroderma pigmentosum, group D; Diabetes mellitus, noninsulin-dependent; Polio, susceptibility to; Hemophilia; Hypercoagulation; Idiopathic thrombocytopenic purpura; Immunodeficiencies; Graft vesus host; Von Hippel-Lindau (VHL) syndrome; Cirrhosis; Fertility; Systemic lupus erythematosus, Autoimmune disease, Asthma, Emphysema, Scleroderma, allergy, ARDS and other diseases and disorders associated with the like.

The novel nucleic acid encoding the OB binding protein-2-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV9 protein has multiple hydrophilic regions, each of which can be used as an immunogen. In one embodiment, a contemplated NOV9 epitope is from about amino acids 10 to 30. In another embodiment, a contemplated NOV9 epitope is from about amino acids 40 to 60. In other specific embodiments, contemplated NOV9 epitopes are from about amino acids 60 to 100, 100 to 120, 120 to 140, 145 to 170, 175 to 230, 260 to 290, 290 to 320, and 360 to 450.

NOV10

A disclosed NOV10 nucleic acid of 1811 nucleotides (also referred to as 30675745_{—}0_{—}499_da1) encoding a novel Trypsin-like protein is shown in Table 10A. An open reading frame was identified beginning with a ATG initiation codon at nucleotides 368-370 and ending with a TAG codon at nucleotides 1553-1555. Putative untranslated regions upstream from the initiation codon and downstream from the termination codon are underlined in Table 10A. The start and stop codons are in bold letters.

TABLE 10A
NOV10 Nucleotide Sequence

(SEQ ID NO:30)

ACAAATCCTTCTGTTGAACTCTACTGTGTCAGGCCAGCCTGAGTTCATTTCTCCTTGAGCAGGAACAGTT
CATGGACGAACTCTGAGGACCATTCTGAGGACAAGAGGCATCCAGTGTCATGAGTGGAACATGCAGCATT
TTATGGCTACAGAGTTAAGGCAAGGGTTGAATTCCACGAGTCAAAAAGCAGCCCTTTTCAGAGACCCAAC
TCTCTGGGGTGCTCAGGGGCTTGGGCTGGATTGAGAAGAAAACTGACAAGAGTAAGCTGCCCTCTCTTCT
CTGGCCATCTCACAAACCACAGTGCGGGCCAACTGGTCCTGCCTCTTTACCACACAGAACCAAGCACTAG
GGATAAGACAGCTGCCCATGGTGTCCGCGGCGGGTCTCTCTGGGGATGGCAAGATGCGAGGGGTGCTCCT
GGTGCTGCTCGGCCTTCTCTATTCTTCCACCAGTTGTGGCGTCCAGAAAGCTTCCGTTTTCTACGGTCCT
GACCCCAAGGAGGGCTTGGTCAGCAGCATGGAGTTCCCGTGGGTGGTGTCGCTGCAGGACTCCCAGTACA
CACACCTGGCTTTCGGCTGCATCCTGAGCGAGTTCTGGGTCCTCAGCATCGCATCCGCCATTCAGAACAG
GAAGGACATTGTCGTTATAGTGGGTATAAGTAACATGGATCCTAGCAAGATTGCTCACACAGAGTATCCA
GTCAATACCATCATCATCCATGAGGACTTTGATAACAACTCCATGAGCAACAACATAGCCCTCCTGAAGA
CAGACACAGCGATGCATTTTGGCAACCTGGTCCAGTCCATCTGCTTCCTCGGCAGAATGCTGCATACACC
ACCAGTCTTGCAGAACTGCTGGGTGTCAGGATGGAATCCCACATCTGCAACAGGAAATCACATGACGATG
AGTGTCCTGAGGAAAATCTTCGTGAAAGATCTTGACATGTGTCCCCTATACAAACTCCAGAAGACAGAAT
GCGGCAGCCACACGAAAGAGGAAACCAAGACTGCCTGCTTGGGGGACCCAGGAAGCCCAATGATGTGCCA
GCTACAGCAGTTCGATCTGTGGGTTCTGAGAGGAATCCTGAACTTCGGTGGTGAGACGTGCCCTGGCCTG
TTTCTGTACACCAAGGTGGAAGACTACAGCAAATGGATCACATCCAAGGCTGAGAGGGCCGGCCCTCCCC
TGTCCTCACTCCACCACTGGGAAAAGTTGATTTCTTTCTCCCACCATGGACCAAATGCCGCCATGACACA
GAAGACATATTCTGATTCTGAACTGGGCCATGTTGGATCATACTTGCAGGGACAAAGAAGGACCATCACG
CATTCACGACTAGGAAACAGCTCTAGAGATAGTCTAGATGTTAGGGAGAAGGATGTAAAGGAATCAGGCA
GGTCTCCTGAGGCGTCTGTACAACCCTTATACTATGACTATTACGGTGGGGAGGTGGGGGAAGGTAGGAT
TTTTGCAGGTCAGAACAGGTTGTATCAGCCCGAAGAAATCATCTTGGTTTCCTTCGTGCTTGTTTTCTTT
TGCAGCAGTATCTAGTCCAGGAGCTACCCCACCAAACTGAAGAGTAAACTGAGAATGCTGAGTGCCAGGC
ATTCACCATGCTGTTTTGATGTCTGTTTTTGATAGTTGCACACTGGGGCTGCCACGGATAAGCCCATGGC
ATACACTGGGCTGGCTCTCCCTCCTCTATCCCTCTCCCAGGTGTGGGAAGGTCACTTTCACTATGCTTGT
GAACTAAATGCTGGCTAACAAGTGTCAAAAAAAAAAAAAAAAAAAAAAAAAAAAAAAAAAA

The disclosed NOV10 nucleic acid sequence maps to chromosome 16 and has 1129 of 1129 bases (100%) identical to a gb:GENBANK-ID:HSM802210|acc:AL137484 mRNA from Homo sapiens (Homo sapiens mRNA; cDNA DKFZp434I2330 (from clone DKFZp434I2330); partial cds).

A disclosed NOV10 protein (SEQ ID NO:31) encoded by SEQ ID NO:30 has 395 amino acid residues, and is presented using the one-letter code in Table 10B. Signal P, Psort and/or Hydropathy results predict that NOV10 contains a signal peptide, and is likely to be localized extracellularly with a certainty of 0.9190. The most likely cleavage site for a NOV10 peptide is between amino acids 30 and 31, at: TSC-GV.

TABLE 10B
Encoded NOV10 protein sequence.

(SEQ ID NO:31)

MVSAAGLSGDGKMRGVLLVLLGLLYSSTSCGVQKASVFYGPDPKEGLVSSMEFPWVVSLQDSQYTHLAFG
CILSEFWVLSIASAIQNRKDIVVIVGISNMDPSKIAHTEYPVNTIIIHEDFDNNSMSNNIALLKTDTAMH
FGNLVQSICFLGRMLHTPPVLQNCWVSGWNPTSATGNHMTMSVLRKIFVKDLDMCPLYKLQKTECGSHTK
EETKTACLGDPGSPMMCQLQQFDLWVLRGILNFGGETCPGLFLYTKVEDYSKWITSKAERAGPPLSSLHH
WEKLISFSHHGPNAAMTQKTYSDSELGHVGSYLQGQRRTITHSRLGNSSRDSLDVREKDVKESGRSPEAS
VQPLYYDYYGGBVGEGRIFAGQNRLYQPEEITLVSFVLVFFCSSI

Additional SNP variants of NOV10 are disclosed in Example 3. The NOV10 amino acid sequence has 79 of 263 amino acid residues (30%) identical to, and 128 of 263 amino acid residues (48%) similar to, the 269 amino acid residue ptnr:SWISSPROT-ACC:Q29461 protein from Bos taurus (Bovine) [ELASTASE 2 PRECURSOR (EC 3.4.21.71)].

NOV10 is expressed in at least the testis because of the expression pattern of a closely related homolog [(GENEBANK-ID: gb:GENEBANK-ID:HSM802210|acc:AL137484) (Homo sapiens mRNA; cDNA DKFZp434I2330 (from clone DKFZp434I2330)]. Additional sites of tissue expression for NOV10 are disclosed in Example 1.

NOV10 also has homology to the amino acid sequences shown in the BLASTP data listed in Table 10C.

TABLE 10C
BLAST results for NOV10

| Gene Index/ | Protein/ | Length | Identity | Positives | |
|---|---|---|---|---|---|
| Identifier | Organism | (aa) | (%) | (%) | Expect |
| gi\|11360059\|pir\|\| | hypothetical | 290 | 278/290 | 278/290 | e−167 |
| T46470;gi\|6808103\| | protein | | (95%) | (95%) | |
| emb\|CAB70765.1\| | DKFZp434I2330.1 - | | | | |
| (AL137484) | human | | | | |
| | (fragment) | | | | |
| | [Homo | | | | |
| | sapiens] | | | | |
| gi\|14775106\|ref\|X | hypothetical | 270 | 256/270 | 257/270 | e−153 |
| P_048011.1\| | protein | | (94%) | (94%) | |
| | XP_048011 | | | | |
| | [Homo | | | | |
| | sapiens] | | | | |
| gi\|12838178\|dbj\|B | putative [Mus | 624 | 73/235 | 108/235 | 3e−17 |
| AB24114.1\| | musculus] | | (31%) | (45%) | |
| (AK005546) | | | | | |
| gi\|12839985\|dbj\|BAB | putative [Mus | 326 | 77/266 | 128/266 | 7e−17 |
| 24725.1\|(AK006746) | musculus] | | (28%) | (47%) | |
| gi\|13811665\|gb\|AA | coagulation | 624 | 72/235 | 107/235 | 1e−16 |
| K40233.1\|AF356627_1 | factor XI | | (30%) | (44%) | |
| (AF356627) | [Mus | | | | |
| | musculus] | | | | |

The homology of these sequences is shown graphically in the ClustalW analysis shown in Table 10D.

[image]

[image]

[image]

Table 10E lists the domain description from DOMAIN analysis results against NOV10. This indicates that the NOV10 sequence has properties similar to those of other proteins known to contain this domain.

TABLE 10E
Domain Analysis of NOV10

gnl|Pfam|pfam00089, trypsin, Trypsin (SEQ ID NO: 146)
Length =217 residues, 95.4% aligned
Score =90.5 bits (223), Expect=1e-19

| | | | | |
|---|---|---|---|---|
| NOV10: | 52 | EFPWVVSLQDSQYTHLAFGCILSEFWVLSIASAIQNRKDIVVIVGISNMDPSKIAHTEYP | 111 | |
| | | \|\|\| \|\|\|\| \| \| \| ++\|\| \|\|\|+ \| + + \|++\| \|+ ++ ++ | | |
| 00089: | 11 | SFPWQVSLQVSS-GHFCGGSLISENWVLTAAHCVSGASSVRVVLGEHNLGTTEGTEQKFD | 69 | |
| NOV10: | 112 | VNTIIIHEDFDNNSMSNNIALLKTDTAMHFGNLVQSICFLGRMLHTPPVLQNCWVSGWNP | 171 | |
| | | \| \|\|+\| +++ + +\|+\|\|\|\|\| + + \|+ \|+ \|\| \| \|\| \| \|\|\|\| | | |
| 00089: | 70 | VKKIIVHPNYNPD--TNDIALLKLKSPVTLGDTVRPIC-LPSASSDLPVGTTCSVSGWGR | 126 | |
| NOV10: | 172 | TSATGNHMTMSVLRKIFVKDLDMC----PLYKLQKTECGSHTKEETKTACLGDPGSPMMC | 227 | |
| | | \| \| \|+ + + + + \| \| \| \|\| \|\| \| \|++\| | | |
| 00089: | 127 | TKNLGTSDTLQEV-VVPIVSRETCRSAYGGTVTDTMICA--GALGGKDACQGDSGGPLVC | 183 | |
| NOV10: | 228 | QLQQFDLWVLRGILNFGGETCPG---LFLYTKVEDYSKWI | 264 | (SEQ ID NO:31) |
| | | + \| \|\| \| \| +\|\|+\| \| \|\| | | |
| 00089: | 184 | SDGE-----LVGI-VSWGYGCAVGNYPGVYTRVSRYLDWI | 217 | (SEQ ID NO:147) |

Proteins with gn1|Pfam|pfam00089 Trypsin-like domains include all the proteins in families S1, S2A, S2B, S2C, and S5 in the classification of peptidases. Also included are proteins that are clearly members, but that lack peptidase activity, such as haptoglobin and protein Z (PRTZ*).

Trypsin-Like Proteases may act as metastatic agents in certain cancers, e.g., breast cancer. The ability of tumors to metastasize from their original location to the brain, bone and lymphatic tissue, is not well addressed by current therapies. Normal cells exist in contact with a complex protein network, called the extracellular matrix (ECM). The ECM is a barrier to cell movement and cancer cells must devise ways to break their attachments, degrade, and move through the ECM in order to metastasize. Proteases are enzymes that degrade other proteins and have long been thought to aid in freeing the tumor cells from their original location by chewing up the ECM. Recent studies have suggested that they may promote cell shape changes and motility through the activation of a protein in the tumor cell membrane called Protease-Activated Receptor-2 (PAR2). This leads to a cascade of intracellular reactions that activates the motility apparatus of the cell. Thus, the ECM-degrading proteases serve two functions by, (1) reducing the extracellular resistance to cell movement, and (2) activating the motility processes inside of cells though specific receptors.

The above defined information for NOV10 suggests that this protein may function as a member of a Trypsin-like protein family. Therefore, the NOV10 nucleic acids and proteins of the invention are useful in potential therapeutic and diagnostic applications. For example, a cDNA encoding the NOV10 protein may be useful in gene therapy, and the NOV10 protein may be useful when administered to a subject in need thereof. By way of nonlimiting example, the compositions of the present invention will have efficacy for treatment of patients suffering from cancers. The NOV10 nucleic acid encoding Trypsin-like protein, and the Trypsin-like protein of the invention, or fragments thereof, may further be useful in diagnostic applications, wherein the presence or amount of the nucleic acid or the protein are to be assessed. These proteins and nucleic acids are further useful in the generation of antibodies for use in therapeutic or diagnostic methods. These antibodies may be generated according to methods known in the art, using prediction from hydrophobicity charts, as described in the “Anti-NOVX Antibodies” section below. The disclosed NOV10 protein has multiple hydrophilic regions, each of which can be used as an immunogen. In one embodiment, a contemplated NOV10 epitope is from about amino acids 25 to 65. In another embodiment, a NOV10 epitope is from about amino acids 85 to 135. In further specific embodiments, NOV10 epitopes are from about amino acids 155 to 180, from about amino acids 185 to 220 and from about amino acids 235 to 350.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS:1, 3, 5, 7, 9, 11, 13, 15, 17, 19, 21, 23, 25, 27, 28, and 30, or a complement of this aforementioned nucleotide sequence, can be isolated using standard molecular biology techniques and the sequence information provided herein. Using all or a portion ofthe nucleic acid sequence of SEQ ID NOS: 1, 3, 5, 7, 9, 11, 13, 15, 17, 19, 21, 23, 25, 27, 28, and 30 as a hybridization probe, NOVX molecules can be isolated using standard hybridization and cloning techniques (e.g., as described in Sambrook, et al., (eds.), Molecular Cloning: A Laboratory Manual 2^{nd} Ed., Cold Spring Harbor Laboratory Press, Cold Spring Harbor, N.Y., 1989; and Ausubel, et al., (eds.), Current Protocols in Molecular Biology, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS: 1, 3, 5, 7, 9, 11, 13, 15, 17, 19, 21, 23, 25, 27, 28, and 30, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 1, 3, 5, 7, 9, 11, 13, 15, 17, 19, 21, 23, 25, 27, 28, and 30, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown NOS: 1, 3, 5, 7, 9, 11, 13, 15, 17, 19, 21, 23, 25, 27, 28, or 30 is one that is sufficiently complementary to the nucleotide sequence shown NOS: 1, 3, 5, 7, 9, 11, 13, 15, 17, 19, 21, 23, 25, 27, 28, or 30 that it can hydrogen bond with little or no mismatches to the nucleotide sequence shown SEQ ID NOS: 1, 3, 5, 7, 9, 11, 13, 15, 17, 19, 21, 23, 25, 27, 28, and 30 thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., Current Protocols in Molecular Biology, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS: 1, 3, 5, 7, 9, 11, 13, 15, 17, 19, 21, 23, 25, 27, 28, and 30, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS: 1, 3, 5, 7, 9, 11, 13, 15, 17, 19, 21, 23, 25, 27, 28, and 30; or an anti-sense strand nucleotide sequence of SEQ ID NOS: 1, 3, 5, 7, 9, 11, 13, 15, 17, 19, 21, 23, 25, 27, 28, and 30; or of a naturally occurring mutant of SEQ ID NOS: 1, 3, 5, 7, 9, 11, 13, 15, 17, 19, 21, 23, 25, 27, 28, and 30.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS: 1, 3, 5, 7, 9, 11, 13, 15, 17, 19, 21, 23, 25, 27, 28, and 30, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS: 1, 3, 5, 7, 9, 11, 13, 15, 17, 19, 21, 23, 25, 27, 28, and 30 due to degeneracy of the genetic code and thus encode the same NOVX proteins as that encoded by the nucleotide sequences shown in SEQ ID NOS: 1, 3, 5, 7, 9, 11, 13, 15, 17, 19, 21, 23, 25, 27, 28, and 30. In another embodiment, an isolated nucleic acid molecule of the invention has a nucleotide sequence encoding a protein having an amino acid sequence shown in SEQ IDNOS:2, 4, 6, 8, 10, 12, 14, 16, 18, 20, 22,24, 26, 29, and 31.

In addition to the human NOVX nucleotide sequences shown in SEQ ID NOS: 1, 3, 5, 7, 9, 11, 13, 15, 17, 19, 21, 23, 25, 27, 28, and 30, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS: 1, 3, 5, 7, 9, 11, 13, 15, 17, 19, 21, 23, 25, 27, 28, and 30 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS: 1, 3, 5, 7, 9, 11, 13, 15, 17, 19, 21, 23, 25, 27, 28, and 30. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), Current Protocols in Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS: 1, 3, 5, 7, 9, 11, 13, 15, 17, 19, 21, 23, 25, 27, 28, and 30, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS: 1, 3, 5, 7, 9, 11, 13, 15, 17, 19, 21, 23, 25, 27, 28, and 30, or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, Current Protocols in Molecular Biology, John Wiley & Sons, NY, and Kriegler, 1990; Gene Transfer and Expression, A Laboratory Manual, Stockton Press, NY.

In a third embodiment, a nucleic acid that is hybridizable to the nucleic acid molecule comprising the nucleotide sequences SEQ ID NOS: 1, 3, 5, 7, 9, 11, 13, 15, 17, 19, 21, 23, 25, 27, 28, and 30, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, Current Protocols in Molecular Biology, John Wiley & Sons, NY, and Kriegler, 1990, Gene Transfer and Expression, A Laboratory Manual, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS: 1, 3, 5, 7, 9, 11, 13, 15, 17, 19, 21, 23, 25, 27, 28, and 30, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS:2, 4, 6, 8, 10, 12, 14, 16, 18, 20, 22, 24, 26, 29, and 31. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS: 1, 3, 5, 7, 9, 11, 13, 15, 17, 19, 21, 23, 25, 27, 28, and 30 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2, 4, 6, 8, 10, 12, 14, 16, 18, 20, 22, 24, 26, 29, and 31. Preferably, the protein encoded by the nucleic acid molecule is at least about 60% homologous to SEQ ID NOS: 2, 4, 6, 8, 10, 12, 14, 16, 18, 20, 22, 24, 26, 29, and 31; more preferably at least about 70% homologous SEQ ID NOS: 2, 4, 6, 8, 10, 12, 14, 16, 18, 20, 22, 24, 26, 29, and 31; still more preferably at least about 80% homologous to SEQ ID NOS: 2, 4, 6, 8, 10, 12, 14, 16, 18, 20, 22, 24, 26, 29, and 31; even more preferably at least about 90% homologous to SEQ ID NOS: 2, 4, 6, 8, 10, 12, 14, 16, 18, 20, 22, 24, 26, 29, and 31; and most preferably at least about 95% homologous to SEQ ID NOS: 2, 4, 6, 8,10, 12, 14, 16,18, 20, 22, 24, 26, 29, and 31.

An isolated nucleic acid molecule encoding an NOVX protein homologous to the protein of SEQ ID NOS: 2, 4, 6, 8, 10, 12, 14, 16, 18, 20, 22, 24, 26, 29, and 31 can be created by introducing one or more nucleotide substitutions, additions or deletions into the nucleotide sequence of SEQ ID NOS: 1, 3, 5, 7, 9, 11, 13, 15, 17, 19, 21, 23, 25, 27, 28, and 30, such that one or more amino acid substitutions, additions or deletions are introduced into the encoded protein.

Mutations can be introduced into SEQ ID NOS: 1, 3, 5, 7, 9, 11, 13, 15, 17, 19, 21, 23, 25, 27, 28, and 30 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS: 1, 3, 5, 7, 9, 11, 13, 15, 17, 19, 21, 23, 25, 27, 28, and 30, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS: 1, 3, 5, 7, 9, 11, 13, 15, 17, 19, 21, 23, 25, 27, 28, and 30, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2, 4, 6, 8, 10, 12, 14, 16, 18, 20, 22, 24, 26, 29, and 31, or antisense nucleic acids complementary to an NOVX nucleic acid sequence of SEQ ID NOS: 1, 3, 5, 7, 9, 11, 13, 15, 17, 19, 21, 23, 25, 27, 28, and 30, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS: 1, 3, 5, 7, 9, 11, 13, 15, 17, 19, 21, 23, 25, 27, 28, and 30). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS:2, 4, 6, 8, 10, 12, 14, 16, 18, 20, 22, 24, 26, 29, and 31. The invention also includes a mutant or variant protein any of whose residues may be changed from the corresponding residues shown in SEQ ID NOS: 2, 4, 6, 8, 10, 12, 14, 16, 18, 20, 22, 24, 26, 29, and 31 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS: 2, 4, 6, 8, 10, 12, 14, 16, 18, 20, 22, 24, 26, 29, and 31) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS: 2, 4, 6, 8, 10, 12, 14, 16, 18, 20, 22, 24, 26, 29, and 31. In other embodiments, the NOVX protein is substantially homologous to SEQ ID NOS: 2, 4, 6, 8, 10, 12, 14, 16, 18, 20, 22, 24, 26, 29, and 31, and retains the functional activity of the protein of SEQ ID NOS: 2, 4, 6, 8, 10, 12, 14, 16, 18, 20, 22, 24, 26, 29, and 31, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS: 2, 4, 6, 8, 10, 12, 14, 16, 18, 20, 22, 24, 26, 29, and 31, and retains the functional activity of the NOVX proteins of SEQ ID NOS: 2, 4, 6, 8, 10, 12, 14, 16, 18, 20, 22, 24, 26, 29, and 31.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1, 3, 5, 7, 9, 11, 13, 15, 17, 19, 21, 23, 25, 27, 28, and 30. The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS: 2, 4, 6, 8, 10, 12, 14, 16, 18, 20, 22, 24, 26, 29, and 31, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) Current Protocols in Molecular Biology, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Futhermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: Monoclonal Antibodies and Cancer Therapy, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: Monoclonal Antibodies and Cancer Therapy, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., Gene Expression Technology: Methods in Enzymology 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the □-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS:1, 3, 5, 7, 9, 11, 13, 15, 17, 19, 21, 23, 25, 27, 28, and 30 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: Manipulating the Mouse Embryo, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS: 1, 3, 5, 7, 9, 11, 13, 15, 17, 19, 21, 23, 25, 27, 28, and 30), but more preferably, is a non-human homologue of a human NOVX gene. For example, a mouse homologue of human NOVX gene of SEQ ID NOS: 1, 3, 5, 7, 9, 11, 13, 15, 17, 19, 21, 23, 25, 27, 28, and 30 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: Teratocarcinomas and Embryonic Stem Cells: A Practical Approach, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g. GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS: 1, 3, 5, 7, 9, 11, 13, 15, 17, 19, 21, 23, 25, 27, 28, and 30, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS: 1, 3, 5, 7, 9, 11, 13, 15, 17, 19, 21, 23, 25, 27, 28, and 30 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS: 1, 3, 5, 7, 9, 11, 13, 15, 17, 19, 21, 23, 25, 27, 28, and 30, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (ie., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on a Perkin-Elmer Biosystems ABI PRISM® 7700 Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoinflammatory diseases), Panel CNSD.01 (containing samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1×TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

GENERAL_SCREENING_PANEL_V1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

PANEL 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

AI_comprehensive Panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims was purchased from Clinomics. Emphysema patients ranged in age from 40-70 and all were smokers, this age range was chosen to focus on patients with cigarette-linked emphysema and to avoid those patients with alpha-1 anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

Syn=Synovial

Normal=No apparent disease

Rep22/Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

−M=Male

−F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

| | |
|---|---|
| Patient 2 | Diabetic Hispanic, overweight, not on insulin |
| Patient 7-9 | Nondiabetic Caucasian and obese (BMI > 30) |
| Patient 10 | Diabetic Hispanic, overweight, on insulin |
| Patient 11 | Nondiabetic African American and overweight |
| Patient 12 | Diabetic Hispanic on insulin |

Adiocyte differentiation was induced in donor progenitor cells obtained from Osirus (a division of Clonetics/BioWhittaker) in triplicate except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

| | | |
|---|---|---|
| Donor 2 and 3 U | Mesenchymal | Undifferentiated Adipose |
| | Stem cells | |
| Donor 2 and 3 AM | Adipose | AdiposeMidway Differentiated |
| Donor 2 and 3 AD | Adipose | Adipose Differentiated |

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) pateins, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Broddmann Area 21), parietal cortex (Broddmann area 7), and occipital cortex (Brodmann area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

NOV2a and NOV2c (JGIGC_ORDERED_CIT-HSPC_{—}246B18_{—}20000718_DA1/CG55265-01 and CG55265-03: Fibronectin/LLR/Ig Domain-containing Protein)

Expression of gene JGIGC_ORDERED_CIT-HSPC_{—}246B18_{—}20000718_DA1 and variant CG55265-03 was assessed using the primer-probe set Ag4317 described in Table AA Results from RTQ-PCR runs are shown in Tables AB and AC.

TABLE AA

Probe Name Ag4317

| | | | | Start |
|---|---|---|---|---|
| Primers | Sequences | TM | Length | Position |
| Forward | 5′-GATCCTTGGAAACAACCAGATC-3′ | 59.8 | 22 | 518 |
| | (SEQ ID NO:148) | | | |
| Probe | FAM-5′- | 70.7 | 24 | 569 |
| | CTTCCTGTCCACCGTGGAGGACCT-3′- | | | |
| | TAMRA (SEQ ID NO:149) | | | |
| Reverse | 5′-CTCCAGGTTGTTGTAGGACAGA-3′ | 59.3 | 22 | 596 |
| | (SEQ ID NO:150) | | | |

TABLE AB
Panel General_screening_panel_v1.4

| | Relative | | Relative |
|---|---|---|---|
| | Expression (%) | | Expression (%) |
| | tm7654f_{—} | | tm7654f_{—} |
| Tissue Name | ag4317_a1 | Tissue Name | ag4317_a1 |
| D6005-01_Human adipose | 0.8 | Renal ca._TK-10 | 13.2 |
| 112193_Metastatic | 0.0 | Bladder | 1.8 |
| melanoma | | | |
| 112192_Metastatic | 0.1 | Gastric ca.(liver met)_NCI- | 3.0 |
| melanoma | | N87 | |
| 95280_Epidermis (metastatic | 6.8 | 112197_Stomach | 0.1 |
| melanoma) | | | |
| 95279_Epidermis (metastatic | 0.9 | 94938_Colon | 0.0 |
| melanoma) | | Adenocarcinoma | |
| Melanoma (met)_SK-MEL-5 | 10.5 | Colon ca._SW480 | 8.1 |
| 112196_Tongue (oncology) | 0.1 | Colon ca.(SW480 | 6.4 |
| | | met)_SW620 | |
| 113461_Testis Pool | 0.7 | Colon ca._HT29 | 0.5 |
| Prostate ca. (bone met)_PC-3 | 4.0 | Colon ca._HCT-116 | 33.8 |
| 113455_Prostate Pool | 0.7 | Colon ca._CaCo-2 | 23.6 |
| 103396_Placenta | 0.3 | 83219_CC Well to Mod | 0.6 |
| | | Diff (ODO3866) | |
| 113463_Uterus Pool | 0.2 | 94936_Colon | 0.0 |
| | | Adenocarcinoma | |
| Ovarian | 100.0 | 94930_Colon | 0.0 |
| carcinoma_OVCAR-3 | | | |
| Ovarian | 13.0 | 94935_Colon | 0.0 |
| carcinoma(ascites)_SK-OV-3 | | Adenocarcinoma | |
| 95297_Adenocarcinoma | 4.1 | 113468_Colon Pool | 2.1 |
| (ovary) | | | |
| Ovarian | 7.0 | 113457_Small Intestine | 0.4 |
| carcinoma_OVCAR-5 | | Pool | |
| Ovarian carcinoma_IGROV- | 16.9 | 113460_Stomach Pool | 0.8 |
| 1 | | | |
| Ovarian | 5.4 | 113467_Bone Marrow | 0.3 |
| carcinoma_OVCAR-8 | | Pool | |
| 103368_Ovary | 0.8 | 103371_Fetal Heart | 0.2 |
| MCF7_breast | 3.0 | 113451_Heart Pool | 0.2 |
| carcinoma(pleural effusion) | | | |
| Breast ca. (pleural | 0.3 | 113466_Lymph Node Pool | 3.0 |
| effusion)_MDA-MB-231 | | | |
| 112189_ductal cell | 2.9 | 103372_Fetal Skeletal | 0.4 |
| carcinoma(breast) | | Muscle | |
| Breast ca. (pleural | 13.6 | 113456_Skeletal Muscle | 0.3 |
| effusion)_T47D | | Pool | |
| Breast carcinoma_MDA-N | 6.3 | 113459_Spleen Pool | 1.7 |
| 113452_Breast Pool | 1.1 | 113462_Thymus Pool | 5.6 |
| 103398_Trachea | 0.8 | CNS ca. (glio/astro)_U87- | 0.5 |
| | | MG | |
| 112354_lung | 0.3 | CNS ca. (glio/astro)_U- | 0.0 |
| | | 118-MG | |
| 103374_Fetal Lung | 2.2 | CNS ca. (neuro; met)_SK- | 24.3 |
| | | N-AS | |
| 94921_Small cell carcinoma | 6.7 | 95264_Brain astrocytoma | 2.6 |
| of the lung | | | |
| Lung ca.(small cell)_LX-1 | 1.3 | CNS ca. (astro)_SNB-75 | 11.3 |
| 94919_Small cell carcinoma | 1.0 | CNS ca. (glio)_SNB-19 | 13.3 |
| of the lung | | | |
| Lung ca.(s. cell var.)_SHP-77 | 0.0 | CNS ca. (glio)_SF-295 | 12.1 |
| 95268_Lung (Large cell | 19.0 | 113447_Brain (Amygdala) | 8.4 |
| carcinoma) | | Pool | |
| 94920_Small cell carcinoma | 5.9 | 103382_Brain (cerebellum) | 30.9 |
| of the lung | | | |
| Lung ca.(non-s. cell)_NCI- | 8.5 | 64019-1_brain(fetal) | 28.6 |
| H23 | | | |
| Lung ca.(large cell)_NCI- | 3.1 | 113448_Brain | 11.8 |
| H460 | | (Hippocampus) Pool | |
| Lung ca.(non-s. cell)_HOP-62 | 2.0 | 113464_Cerebral Cortex | 9.5 |
| | | Pool | |
| Lung ca.(non-s. cl)_NCI- | 7.0 | 113449_Brain (Substantia | 12.6 |
| H522 | | nigra) Pool | |
| 103392_Liver | 0.3 | 113450_Brain (Thalamus) | 8.2 |
| | | Pool | |
| 103393_Fetal Liver | 1.2 | 103384_Brain (whole) | 11.9 |
| Liver | 0.1 | 113458_Spinal Cord Pool | 2.7 |
| ca.(hepatoblast)_HepG2 | | | |
| 113465_Kidney Pool | 1.2 | 103375_Adrenal Gland | 0.8 |
| 103373_Fetal Kidney | 1.1 | 113454_Pituitary gland | 0.3 |
| | | Pool | |
| Renal ca._786-0 | 0.3 | 103397_Salivary Gland | 0.7 |
| 112188_renal cell carcinoma | 4.8 | 103369_Thyroid (female) | 0.7 |
| Renal ca._ACHN | 0.9 | Pancreatic ca._CAPAN2 | 1.5 |
| 112190_Renal cell | 4.6 | 113453_Pancreas Pool | 1.9 |
| carcinoma | | | |

TABLE AC
Panel 4.1D

| | Relative | | Relative |
|---|---|---|---|
| | Expression (%) | | Expression (%) |
| | 4.1dx4tm6582 | | 4.1dx4tm6582 |
| Tissue Name | f_ag4317_b2 | Tissue Name | f_ag4317_b2 |
| 93768_Secondary Th1_anti- | 6.4 | 93100_HUVEC | 0.6 |
| CD28/anti-CD3 | | (Endothelial)_IL-1b | |
| 93769_Secondary Th2_anti- | 7.1 | 93779_HUVEC | 1.4 |
| CD28/anti-CD3 | | (Endothelial)_IFN gamma | |
| 93770_Secondary Tr1_anti- | 3.5 | 93102_HUVEC | 0.0 |
| CD28/anti-CD3 | | (Endothelial)_TNF alpha + | |
| | | IFN gamma | |
| 93573_Secondary | 8.3 | 93101_HUVEC | 0.0 |
| Th1_resting day 4-6 in IL-2 | | (Endothelial)_TNF alpha + | |
| | | IL4 | |
| 93572_Secondary | 8.5 | 93781_HUVEC | 4.2 |
| Th2_resting day 4-6 in IL-2 | | (Endothelial)_IL-11 | |
| 93571_Secondary | 8.8 | 93583_Lung | 4.8 |
| Tr1_resting day 4-6 in IL-2 | | Microvascular Endothelial | |
| | | Cells_none | |
| 93568_primary Th1_anti- | 13.8 | 93584_Lung | 1.4 |
| CD28/anti-CD3 | | Microvascular Endothelial | |
| | | Cells_TNFa (4 ng/ml) and | |
| | | IL1b (1 ng/ml) | |
| 93569_primary Th2_anti- | 12.9 | 92662_Microvascular | 1.5 |
| CD28/anti-CD3 | | Dermal endothelium_none | |
| 93570_primary Tr1_anti- | 12.5 | 92663_Microsvasular | 0.6 |
| CD28/anti-CD3 | | Dermal endothelium_TNFa | |
| | | (4 ng/ml) and IL1b (1 | |
| | | ng/ml) | |
| 93565_primary Th1_resting | 7.9 | 93773_Bronchial | 0.0 |
| dy 4-6 in IL-2 | | epithelium_TNFa (4 ng/ml) | |
| | | and IL1b (1 ng/ml)** | |
| 93566_primary Th2_resting | 8.7 | 93347_Small Airway | 0.0 |
| dy 4-6 in IL-2 | | Epithelium_none | |
| 93567_primary Tr1_resting | 10.5 | 93348_Small Airway | 0.0 |
| dy 4-6 in IL-2 | | Epithelium_TNFa (4 | |
| | | ng/ml) and IL1b (1 ng/ml) | |
| 93351_CD45RA CD4 | 2.9 | 92668_Coronery Artery | 0.0 |
| lymphocyte_anti-CD28/anti- | | SMC_resting | |
| CD3 | | | |
| 93352_CD45RO CD4 | 8.6 | 92669_Coronery Artery | 0.6 |
| lymphocyte_anti-CD28/anti- | | SMC_TNFa (4 ng/ml) and | |
| CD3 | | IL1b (1 ng/ml) | |
| 93251_CD8 | 8.3 | 93107_astrocytes_resting | 0.7 |
| Lymphocytes_anti- | | | |
| CD28/anti-CD3 | | | |
| 93353_chronic CD8 | 10.1 | 93108_astrocytes_TNFa (4 | 0.0 |
| Lymphocytes 2ry_resting dy | | ng/ml) and IL1b (1 ng/ml) | |
| 4-6 in IL-2 | | | |
| 93574_chronic CD8 | 10.8 | 92666_KU-812 | 5.9 |
| Lymphocytes 2ry_activated | | (Basophil)_resting | |
| CD3/CD28 | | | |
| 93354_CD4_none | 5.0 | 92667_KU-812 | 2.9 |
| | | (Basophil)_PMA/ionoycin | |
| 93252_Secondary | 5.6 | 93579_CCD1106 | 0.5 |
| Th1/Th2/Tr1_anti-CD95 | | (Keratinocytes)_none | |
| CH11 | | | |
| 93103_LAK cells_resting | 8.4 | 93580_CCD1106 | 0.0 |
| | | (Keratinocytes)_TNFa and | |
| | | IFNg** | |
| 93788_LAK cells_IL-2 | 5.6 | 93791_Liver Cirrhosis | 0.4 |
| 93787_LAK cells_IL-2 + IL- | 1.2 | 93577_NCI-H292 | 4.3 |
| 12 | | | |
| 93789_LAK cells_IL-2 + IFN | 3.5 | 93358_NCI-H292_IL-4 | 2.4 |
| gamma | | | |
| 93790_LAK cells IL-2 + IL- | 5.1 | 93360_NCI-H292_IL-9 | 3.1 |
| 18 | | | |
| 93104_LAK | 4.6 | 93359_NCI-H292_IL-13 | 4.6 |
| cells_PMA/ionomycin and | | | |
| IL-18 | | | |
| 93578_NK Cells IL- | 13.6 | 93357_NCI-H292_IFN | 14.7 |
| 2_resting | | gamma | |
| 93109_Mixed Lymphocyte | 7.4 | 93777_HPAEC_- | 0.0 |
| Reaction_Two Way MLR | | | |
| 93110_Mixed Lymphocyte | 3.1 | 93778_HPAEC_IL-1 | 0.0 |
| Reaction_Two Way MLR | | 3eta/TNA alpha | |
| 93111_Mixed Lymphocyte | 5.4 | 93254_Normal Human | 0.0 |
| Reaction_Two Way MLR | | Lung Fibroblast_none | |
| 93112_Mononuclear Cells | 5.6 | 93253_Normal Human | 1.6 |
| (PBMCs)_resting | | Lung Fibroblast_TNFa (4 | |
| | | ng/ml) and IL-1b (1 ng/ml) | |
| 93113_Mononuclear Cells | 3.4 | 93257_Normal Human | 0.5 |
| (PBMCs)_PWM | | Lung Fibroblast_IL-4 | |
| 93114_Mononuclear Cells | 8.1 | 93256_Normal Human | 0.6 |
| (PBMCs)_PHA-L | | Lung Fibroblast_IL-9 | |
| 93249_Ramos (B cell)_none | 0.4 | 93255_Normal Human | 1.0 |
| | | Lung Fibroblast_IL-13 | |
| 93250_Ramos (B | 0.0 | 93258_Normal Human | 0.0 |
| cell)_ionomycin | | Lung Fibroblast_IFN | |
| | | gamma | |
| 93349_B lymphocytes_PWM | 2.6 | 93106_Dermal Fibroblasts | 0.0 |
| | | CCD1070_resting | |
| 93350_B | 0.6 | 93361_Dermal Fibroblasts | 7.6 |
| lymphoytes_CD40L and IL-4 | | CCD1070_TNF alpha 4 | |
| | | ng/ml | |
| 92665_EOL-1 | 5.2 | 93105_Dermal Fibroblasts | 1.4 |
| (Eosinophil)_dbcAMP | | CCD1070_IL-1 beta 1 | |
| differentiated | | ng/ml | |
| 93248_EOL-1 | 4.4 | 93772_dermal | 1.2 |
| (Eosinophil)_dbcAMP/PMAi | | fibroblast_IFN gamma | |
| onomycin | | | |
| 93356_Dendritic Cells_none | 1.7 | 93771_dermal | 2.3 |
| | | fibroblast_IL-4 | |
| 93355_Dendritic Cells_LPS | 0.7 | 93892_Dermal | 2.4 |
| 100 ng/ml | | fibroblasts_none | |
| 93775_Dendritic Cells_anti- | 2.7 | 99202 Neutrophils_TNFa + | 9.7 |
| CD40 | | LPS | |
| 93774_Monocytes_resting | 2.4 | 99203_Neutrophils_none | 22.6 |
| 93776_Monocytes_LPS 50 | 3.4 | 735010_Colon_normal | 5.0 |
| ng/ml | | | |
| 93581_Macrophages_resting | 0.7 | 735019_Lung_none | 0.0 |
| 93582_Macrophages_LPS | 0.5 | 64028-1_Thymus_none | 31.9 |
| 100 ng/ml | | | |
| 93098_HUVEC | 0.6 | 64030-1_Kidney_none | 100.0 |
| (Endothelial)_none | | | |
| 93099_HUVEC | 0.5 | | |
| (Endothelial)_starved | | | |

Panel General_screening_panel_v1.4 Summary Expression of the JGIGC_ORDERED_CIT-HSPC_{—}246B18_{—}20000718_DA1 gene is highest in an ovarian cancer cell line (CT=27.2). Significant expression levels are also detected in a cluster of ovarian derived cell when compared to normal ovarian tissue. Thus, expression of the JGIGC_ORDERED_CIT-HSPC_{—}246B18_{—}20000718_DA1 gene could be used to distinguish ovarian cancer tissue from normal tissue. There is also significant expression in cell lines derived from melanoma and colon, renal, lung, prostate, and breast cancers when compared to expression of the JGIGC_ORDERED_CIT-HSPC_{—}246B18_{—}20000718_DA1 gene in normal tissue. Thus expression of this gene could be used as a marker for melanoma and colon, renal, lung, prostate and breast cancers. Furthermore, therapeutic modulation of the expression or activity of the JGIGC_ORDERED_CIT-HSPC_{—}246B18_{—}20000718_DA1 protein product could be beneficial in the treatment of these cancers.

Among tissues with metabolic function, the JGIGC_ORDERED_CIT-HSPC_{—}246B18_{—}20000718_DA1 gene is expressed at low but significant levels in tissues derived from adipose, fetal liver, pancreas and the thyroid gland. The expression of this gene is higher in fetal liver (CT=33.6) relative to adult liver (CT=35.5), and may thus serve as a marker to differentiate the two

The JGIGC_ORDERED_CIT-HSPC_{—}246B18_{—}20000718_DA1 gene is also expressed in the fetal lung (CT=32.7) and is detected at much lower levels in the adult lung. This suggests that the gene could be used to differentiate between fetal and adult lung tissue.

The protein encoded by the JGIGC_ORDERED_CIT-HSPC_{—}246B18_{—}20000718_DA1 gene is a homolog of a Leucine-rich-repeat protein and shows moderate expression during development and across all brain regions including the amygdala, cerebellum, hippocampus, cerebral cortex, substantia nigra, thalamus, and spinal cord. In Drosophilia, the LRR region of axon guidance proteins has been shown to be critical for function (especially in axon repulsion). Therefore, the expression profile of the JGIGC_ORDERED_CIT-HSPC_{—}246B18_{—}20000718_DA1 gene makes its protein product an excellent candidate neuronal guidance protein for axons, dendrites and/or growth cones in general. Therapeutic modulation of the levels of this protein, or possible signaling via this protein may be of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease). Furthermore, among non-cancerous tissues, this gene shows highest expression in the cerebellum (CT=28.9) where loss of neurons is a hallmark of the various spinocerebellar ataxias. Thus, therapies targeted to the protein encoded by this gene may be particularly useful in these diseases.

Panel 4.1D Summary The JGIGC_ORDERED_CIT-HSPC_{—}246B18_{—}20000718_DA1 gene is expressed at moderate levels in many of the samples in this panel. Highest expression is seen in the kidney (CT=30.8), thymus, and neutrophils, with low but significant expression in T lymphocytes, including all subsets studied and all conditions of stimulation. The protein encoded by the JGIGC_ORDERED_CIT-HSPC_{—}246B18_{—}20000718_DA1 gene is homologous to a LRR, FN, and Ig domain-containing membrane protein and may be useful as a target for the discovery of therapeutic antibodies that could reduce or eliminate inflammatory and autoimmune disease symptoms in patients with rheumatoid arthritis, asthma, allergies, lupus, and inflammatory bowel disease.

Slit is a repellent axon guidance cue produced by the midline glia in Drosophila that is required to regulate the formation of contralateral projections and the lateral position of longitudinal tracts. Four sequence motifs comprise the structure of Slit: a leucine-rich repeat (LRR), epidermal growth factor-like (EGF) repeats, a laminin-like globular (G)-domain, and a cysteine domain. Here it is demonstrated that the LRR is required for repellent signaling and in vitro binding to Robo. Repellent signaling by slit is reduced by point mutations that encode single amino acid changes in the LRR domain. By contrast to the EGF or G-domains, the LRR domain is required in transgenes to affect axon guidance. Finally, we show that the midline repellent receptor, Robo, binds Slit proteins with internal deletions that also retain repellent activity. However, Robo does not bind Slit protein missing the LRR. Taken together, our data demonstrate that Robinding and repellent signaling by Slit require the LRR region. See generally Battye R, et al., J Neurosci 21:4290-8 (2001); PMID: 11404414.

Drosophila Capricious (CAPS) is a transmembrane protein with leucine-rich repeat (LRR) motifs, expressed on small subsets of neurons and muscles, including muscle 12 and the motoneurons that innervate it (muscle 12 MNs). Panmuscle ectopic expression of CAPS alters the target specificity of muscle 12 MNs, indicating that CAPS can function in muscles as a target recognition molecule. This study first examined the effect of ectopic panneural expression of CAPS on the motoneuronal circuit. It was found that panneural expression of CAPS alters the pathfinding of muscle 12 MNs. The defect appeared to be caused by changes in the steering behavior of muscle 12 MNs at a specific choice point along their pathway to the target muscle. These results revealed a novel function of CAPS in axon pathfinding. Then deletion analyses of CAPS were performed. CAPS lacking the intracellular domain in all neurons or in all muscles was expressed and studied for their ability to induce the pathfinding and targeting phenotypes. The function of muscularly expressed CAPS in target recognition is intracellular domain dependent, whereas the function of neurally expressed CAPS in pathfinding is not, suggesting that CAPS may function in neurons and muscles in a different manner. The requirement of the intracellular domain for the function of muscularly expressed CAPS suggests the presence of a signaling event within muscle cells that is essential for selective synapse formation. See generally Taniguchi et al, J Neurobiol. 42:104-116 (2000); PMID: 10623905

NOV4a (14578444_{—}0_{—}47/CG51018-01: Matrilin 2)

Expression of gene 14578444_{—}0_{—}47 was assessed using the primer-probe set Ag2764 described in Table BA Results from RTQ-PCR runs are shown in Tables BB, BC, BD, and BE.

TABLE BA
Probe Name Ag2764

| | | | | Start |
|---|---|---|---|---|
| Primers | Sequences | TM | Length | Position |
| Forward | 5′-TTTGCAGTGCAACACAGATATC-3′ | 58.8 | 22 | 2695 |
| | (SEQ ID NO:151) | | | |
| Probe | TET-5′- | 65.8 | 26 | 2737 |
| | TTACGGTCTACACAAAAGCTTTCCCA-3′ | | | |
| | TAMRA (SEQ ID NO:152) | | | |
| Reverse | 5′-GCTTCCTGAAGGTTTTGTTGA-3′ | 59.3 | 21 | 2764 |
| | (SEQ ID NO:153) | | | |

TABLE BB
Panel 1.3D

| | Relative | | Relative |
|---|---|---|---|
| | Expression (%) | | Expression (%) |
| | 1.3dx4tm4868 | | 1.3dx4tm4868 |
| Tissue Name | t_ag2764_a1 | Tissue Name | t_ag2764_a1 |
| Liver adenocarcinoma | 4.9 | Kidney (fetal) | 54.3 |
| Pancreas | 1.6 | Renal ca. 786-0 | 1.6 |
| Pancreatic ca. CAPAN 2 | 0.5 | Renal ca. A498 | 6.3 |
| Adrenal gland | 6.7 | Renal ca. RXF 393 | 14.0 |
| Thyroid | 100.0 | Renal ca. ACHN | 18.5 |
| Salivary gland | 6.4 | Renal ca. UO-31 | 13.8 |
| Pituitary gland | 3.8 | Renal ca. TK-10 | 5.4 |
| Brain (fetal) | 0.7 | Liver | 3.2 |
| Brain (whole) | 3.7 | Liver (fetal) | 4.6 |
| Brain (amygdala) | 8.2 | Liver ca. (hepatoblast) | 0.6 |
| | | HepG2 | |
| Brain (cerebellum) | 5.4 | Lung | 4.9 |
| Brain (hippocampus) | 7.4 | Lung (fetal) | 8.2 |
| Brain (substantia nigra) | 2.5 | Lung ca. (small cell) LX-1 | 0.0 |
| Brain (thalamus) | 5.4 | Lung ca. (small cell) NCI- | 7.7 |
| | | H69 | |
| Cerebral Cortex | 8.1 | Lung ca. (s. cell var.) SHP- | 4.8 |
| | | 77 | |
| Spinal cord | 37.2 | Lung ca. (large cell)NCI- | 0.0 |
| | | H460 | |
| CNS ca. (glio/astro) U87- | 5.4 | Lung ca. (non-sm. cell) | 1.4 |
| MG | | A549 | |
| CNS ca. (glio/astro) U-118- | 24.5 | Lung ca. (non-s. cell) NCI- | 3.4 |
| MG | | H23 | |
| CNS ca. (astro) SW1783 | 33.7 | Lung ca (non-s. cell) HOP- | 0.9 |
| | | 62 | |
| CNS ca.* (neuro; met) SK- | 0.1 | Lung ca. (non-s. cl) NCI- | 0.5 |
| N-AS | | H522 | |
| CNS ca. (astro) SF-539 | 13.1 | Lung ca. (squam.) SW 900 | 0.4 |
| CNS ca. (astro) SNB-75 | 7.0 | Lung ca. (squam.) NCI- | 3.3 |
| | | H596 | |
| CNS ca. (glio) SNB-19 | 0.7 | Mammary gland | 29.7 |
| CNS ca. (glio) U251 | 5.6 | Breast ca.* (pl. effusion) | 6.0 |
| | | MCF-7 | |
| CNS ca. (glio) SF-295 | 1.0 | Breast ca.* (pl. ef) MDA- | 6.3 |
| | | MB-231 | |
| Heart (fetal) | 8.9 | Breast ca.* (pl. effusion) | 1.8 |
| | | T47D | |
| Heart | 20.0 | Breast ca. BT-549 | 7.4 |
| Fetal Skeletal | 54.0 | Breast ca. MDA-N | 0.0 |
| Skeletal muscle | 6.9 | Ovary | 65.4 |
| Bone marrow | 0.9 | Ovarian ca. OVCAR-3 | 0.9 |
| Thymus | 7.3 | Ovarian ca. OVCAR-4 | 0.1 |
| Spleen | 2.3 | Ovarian ca. OVCAR-5 | 2.0 |
| Lymph node | 5.1 | Ovarian ca. OVCAR-8 | 16.3 |
| Colorectal | 6.7 | Ovarian ca. IGROV-1 | 0.6 |
| Stomach | 7.4 | Ovarian ca.* (ascites) SK- | 1.1 |
| | | OV-3 | |
| Small intestine | 34.8 | Uterus | 55.1 |
| Colon ca. SW480 | 0.7 | Placenta | 14.8 |
| Colon ca.* (SW480 | 0.1 | Prostate | 20.4 |
| met)SW620 | | | |
| Colon ca. HT29 | 0.7 | Prostate ca.* (bone | 5.5 |
| | | met)PC-3 | |
| Colon ca. HCT-116 | 0.4 | Testis | 13.3 |
| Colon ca. CaCo-2 | 1.5 | Melanoma Hs688(A).T | 0.9 |
| 83219 CC Well to Mod Diff | 0.9 | Melanoma* (met) | 0.8 |
| (ODO3866) | | Hs688(B).T | |
| Colon ca. HCC-2998 | 1.0 | Melanoma UACC-62 | 0.1 |
| Gastric ca.* (liver met) NCI- | 10.0 | Melanoma M14 | 0.6 |
| N87 | | | |
| Bladder | 10.0 | Melanoma LOX IMVI | 0.1 |
| Trachea | 26.1 | Melanoma* (met) SK- | 0.8 |
| | | MEL-5 | |
| Kidney | 12.3 | Adipose | 13.2 |

TABLE BC
Panel 2D

| | Relative | | Relative |
|---|---|---|---|
| | Expression (%) | | Expression (%) |
| | 2dx4tm4648t_{—} | | 2dx4tm4648t_{—} |
| Tissue Name | ag2764_a2 | Tissue Name | ag2764_a2 |
| Normal Colon GENPAK | 21.3 | Kidney NAT Clontech | 1.3 |
| 061003 | | 8120608 | |
| 83219 CC Well to Mod Diff | 0.3 | Kidney Cancer Clontech | 0.3 |
| (ODO3866) | | 8120613 | |
| 83220 CC NAT (ODO3866) | 4.1 | Kidney NAT Clontech | 1.0 |
| | | 8120614 | |
| 83221 CC Gr. 2 rectosigmoid | 0.5 | Kidney Cancer Clontech | 0.9 |
| (ODO3868) | | 9010320 | |
| 83222 CC NAT (ODO3868) | 2.2 | Kidney NAT Clontech | 1.2 |
| | | 9010321 | |
| 83235 CC Mod Diff | 1.0 | Normal Uterus GENPAK | 12.3 |
| (ODO3920) | | 061018 | |
| 83236 CC NAT (ODO3920) | 5.7 | Uterus Cancer GENPAK | 17.5 |
| | | 064011 | |
| 83237 CC Gr. 2 ascend colon | 4.3 | Normal Thyroid Clontech | 100.0 |
| (ODO3921) | | A+ 6570-1 | |
| 83238 CC NAT (ODO3921) | 4.4 | Thyroid Cancer GENPAK | 6.8 |
| | | 064010 | |
| 83241 CC from Partial | 1.0 | Thyroid Cancer | 3.6 |
| Hepatectomy (ODO4309) | | INVITROGEN A302152 | |
| 83242 Liver NAT | 3.8 | Thyroid NAT | 39.9 |
| (ODO4309) | | INVITROGEN A302153 | |
| 87472 Colon mets to lung | 0.4 | Normal Breast GENPAK | 9.1 |
| (OD04451-01) | | 061019 | |
| 87473 Lung NAT | 0.4 | 84877 Breast Cancer | 2.1 |
| (OD04451-02) | | (OD04566) | |
| Normal Prostate Clontech A+ | 77.5 | 85975 Breast Cancer | 2.0 |
| 6546-1 | | (OD04590-01) | |
| 84140 Prostate Cancer | 15.1 | 85976 Breast Cancer Mets | 4.4 |
| (OD04410) | | (OD04590-03) | |
| 84141 Prostate NAT | 21.4 | 87070 Breast Cancer | 2.0 |
| (OD04410) | | Metastasis (OD04655-05) | |
| 87073 Prostate Cancer | 18.4 | GENPAK Breast Cancer | 1.5 |
| (OD04720-01) | | 064006 | |
| 87074 Prostate NAT | 23.8 | Breast Cancer Res. Gen. | 3.6 |
| (OD04720-02) | | 1024 | |
| Normal Lung GENPAK | 5.1 | Breast Cancer Clontech | 2.1 |
| 061010 | | 9100266 | |
| 83239 Lung Met to Muscle | 0.4 | Breast NAT Clontech | 4.9 |
| (ODO4286) | | 9100265 | |
| 83240 Muscle NAT | 0.6 | Breast Cancer | 6.3 |
| (ODO4286) | | INVITROGEN A209073 | |
| 84136 Lung Malignant | 1.1 | Breast NAT | 8.5 |
| Cancer (OD03126) | | INVITROGEN A2090734 | |
| 84137 Lung NAT | 1.6 | Normal Liver GENPAK | 3.5 |
| (OD03126) | | 061009 | |
| 84871 Lung Cancer | 2.2 | Liver Cancer GENPAK | 0.8 |
| (OD04404) | | 064003 | |
| 84872 Lung NAT | 2.1 | Liver Cancer Research | 1.4 |
| (OD04404) | | Genetics RNA 1025 | |
| 84875 Lung Cancer | 3.1 | Liver Cancer Research | 0.4 |
| (OD04565) | | Genetics RNA 1026 | |
| 84876 Lung NAT | 0.8 | Paired Liver Cancer Tissue | 1.2 |
| (OD04565) | | Research Genetics RNA | |
| | | 6004-T | |
| 85950 Lung Cancer | 1.0 | Paired Liver Tissue | 0.2 |
| (OD04237-01) | | Research Genetics RNA | |
| | | 6004-N | |
| 85970 Lung NAT | 2.4 | Paired Liver Cancer Tissue | 0.6 |
| (OD04237-02) | | Research Genetics RNA | |
| | | 6005-T | |
| 83255 Ocular Mel Met to | 9.7 | Paired Liver Tissue | 0.3 |
| Liver (ODO4310) | | Research Genetics RNA | |
| | | 6005-N | |
| 83256 Liver NAT | 3.6 | Normal Bladder GENPAK | 4.3 |
| (ODO4310) | | 061001 | |
| 84139 Melanoma Mets to | 2.2 | Bladder Cancer Research | 0.5 |
| Lung (OD04321) | | Genetics RNA 1023 | |
| 84138 Lung NAT | 1.5 | Bladder Cancer | 1.9 |
| (OD04321) | | INVITROGEN A302173 | |
| Normal Kidney GENPAK | 16.8 | 87071 Bladder Cancer | 6.8 |
| 061008 | | (OD04718-01) | |
| 83786 Kidney Ca, Nuclear | 2.7 | 87072 Bladder Normal | 8.3 |
| grade 2 (OD04338) | | Adjacent (OD04718-03) | |
| 83787 Kidney NAT | 9.3 | Normal Ovary Res. Gen. | 6.4 |
| (OD04338) | | | |
| 83788 Kidney Ca Nuclear | 0.6 | Ovarian Cancer GENPAK | 7.2 |
| grade 1/2 (OD04339) | | 064008 | |
| 83789 Kidney NAT | 7.5 | 87492 Ovarv Cancer | 4.4 |
| (OD04339) | | (OD04768-07) | |
| 83790 Kidney Ca, Clear cell | 9.4 | 87493 Ovary NAT | 3.9 |
| type (OD04340) | | (OD04768-08) | |
| 83791 Kidney NAT | 8.6 | Normal Stomach GENPAK | 6.7 |
| (OD04340) | | 061017 | |
| 83792 Kidney Ca, Nuclear | 0.8 | Gastric Cancer Clontech | 1.7 |
| grade 3 (OD04348) | | 9060358 | |
| 83793 Kidney NAT | 5.8 | NAT Stomach Clontech | 2.2 |
| (OD04348) | | 9060359 | |
| 87474 Kidney Cancer | 0.6 | Gastric Cancer Clontech | 7.2 |
| (OD04622-01) | | 9060395 | |
| 87475 Kidney NAT | 1.0 | NAT Stomach Clontech | 3.3 |
| (OD04622-03) | | 9060394 | |
| 85973 Kidney Cancer | 6.9 | Gastric Cancer Clontech | 1.6 |
| (OD04450-01) | | 9060397 | |
| 85974 Kidney NAT | 8.2 | NAT Stomach Clontech | 0.4 |
| (OD04450-03) | | 9060396 | |
| Kidney Cancer Clontech | 1.9 | Gastric Cancer GENPAK | 6.6 |
| 8120607 | | 064005 | |

TABLE BD
Panel 4D

| | Relative | | Relative |
|---|---|---|---|
| | Expression (%) | | Expression (%) |
| | 4dx4tm4524t_{—} | | 4dx4tm4524t_{—} |
| Tissue Name | ag2764_a1 | Tissue Name | ag2764_a1 |
| 93768_Secondary Th1_anti- | 0.1 | 93100_HUVEC | 0.1 |
| CD28/anti-CD3 | | (Endothelial)_IL-1b | |
| 93769_Secondary Th2_anti- | 0.2 | 93779_HUVEC | 0.9 |
| CD28/anti-CD3 | | (Endothelial)_IFN gamma | |
| 93770_Secondary Tr1_anti- | 0.2 | 93102_HUVEC | 0.1 |
| CD28/anti-CD3 | | (Endothelial)_TNF alpha + | |
| | | IFN gamma | |
| 93573_Secondary | 0.0 | 93101_HUVEC | 0.1 |
| Th1_resting day 4-6 in IL-2 | | (Endothelial)_TNF alpha + | |
| | | IL4 | |
| 93572_Secondary | 0.1 | 93781_HUVEC | 0.6 |
| Th2_resting day 4-6 in IL-2 | | (Endothelial)_IL-11 | |
| 93571_Secondary | 0.0 | 93583_Lung | 0.2 |
| Tr1_resting day 4-6 in IL-2 | | Microvascular Endothelial | |
| | | Cells_none | |
| 93568_primary Th1_anti- | 0.2 | 93584_Lung | 0.2 |
| CD28/anti-CD3 | | Microvascular Endothelial | |
| | | Cells_TNFa (4 ng/ml) and | |
| | | IL1b (1 ng/ml) | |
| 93569_primary Th2_anti- | 0.1 | 92662_Microvascular | 0.1 |
| CD28/anti-CD3 | | Dermal endothelium_none | |
| 93570_primary Tr1_anti- | 0.1 | 92663_Microsvasular | 0.2 |
| CD28/anti-CD3 | | Dermal endothelium_TNFa | |
| | | (4 ng/ml) and IL1b (1 | |
| | | ng/ml) | |
| 93565_primary Th1_resting | 0.5 | 93773_Bronchial | 0.3 |
| dy 4-6 in IL-2 | | epithelium_TNFa (4 ng/ml) | |
| | | and IL1b (1 ng/ml)** | |
| 93566_primary Th2_resting | 0.1 | 93347_Small Airway | 0.2 |
| dy 4-6 in IL-2 | | Epithelium_none | |
| 93567_primary Tr1_resting | 0.1 | 93348_Small Airway | 1.5 |
| dy 4-6 in IL-2 | | Epithelium TNFa (4 | |
| | | ng/ml) and IL1b (1 ng/ml) | |
| 93351_CD45RA CD4 | 100.0 | 92668_Coronery Artery | 0.5 |
| lymphocyte anti-CD28/anti- | | SMC_resting | |
| CD3 | | | |
| 93352_CD45RO CD4 | 0.3 | 92669_Coronery Artery | 0.4 |
| lymphocyte anti-CD28/anti- | | SMC_TNFa (4 ng/ml) and | |
| CD3 | | IL1b (1 ng/ml) | |
| 93251_CD8 | 0.1 | 93107_astrocytes_resting | 3.6 |
| Lymphocytes anti- | | | |
| CD28/anti-CD3 | | | |
| 93353_chronic CD8 | 0.1 | 93108_astrocytes_TNFa (4 | 2.3 |
| Lymphocytes 2ry_resting dy | | ng/ml) and IL1b (1 ng/ml) | |
| 4-6 in IL-2 | | | |
| 93574_chronic CD8 | 0.1 | 92666_KU-812 | 0.1 |
| Lymphocytes 2ry_activated | | (Basophil)_resting | |
| CD3/CD28 | | | |
| 93354_CD4_none | 0.1 | 92667_KU-812 | 0.4 |
| | | (Basophil)_PMA/ionoycin | |
| 93252_Secondary | 0.1 | 93579_CCD1106 | 0.5 |
| Th1/Th2/Tr1_anti-CD95 | | (Keratinocytes)_none | |
| CH11 | | | |
| 93103_LAK cells_resting | 0.1 | 93580_CCD1106 | 0.4 |
| | | (Keratinocytes)_TNFa and | |
| | | IFNg** | |
| 93788_LAK cells_IL-2 | 0.2 | 93791_Liver Cirrhosis | 1.4 |
| 93787_LAK cells_IL-2 + IL- | 0.2 | 93792_Lupus Kidney | 1.6 |
| 12 | | | |
| 93789_LAK cells_IL-2 + IFN | 0.4 | 93577_NCI-H292 | 7.6 |
| gamma | | | |
| 93790_LAK cells_IL-2 + IL- | 0.2 | 93358_NCI-H292_IL-4 | 7.2 |
| 18 | | | |
| 93104_LAK | 0.1 | 93360_NCI-H292_IL-9 | 10.8 |
| cells PMA/ionomycin and | | | |
| IL-18 | | | |
| 93578_NK Cells IL- | 0.2 | 93359_NCI-H292_IL-13 | 4.1 |
| 2_resting | | | |
| 93109_Mixed Lymphocyte | 0.3 | 93357_NCI-H292_IFN | 3.9 |
| Reaction_Two Way MLR | | gamma | |
| 93110_Mixed Lymphocyte | 0.1 | 93777_HPAEC_- | 0.2 |
| Reaction_Two Way MLR | | | |
| 93111_Mixed Lymphocyte | 0.0 | 93778_HPAEC_IL-1 | 0.1 |
| Reaction_Two Way MLR | | beta/TNA alpha | |
| 93112_Mononuclear Cells | 0.1 | 93254_Normal Human | 4.8 |
| (PBMCs)_resting | | Lung Fibroblast_none | |
| 93113_Mononuclear Cells | 0.4 | 93253_Normal Human | 1.7 |
| (PBMCs)_PWM | | Lung Fibroblast_TNFa (4 | |
| | | ng/ml) and IL-1b (1 ng/ml) | |
| 93114_Mononuclear Cells | 0.1 | 93257 Normal Human | 7.8 |
| (PBMCs)_PHA-L | | Lung Fibroblast_IL-4 | |
| 93249_Ramos (B cell)_none | 0.1 | 93256_Normal Human | 6.7 |
| | | Lung Fibroblast_IL-9 | |
| 93250_Ramos (B | 0.5 | 93255_Normal Human | 6.0 |
| cell)_ionomycin | | Lung Fibroblast_IL-13 | |
| 93349_B lymphocytes_PWM | 0.7 | 93258_Normal Human | 11.2 |
| | | Lung Fibroblast_IFN | |
| | | gamma | |
| 93350_B | 0.5 | 93106_Dermal Fibroblasts | 10.3 |
| lymphoytes_CD40L and IL-4 | | CCD1070_resting | |
| 92665_EOL-1 | 0.1 | 93361_Dermal Fibroblasts | 7.0 |
| (Eosinophil)_dbcAMP | | CCD1070_TNF alpha 4 | |
| differentiated | | ng/ml | |
| 93248_EOL-1 (Eosinophil)_{—} | 0.1 | 93105_Dermal Fibroblasts | 3.7 |
| dbcAMP/PMAionomycin | | CCD1070_IL-1 beta 1 | |
| | | ng/ml | |
| 93356_Dendritic Cells_none | 0.1 | 93772_dermal | 0.8 |
| | | fibroblast_IFN gamma | |
| 93355_Dendritic Cells_LPS | 0.1 | 93771_dermal | 3.2 |
| 100 ng/ml | | fibroblast_IL-4 | |
| 93775_Dendritic Cells_anti- | 0.1 | 93260_IBD Colitis 2 | 0.8 |
| CD40 | | | |
| 93774_Monocytes_resting | 0.0 | 93261_IBD Crohns | 4.3 |
| 93776_Monocytes_LPS 50 | 0.1 | 735010_Colon_normal | 15.9 |
| ng/ml | | | |
| 93581_Macrophages_resting | 0.1 | 735019_Lung_none | 1.5 |
| 93582_Macrophages_LPS | 0.0 | 64028-1_Thymus_none | 7.7 |
| 100 ng/ml | | | |
| 93098_HUVEC | 0.0 | 64030-1_Kidney_none | 1.3 |
| (Endothelial)_none | | | |
| 93099_HUVEC | 0.3 | | |
| (Endothelial)_starved | | | |

TABLE BE
Panel CNS_neurodegeneration_v1.0

| | Relative | | Relative |
|---|---|---|---|
| | Expression (%) | | Expression (%) |
| | tm7029t_{—} | | tm7029t_{—} |
| Tissue Name | ag2764_b1_s2 | Tissue Name | ag2764_bl_s2 |
| AD 1 Hippo | 13.5 | Control (Path) 3 Temporal | 3.8 |
| | | Ctx | |
| AD 2 Hippo | 39.9 | Control (Path) 4 Temporal | 18.4 |
| | | Ctx | |
| AD 3 Hippo | 9.6 | AD 1 Occipital Ctx | 10.4 |
| AD 4 Hippo | 18.2 | AD 2 Occipital Ctx | 0.0 |
| | | (Missing) | |
| AD 5 hippo | 30.0 | AD 3 Occipital Ctx | 6.9 |
| AD 6 Hippo | 100.0 | AD 4 Occipital Ctx | 14.6 |
| Control 2 Hippo | 24.2 | AD 5 Occipital Ctx | 18.3 |
| Control 4 Hippo | 39.5 | AD 6 Occipital Ctx | 17.1 |
| Control (Path) 3 Hippo | 8.3 | Control 1 Occipital Ctx | 2.1 |
| AD 1 Temporal Ctx | 21.3 | Control 2 Occipital Ctx | 17.6 |
| AD 2 Temporal Ctx | 19.0 | Control 3 Occipital Ctx | 6.3 |
| AD 3 Temporal Ctx | 7.5 | Control 4 Occipital Ctx | 15.3 |
| AD 4 Temporal Ctx | 22.0 | Control (Path) 1 Occipital | 47.9 |
| | | Ctx | |
| AD 5 Inf Temporal Ctx | 43.1 | Control (Path) 2 Occipital | 9.1 |
| | | Ctx | |
| AD 5 SupTemporal Ctx | 51.7 | Control (Path) 3 Occipital | 1.0 |
| | | Ctx | |
| AD 6 Inf Temporal Ctx | 51.7 | Control (Path) 4 Occipital | 9.2 |
| | | Ctx | |
| AD 6 Sup Temporal Ctx | 61.3 | Control 1 Parietal Ctx | 5.2 |
| Control 1 Temporal Ctx | 4.2 | Control 2 Parietal Ctx | 32.5 |
| Control 2 Temporal Ctx | 11.2 | Control 3 Parietal Ctx | 6.8 |
| Control 3 Temporal Ctx | 6.9 | Control (Path) 1 Parietal | 18.8 |
| | | Ctx | |
| Control 4 Temporal Ctx | 11.2 | Control (Path) 2 Parietal | 25.6 |
| | | Ctx | |
| Control (Path) 1 Temporal | 33.7 | Control (Path) 3 Parietal | 1.9 |
| Ctx | | Ctx | |
| Control (Path) 2 Temporal | 18.4 | Control (Path) 4 Parietal | 17.0 |
| Ctx | | Ctx | |

Panel 1.3D Summary The 14578444_{—}0_{—}47 gene is most highly expressed in the thyroid gland (CT=26.2), fetal kidney and fetal skeletal muscle (CTs=27.1) and appears to show an association with normal tissue when compared to samples derived from cancer cell lines. The 14578444_{—}0_{—}47 gene is also expressed at moderate levels in the pancreas, adrenal and pituitary glands, adipose, fetal and adult heart, fetal and adult liver, and adult skeletal muscle (CT=30). The relative overexpression of the 14578444_{—}0_{—}47 gene in fetal skeletal muscle as compared to expression in adult skeletal muscle suggests that the protein product may enhance muscular growth or development in the fetus and thus may also act in a regenerative capacity in the adult. Therefore, therapeutic modulation of the 14578444_{—}0_{—}47 gene could be useful in treatment of muscle related diseases. More specifically, treatment of weak or dystrophic muscle with the protein encoded by this gene could restore muscle mass or function.

The 14578444_{—}0_{—}47 gene is expressed in many tissues originating in the central nervous system, including the amygdala, cerebellum, hippocampus, substantia nigra, thalamus, cerebral cortex, spinal cord, and the developing brain. Please see Panel CNS_neurodegeneration_v1.0 for discussion of potential utility of this gene in the central nervous system.

Panel 2D Summary Highest expression of the 14578444_{—}0_{—}47 gene is detected in normal thyroid tissue (CT=24.7) which is consistent with the results in panel 1.3D. In addition, there is a strong association of the expression of the 14578444_{—}0_{—}47 gene in normal prostate tissue (CT=25). The 14578444_{—}0_{—}47 gene appears to be overexpressed in normal thyroid tissue when compared to expression of the 14578444_{—}0_{—}47 gene in samples derived from matched thyroid cancer tissue. Thus, the expression of this gene could be used to distinguish normal thyroid tissue from the other samples in the panel, and more specifically, from thyroid cancer tissue. Furthermore, therapeutic modulation of the activity or expression of the 14578444_{—}0_{—}47 gene product, through the use of small molecule drugs, antibodies or protein therapeutics, could be effective in the treatment of thyroid and prostate cancers.

Panel 4D Summary The 14578444_{—}0_{—}47 gene is most highly expressed in CD4 lymphocytes activated by anti-CD28 and anti-CD3 cells (CT=23.4). The gene is also highly expressed in resting and cytokine-activated dermal fibroblasts, lung fibroblasts, resting and cytokine-activated mucoepidermoid cell line NCI-H292, and normal colon. The protein encoded by the 14578444_{—}0_{—}47 gene is homologous to a secreted extracellular matrix protein that may be useful as a protein therapeutic to reduce or eliminate the inflammation and autoimmune disease symptoms in patients with psoriasis, chronic obstructive pulmonary disease, asthma, allergies, lupus erythematosus, Crohn's disease, and ulcerative colitis.

Panel CNS_neurodegeneration_v1.0 Summary The 14578444_{—}0_{—}47 gene, a homolog of matrilin-2, appears to be an intercellular matrix protein. While this gene does not appear to be differentially expressed in the Alzheimer's disease brain, the results shown in this panel confirm expression in the brain in independent samples. Glial scarring is a major inhibitor of CNS repair/regeneration involving intra and extra-cellular proteins. Reduction of levels of the protein encoded by the 14578444_{—}0_{—}47 gene may decrease levels of glial scarring in response to CNS injury, and promote healing in spinal cord and/or brain trauma.

A mouse cDNA encoding a novel member of the von Willebrand factor type A-like module superfamily was cloned. The protein precursor of 956 amino acids consists of a putative signal peptide, two von Willebrand factor type A-like domains connected by 10 epidermal growth factor-like modules, a potential oligomerization domain, and a unique segment, and it contains potential N-glycosylation sites. A sequence similarity search indicated the closest relation to the trimeric cartilage matrix protein (CMP). Since they constitute a novel protein family, the term matrilin-2 for the new protein has been introduced, reserving matrilin-1 as an alternative name for CMP. A 3.9-kilobase matrilin-2 mRNA was detected in a variety of mouse organs, including calvaria, uterus, heart, and brain, as well as fibroblast and osteoblast cell lines. Expressed human and rat cDNA sequence tags indicate a high degree of interspecies conservation. A group of 120-150-kDa bands was, after reduction, recognized specifically with an antiserum against the matrilin-2-glutathione S-transferase fusion protein in media of the matrilin-2-expressing cell lines. Assuming glycosylation, this agrees well with the predicted minimum Mr of the mature protein (104,300). Immunolocalization of matrilin-2 in developing skeletal elements showed reactivity in the perichondrium and the osteoblast layer of trabecular bone. CMP binds both collagen fibrils and aggrecan, and because of the similar structure and complementary expression pattern, matrilin-2 is likely to perform similar functions the extracellular matrix assembly of other tissues. See generally Deak F et al., J Bio Chem 272:9268-74 (1997); PMID: 9083061.

NOV5 (SC85803748_A/CG54683-04: GABA Receptor)

Expression of gene SC85803748_A was assessed using the primer-probe sets Ag1307 and Ag1651 described in Tables CA.

TABLE CA

Probe Name Ag1307/Ag1651 (identical sequences)

| | | | | Start |
|---|---|---|---|---|
| Primers | Sequences | TM | Length | Position |
| Forward | 5′-GGTTTGTGCTGCTTCTAACATC-3′ | 58.9 | 22 | 121 |
| | (SEQ ID NO:154) | | | |
| Probe | TET-5′- | 69.3 | 24 | 151 |
| | ACACCAGCGGTGCTCCTCTTCAAT-3′- | | | |
| | TAMRA (SEQ ID NO:155) | | | |
| Reverse | 5′-CATTGAGCATCTTACGGTTTGT-3′ | 59.2 | 22 | 178 |
| | (SEQ ID NO:156) | | | |

Expression of the gene SC85803748_A is low/undetectable (Ct values>35) in all samples in Panel 1.3D, Panel 2.2, Panel 2D and Panel 4D (data not shown).

NOV6a and NOV6b (sggc_draft_ba465b22_{—}20000727/CG55891-01 and CG55891-02: Giant Larvae-like Protein)

Expression of gene sggc_draft_ba465b22_{—}20000727 and variant CG55891-02 was assessed using the primer-probe set Ag2023 described in Table DA. Results from RTQ-PCR runs are shown in Tables DB and DC.

TABLE DA

Probe Name Ag2023

| | | | | Start |
|---|---|---|---|---|
| Primers | Sequences | TM | Length | Position |
| Forward | 5′-CTGGGCATCCAGAAGATCTT-3′ | 59.2 | 20 | 1310 |
| | (SEQ ID NO:157) | | | |
| Probe | FAM-5′- | 69.1 | 25 | 1331 |
| | CTCTGCAAGTACAGCGGCTACCTGG-3′- | | | |
| | TAMRA (SEQ ID NO:158) | | | |
| Reverse | 5′-CCTCGTCATTCAGTTCCAGTAC-3′ | 58.7 | 22 | 1385 |
| | (SEQ ID NO:159) | | | |

TABLE DB
Panel 1.3D

| | Relative | | Relative |
|---|---|---|---|
| | Expression (%) | | Expression (%) |
| | 1.3dtm3202f_{—} | | 1.3dtm3202f_{—} |
| Tissue Name | ag2023 | Tissue Name | ag2023 |
| Liver adenocarcinoma | 42.3 | Kidney (fetal) | 27.4 |
| Pancreas | 24.3 | Renal ca. 786-0 | 3.9 |
| Pancreatic ca. CAPAN 2 | 28.9 | Renal ca. A498 | 6.2 |
| Adrenal gland | 3.8 | Renal ca. RXF 393 | 1.0 |
| Thyroid | 14.2 | Renal ca. ACHN | 3.7 |
| Salivary gland | 14.5 | Renal ca. UO-31 | 0.3 |
| Pituitary gland | 9.7 | Renal ca. TK-10 | 12.6 |
| Brain (fetal) | 0.3 | Liver | 3.6 |
| Brain (whole) | 3.0 | Liver (fetal) | 11.2 |
| Brain (amygdala) | 3.6 | Liver ca. (hepatoblast) | 27.2 |
| | | HepG2 | |
| Brain (cerebellum) | 1.2 | Lung | 36.6 |
| Brain (hippocampus) | 15.0 | Lung (fetal) | 36.6 |
| Brain (substantia nigra) | 4.2 | Lung ca. (small cell) LX-1 | 37.6 |
| Brain (thalamus) | 7.9 | Lung ca. (small cell) NCI- | 20.6 |
| | | H69 | |
| Cerebral Cortex | 1.3 | Lung ca. (s. cell var.) SHP- | 4.4 |
| | | 77 | |
| Spinal cord | 2.4 | Lung ca. (large cell)NCI- | 1.6 |
| | | H460 | |
| CNS ca. (glio/astro) U87- | 0.0 | Lung ca. (non-sm. cell) | 13.9 |
| MG | | A549 | |
| CNS ca. (glio/astro) U-118- | 0.0 | Lung ca. (non-s. cell) NCI- | 0.9 |
| MG | | H23 | |
| CNS ca. (astro) SW1783 | 0.2 | Lung ca (non-s. cell) HOP- | 0.5 |
| | | 62 | |
| CNS ca.* (neuro; met) SK- | 2.5 | Lung ca. (non-s. cl) NCI- | 9.9 |
| N-AS | | H522 | |
| CNS ca. (astro) SF-539 | 0.1 | Lung ca. (squam.) SW 900 | 9.5 |
| CNS ca. (astro) SNB-75 | 7.6 | Lung ca. (squam.) NCI- | 12.1 |
| | | H596 | |
| CNS ca. (glio) SNB-19 | 0.1 | Mammary gland | 20.7 |
| CNS ca. (glio) U251 | 2.1 | Breast ca.* (pl. effusion) | 76.8 |
| | | MCF-7 | |
| CNS ca. (glio) SF-295 | 1.4 | Breast ca.* (pl. ef) MDA- | 1.8 |
| | | MB-231 | |
| Heart (fetal) | 9.3 | Breast ca.* (pl. effusion) | 9.2 |
| | | T47D | |
| Heart | 0.3 | Breast ca. BT-549 | 0.0 |
| Fetal Skeletal | 19.5 | Breast ca. MDA-N | 0.0 |
| Skeletal muscle | 0.5 | Ovary | 1.7 |
| Bone marrow | 0.8 | Ovarian ca. OVCAR-3 | 6.8 |
| Thymus | 2.1 | Ovarian ca. OVCAR-4 | 3.3 |
| Spleen | 44.4 | Ovarian ca. OVCAR-5 | 78.5 |
| Lymph node | 5.0 | Ovarian ca. OVCAR-8 | 3.8 |
| Colorectal | 45.1 | Ovarian ca. IGROV-1 | 9.5 |
| Stomach | 66.0 | Ovarian ca.* (ascites) SK- | 5.5 |
| | | OV-3 | |
| Small intestine | 33.7 | Uterus | 3.0 |
| Colon ca. SW480 | 32.1 | Placenta | 5.9 |
| Colon ca.* (SW480 | 5.9 | Prostate | 21.0 |
| met)SW620 | | | |
| Colon ca. HT29 | 13.3 | Prostate ca.* (bone | 10.4 |
| | | met)PC-3 | |
| Colon ca. HCT-116 | 19.1 | Testis | 10.2 |
| Colon ca. CaCo-2 | 17.2 | Melanoma Hs688(A).T | 0.0 |
| 83219 CC Well to Mod Diff | 23.2 | Melanoma* (met) | 0.0 |
| (ODO3866) | | Hs688(B).T | |
| Colon ca. HCC-2998 | 100.0 | Melanoma UACC-62 | 0.0 |
| Gastric ca.* (liver met) NCI- | 45.7 | Melanoma M14 | 1.6 |
| N87 | | | |
| Bladder | 21.2 | Melanoma LOX IMVI | 0.0 |
| Trachea | 35.6 | Melanoma* (met) SK- | 1.1 |
| | | MEL-5 | |
| Kidney | 22.8 | Adipose | 1.4 |

TABLE DC
Panel 4D

| | Relative | | Relative |
|---|---|---|---|
| | Expression (%) | | Expression (%) |
| | 4dx4tm4241f_{—} | | 4dx4tm4241f_{—} |
| Tissue Name | ag2023_a1 | Tissue Name | ag2023_a1 |
| 93768_Secondary Th1_anti- | 0.0 | 93100_HUVEC | 0.6 |
| CD28/anti-CD3 | | (Endothelial)_IL-1b | |
| 93769_Secondary Th2_anti- | 5.6 | 93779_HUVEC | 1.3 |
| CD28/anti-CD3 | | (Endothelial)_IFN gamma | |
| 93770_Secondary Tr1_anti- | 6.7 | 93102_HUVEC | 0.3 |
| CD28/anti-CD3 | | (Endothelial)_TNF alpha + | |
| | | IFN gamma | |
| 93573_Secondary | 5.6 | 93101_HUVEC | 1.7 |
| Th1_resting day 4-6 in IL-2 | | (Endothelial)_TNF alpha + | |
| | | IL4 | |
| 93572_Secondary | 12.6 | 93781_HUVEC | 1.5 |
| Th2_resting day 4-6 in IL-2 | | (Endothelial)_IL-11 | |
| 93571_Secondary | 4.1 | 93583_Lung | 3.0 |
| Tr1_resting day 4-6 in IL-2 | | Microvascular Endothelial | |
| | | Cells_none | |
| 93568_primary Th1_anti- | 1.4 | 93584_Lung | 0.4 |
| CD28/anti-CD3 | | Microvascular Endothelial | |
| | | Cells_TNFa (4 ng/ml) and | |
| | | IL1b (1 ng/ml) | |
| 93569_primary Th2_anti- | 1.8 | 92662_Microvascular | 1.6 |
| CD28/anti-CD3 | | Dermal endothelium_none | |
| 93570_primary Tr1_anti- | 1.6 | 92663_Microsvasular | 3.2 |
| CD28/anti-CD3 | | Dermal endothelium_TNFa | |
| | | (4 ng/ml) and IL1b (1 | |
| | | ng/ml) | |
| 93565_primary Th1_resting | 4.9 | 93773_Bronchial | 13.8 |
| dy 4-6 in IL-2 | | epithelium_TNFa (4 ng/ml) | |
| | | and IL1b (1 ng/ml)** | |
| 93566_primary Th2_resting | 10.0 | 93347_Small Airway | 12.2 |
| dy 4-6 in IL-2 | | Epithelium_none | |
| 93567_primary Tr1_resting | 4.4 | 93348_Small Airway | 34.9 |
| dy 4-6 in IL-2 | | Epithelium_TNFa (4 | |
| | | ng/ml) and IL1b (1 ng/ml) | |
| 93351_CD45RA CD4 | 5.9 | 92668_Coronery Artery | 0.0 |
| lymphocyte_anti-CD28/anti- | | SMC_resting | |
| CD3 | | | |
| 93352_CD45RO CD4 | 6.3 | 92669 Coronery Artery | 0.0 |
| lymphocyte_anti-CD28/anti- | | SMC_TNFa (4 ng/ml) and | |
| CD3 | | IL1b (1 ng/ml) | |
| 93251_CD8 | 7.4 | 93107_astrocytes_resting | 0.8 |
| Lymphocytes_anti- | | | |
| CD28/anti-CD3 | | | |
| 93353_chronic CD8 | 3.8 | 93108_astrocytes_TNFa (4 | 0.0 |
| Lymphocytes 2ry_resting dy | | ng/ml) and IL1b (1 ng/ml) | |
| 4-6 in IL-2 | | | |
| 93574_chronic CD8 | 1.7 | 92666_KU-812 | 8.9 |
| Lymphocytes 2ry_activated | | (Basophil)_resting | |
| CD3/CD28 | | | |
| 93354_CD4_none | 8.5 | 92667_KU-812 | 9.8 |
| | | (Basophil)_PMA/ionoycin | |
| 93252_Secondary | 4.6 | 93579_CCD1106 | 1.8 |
| Th1/Th2/Tr1_anti-CD95 | | (Keratinocytes)_none | |
| CH11 | | | |
| 93103_LAK cells_resting | 4.1 | 93580_CCD1106 | 2.8 |
| | | (Keratinocytes)_TNFa and | |
| | | IFNg ** | |
| 93788_LAK cells_IL-2 | 5.4 | 93791_Liver Cirrhosis | 14.1 |
| 93787_LAK cells_IL-2 + IL- | 7.9 | 93792_Lupus Kidney | 68.7 |
| 12 | | | |
| 93789_LAK cells_IL-2 + IFN | 6.4 | 93577_NCI-H292 | 61.5 |
| gamma | | | |
| 93790_LAK cells_IL-2 + IL- | 11.1 | 93358_NCI-H292_IL-4 | 100.0 |
| 18 | | | |
| 93104_LAK | 5.3 | 93360_NCI-H292_IL-9 | 58.9 |
| cells_PMA/ionomycin and | | | |
| IL-18 | | | |
| 93578_NK Cells IL- | 17.5 | 93359_NCI-H292_IL-13 | 93.6 |
| 2_resting | | | |
| 93109_Mixed Lymphocyte | 9.1 | 93357_NCI-H292_IFN | 52.9 |
| Reaction_Two Way MLR | | gamma | |
| 93110_Mixed Lymphocyte | 3.9 | 93777_HPAEC_- | 0.3 |
| Reaction_Two Way MLR | | | |
| 93111_Mixed Lymphocyte | 4.0 | 93778_HPAEC_IL-1 | 0.3 |
| Reaction_Two Way MLR | | beta/TNA alpha | |
| 93112_Mononuclear Cells | 7.0 | 93254_Normal Human | 0.0 |
| (PBMCs)_resting | | Lung Fibroblast_none | |
| 93113_Mononuclear Cells | 7.0 | 93253_Normal Human | 0.0 |
| (PBMCs)_PWM | | Lung Fibroblast_TNFa (4 | |
| | | ng/ml) and IL-1b (1 ng/ml) | |
| 93114_Mononuclear Cells | 1.7 | 93257_Normal Human | 0.0 |
| (PBMCs)_PHA-L | | Lung Fibroblast_IL-4 | |
| 93249_Ramos (B cell)_none | 0.8 | 93256_Normal Human | 0.0 |
| | | Lung Fibroblast_IL-9 | |
| 93250_Ramos (B | 2.6 | 93255_Normal Human | 0.0 |
| cell)_ionomycin | | Lung Fibroblast_IL-13 | |
| 93349_B lymphocytes_PWM | 1.0 | 93258_Normal Human | 0.0 |
| | | Lung Fibroblast_IFN | |
| | | gamma | |
| 93350_B | 3.3 | 93106_Dermal Fibroblasts | 0.0 |
| lymphoytes_CD40L and IL-4 | | CCD1070_resting | |
| 92665_EOL-1 | 12.9 | 93361_Dermal Fibroblasts | 4.7 |
| (Eosinophil)_dbcAMP | | CCD1070_TNF alpha 4 | |
| differentiated | | ng/ml | |
| 93248_EOL-1 | 14.2 | 93105 Dermal Fibroblasts | 0.0 |
| (Eosinophil)_dbcAMP/PMAi | | CCD1070_IL-1 beta 1 | |
| onomycin | | ng/ml | |
| 93356_Dendritic Cells_none | 1.4 | 93772_dermal | 0.0 |
| | | fibroblast_IFN gamma | |
| 93355_Dendritic Cells_LPS | 2.9 | 93771_dermal | 0.0 |
| 100 ng/ml | | fibroblast_IL-4 | |
| 93775_Dendritic Cells_anti- | 1.5 | 93260_IBD Colitis 2 | 5.0 |
| CD40 | | | |
| 93774_Monocytes_resting | 1.7 | 93261_IBD Crohns | 29.0 |
| 93776_Monocytes_LPS 50 | 0.0 | 735010_Colon_normal | 53.5 |
| ng/ml | | | |
| 93581_Macrophages_resting | 3.2 | 735019_Lung_none | 13.3 |
| 93582_Macrophages_LPS | 2.9 | 64028-1_Thymus_none | 97.5 |
| 100 ng/ml | | | |
| 93098_HUVEC | 3.6 | 64030-1_Kidney_none | 4.9 |
| (Endothelial)_none | | | |
| 93099_HUVEC | 0.8 | | |
| (Endothelial)_starved | | | |

Panel 1.3D Summary Expression of the sggc_draft_ba465b22_{—}20000727 gene is highest in a sample from a colon cancer cell line (CT=27.9). In addition, there appears to be substantial expression in samples derived from a series of colon cancer cell lines, ovarian cancer cell lines and a breast cancer cell line. Thus, the expression of this gene could be used to distinguish the HCC-2889 cell line from the other samples in the panel. In addition, therapeutic modulation of the protein encoded by the sggc_draft_ba465b22_{—}20000727 gene, through the use of small molecule drugs, antibodies or protein therapeutics, may be useful in the treatment of colon, ovarian or breast cancer.

Among tissues with metabolic function, the sggc_draft_ba465b22_{—}20000727 gene is expressed at moderate levels in adipose, fetal and adult liver, pancreas, and the adrenal, thyroid, and pituitary glands. In addition, the sggc_draft_ba465b22_{—}20000727 gene is expressed in fetal heart and skeletal muscle (CTs=31), but expressed at much lower levels in adult heart and skeletal muscle. This expression profile suggests that expression of the sggc_draft_ba465b22_{—}20000727 gene could be used to differentiate between adult and fetal heart and skeletal muscle tissues. Furthermore, the presence of expression of the sggc_draft_ba465b22_{—}20000727 gene in fetal skeletal muscle when compared to adult skeletal muscle suggests that the protein product may enhance muscular growth or development in the fetus and thus may also act in a regenerative capacity in the adult. Therefore, therapeutic modulation of the sggc_draft_ba465b22_{—}20000727 gene could be useful in treatment of muscle related diseases. More specifically treatment of weak or dystrophic muscle with the protein encoded by this gene could restore muscle mass or function.

The sggc_draft_ba465b22_{—}20000727 gene is also widely expressed among tissues originating in the central nervous system, including the amygdala, cerebellum, hippocampus, thalamus, cerebral cortex, substantia nigra and the spinal cord. The protein encoded by the sggc_draft_ba465b22_{—}20000727 gene is a homolog of the giant larvae-like protein, which is involved in the development of neurons from stem cells. Therefore, therapeutic modulation of the protein product of sggc_draft_ba465b22_{—}20000727 could be used in stem cell therapy in the treatment of diseases characterized by neuronal loss (Alzheimer's, Parkinson's, Huntington's, stroke, head and/or spinal cord trauma).

Panel 4D Summary The sggc_draft_ba465b22_{—}20000727 gene is widely expressed across the samples in this panel. Highest expression of the sggc_draft_ba465b22_{—}20000727 gene is observed in the IL-4 stimulated mucoepidermoid cell line NCI-H292 (CT=28.2), with moderate expression detected in the mucoepidermoid cell line NCI-H292 under resting conditions and following stimulation with IL-9, IL-13, or IFN-gamma. The protein encoded by the sggc_draft_ba465b22_{—}20000727 gene may be involved in regulation of cytoskeletal functions in these cells, as is known for the drosophila gene product. Therefore, the sggc_draft_ba465b22_{—}20000727 gene product could potentially be used as a target for the discovery of small molecule drugs that reduce or eliminate the symptoms in patients with chronic obstructive lung disease.

Loss of cell polarity and tissue architecture are characteristics of malignant cancers derived from epithelial tissues. Evidence from Drosophila has been provided that a group of membrane-associated proteins act in concert to regulate both epithelial structure and cell proliferation. Scribble (Scrib) is a cell junction-localized protein required for polarization of embryonic and imaginal disc and follicular epithelia. The tumor suppressors lethal giant larvae (Igl) and discs-large (dlg) have identical effects on all three epithelia, and that scrib also acts as a tumor suppressor. Scrib and Dlg colocalize and overlap with Lgl in epithelia; activity of all three genes is required for cortical localization of Lgl and junctional localization of Scrib and Dlg. scrib, dlg, and lgl show strong genetic interactions. Data indicate that the three tumor suppressors act together in a common pathway to regulate cell polarity and growth control. See generally, Bilder et al., Science Jul. 7, 2000;289(5476):113-6 (2000); PMID: 10884224.

NOV8a and NOV8b (138531995 and CG111627-01: Nucleotide Sugar Transporter)

Expression of gene 138531995 and variant CG111627-01 was assessed using the primer-probe set Ag1867 described in Table EA. Results from RTQ-PCR runs are shown in Tables EB, EC, ED, EE, and EF.

TABLE EA

Probe Name Ag1867

| | | | | Start |
|---|---|---|---|---|
| Primers | Sequences | TM | Length | Position |
| Forward | 5′-GGTGCCAATACGAAGCTCTTA-3′ | 59.4 | 21 | 593 |
| | (SEQ ID NO:160) | | | |
| Probe | FAM-5′- | 69.8 | 23 | 614 |
| | AGTTCGTCAGCTTCCCCACCCAG-3′- | | | |
| | TAMRA (SEQ ID NO:161) | | | |
| Reverse | 5′-CATGACAGGGATCACCTTAGAG-3′ | 58.7 | 22 | 651 |
| | (SEQ ID NO:162) | | | |

TABLE EB
Panel General_screening_panel_v1.4

| | Relative | | Relative |
|---|---|---|---|
| | Expression (%) | | Expression (%) |
| | tm7173f_{—} | | tm7173f_{—} |
| Tissue Name | ag1867_b1 | Tissue Name | ag1867_b1 |
| D6005-01_Human adipose | 3.5 | Renal ca._TK-10 | 22.3 |
| 112193_Metastatic | 21.0 | Bladder | 10.4 |
| melanoma | | | |
| 112192_Metastatic | 25.3 | Gastric ca.(liver met)_NCI- | 35.9 |
| melanoma | | N87 | |
| 95280_Epidermis (metastatic | 66.5 | 112197_Stomach | 56.1 |
| melanoma) | | | |
| 95279_Epidermis (metastatic | 42.8 | 94938_Colon | 14.1 |
| melanoma) | | Adenocarcinoma | |
| Melanoma (met)_SK-MEL-5 | 65.1 | Colon ca._SW480 | 32.3 |
| 112196_Tongue (oncology) | 14.8 | Colon ca.(SW480 | 22.9 |
| | | met)_SW620 | |
| 113461_Testis Pool | 3.8 | Colon ca._HT29 | 32.5 |
| Prostate ca. (bone met)_PC-3 | 44.4 | Colon ca._HCT-116 | 29.6 |
| 113455_Prostate Pool | 2.9 | Colon ca._CaCo-2 | 17.4 |
| 103396_Placenta | 6.0 | 83219_CC Well to Mod | 14.4 |
| | | Diff (ODO3866) | |
| 113463_Uterus Pool | 2.1 | 94936_Colon | 6.8 |
| | | Adenocarcinoma | |
| Ovarian | 24.2 | 94930_Colon | 8.4 |
| carcinoma_OVCAR-3 | | | |
| Ovarian | 35.6 | 94935_Colon | 9.8 |
| carcinoma(ascites)_SK-OV-3 | | Adenocarcinoma | |
| 95297_Adenocarcinoma | 19.3 | 113468_Colon Pool | 9.0 |
| (ovary) | | | |
| Ovarian | 49.8 | 113457_Small Intestine | 4.6 |
| carcinoma_OVCAR-5 | | Pool | |
| Ovarian carcinoma_IGROV- | 19.3 | 113460_Stomach Pool | 4.3 |
| 1 | | | |
| Ovarian | 16.0 | 113467_Bone Marrow | 3.7 |
| carcinoma_OVCAR-8 | | Pool | |
| 103368_Ovary | 7.9 | 103371_Fetal Heart | 7.7 |
| MCF7_breast | 48.1 | 113451_Heart Pool | 4.3 |
| carcinoma(pleural effusion) | | | |
| Breast ca. (pleural | 65.0 | 113466_Lymph Node Pool | 8.0 |
| effusion)_MDA-MB-231 | | | |
| 112189_ductal cell | 65.2 | 103372_Fetal Skeletal | 4.9 |
| carcinoma(breast) | | Muscle | |
| Breast ca. (pleural | 100.0 | 113456_Skeletal Muscle | 4.7 |
| effusion)_T47D | | Pool | |
| Breast carcinoma_MDA-N | 45.1 | 113459_Spleen Pool | 2.6 |
| 113452_Breast Pool | 8.0 | 113462_Thymus Pool | 6.3 |
| 103398_Trachea | 8.6 | CNS ca. (glio/astro)_U87- | 37.7 |
| | | MG | |
| 112354_lung | 1.9 | CNS ca. (glio/astro)_U- | 44.4 |
| | | 118-MG | |
| 103374_Fetal Lung | 11.5 | CNS ca. (neuro; met) SK- | 35.7 |
| | | N-AS | |
| 94921_Small cell carcinoma | 5.9 | 95264_Brain astrocytoma | 42.0 |
| of the lung | | | |
| Lung ca.(small cell)_LX-1 | 18.6 | CNS ca. (astro)_SNB-75 | 40.3 |
| 94919_Small cell carcinoma | 18.5 | CNS ca. (glio)_SNB-19 | 17.6 |
| of the lung | | | |
| Lung ca.(s. cell var.)_SHP-77 | 22.5 | CNS ca. (glio)_SF-295 | 39.7 |
| 95268_Lung (Large cell | 19.1 | 113447_Brain (Amygdala) | 4.5 |
| carcinoma) | | Pool | |
| 94920_Small cell carcinoma | 8.4 | 103382_Brain (cerebellum) | 18.1 |
| of the lung | | | |
| Lung ca.(non-s. cell)_NCI- | 24.4 | 64019-1_brain(fetal) | 11.2 |
| H23 | | | |
| Lung ca.(large cell)_NCI- | 16.8 | 113448_Brain | 3.9 |
| H460 | | (Hippocampus) Pool | |
| Lung ca.(non-s. cell)_HOP-62 | 33.1 | 113464_Cerebral Cortex | 5.3 |
| | | Pool | |
| Lung ca.(non-s. cl)_NCI- | 28.4 | 113449_Brain (Substantia | 5.6 |
| H522 | | nigra) Pool | |
| 103392_Liver | 3.8 | 113450_Brain (Thalamus) | 6.2 |
| | | Pool | |
| 103393_Fetal Liver | 8.8 | 103384_Brain (whole) | 7.9 |
| Liver | 28.1 | 113458_Spinal Cord Pool | 6.7 |
| ca.(hepatoblast)_HepG2 | | | |
| 113465_Kidney Pool | 7.4 | 103375_Adrenal Gland | 13.4 |
| 103373_Fetal Kidney | 9.2 | 113454_Pituitary gland | 1.1 |
| | | Pool | |
| Renal ca._786-0 | 28.0 | 103397_Salivary Gland | 9.6 |
| 112188_renal cell carcinoma | 10.9 | 103369_Thyroid (female) | 6.1 |
| Renal ca._ACHN | 17.1 | Pancreatic ca._CAPAN2 | 23.4 |
| 112190_Renal cell | 24.9 | 113453_Pancreas Pool | 9.7 |
| carcinoma | | | |

TABLE EC
Panel 2.2

| | Relative | | Relative |
|---|---|---|---|
| | Expression (%) | | Expression (%) |
| | 2.2x4tm6390f_{—} | | 2.2x4tm6390f_{—} |
| Tissue Name | ag1867_a2 | Tissue Name | ag1867_a2 |
| Normal Colon GENPAK | 11.7 | 83793 Kidney NAT | 49.8 |
| 061003 | | (OD04348) | |
| 97759 Colon cancer | 88.6 | 98938 Kidney malignant | 13.1 |
| (OD06064) | | cancer (OD06204B) | |
| 97760 Colon cancer NAT | 36.8 | 98939 Kidney normal | 18.9 |
| (OD06064) | | adjacent tissue | |
| | | (OD06204E) | |
| 97778 Colon cancer | 6.4 | 85973 Kidney Cancer | 43.9 |
| (OD06159) | | (OD04450-01) | |
| 97779 Colon cancer NAT | 16.2 | 85974 Kidney NAT | 14.3 |
| (OD06159) | | (OD04450-03) | |
| 98861 Colon cancer | 6.7 | Kidney Cancer Clontech | 6.1 |
| (OD06297-04) | | 8120613 | |
| 98862 Colon cancer NAT | 13.1 | Kidney NAT Clontech | 30.1 |
| (OD06297-015) | | 8120614 | |
| 83237 CC Gr.2 ascend colon | 11.0 | Kidney Cancer Clontech | 6.9 |
| (ODO3921) | | 9010320 | |
| 83238 CC NAT (ODO3921) | 5.8 | Kidney NAT Clontech | 15.8 |
| | | 9010321 | |
| 97766 Colon cancer | 14.2 | Kidney Cancer Clontech | 51.5 |
| metastasis (OD06104) | | 8120607 | |
| 97767 Lung NAT | 10.5 | Kidney NAT Clontech | 23.8 |
| (OD06104) | | 8120608 | |
| 87472 Colon mets to lung | 14.6 | Normal Uterus GENPAK | 17.4 |
| (OD04451-01) | | 061018 | |
| 87473 Lung NAT | 10.6 | Uterus Cancer GENPAK | 100.0 |
| (OD04451-02) | | 064011 | |
| Normal Prostate Clontech A+ | 12.7 | Normal Thyroid Clontech | 4.9 |
| 6546-1 (8090438) | | A+ 6570-1 (7080817) | |
| 84140 Prostate Cancer | 7.1 | Thyroid Cancer GENPAK | 13.5 |
| (OD04410) | | 064010 | |
| 84141 Prostate NAT | 4.9 | Thyroid Cancer | 20.7 |
| (OD04410) | | INVITROGEN A302152 | |
| Normal Ovary Res. Gen. | 24.5 | Thyroid NAT | 3.0 |
| | | INVITROGEN A302153 | |
| 98863 Ovarian cancer | 15.0 | Normal Breast GENPAK | 18.4 |
| (OD06283-03) | | 061019 | |
| 98865 Ovarian cancer | 7.4 | 84877 Breast Cancer | 5.9 |
| NAT/fallopian tube | | (OD04566) | |
| (OD06283-07) | | | |
| Ovarian Cancer GENPAK | 7.1 | Breast Cancer Res. Gen. | 25.3 |
| 064008 | | 1024 | |
| 97773 Ovarian cancer | 6.3 | 85975 Breast Cancer | 17.7 |
| (OD06145) | | (OD04590-01) | |
| 97775 Ovarian cancer NAT | 7.8 | 85976 Breast Cancer Mets | 17.0 |
| (OD06145) | | (OD04590-03) | |
| 98853 Ovarian cancer | 9.4 | 87070 Breast Cancer | 18.1 |
| (OD06455-03) | | Metastasis (OD04655-05) | |
| 98854 Ovarian NAT | 7.0 | GENPAK Breast Cancer | 16.8 |
| (OD06455-07) Fallopian | | 064006 | |
| tube | | | |
| Normal Lung GENPAK | 8.3 | Breast Cancer Clontech | 23.8 |
| 061010 | | 9100266 | |
| 92337 Invasive poor diff. | 6.8 | Breast NAT Clontech | 12.2 |
| lung adeno (ODO4945-01 | | 9100265 | |
| 92338 Lung NAT | 6.5 | Breast Cancer | 21.8 |
| (ODO4945-03) | | INVITROGEN A209073 | |
| 84136 Lung Malignant | 13.8 | Breast NAT | 21.9 |
| Cancer (OD03126) | | INVITROGEN A2090734 | |
| 84137 Lung NAT | 4.3 | 97763 Breast cancer | 40.3 |
| (OD03126) | | (OD06083) | |
| 90372 Lung Cancer | 10.7 | 97764 Breast cancer node | 18.9 |
| (OD05014A) | | metastasis (OD06083) | |
| 90373 Lung NAT | 15.7 | Normal Liver GENPAK | 16.9 |
| (OD05014B) | | 061009 | |
| 97761 Lung cancer | 7.2 | Liver Cancer Research | 16.2 |
| (OD06081) | | Genetics RNA 1026 | |
| 97762 Lung cancer NAT | 3.5 | Liver Cancer Research | 27.3 |
| (OD06081) | | Genetics RNA 1025 | |
| 85950 Lung Cancer | 12.0 | Paired Liver Cancer Tissue | 17.1 |
| (OD04237-01) | | Research Genetics RNA | |
| | | 6004-T | |
| 85970 Lung NAT | 10.8 | Paired Liver Tissue | 13.6 |
| (OD04237-02) | | Research Genetics RNA | |
| | | 6004-N | |
| 83255 Ocular Mel Met to | 56.4 | Paired Liver Cancer Tissue | 74.8 |
| Liver (ODO4310) | | Research Genetics RNA | |
| | | 6005-T | |
| 83256 Liver NAT | 9.3 | Paired Liver Tissue | 58.3 |
| (ODO4310) | | Research Genetics RNA | |
| | | 6005-N | |
| 84139 Melanoma Mets to | 39.3 | Liver Cancer GENPAK | 11.0 |
| Lung (OD04321) | | 064003 | |
| 84138 Lung NAT | 7.6 | Normal Bladder GENPAK | 8.7 |
| (OD04321) | | 061001 | |
| Normal Kidney GENPAK | 8.3 | Bladder Cancer Research | 11.7 |
| 061008 | | Genetics RNA 1023 | |
| 83786 Kidney Ca, Nuclear | 25.3 | Bladder Cancer | 14.1 |
| grade 2 (OD04338) | | INVITROGEN A302173 | |
| 83787 Kidney NAT | 13.3 | Normal Stomach GENPAK | 26.5 |
| (OD04338) | | 061017 | |
| 83788 Kidney Ca Nuclear | 23.6 | Gastric Cancer Clontech | 14.2 |
| grade 1/2 (OD04339) | | 9060397 | |
| 83789 Kidney NAT | 13.8 | NAT Stomach Clontech | 17.2 |
| (OD04339) | | 9060396 | |
| 83790 Kidney Ca, Clear cell | 10.9 | Gastric Cancer Clontech | 8.5 |
| type (OD04340) | | 9060395 | |
| 83791 Kidney NAT | 11.2 | NAT Stomach Clontech | 26.6 |
| (OD04340) | | 9060394 | |
| 83792 Kidney Ca, Nuclear | 14.7 | Gastric Cancer GENPAK | 6.6 |
| grade 3 (OD04348) | | 064005 | |

TABLE ED
Panel 3.1

| | Relative | | Relative |
|---|---|---|---|
| | Expression (%) | | Expression (%) |
| | tm7536f_{—} | | tm7536f_{—} |
| Tissue Name | ag1867 | Tissue Name | ag1867 |
| 94905_Daoy_Medulloblastoma/ | 4.9 | 94954_Ca Ski_Cervical | 53.6 |
| Cerebellum_sscDNA | | epidermoid carcinoma | |
| | | (metastasis)_sscDNA | |
| 94906_TE671_Medulloblastom/ | 3.4 | 94955_ES-2_Ovarian clear | 21.6 |
| Cerebellum_sscDNA | | cell carcinoma_sscDNA | |
| 94907_D283 | 26.8 | 94957_Ramos/6 h stim_{—} | 6.2 |
| Med_Medulloblastoma/ | | Stimulated with | |
| Cerebellum_sscDNA | | PMA/ionomycin | |
| | | 6 h_sscDNA | |
| 94908_PFSK-1_Primitive | 27.2 | 94958_Ramos/14 h stim_{—} | 6.0 |
| Neuroectodermal/Cerebellum_{—} | | Stimulated with | |
| sscDNA | | PMA/ionomycin | |
| | | 14 h_sscDNA | |
| 94909_XF- | 70.2 | 94962_MEG-01_Chronic | 28.5 |
| 498_CNS_sscDNA | | myelogenous leukemia | |
| | | (megokaryoblast)_sscDNA | |
| 94910_SNB- | 23.8 | 94963_Raji_Burkitt's | 4.7 |
| 78_CNS/glioma_sscDNA | | lymphoma_sscDNA | |
| 94911_SF- | 20.2 | 94964_Daudi_Burkitt's | 6.9 |
| 268_CNS/glioblastoma_{—} | | lymphoma_sscDNA | |
| sscDNA | | | |
| 94912_T98G_Glioblastoma_{—} | 25.9 | 94965_U266_B-cell | 22.8 |
| sscDNA | | plasmacytoma/myeloma_{—} | |
| | | sscDNA | |
| 96776_SK-N- | 36.6 | 94968_CA46_Burkitt's | 4.0 |
| SH_Neuroblastoma | | lymphoma_sscDNA | |
| (metastasis)_sscDNA | | | |
| 94913_SF- | 16.8 | 94970_RL_non-Hodgkin's | 4.2 |
| 295_CNS/glioblastoma_{—} | | B-cell lymphoma_sscDNA | |
| sscDNA | | | |
| 94914_Cerebellum_sscDNA | 12.4 | 94972_JM1_pre-B-cell | 4.6 |
| | | lymphoma/leukemia_{—} | |
| | | sscDNA | |
| 96777_Cerebellum_sscDNA | 8.3 | 94973_Jurkat_T cell | 8.8 |
| | | leukemia_sscDNA | |
| 94916_NCI- | 57.0 | 94974_TF- | 22.8 |
| H292_Mucoepidermoid lung | | 1_Erythroleukemia_{—} | |
| carcinoma_sscDNA | | sscDNA | |
| 94917_DMS-114_Small cell | 18.7 | 94975_HUT 78_T-cell | 29.3 |
| lung cancer_sscDNA | | lymphoma_sscDNA | |
| 94918_DMS-79_Small cell | 27.4 | 94977_U937_Histiocytic | 54.3 |
| lung | | lymphoma_sscDNA | |
| cancer/neuroendocrine_{—} | | | |
| sscDNA | | | |
| 94919_NCI-H146_Small cell | 41.5 | 94980_KU- | 18.6 |
| lung | | 812_Myelogenous | |
| cancer/neuroendocrine_{—} | | leukemia_sscDNA | |
| sscDNA | | | |
| 94920_NCI-H526_Small cell | 29.1 | 94981_769-P_Clear cell | 21.5 |
| lung | | renal carcinoma_sscDNA | |
| cancer/neuroendocrine_{—} | | | |
| sscDNA | | | |
| 94921_NCI-N417_Small cell | 13.3 | 94983_Caki-2_Clear cell | 27.9 |
| lung | | renal carcinoma_sscDNA | |
| cancer/neuroendocrine_{—} | | | |
| sscDNA | | | |
| 94923_NCI-H82_Small cell | 13.4 | 94984_SW 839_Clear cell | 18.2 |
| lung | | renal carcinoma_sscDNA | |
| cancer/neuroendocrine_{—} | | | |
| sscDNA | | | |
| 94924_NCI- | 23.8 | 94986_G401_Wilms' | 7.9 |
| H157_Squamous cell lung | | tumor_sscDNA | |
| cancer (metastasis)_sscDNA | | | |
| 94925_NCI-H1155_Large | 28.7 | 94987_Hs766T_Pancreatic | 28.7 |
| cell lung | | carcinoma (LN | |
| cancer/neuroendocrine_{—} | | metastasis)_sscDNA | |
| sscDNA | | | |
| 94926_NCI-H1299_Large | 17.1 | 94988_CAPAN- | 12.2 |
| cell lung | | 1_Pancreatic | |
| cancer/neuroendocrine_{—} | | adenocarcinoma (liver | |
| sscDNA | | metastasis)_sscDNA | |
| 94927_NCI-H727_Lung | 26.2 | 94989_SU86.86_Pancreatic | 12.5 |
| carcinoid_sscDNA | | carcinoma (liver | |
| | | metastasis)_sscDNA | |
| 94928_NCI-UMC-11_Lung | 100.0 | 94990_BxPC-3_Pancreatic | 8.8 |
| carcinoid_sscDNA | | adenocarcinoma_sscDNA | |
| 94929_LX-1_Small cell lung | 19.1 | 94991_HPAC_Pancreatic | 24.0 |
| cancer_sscDNA | | adenocarcinoma_sscDNA | |
| 94930_Colo-205_Colon | 13.8 | 94992_MIA PaCa- | 7.4 |
| cancer_sscDNA | | 2_Pancreatic | |
| | | carcinoma_sscDNA | |
| 94931_KM12_Colon | 21.0 | 94993_CFPAC- | 47.0 |
| cancer_sscDNA | | 1_Pancreatic ductal | |
| | | adenocarcinoma_sscDNA | |
| 94932_KM20L2_Colon | 25.0 | 94994_PANC- | 43.5 |
| cancer_sscDNA | | 1_Pancreatic epithelioid | |
| | | ductal carcinoma_sscDNA | |
| 94933_NCI-H716_Colon | 49.7 | 94996_T24_Bladder | 19.5 |
| cancer_sscDNA | | carcinma (transitional | |
| | | cell)_sscDNA | |
| 94935_SW-48_Colon | 20.0 | 94997_5637_Bladder | 11.9 |
| adenocarcinoma_sscDNA | | carcinoma_sscDNA | |
| 94936_SW1116_Colon | 9.7 | 94998_HT-1197_Bladder | 23.5 |
| adenocarcinoma_sscDNA | | carcinoma_sscDNA | |
| 94937_LS 174T_Colon | 23.8 | 94999_UM-UC-3_Bladder | 9.7 |
| adenocarcinoma_sscDNA | | carcinma (transitional | |
| | | cell)_sscDNA | |
| 94938_SW-948_Colon | 15.3 | 95000_A204_Rhabdomyosarcoma_{—} | 26.2 |
| adenocarcinoma_sscDNA | | sscDNA | |
| 94939_SW-480_Colon | 20.9 | 95001_HT- | 32.5 |
| adenocarcinoma_sscDNA | | 1080_Fibrosarcoma_{—} | |
| | | sscDNA | |
| 94940_NCI-SNU-5_Gastric | 21.5 | 95002_MG- | 92.0 |
| carcinoma_sscDNA | | 63_Osteosarcoma | |
| | | (bone)_sscDNA | |
| 112197_KATO | 24.7 | 95003_SK-LMS- | 38.2 |
| III_Stomach_sscDNA | | 1 _Leiomyosarcoma | |
| | | (vulva)_sscDNA | |
| 94943_NCI-SNU-16_Gastric | 25.0 | 95004_SJRH30_{—} | 10.6 |
| carcinoma_sscDNA | | Rhabdomyosarcoma (met to | |
| | | bone marrow)_sscDNA | |
| 94944_NCI-SNU-1_Gastric | 18.7 | 95005_A431_Epidermoid | 27.7 |
| carcinoma_sscDNA | | carcinoma_sscDNA | |
| 94946_RF-1_Gastric | 8.2 | 95007_WM266- | 42.0 |
| adenocarcinoma_sscDNA | | 4_Melanoma_sscDNA | |
| 94947_RF-48_Gastric | 8.2 | 112195_DU | 51.4 |
| adenocarcinoma_sscDNA | | 145_Prostate_sscDNA | |
| 96778_MKN-45_Gastric | 28.1 | 95012_MDA-MB- | 14.0 |
| carcinoma_sscDNA | | 468_Breast | |
| | | adenocarcinoma_sscDNA | |
| 94949_NCI-N87_Gastric | 25.7 | 112196_SSC- | 16.5 |
| carcinoma_sscDNA | | 4_Tongue_sscDNA | |
| 94951_OVCAR-5_Ovarian | 11.8 | 112194_SSC- | 41.8 |
| carcinoma_sscDNA | | 9_Tongue_sscDNA | |
| 94952_RL95-2_Uterine | 10.4 | 112191_SSC- | 18.3 |
| carcinoma_sscDNA | | 15_Tongue_sscDNA | |
| 94953_HelaS3_Cervical | 21.2 | 95017_CAL 27_Squamous | 26.4 |
| adenocarcinoma_sscDNA | | cell carcinoma of | |
| | | tongue_sscDNA | |

TABLE EE
Panel 4D

| | Relative | | Relative |
|---|---|---|---|
| | Expression (%) | | Expression (%) |
| | 4dx4tm5555f_{—} | | 4dx4tm5555f_{—} |
| Tissue Name | ag1867_a2 | Tissue Name | ag1867_a2 |
| 93768_Secondary Th1_anti- | 30.7 | 93100_HUVEC | 24.0 |
| CD28/anti-CD3 | | (Endothelial)_IL-1b | |
| 93769_Secondary Th2_anti- | 26.5 | 93779_HUVEC | 30.2 |
| CD28/anti-CD3 | | (Endothelial)_IFN gamma | |
| 93770_Secondary Tr1_anti- | 33.7 | 93102_HUVEC | 17.9 |
| CD28/anti-CD3 | | (Endothelial)_TNF alpha + | |
| | | IFN gamma | |
| 93573_Secondary | 24.6 | 93101_HUVEC | 23.1 |
| Th1_resting day 4-6 in IL-2 | | (Endothelial)_TNF alpha + | |
| | | IL4 | |
| 93572_Secondary | 19.0 | 93781_HUVEC | 21.0 |
| Th2_resting day 4-6 in IL-2 | | (Endothelial)_IL-11 | |
| 93571_Secondary | 18.5 | 93583_Lung | 29.0 |
| Tr1_resting day 4-6 in IL-2 | | Microvascular Endothelial | |
| | | Cells_none | |
| 93568_primary Th1_anti- | 16.3 | 93584_Lung | 20.9 |
| CD28/anti-CD3 | | Microvascular Endothelial | |
| | | Cells_TNFa (4 ng/ml) and | |
| | | IL1b (1 ng/ml) | |
| 93569_primary Th2_anti- | 26.5 | 92662_Microvascular | 32.2 |
| CD28/anti-CD3 | | Dermal endothelium_none | |
| 93570_primary Tr1_anti- | 34.3 | 92663_Microsvasular | 19.4 |
| CD28/anti-CD3 | | Dermal endothelium_TNFa | |
| | | (4 ng/ml) and IL1b (1 | |
| | | ng/ml) | |
| 93565_primary Th1_resting | 47.9 | 93773_Bronchial | 34.1 |
| dy 4-6 in IL-2 | | epithelium_TNFa (4 ng/ml) | |
| | | and IL1b (1 ng/ml)** | |
| 93566_primary Th2_resting | 20.2 | 93347_Small Airway | 22.7 |
| dy 4-6 in IL-2 | | Epithelium_none | |
| 93567_primary Tr1_resting | 23.2 | 93348_Small Airway | 78.6 |
| dy 4-6 in IL-2 | | Epithelium_TNFa (4 | |
| | | ng/ml) and IL1b (1 ng/ml) | |
| 93351_CD45RA CD4 | 33.8 | 92668_Coronery Artery | 45.5 |
| lymphocyte_anti-CD28/anti- | | SMC_resting | |
| CD3 | | | |
| 93352_CD45RO CD4 | 34.9 | 92669_Coronery Artery | 42.8 |
| lymphocyte_anti-CD28/anti- | | SMC_TNFa (4 ng/ml) and | |
| CD3 | | IL1b (1 ng/ml) | |
| 93251_CD8 | 29.9 | 93107_astrocytes_resting | 57.9 |
| Lymphocytes_anti- | | | |
| CD28/anti-CD3 | | | |
| 93353_chronic CD8 | 26.3 | 93108_astrocytes_TNFa (4 | 74.9 |
| Lymphocytes 2ry_resting dy | | ng/ml) and IL1b (1 ng/ml) | |
| 4-6 in IL-2 | | | |
| 93574_chronic CD8 | 22.9 | 92666_KU-812 | 33.3 |
| Lymphocytes 2ry_activated | | (Basophil)_resting | |
| CD3/CD28 | | | |
| 93354_CD4_none | 8.1 | 92667_KU-812 | 75.5 |
| | | (Basophil)_PMA/ionoycin | |
| 93252_Secondary | 20.2 | 93579_CCD1106 | 55.8 |
| Th1/Th2/Tr1_anti-CD95 | | (Keratinocytes)_none | |
| CH11 | | | |
| 93103_LAK cells_resting | 9.2 | 93580_CCD1106 | 100.0 |
| | | (Keratinocytes)_TNFa and | |
| | | IFNg** | |
| 93788_LAK cells_IL-2 | 37.3 | 93791_Liver Cirrhosis | 19.3 |
| 93787_LAK cells_IL-2 + IL- | 32.5 | 93792_Lupus Kidney | 24.3 |
| 12 | | | |
| 93789_LAK cells_IL-2 + IFN | 39.7 | 93577_NCI-H292 | 75.5 |
| gamma | | | |
| 93790_LAK cells_IL-2 + IL- | 28.9 | 93358_NCI-H292_IL-4 | 89.5 |
| 18 | | | |
| 93104_LAK | 11.2 | 93360_NCI-H292_IL-9 | 93.5 |
| cells_PMA/ionomycin and | | | |
| IL-18 | | | |
| 93578_NK Cells IL- | 21.2 | 93359_NCI-H292_IL-13 | 66.4 |
| 2_resting | | | |
| 93109_Mixed Lymphocyte | 22.7 | 93357_NCI-H292_IFN | 48.5 |
| Reaction_Two Way MLR | | gamma | |
| 93110_Mixed Lymphocyte | 22.3 | 93777_HPAEC_- | 24.8 |
| Reaction_Two Way MLR | | | |
| 93111_Mixed Lymphocyte | 18.3 | 93778_HPAEC_IL-1 | 29.0 |
| Reaction_Two Way MLR | | beta/TNA alpha | |
| 93112_Mononuclear Cells | 8.1 | 93254_Normal Human | 79.4 |
| (PBMCs)_resting | | Lung Fibroblast_none | |
| 93113_Mononuclear Cells | 23.0 | 93253_Normal Human | 97.2 |
| (PBMCs)_PWM | | Lung Fibroblast_TNFa (4 | |
| | | ng/ml) and IL-1b (1 ng/ml) | |
| 93114_Mononuclear Cells | 9.9 | 93257_Normal Human | 67.0 |
| (PBMCs)_PHA-L | | Lung Fibroblast_IL-4 | |
| 93249_Ramos (B cell)_none | 25.9 | 93256_Normal Human | 51.7 |
| | | Lung Fibroblast_IL-9 | |
| 93250_Ramos (B | 14.2 | 93255_Normal Human | 44.9 |
| cell)_ionomycin | | Lung Fibroblast_IL-13 | |
| 93349_B lymphocytes_PWM | 25.3 | 93258_Normal Human | 66.6 |
| | | Lung Fibroblast_IFN | |
| | | gamma | |
| 93350_B | 28.3 | 93106_Dermal Fibroblasts | 66.7 |
| lymphoytes_CD40L and IL-4 | | CCD1070_resting | |
| 92665_EOL-1 | 36.8 | 93361_Dermal Fibroblasts | 71.1 |
| (Eosinophil)_dbcAMP | | CCD1070_TNF alpha 4 | |
| differentiated | | ng/ml | |
| 93248_EOL-1 (Eosinophil)_{—} | 21.5 | 93105_Dermal Fibroblasts | 49.5 |
| dbcAMP/PMAionomycin | | CCD1070_IL-1 beta 1 | |
| | | ng/ml | |
| 93356_Dendritic Cells_none | 15.1 | 93772_dermal | 37.8 |
| | | fibroblast_IFN gamma | |
| 93355_Dendritic Cells_LPS | 12.4 | 93771_dermal | 49.6 |
| 100 ng/ml | | fibroblast_IL-4 | |
| 93775_Dendritic Cells_anti- | 16.3 | 93260_IBD Colitis 2 | 4.8 |
| CD40 | | | |
| 93774_Monocytes_resting | 13.6 | 93261_IBD Crohns | 6.1 |
| 93776_Monocytes_LPS 50 | 25.3 | 735010_Colon_normal | 75.0 |
| ng/ml | | | |
| 93581_Macrophages_resting | 19.4 | 735019_Lung_none | 20.4 |
| 93582_Macrophages_LPS | 12.0 | 64028-1_Thymus_none | 24.7 |
| 100 ng/ml | | | |
| 93098_HUVEC | 41.1 | 64030-1_Kidney_none | 23.9 |
| (Endothelial)_none | | | |
| 93099_HUVEC | 57.2 | | |
| (Endothelial)_starved | | | |

TABLE EF
Panel 5

| | Relative | | Relative |
|---|---|---|---|
| | Expression (%) | | Expression (%) |
| | 5dtm7486f_{—} | | 5dtm7486f_{—} |
| Tissue Name | ag1867_s1 | Tissue Name | ag1867_s1 |
| 97457_Patient-02go_adipose | 13.6 | 94709_Donor 2 AM - | 66.9 |
| | | A_adipose | |
| 97476_Patient-07sk_skeletal | 8.5 | 94710_Donor 2 AM - | 39.5 |
| muscle | | B_adipose | |
| 97477_Patient-07ut_uterus | 14.1 | 94711_Donor 2 AM - | 28.1 |
| | | C_adipose | |
| 97478_Patient-07pl_placenta | 14.4 | 94712_Donor 2 AD - | 45.7 |
| | | A_adipose | |
| 97481_Patient-08sk_skeletal | 12.9 | 94713_Donor 2 AD - | 56.6 |
| muscle | | B_adipose | |
| 97482_Patient-08ut_uterus | 8.1 | 94714_Donor 2 AD - | 49.3 |
| | | C_adipose | |
| 97483_Patient-08pl_placenta | 9.9 | 94742_Donor 3 U - | 34.2 |
| | | A_Mesenchymal Stem | |
| | | Cells | |
| 97486_Patient-09sk_skeletal | 3.9 | 94743_Donor 3 U - | 40.9 |
| muscle | | B_Mesenchymal Stem | |
| | | Cells | |
| 97487_Patient-09ut_uterus | 11.2 | 94730_Donor 3 AM - | 59.9 |
| | | A_adipose | |
| 97488_Patient-09pl_placenta | 5.3 | 94731_Donor 3 AM - | 42.3 |
| | | B_adipose | |
| 97492_Patient-10ut_uterus | 19.8 | 94732_Donor 3 AM - | 42.6 |
| | | C_adipose | |
| 97493_Patient-10pl_placenta | 16.4 | 94733_Donor 3 AD - | 77.4 |
| | | A_adipose | |
| 97495_Patient-11go_adipose | 9.0 | 94734_Donor 3 AD - | 38.4 |
| | | B_adipose | |
| 97496_Patient-11sk_skeletal | 8.4 | 94735_Donor 3 AD - | 54.3 |
| muscle | | C_adipose | |
| 97497_Patient-11ut_uterus | 16.0 | 77138_Liver_HepG2untreated | 100.0 |
| 97498_Patient-11pl_placenta | 7.6 | 73556_Heart_Cardiac | 31.9 |
| | | stromal cells (primary) | |
| 97500_Patient-12go_adipose | 14.5 | 81735_Small Intestine | 19.8 |
| 97501_Patient-12sk_skeletal | 21.3 | 72409_Kidney_Proximal | 17.0 |
| muscle | | Convoluted Tubule | |
| 97502_Patient-12ut_uterus | 22.5 | 82685_Small | 7.7 |
| | | intestine_Duodenum | |
| 97503_Patient-12pl_placenta | 7.1 | 90650_Adrenal_Adrenocortical | 7.4 |
| | | adenoma | |
| 94721_Donor 2 U - | 39.8 | 72410_Kidney_HRCE | 54.7 |
| A_Mesenchymal Stem Cells | | | |
| 94722_Donor 2 U - | 33.7 | 72411_Kidney_HRE | 52.5 |
| B_Mesenchymal Stem Cells | | | |
| 94723_Donor 2 U- | 34.2 | 73139_Uterus_Uterine | 30.1 |
| C_Mesenchymal Stem Cells | | smooth muscle cells | |

Panel General_screening_panel_v1.4 Summary The 138531995 gene is ubiquitously expressed in all the samples in this panel, with highest expression in a breast cancer cell line—T47D (CT=23.5). In addition there is substantial expression of this gene in samples derived from other breast cancer cell lines as well as cell lines derived from ovarian cancer, lung cancer, melanoma, renal cancer, colon cancer, gastric cancer and brain cancer. Thus, the expression of this gene could be used to distinguish samples derived from T47D cells from other samples in this panel. In addition, therapeutic modulation of this gene, through the use of small molecule drugs, antibodies or protein therapeutics might useful in the treatment of colon cancer, lung cancer, ovarian cancer, renal cancer, breast cancer, gastric cancer, brain cancer or melanoma.

The 138531995 gene is widely expressed among tissues with metabolic function including adipose, fetal and adult skeletal muscle, fetal and adult liver, fetal and adult heart, the pancreas, and the adrenal, thyroid and pituitary glands. Please see Panel 5 for discussion of potential utility in metabolic function.

The 138531995 gene is homologous to the nucleotide sugar transporters, and is expressed at high levels in the brain (CT=26.0). The nucleotide sugar transporters are indispensable for cellular glycoconjugate synthesis and may have regulatory roles in producing the structural variety of cellular glycoconjugates. Therefore, the protein encoded by the 138531995 gene may be involved in the process of synaptogenesis, and therapeutic up-regulation of the gene or its protein product may be beneficial in the repair process following spinal cord trauma, head trauma, or stroke

Panel 2.2 Summary Highest expression of the 138531995 gene is seen in a sample from a uterine cancer (CT=27.3). In addition, there is substantial expression in samples derived from melanoma and a colon cancer. Of note is the differential expression between some samples of kidney cancer and thyroid cancer, when compared to their respective normal adjacent tissue. Thus, the expression of this gene could be used to distinguish uterine cancer from other samples in the panel. In addition, therapeutic modulation of this gene, through the use of small molecule drugs, antibodies or protein therapeutics might be of benefit in the treatment of kidney cancer, uterine cancer, colon cancer, and melanoma.

Panel 3.1 Summary The 138531995 gene appears to be widely expressed in all of the samples of panel 3.1. This gene is most highly expressed in a sample derived from a lung characinoid cell line (NCI-UMC-11). Thus, the expression of this gene could be used to distinguish NIC-UMC-11 from other samples present in this panel.

Panel 4D Summary The 138531995 gene is expressed in every sample in this panel, with highest expression in keratinocytes stimulated with TNF-alpha and IL-1beta (CT=26.1). Significant expression is also detected in the KU-812 basophil cell line treated with phorbol ester and ionomycin, and in small airway epithelium treated with TNF-alpha and IL-1-beta. The 138531995 gene product is homologous to nucleotide sugar transporters and extracellular epitopes on the membrane protein encoded by the 138531995 gene could potentially be useful as targets for the preparation of therapeutic antibodies that reduce or eliminate the symptoms in patients with psoriasis, allergies, and asthma.

Panel 5 Summary The expression of the 138531995 gene, a putative nucleotide sugar transporter, is highest in panel 5D in the hepatoma cell line HepG2 (CT=26.3). It also shows high expression in mesenchymal stem cells, midway differentiated adipocytes and fully differentiated adipocytes from two human donors. A skeletal muscle sample from a diabetic patient (CT=28.5) shows slightly higher levels of expression of the gene than muscle from patients without diabetes (CTs=30). This may indicate higher levels of glycosylation of proteins in the diabetic patient, since the product of this gene may transport sugars from the cytosol to the Golgi for protein glycosylation. This gene may therefore be involved in carbohydrate metabolism, lipid metabolism and deposition, and lipid disorders such as diabetes.

NOV9 (AC018755_da1/CG53521-01: OB Binding Protein 2)

Expression of gene AC018755_da1 was assessed using the primer-probe set Ag1913 described in Table FA. Results from RTQ-PCR runs are shown in Tables FB, FC, and FD.

TABLE FA

Probe Name Ag1913

| | | | | Start |
|---|---|---|---|---|
| Primers | Sequences | TM | Length | Position |
| Forward | 5′-GAGAACTGTCCAGCTCAATGTC-3′ | 59 | 22 | 675 |
| | (SEQ ID NO:163) | | | |
| Probe | FAM-5′- | 69.1 | 26 | 704 |
| | CTCCACAGACCATCACCATCTTCAGG-3′- | | | |
| | TAMRA (SEQ ID NO:164) | | | |
| Reverse | 5′-TATGAGGTGTTTTGCAGGATCT-3′ | 58.7 | 22 | 746 |
| | (SEQ ID NO:165) | | | |

TABLE FB
Panel 1.3D

| | Relative | | Relative |
|---|---|---|---|
| | Expression (%) | | Expression (%) |
| | 1.3Dtm2787f_{—} | | 1.3Dtm2787f_{—} |
| Tissue Name | ag1913 | Tissue Name | ag1913 |
| Liver adenocarcinoma | 0.0 | Kidney (fetal) | 2.3 |
| Pancreas | 0.7 | Renal ca. 786-0 | 0.0 |
| Pancreatic ca. CAPAN 2 | 0.0 | Renal ca. A498 | 0.0 |
| Adrenal gland | 0.5 | Renal ca. RXF 393 | 0.0 |
| Thyroid | 0.8 | Renal ca. ACHN | 0.4 |
| Salivary gland | 0.5 | Renal ca. UO-31 | 0.0 |
| Pituitary gland | 0.8 | Renal ca. TK-10 | 0.0 |
| Brain (fetal) | 0.4 | Liver | 0.4 |
| Brain (whole) | 0.0 | Liver (fetal) | 12.2 |
| Brain (amygdala) | 1.3 | Liver ca. (hepatoblast) | 0.0 |
| | | HepG2 | |
| Brain (cerebellum) | 0.0 | Lung | 16.5 |
| Brain (hippocampus) | 2.0 | Lung (fetal) | 17.6 |
| Brain (substantia nigra) | 1.3 | Lung ca. (small cell) LX-1 | 0.0 |
| Brain (thalamus) | 2.5 | Lung ca. (small cell) NCI- | 0.0 |
| | | H69 | |
| Cerebral Cortex | 6.0 | Lung ca. (s. cell var.) SHP- | 0.0 |
| | | 77 | |
| Spinal cord | 2.9 | Lung ca. (large cell)NCI- | 0.0 |
| | | H460 | |
| CNS ca. (glio/astro) U87- | 0.0 | Lung ca. (non-sm. cell) | 0.0 |
| MG | | A549 | |
| CNS ca. (glio/astro) U-118- | 0.0 | Lung ca. (non-s.cell) NCI- | 0.0 |
| MG | | H23 | |
| CNS ca. (astro) SW1783 | 0.0 | Lung ca (non-s. cell) HOP- | 0.0 |
| | | 62 | |
| CNS ca.* (neuro; met) SK- | 0.0 | Lung ca. (non-s. cl) NCI- | 0.0 |
| N-AS | | H522 | |
| CNS ca. (astro) SF-539 | 0.0 | Lung ca. (squam.) SW 900 | 0.0 |
| CNS ca. (astro) SNB-75 | 47.0 | Lung ca. (squam.) NCI- | 0.0 |
| | | H596 | |
| CNS ca. (glio) SNB-19 | 0.0 | Mammary gland | 2.2 |
| CNS ca. (glio) U251 | 0.0 | Breast ca.* (pl. effusion) | 0.0 |
| | | MCF-7 | |
| CNS ca. (glio) SF-295 | 0.0 | Breast ca.* (pl. ef) MDA- | 0.0 |
| | | MB-231 | |
| Heart (fetal) | 9.7 | Breast ca.* (pl. effusion) | 0.0 |
| | | T47D | |
| Heart | 0.4 | Breast ca. BT-549 | 0.0 |
| Fetal Skeletal | 9.7 | Breast ca. MDA-N | 0.0 |
| Skeletal muscle | 0.0 | Ovary | 3.7 |
| Bone marrow | 100.0 | Ovarian ca. OVCAR-3 | 0.0 |
| Thymus | 1.3 | Ovarian ca. OVCAR-4 | 0.0 |
| Spleen | 24.3 | Ovarian ca. OVCAR-5 | 0.0 |
| Lymph node | 9.7 | Ovarian ca. OVCAR-8 | 0.0 |
| Colorectal | 4.2 | Ovarian ca. IGROV-1 | 0.0 |
| Stomach | 6.2 | Ovarian ca.* (ascites) SK- | 0.0 |
| | | OV-3 | |
| Small intestine | 0.5 | Uterus | 0.6 |
| Colon ca. SW480 | 0.0 | Placenta | 9.4 |
| Colon ca.* (SW480 | 0.0 | Prostate | 1.3 |
| met)SW620 | | | |
| Colon ca. HT29 | 0.0 | Prostate ca.* (bone | 0.0 |
| | | met)PC-3 | |
| Colon ca. HCT-116 | 0.0 | Testis | 0.8 |
| Colon ca. CaCo-2 | 0.6 | Melanoma Hs688(A).T | 0.0 |
| 83219 CC Well to Mod Diff | 5.1 | Melanoma* (met) | 0.0 |
| (ODO3866) | | Hs688(B).T | |
| Colon ca. HCC-2998 | 0.0 | Melanoma UACC-62 | 0.0 |
| Gastric ca.* (liver met) NCI- | 0.0 | Melanoma M14 | 0.0 |
| N87 | | | |
| Bladder | 1.6 | Melanoma LOX IMVI | 0.0 |
| Trachea | 1.9 | Melanoma* (met) SK- | 0.0 |
| | | MEL-5 | |
| Kidney | 0.0 | Adipose | 3.8 |

TABLE FC
Panel 2.2

| | Relative | | Relative |
|---|---|---|---|
| | Expression (%) | | Expression (%) |
| | 2.2x4tm6417f_{—} | | 2.2x4tm6417f_{—} |
| Tissue Name | ag1913_a1 | Tissue Name | ag1913_a1 |
| Normal Colon GENPAK | 4.7 | 83793 Kidney NAT | 34.7 |
| 061003 | | (OD04348) | |
| 97759 Colon cancer | 52.3 | 98938 Kidney malignant | 0.0 |
| (OD06064) | | cancer (OD06204B) | |
| 97760 Colon cancer NAT | 24.9 | 98939 Kidney normal | 4.8 |
| (OD06064) | | adjacent tissue | |
| | | (OD06204E) | |
| 97778 Colon cancer | 0.0 | 85973 Kidney Cancer | 4.8 |
| (OD06159) | | (OD04450-01) | |
| 97779 Colon cancer NAT | 19.6 | 85974 Kidney NAT | 0.0 |
| (OD06159) | | (OD04450-03) | |
| 98861 Colon cancer | 2.7 | Kidney Cancer Clontech | 1.1 |
| (OD06297-04) | | 8120613 | |
| 98862 Colon cancer NAT | 0.0 | Kidney NAT Clontech | 0.0 |
| (OD06297-015) | | 8120614 | |
| 83237 CC Gr.2 ascend colon | 2.6 | Kidney Cancer Clontech | 2.2 |
| (ODO3921) | | 9010320 | |
| 83238 CC NAT (ODO3921) | 3.5 | Kidney NAT Clontech | 8.8 |
| | | 9010321 | |
| 97766 Colon cancer | 3.4 | Kidney Cancer Clontech | 24.8 |
| metastasis (OD06104) | | 8120607 | |
| 97767 Lung NAT | 22.9 | Kidney NAT Clontech | 0.0 |
| (OD06104) | | 8120608 | |
| 87472 Colon mets to lung | 4.5 | Normal Uterus GENPAK | 27.5 |
| (OD04451-01) | | 061018 | |
| 87473 Lung NAT | 27.8 | Uterus Cancer GENPAK | 4.8 |
| (OD04451-02) | | 064011 | |
| Normal Prostate Clontech A+ | 0.0 | Normal Thyroid Clontech | 4.4 |
| 6546-1 (8090438) | | A+ 6570-1 (7080817) | |
| 84140 Prostate Cancer | 0.0 | Thyroid Cancer GENPAK | 0.0 |
| (OD04410) | | 064010 | |
| 84141 Prostate NAT | 15.8 | Thyroid Cancer | 0.0 |
| (OD04410) | | INVITROGEN A302152 | |
| Normal Ovary Res. Gen. | 10.1 | Thyroid NAT | 4.4 |
| | | INVITROGEN A302153 | |
| 98863 Ovarian cancer | 14.8 | Normal Breast GENPAK | 21.5 |
| (OD06283-03) | | 061019 | |
| 98865 Ovarian cancer | 16.1 | 84877 Breast Cancer | 8.4 |
| NAT/fallopian tube | | (OD04566) | |
| (OD06283-07) | | | |
| Ovarian Cancer GENPAK | 3.0 | Breast Cancer Res. Gen. | 22.3 |
| 064008 | | 1024 | |
| 97773 Ovarian cancer | 12.7 | 85975 Breast Cancer | 23.4 |
| (OD06145) | | (OD04590-01) | |
| 97775 Ovarian cancer NAT | 21.3 | 85976 Breast Cancer Mets | 15.4 |
| (OD06145) | | (OD04590-03) | |
| 98853 Ovarian cancer | 0.0 | 87070 Breast Cancer | 9.3 |
| (OD06455-03) | | Metastasis (OD04655-05) | |
| 98854 Ovarian NAT | 3.6 | GENPAK Breast Cancer | 10.7 |
| (OD06455-07) Fallopian | | 064006 | |
| tube | | | |
| Normal Lung GENPAK | 23.2 | Breast Cancer Clontech | 33.8 |
| 061010 | | 9100266 | |
| 92337 Invasive poor diff. | 4.1 | Breast NAT Clontech | 22.9 |
| lung adeno (ODO4945-01 | | 9100265 | |
| 92338 Lung NAT | 26.8 | Breast Cancer | 9.3 |
| (ODO4945-03) | | INVITROGEN A209073 | |
| 84136 Lung Malignant | 21.3 | Breast NAT | 0.0 |
| Cancer (OD03126) | | INVITROGEN A2090734 | |
| 84137 Lung NAT | 14.5 | 97763 Breast cancer | 3.9 |
| (OD03126) | | (OD06083) | |
| 90372 Lung Cancer | 1.6 | 97764 Breast cancer node | 19.4 |
| (OD05014A) | | metastasis (OD06083) | |
| 90373 Lung NAT | 35.7 | Normal Liver GENPAK | 21.1 |
| (OD05014B) | | 061009 | |
| 97761 Lung cancer | 0.0 | Liver Cancer Research | 18.8 |
| (OD06081) | | Genetics RNA 1026 | |
| 97762 Lung cancer NAT | 0.0 | Liver Cancer Research | 25.5 |
| (OD06081) | | Genetics RNA 1025 | |
| 85950 Lung Cancer | 13.5 | Paired Liver Cancer Tissue | 52.0 |
| (OD04237-01) | | Research Genetics RNA | |
| | | 6004-T | |
| 85970 Lung NAT | 50.3 | Paired Liver Tissue | 23.7 |
| (OD04237-02) | | Research Genetics RNA | |
| | | 6004-N | |
| 83255 Ocular Mel Met to | 0.0 | Paired Liver Cancer Tissue | 20.2 |
| Liver (ODO4310) | | Research Genetics RNA | |
| | | 6005-T | |
| 83256 Liver NAT | 28.6 | Paired Liver Tissue | 100.0 |
| (ODO4310) | | Research Genetics RNA | |
| | | 6005-N | |
| 84139 Melanoma Mets to | 0.0 | Liver Cancer GENPAK | 8.8 |
| Lung (OD04321) | | 064003 | |
| 84138 Lung NAT | 74.0 | Normal Bladder GENPAK | 23.6 |
| (OD04321) | | 061001 | |
| Normal Kidney GENPAK | 0.0 | Bladder Cancer Research | 15.3 |
| 061008 | | Genetics RNA 1023 | |
| 83786 Kidney Ca, Nuclear | 19.8 | Bladder Cancer | 9.8 |
| grade 2 (OD04338) | | INVITROGEN A302173 | |
| 83787 Kidney NAT | 1.2 | Normal Stomach GENPAK | 4.9 |
| (OD04338) | | 061017 | |
| 83788 Kidney Ca Nuclear | 22.1 | Gastric Cancer Clontech | 7.2 |
| grade 1/2 (OD04339) | | 9060397 | |
| 83789 Kidney NAT | 9.4 | NAT Stomach Clontech | 14.4 |
| (OD04339) | | 9060396 | |
| 83790 Kidney Ca, Clear cell | 0.0 | Gastric Cancer Clontech | 26.5 |
| type (OD04340) | | 9060395 | |
| 83791 Kidney NAT | 3.4 | NAT Stomach Clontech | 38.2 |
| (OD04340) | | 9060394 | |
| 83792 Kidney Ca, Nuclear | 9.7 | Gastric Cancer GENPAK | 0.0 |
| grade 3 (OD04348) | | 064005 | |

TABLE FD
Panel 4D

| | Relative | | Relative |
|---|---|---|---|
| | Expression (%) | | Expression (%) |
| | 4dx4tm4409f_{—} | | 4dx4tm4409f_{—} |
| Tissue Name | ag1913_a1 | Tissue Name | ag1913_a1 |
| 93768_Secondary Th1_anti- | 0.0 | 93100_HUVEC | 0.0 |
| CD28/anti-CD3 | | (Endothelial)_IL-1b | |
| 93769_Secondary Th2_anti- | 0.0 | 93779_HUVEC | 0.0 |
| CD28/anti-CD3 | | (Endothelial)_IFN gamma | |
| 93770_Secondary Tr1_anti- | 0.0 | 93102_HUVEC | 0.0 |
| CD28/anti-CD3 | | (Endothelial)_TNF alpha + | |
| | | IFN gamma | |
| 93573_Secondary | 0.0 | 93101_HUVEC | 0.0 |
| Th1_resting day 4-6 in IL-2 | | (Endothelial)_TNF alpha + | |
| | | IL4 | |
| 93572_Secondary | 0.0 | 93781_HUVEC | 0.0 |
| Th2_resting day 4-6 in IL-2 | | (Endothelial)_IL-11 | |
| 93571_Secondary | 0.0 | 93583_Lung | 0.0 |
| Tr1_resting day 4-6 in IL-2 | | Microvascular Endothelial | |
| | | Cells_none | |
| 93568_primary Th1_anti- | 0.0 | 93584_Lung | 0.0 |
| CD28/anti-CD3 | | Microvascular Endothelial | |
| | | Cells_TNFa (4 ng/ml) and | |
| | | IL1b (1 ng/ml) | |
| 93569_primary Th2_anti- | 0.0 | 92662_Microvascular | 0.2 |
| CD28/anti-CD3 | | Dermal endothelium_none | |
| 93570_primary Tr1_anti- | 0.0 | 92663_Microsvasular | 0.0 |
| CD28/anti-CD3 | | Dermal endothelium_TNFa | |
| | | (4 ng/ml) and IL1b (1 | |
| | | ng/ml) | |
| 93565_primary Th1_resting | 0.0 | 93773_Bronchial | 0.0 |
| dy 4-6 in IL-2 | | epithelium_TNFa (4 ng/ml) | |
| | | and IL1b (1 ng/ml)** | |
| 93566_primary Th2_resting | 0.0 | 93347_Small Airway | 0.0 |
| dy 4-6 in IL-2 | | Epithelium_none | |
| 93567_primary Tr1_resting | 0.0 | 93348_Small Airway | 0.0 |
| dy 4-6 in IL-2 | | Epithelium_TNFa (4 | |
| | | ng/ml) and IL1b (1 ng/ml) | |
| 93351_CD45RA CD4 | 0.0 | 92668_Coronery Artery | 0.0 |
| lymphocyte_anti-CD28/anti- | | SMC_resting | |
| CD3 | | | |
| 93352_CD45RO CD4 | 0.6 | 92669_Coronery Artery | 0.0 |
| lymphocyte_anti-CD28/anti- | | SMC_TNFa (4 ng/ml) and | |
| CD3 | | IL1b (1 ng/ml) | |
| 93251_CD8 | 0.0 | 93107_astrocytes_resting | 0.0 |
| Lymphocytes_anti- | | | |
| CD28/anti-CD3 | | | |
| 93353_chronic CD8 | 0.6 | 93108_astrocytes_TNFa (4 | 0.0 |
| Lymphocytes 2ry_resting dy | | ng/ml) and IL1b (1 ng/ml) | |
| 4-6 in IL-2 | | | |
| 93574_chronic CD8 | 0.0 | 92666_KU-812 | 0.1 |
| Lymphocytes 2ry_activated | | (Basophil)_resting | |
| CD3/CD28 | | | |
| 93354_CD4_none | 0.5 | 92667_KU-812 | 3.4 |
| | | (Basophil)_PMA/ionoycin | |
| 93252_Secondary | 0.0 | 93579_CCD1106 | 0.0 |
| Th1/Th2/Tr1_anti-CD95 | | (Keratinocytes)_none | |
| CH11 | | | |
| 93103_LAK cells_resting | 26.1 | 93580_CCD1106 | 0.0 |
| | | (Keratinocytes)_TNFa and | |
| | | IFNg ** | |
| 93788_LAK cells_IL-2 | 0.3 | 93791_Liver Cirrhosis | 1.0 |
| 93787_LAK cells_IL-2 + IL- | 0.3 | 93792_Lupus Kidney | 0.5 |
| 12 | | | |
| 93789_LAK cells_IL-2 + IFN | 2.6 | 93577_NCI-H292 | 0.0 |
| gamma | | | |
| 93790_LAK cells_IL-2 + IL- | 0.9 | 93358_NCI-H292_IL-4 | 0.1 |
| 18 | | | |
| 93104_LAK | 5.9 | 93360_NCI-H292_IL-9 | 0.3 |
| cells_PMA/ionomycin and | | | |
| IL-18 | | | |
| 93578_NK Cells IL- | 1.5 | 93359_NCI-H292_IL-13 | 0.0 |
| 2_resting | | | |
| 93109_Mixed Lymphocyte | 12.1 | 93357_NCI-H292_IFN | 0.3 |
| Reaction_Two Way MLR | | gamma | |
| 93110_Mixed Lymphocyte | 6.4 | 93777_HPAEC_- | 0.0 |
| Reaction_Two Way MLR | | | |
| 93111_Mixed Lymphocyte | 0.3 | 93778_HPAEC_IL-1 | 0.0 |
| Reaction_Two Way MLR | | beta/TNA alpha | |
| 93112_Mononuclear Cells | 6.7 | 93254_Normal Human | 0.0 |
| (PBMCs)_resting | | Lung Fibroblast_none | |
| 93113_Mononuclear Cells | 13.0 | 93253_Normal Human | 0.0 |
| (PBMCs)_PWM | | Lung Fibroblast_TNFa (4 | |
| | | ng/ml) and IL-1b (1 ng/ml) | |
| 93114_Mononuclear Cells | 9.0 | 93257_Normal Human | 0.0 |
| (PBMCs)_PHA-L | | Lung Fibroblast_IL-4 | |
| 93249_Ramos (B cell)_none | 0.0 | 93256_Normal Human | 0.0 |
| | | Lung Fibroblast_IL-9 | |
| 93250_Ramos (B | 0.0 | 93255_Normal Human | 0.0 |
| cell)_ionomycin | | Lung Fibroblast_IL-13 | |
| 93349_B lymphocytes_PWM | 5.7 | 93258_Normal Human | 0.0 |
| | | Lung Fibroblast_IFN | |
| | | gamma | |
| 93350_B | 6.4 | 93106_Dermal Fibroblasts | 0.0 |
| lymphoytes_CD40L and IL-4 | | CCD1070_resting | |
| 92665_EOL-1 | 5.1 | 93361_Dermal Fibroblasts | 0.0 |
| (Eosinophil)_dbcAMP | | CCD1070_TNF alpha 4 | |
| differentiated | | ng/ml | |
| 93248_EOL-1 (Eosinophil)_{—} | 14.2 | 93105_Dermal Fibroblasts | 0.0 |
| dbcAMP/PMAionomycin | | CCD1070_IL-1 beta 1 | |
| | | ng/ml | |
| 93356_Dendritic Cells_none | 26.5 | 93772_dermal | 0.0 |
| | | fibroblast_IFN gamma | |
| 93355_Dendritic Cells_LPS | 39.9 | 93771_dermal | 0.0 |
| 100 ng/ml | | fibroblast_IL-4 | |
| 93775_Dendritic Cells_anti- | 36.2 | 93260_IBD Colitis 2 | 0.2 |
| CD40 | | | |
| 93774_Monocytes_resting | 100.0 | 93261_IBD Crohns | 0.6 |
| 93776_Monocytes_LPS 50 | 80.6 | 735010_Colon_normal | 3.7 |
| ng/ml | | | |
| 93581_Macrophages_resting | 19.4 | 735019_Lung_none | 3.3 |
| 93582_Macrophages_LPS | 11.7 | 64028-1_Thymus_none | 0.3 |
| 100 ng/ml | | | |
| 93098_HUVEC | 0.0 | 64030-1_Kidney_none | 2.2 |
| (Endothelial)_none | | | |
| 93099_HUVEC | 0.0 | | |
| (Endothelial)_starved | | | |

Panel 1.3D Summary The AC018755_da1 gene is most highly expressed in bone marrow (CT=29). Thus, the expression of this gene could be used to distinguish bone marrow from other samples in this panel.

Expression of this gene appears to be restricted to a subset of normal tissue samples. Among tissues with metabolic function, the AC018755_da1 gene is expressed in adipose and is also expressed in fetal skeletal muscle, heart, and liver, but is expressed at much lower levels in the corresponding mature tissues. This expression profile suggests that expression of the AC018755_da1 gene could be used to differentiate betweent the two sources of tissue in heart, liver and skeletal muscle. The difference in expression in fetal and adult tissue may also indicate an involvement of the gene product in the differentiation processes leading to the formation of the adult organs. In addition, the higher expression in fetal skeletal muscle when compared to adult skeletal muscle suggests that the AC018755_da1 gene product could be involved in muscular growth or development in the fetus and could potentially act in a regenerative capacity in the adult. Thus, therapeutic modulation of the AC018755_da1 gene could be useful in the treatment of muscle related diseases and treatment with the protein product could restore muscle mass or function to weak or dystrophic muscle. Furthermore, expression of the AC018755_da1 gene in the placenta may suggest that the AC018755_da1 protein is involved in the regulation of metabolic processes.

Expression of the AC018755_da1 gene in tissues associated with the central nervous system is limited to low but significant expression in the hippocampus, thalamus, cerebral cortex and spinal cord. This expression pattern suggests that the protein encoded by the AC018755_da1 gene could play a role in CNS processes. The sialic acid-binding immunoglobulin-like lectins, to which the AC018755_da1 gene product is homologous, are a subgroup of the immunoglobulin (Ig) superfamily that mediate protein-carbohydrate interactions. They specifically interact with sialic acids in glycoproteins and glycolipids, with each SIGLEC having a particular preference for both the nature of the sialic acid and its glycosidic linkage to adjacent sugars. Specific sialic acids are known to mediate cell-cell adhesion between neurons at synaptic contacts. These sialic acid mediated synaptic interactions are modified during the synaptic restructuring that takes place during the process of learning and memory formation. Therefore, drugs that inhibit the protein encoded by the AC018755_da1 gene could serve to treat disorders of memory that occur with CNS diseases such as Alzheimer's disease or with normal aging.

Panel 2.2 Summary Highest expression of the AC018755_da1 gene is detected in normal liver tissue adjacent to a liver tumor (CT=33.1). In addition, there is substantial expression in samples derived from normal adjacent lung tissue (2 samples) and a colon cancer. Thus, AC018755_da1 gene could be used to distinguish normal adjacent liver from other samples in the panel. In addition, the expression of this gene could also be used to distinguish normal lung margin when compared to their partner samples in the panel.

Panel 4D Summary The AC018755_da1 gene is highly expressed in both resting (CT=27.7) and LPS-activated monocytes, resting and LPS-activated macrophages, and resting and anti-CD40- or LPS-activated dendritic cells. The AC018755_da1 gene is also expressed at lower levels in a (dibutyryl-cAMP+phorbol ester+ionomycin)-differentiated eosinophil cell line EOL-1, and in resting lymphokine-activated killer cells. The protein encoded by the AC018755_da1 gene is homologous to an integral membrane protein and could be used as a target for antagonist therapeutic antibodies that block the signaling functions of the AC018755_da1 gene product in these inflammatory mediator cells. In addition, the soluble extracellular domain of the putative single-pass membrane protein may be useful as a therapeutic protein that binds the AC018755_da1 gene product ligand(s) and inhibits the pro-inflammaory signaling function of the protein encoded by the AC018755_da1 gene. Such therapeutic antibodies and soluble extracellular domain therapeutic proteins may reduce or eliminate the inflammatory and autoimmune disease symptoms in patients with rheumatoid arthritis, lupus erythematosus, inflammatory bowel disease, Crohn's disease, allergies, and asthma.

The addition of poly-alpha2,8-N-acetylneuraminic acid (polysialic acid; PSA) to the neural cell adhesion molecule NCAM plays a crucial role in neural development [1-3], neural regeneration [4], and plastic processes in the vertebrate brain associated with neurite outgrowth [5], axonal pathfinding [6], and learning and memory [7,-9]. PSA levels are decreased in people affected by schizophrenia [10], and PSA has been identified as a specific marker for some neuroendocrine and lymphoblastoid tumours [11-13]; expression of PSA on the surface of these tumour cells modulates their metastatic potential [11-13]. Studies aimed at understanding PSA biosynthesis and the dynamics of its production have largely been promoted by the cloning of polysialyltransferases (PST-1 in hamster; PST in human and mouse) [14-16]. However, the number of enzymes involved in the biosynthesis of PSA has not been identified. Using incompletely glycosylated NCAM variants and soluble recombinant glycosyltransferases, the site at which PST-1 acts to polysialylate NCAM in vitro was reconstructed. The data presented clearly demonstrate that polysialylation of NCAM is catalyzed by a single enzyme, PST-1, and that terminal sialylation of the N-glycan core is sufficient to generate the PSA acceptor site. Results also show that PST-1 can act on core structures with the terminal sialic acid connected to galactose via an alpha2,3 or alpha2,6 linkage. See generally, Muhlenhoff et al., Curr Biol. 6:1188-91 (1996); PMID: 8805371

NOV10 (30675745.0.499_da1/CG52083-01: Trypsin-like)

Expression of gene 30675745.0.499_da1 was assessed using the primer-probe set Ag1876 described in Table GA. Results from RTQ-PCR runs are shown in Tables GB and GC.

TABLE GA

Probe Name Ag1876

| | | | | Start |
|---|---|---|---|---|
| Primers | Sequences | TM | Length | Position |
| Forward | 5′-AGCAAGATTGCTCACACAGAGT-3′ | 59.2 | 22 | 668 |
| | (SEQ ID NO:166) | | | |
| Probe | TET-5′- | 69.1 | 28 | 692 |
| | CCAGTCAATACCATCATCATCCATGAGG- | | | |
| | 3′-TAMRA (SEQ ID NO:167) | | | |
| Reverse | 5′-TATGTTGTTGCTCATGGAGTTG-3′ | 58.7 | 22 | 730 |
| | (SEQ ID NO:168) | | | |

TABLE GB
Panel 1.3D

| | Relative | | Relative |
|---|---|---|---|
| | Expression (%) | | Expression (%) |
| | 1.3dx4tm5422 | | 1.3dx4tm5422 |
| Tissue Name | t_ag1876_a1 | Tissue Name | t_ag1876_a1 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.4 | Renal ca. A498 | 0.5 |
| Adrenal gland | 0.7 | Renal ca. RXF 393 | 0.0 |
| Thyroid | 0.0 | Renal ca. ACHN | 0.6 |
| Salivary gland | 0.4 | Renal ca. UO-31 | 0.0 |
| Pituitary gland | 0.0 | Renal ca. TK-10 | 0.4 |
| Brain (fetal) | 0.3 | Liver | 0.0 |
| Brain (whole) | 4.2 | Liver (fetal) | 0.0 |
| Brain (amygdala) | 2.5 | Liver ca. (hepatoblast) | 0.0 |
| | | HepG2 | |
| Brain (cerebellum) | 0.8 | Lung | 0.0 |
| Brain (hippocampus) | 2.7 | Lung (fetal) | 0.0 |
| Brain (substantia nigra) | 1.4 | Lung ca. (small cell) LX-1 | 1.3 |
| Brain (thalamus) | 1.6 | Lung ca. (small cell) NCI- | 0.0 |
| | | H69 | |
| Cerebral Cortex | 0.6 | Lung ca. (s. cell var.) SHP- | 0.0 |
| | | 77 | |
| Spinal cord | 0.4 | Lung ca. (large cell)NCI- | 0.4 |
| | | H460 | |
| CNS ca. (glio/astro) U87- | 0.0 | Lung ca. (non-sm. cell) | 0.5 |
| MG | | A549 | |
| CNS ca. (glio/astro) U-118- | 0.2 | Lung ca. (non-s. cell) NCI- | 0.0 |
| MG | | H23 | |
| CNS ca. (astro) SW1783 | 0.0 | Lung ca (non-s. cell) HOP- | 0.0 |
| | | 62 | |
| CNS ca.* (neuro; met) SK- | 0.7 | Lung ca. (non-s. cl) NCI- | 0.3 |
| N-AS | | H522 | |
| CNS ca. (astro) SF-539 | 0.0 | Lung ca. (squam.) SW 900 | 0.8 |
| CNS ca. (astro) SNB-75 | 0.0 | Lung ca. (squam.) NCI- | 0.0 |
| | | H596 | |
| CNS ca. (glio) SNB-19 | 2.2 | Mammary gland | 0.0 |
| CNS ca. (glio) U251 | 1.3 | Breast ca.* (pl. effusion) | 2.9 |
| | | MCF-7 | |
| CNS ca. (glio) SF-295 | 0.4 | Breast ca.* (pl. ef) MDA- | 0.0 |
| | | MB-231 | |
| Heart (fetal) | 0.0 | Breast ca.* (pl. effusion) | 0.0 |
| | | T47D | |
| Heart | 0.0 | Breast ca. BT-549 | 0.6 |
| Fetal Skeletal | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 1.0 | Ovarian ca. OVCAR-4 | 0.0 |
| Spleen | 0.0 | Ovarian ca. OVCAR-5 | 0.3 |
| Lymph node | 0.5 | Ovarian ca. OVCAR-8 | 0.8 |
| Colorectal | 0.0 | Ovarian ca. IGROV-1 | 0.0 |
| Stomach | 0.4 | Ovarian ca.* (ascites) SK- | 0.6 |
| | | OV-3 | |
| Small intestine | 0.7 | Uterus | 0.0 |
| Colon ca. SW480 | 0.0 | Placenta | 5.5 |
| Colon ca.* (SW480 | 0.6 | Prostate | 0.0 |
| met)SW620 | | | |
| Colon ca. HT29 | 0.0 | Prostate ca.* (bone | 0.0 |
| | | met)PC-3 | |
| Colon ca. HCT-116 | 0.3 | Testis | 100.0 |
| Colon ca. CaCo-2 | 0.0 | Melanoma Hs688(A).T | 0.0 |
| 83219 CC Well to Mod Diff | 0.0 | Melanoma* (met) | 0.0 |
| (ODO3866) | | Hs688(B).T | |
| Colon ca. HCC-2998 | 0.2 | Melanoma UACC-62 | 1.4 |
| Gastric ca.* (liver met) NCI- | 0.0 | Melanoma M14 | 0.0 |
| N87 | | | |
| Bladder | 0.0 | Melanoma LOX IMVI | 0.0 |
| Trachea | 32.3 | Melanoma* (met) SK- | 0.0 |
| | | MEL-5 | |
| Kidney | 0.0 | Adipose | 8.2 |

TABLE GC
Panel CNS_neurodegeneration_v1.0

| | Relative | | Relative |
|---|---|---|---|
| | Expression (%) | | Expression (%) |
| | tm6994t_{—} | | tm6994t_{—} |
| Tissue Name | ag1876_b1_s1 | Tissue Name | ag1876_b1_s1 |
| AD 1 Hippo | 5.1 | Control (Path) 3 Temporal | 5.1 |
| | | Ctx | |
| AD 2 Hippo | 37.1 | Control (Path) 4 Temporal | 13.5 |
| | | Ctx | |
| AD 3 Hippo | 3.3 | AD 1 Occipital Ctx | 1.0 |
| AD 4 Hippo | 9.4 | AD 2 Occipital Ctx | 0.0 |
| | | (Missing) | |
| AD 5 Hippo | 100.0 | AD 3 Occipital Ctx | 2.8 |
| AD 6 Hippo | 45.4 | AD 4 Occipital Ctx | 25.6 |
| Control 2 Hippo | 8.0 | AD 5 Occipital Ctx | 21.4 |
| Control 4 Hippo | 2.0 | AD 6 Occipital Ctx | 6.2 |
| Control (Path) 3 Hippo | 5.7 | Control 1 Occipital Ctx | 0.0 |
| AD 1 Temporal Ctx | 5.0 | Control 2 Occipital Ctx | 35.5 |
| AD 2 Temporal Ctx | 6.9 | Control 3 Occipital Ctx | 4.7 |
| AD 3 Temporal Ctx | 2.0 | Control 4 Occipital Ctx | 6.4 |
| AD 4 Temporal Ctx | 17.2 | Control (Path) 1 Occipital | 40.1 |
| | | Ctx | |
| AD 5 Inf Temporal Ctx | 77.8 | Control (Path) 2 Occipital | 5.8 |
| | | Ctx | |
| AD 5 Sup Temporal Ctx | 19.1 | Control (Path) 3 Occipital | 0.0 |
| | | Ctx | |
| AD 6 Inf Temporal Ctx | 15.0 | Control (Path) 4 Occipital | 22.4 |
| | | Ctx | |
| AD 6 Sup Temporal Ctx | 21.8 | Control 1 Parietal Ctx | 0.0 |
| Control 1 Temporal Ctx | 1.4 | Control 2 Parietal Ctx | 10.0 |
| Control 2 Temporal Ctx | 17.1 | Control 3 Parietal Ctx | 5.9 |
| Control 3 Temporal Ctx | 10.8 | Control (Path) 1 Parietal | 25.4 |
| | | Ctx | |
| Control 3 Temporal Ctx | 4.2 | Control (Path) 2 Parietal | 26.1 |
| | | Ctx | |
| Control (Path) 1 Temporal | 27.6 | Control (Path) 3 Parietal | 1.4 |
| Ctx | | Ctx | |
| Control (Path) 2 Temporal | 41.9 | Control (Path) 4 Parietal | 22.3 |
| Ctx | | Ctx | |

Panel 1.3D Summary The 30675745.0.499_da1 gene is most highly expressed in the testis (CT=30). In addition, there appears to be substantial expression in samples derived from adipose, placenta and trachea. Thus the expression of this gene could be used to distinguish these samples from other samples in the panel.

Panel CNS_neurodegeneration_v1.0 Summary The 30675745.0.499_da1 gene appears to be more highly expressed in the hippocampus of some patients with Alzheimer's disease than in the hippocampus of normal control brains. The hippocampus is a critical focus of the neurodegeneration that occurs as a central pathology of Alzheimer's diasease. Proteolytic activity plays an important role in numerous aspects of Alzheimer's disease, both favorably and unfavorably. For example, increased expression of serine proteases is thought to have detrimental effects on hippocampal function in Alzheimer's disease, whereas impairment of proteolytic degradation of amyloid beta peptide (Abeta) may be a key factor in the progression of the disease. The protease homology of the protein encoded by the 30675745.0.499_da1 gene suggests that it may contribute to Alzheimer's disease pathology and that agents that inhibit the 30675745.0.499_da1 protein's activity may potentially have therapeutic value in the treatment of Alzheimer's disease and other neurodegenerative diseases.

Expression of the 30675745.0.499_da1 gene is low/undetectable (Ct values>35) in all samples in Panel 4D (data not shown).

The human kallikrein gene family consists of 15 serine proteases. The expression of the kallikrein genes in human cerebral cortex and hippocampus was examined by RT-PCR and compared their expression between Alzheimer's disease (AD) and control tissue. KLK1, 4, 5, 6, 7, 8, 10, 11, 13 and 14 are expressed in both cerebral cortex and hippocampus. KLK9 is expressed in cortex but not hippocampus, whereas KLK2, 3, 12 and 15 are not expressed in either tissue. An 11.5-fold increase in KLK8 mRNA levels in AD hippocampus compared to controls. The KLK8 gene product, neuropsin, processes extracellular matrix and is important for neuronal plasticity. Therefore, the increase in KLK8 could have detrimental effects on hippocampal function in AD. See generally Shimizu-Okabe et al., Neuroreport. 12:2747-51 (2001); PMID: 11522960.

The formation, aggregation and deposition of amyloid beta peptide (Abeta) is implicated in the aetiology of Alzheimer's disease. Impairment of proteolytic degradation of Abeta may be a key factor in the progression of the disease. RP-HPLC and thioflavin T fluorescence has been used to demonstrate that Abeta42 is rapidly cleaved by the protease plasmin and that cleavage prevented the aggregation of Abeta42, and its cleavage products, into beta-pleated sheet structures. Plasmin may fulfil a similar role in vivo. See generally, Exley et al., Neuroreport Sep. 17, 2001;12(13):2967-70; PMID: 11588612.

### Example 2

Identification of NOVX Clones

The novel NOVX target sequences identified in the present invention were subjected to the exon linking process to confirm the sequence. PCR primers were designed by starting at the most upstream sequence available, for the forward primer, and at the most downstream sequence available for the reverse primer. Table HA shows the sequences of the PCR primers used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus.

Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. Table HB shows a list of these bacterial clones. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

TABLE HA

PCR Primers for Exon Linking

| | | SEQ | | SEQ |
|---|---|---|---|---|
| NOVX | | ID | | ID |
| Clone | Primer 1(5′-3′) | NO | Primer 2(5′-3′) | NO |
| NOV1a | TGGCTTATTCAGAAGAGCATAAAGG | 169 | AGTGACTAGAGATCCTCCAGGTCAGTT | 170 |
| NOV1b | TGGCTTATTCAGAAGAGCATAAAGG | 171 | AGTGACTAGAGATCCTCCAGGTCAGTT | 172 |
| NOV4a | CGCGTGACCTTGCCCCTCTTG | 173 | CGTCATCCTGAGCCCGTCCGTC | 174 |
| NOV6a | GTTTCGGGCCCTGTGCGG | 175 | GTGGTGCCCATTTGTTTTCCTCAGAGT | 176 |
| NOV6b | GGTCATGGAAGAACGGGAAGAGGT | 177 | CTGGGGAGGGTCAAAGAAGGAGCT | 178 |
| NOV7 | CTCCCACTCCTGCTGCTTCTGACT | 179 | AAGGCTGGGCCTAACCCAGTCTCAT | 180 |
| NOV9 | GTCCCTGCAGGAGAAGCCAGTGTAC | 181 | CTGGGCAAATCCTCACTTGCTTGTCT | 182 |
| NOV10 | CCTCTTTACCACACAGAACCAAGCAC | 183 | AGCCCCAGTGTGCAACTATCAAAAAC | 184 |
| | T | | | |

### Example 3

SNP Analysis of NOVX Clones

SeqCalling™ Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations. Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing (Pyrosequencing, Westborough, Mass.). Detailed protocols for Pyrosequencing can be found in: Alderborn et al. Determination of Single Nucleotide Polymorphisms by Real-time Pyrophosphate DNA Sequencing. (2000). Genome Research. 10, Issue 8, August. 1249-1265. In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels. The DNA and protein sequences for the novel single nucleotide polymorphic variants are reported. Variants are reported individually but any combination of all or a select subset of variants are also included. In addition, the positions of the variant bases and the variant amino acid residues are underlined.

Results

Variants are reported individually but any combination of all or a select subset of variants are also included as contemplated NOVX embodiments of the invention.

NOV1a SNP Data:

The DNA and protein sequences for the novel single nucleotide polymorphic variants of the TNF Receptor Associated Factor 5-like gene of CuraGen Acc. No. wugc_draft_h_nh0318116_{—}20000809_da1_(NOV1a) are reported in Table IA. Variants are reported individually but any combination of all or a select subset of variants are also included. In summary, NOV1a has 1 SNP variant (variant 13375596), whose variant positions for its nucleotide and amino acid sequences is numbered according to SEQ ID NOs:1 and 2, respectively. The nucleotide and amino acid sequence of the NOV1 a variant differs as shown in Table IA.

TABLE IA
cSNP and Coding Variants for NOV1a

| NT Position | Wild Type | | Amino Acid | Amino Acid |
|---|---|---|---|---|
| of cSNP | NT | Variant NT | position | Change |
| 1099 | A | G | 349 | Arg→Gly |

NOV4a SNP Data:

The DNA and protein sequences for the novel single nucleotide polymorphic variants of the Matrilin 2-like gene of CuraGen Acc. No. 14578444_{—}0_{—}47_(NOV4a) are reported in Table IB. Variants are reported individually but any combination of all or a select subset of variants are also included. NOV4a has 3 SNP variants (variant 13374217; variant c110.4365; and variant 110.4364), whose variant positions for its nucleotide and amino acid sequences is numbered according to SEQ ID NOs:13 and 14, respectively. The nucleotide sequence of the NOV4a variant differs as shown in Table IB.

TABLE IB
cSNP and Coding Variants for NOV4a

| | NT | | | | |
|---|---|---|---|---|---|
| | Position | Wild Type | Variant | Amino Acid | Amino Acid |
| Variant | of cSNP | NT | NT | position | Change |
| 13374217 | 462 | T | C | — | silent |
| c110.4365 | 3043 | A | G | — | no change |
| | | | | | (outside of |
| | | | | | coding region) |
| c110.4364 | 3323 | C | T | — | no change |
| | | | | | (outside of |
| | | | | | coding region) |

NOV7 SNP Data:

The DNA and protein sequences for the novel single nucleotide polymorphic variants of the Macrophage Stimulating Protein Precursor-like gene of CuraGen Acc. No. dj1182a14_da1_(NOV7) are reported in Table IC. Variants are reported individually but any combination of all or a select subset of variants are also included. NOV7 has one SNP variant (variant cg34a.348), whose variant position for its nucleotide and amino acid sequences is numbered according to SEQ ID NOs:23 and 24, respectively. The nucleotide sequence of the NOV7 variant differs as shown in Table IC.

TABLE IC
cSNP and Coding Variants for NOV7

| NT Position | Wild Type | | Amino Acid | Amino Acid |
|---|---|---|---|---|
| of cSNP | NT | Variant NT | position | Change |
| C | T | 997 | 333 | Arg→Trp |

NOV8a SNP Data:

The DNA and protein sequences for the novel single nucleotide polymorphic variants of the Nucleotide sugar transporter-like gene of CuraGen Acc. No. 138531995_(NOV8a) are reported in Table ID. Variants are reported individually but any combination of all or a select subset of variants are also included. NOV8a has one variant (variant 13375602), whose variant position for its nucleotide and amino acid sequences is numbered according to SEQ ID NOs:25 and 26, respectively. The nucleotide sequence of the NOV8a variant differs as shown in Table ID.

TABLE ID
cSNP and Coding Variants for NOV8a

| NT Position | Wild Type | | Amino Acid | Amino Acid |
|---|---|---|---|---|
| of cSNP | NT | Variant NT | position | Change |
| G | T | 1296 | 378 | Ala→Ser |

NOV9 SNP Data:

The DNA and protein sequences for the novel single nucleotide polymorphic variants of the OB binding protein 2-like gene of CuraGen Acc. No. AC018755_da1_(NOV9) are reported in Table IE. Variants are reported individually but any combination of all or a select subset of variants are also included. NOV9 has 3 SNP variants (variant 13375603; variant 13375604; and variant 13375605), whose variant position for its nucleotide and amino acid sequences is numbered according to SEQ ID NOs:28 and 29, respectively. The nucleotide sequence of the NOVX variant differs as shown in Table IE.

TABLE IE
cSNP and Coding Variants for NOV9

| | NT | | | | |
|---|---|---|---|---|---|
| | Position | Wild Type | Variant | Amino Acid | Amino Acid |
| Variant | of cSNP | NT | NT | position | Change |
| 13375603 | 215 | T | C | 72 | Val→Ala |
| 13375604 | 1008 | C | T | — | silent |
| 13375605 | 1207 | C | G | 403 | Pro→Ala |

NOV10 SNP Data:

The DNA and protein sequences for the novel single nucleotide polymorphic variants of the Trypsin-like gene of CuraGen Acc. No. 30675745.0.499_da1_(NOV10) are reported in Table IF. Variants are reported individually but any combination of all or a select subset of variants are also included. NOV10 has 5 variants (variant 13373902; variant 13373903; variant 13373904; variant 13373905; variant 13373906), whose variant position for its nucleotide and amino acid sequences is numbered according to SEQ ID NOs:30 and 31, respectively. The nucleotide sequences of the NOV10 variants differ as shown in Table IF.

TABLE IF
cSNP and Coding Variants for NOV10

| | NT | | | | |
|---|---|---|---|---|---|
| | Position | Wild Type | Variant | Amino Acid | Amino Acid |
| Variant | of cSNP | NT | NT | position | Change |
| 13373902 | 246 | A | G | — | no change |
| | | | | | (outside of the |
| | | | | | coding region) |
| 13373903 | 271 | C | T | — | no change |
| | | | | | (outside of the |
| | | | | | coding region) |
| 13373904 | 374 | T | C | 3 | Ser→Pro |
| 13373905 | 442 | T | C | — | silent |
| 13373906 | 739 | C | T | — | silent |

### Example 4

SAGE Analysis for NOVX

Serial Analysis of Gene Expression, or SAGE, is an experimental technique designed to gain a quantitative measure of gene expression. The SAGE technique itself includes several steps utilizing molecular biological, DNA sequencing and bioinformatics techniques. These steps (reviewed in Adams MD, “Serial analysis of gene expression: ESTs get smaller.” Bioessays. 18(4):261-2 (1996)) have been used to produce 9 or 10 base “tags”, which are then, in some manner, assigned gene descriptions. For experimental reasons, these tags are immediately adjacent to the 3′ end of the 3′-most NlaIII restriction site in cDNA sequences. The Cancer Genome Anatomy Project, or CGAP, is an NCI-initiated and sponsored project, which hopes to delineate the molecular fingerprint of the cancer cell. It has created a database of those cancer-related projects that used SAGE analysis in order to gain insight into the initiation and development of cancer in the human body. The SAGE expression profiles reported in this invention are generated by first identifying the Unigene accession ID associated with the given MTC gene by querying the Unigene database at http://www.ncbi.nlm.nih.gov/UniGene/. This page has then a link to the SAGE: Gene to Tag mapping (http://www.ncbi.nlm.nih.gov/SAGE/SAGEcid.cgi?cid=“unigeneID”).

This generated the reports that are included in this application, which list the number of tags found for the given gene in a given sample along with the relative expression. This information is then used to understand whether the gene has a more general role in tumorogenesis and/or tumor progression. A list of the SAGE libraries generated by CGAP and used in the analysis can be found at http://www.ncbi.nlm.nih.gov/SAGE/sagelb.cgi.

SAGE Library Data and Reliable Tag Summary for NOV8b

Reliable tags found in SAGE libraries:

AGCCTGTTGC

| | Tags per | Tag | Total |
|---|---|---|---|
| Library Name | million | counts | tags |
| SAGE Duke 1273 | 51 | 2 | 38836 |
| SAGE duke thalamus | 41 | 1 | 24371 |
| SAGE 293-CTRL | 23 | 1 | 43442 |
| SAGE HCT116 | 33 | 2 | 60322 |
| SAGE Cace2 | 64 | 4 | 61601 |
| SAGE Chen LNCaP | 16 | 1 | 62267 |
| SAGE Chen LNCaP no-DHT | 15 | 1 | 64631 |
| SAGE Chen Normal Pr | 15 | 1 | 66193 |
| SAGE Chen Turner PR | 29 | 2 | 68384 |
| SAGE CAPAN2 | 43 | 1 | 23222 |
| SAGE Panel | 80 | 2 | 24879 |
| SAGE HX | 31 | 1 | 32157 |
| SAGE H126 | 61 | 2 | 32420 |
| SAGE Duke H54lacZ | 119 | 8 | 67101 |
| SAGE Duke H54 EGFR III | 227 | 13 | 57164 |
| SAGE Duke H392 | 69 | 4 | 57529 |
| SAGE Duke GEM H100 | 85 | 6 | 70061 |
| SAGE SW837 | 32 | 2 | 60986 |
| SAGE RKO | 38 | 2 | 52064 |
| SAGE CPDR LNCaP-T | 22 | 1 | 44122 |
| SAGE 293-IND | 40 | 1 | 24481 |
| SAGE PR317 normal prostate | 16 | 1 | 59419 |
| SAGE PR317 prostate tumor | 30 | 2 | 65109 |
| SAGE pooled GMB | 48 | 3 | 61841 |
| SAGE BB542 white matter | 10 | 1 | 94806 |
| SAGE Normal pool (oth) | 15 | 1 | 63064 |
| SAGE Pane 91-16113 | 29 | 1 | 33941 |
| SAGE QVCA432-2 | 349 | 1 | 2861 |
| SAGE OV1063-3 | 102 | 4 | 38938 |
| SAGE Tul02 | 121 | 7 | 57636 |
| SAGE Tu98 | 20 | 1 | 49005 |
| SAGE Duke Mhh-1 | 41 | 2 | 48488 |
| SAGE SciencePark MCF7 control 3h | 169 | 1 | 5903 |
| SAGE SciencePark MCF7 control oh | 130 | 8 | 61079 |
| SAGE SciencePark MCF7 estradiol 3h | 50 | 3 | 59978 |
| SAGE SciencePark MCF7 estraadiol 18h | 132 | 8 | 60435 |
| SAGE lacZ | 53 | 1 | 18528 |
| SAGE 95-347 | 74 | 5 | 67240 |
| SAGE 95-259 | 152 | 6 | 39473 |
| SAGE 95-260 | 66 | 3 | 45179 |
| SAGE 95-348 | 99 | 6 | 60484 |
| SAGE Ped GBM1062 | 33 | 2 | 59935 |
| SAGE Hose 4 | 103 | 5 | 48413 |
| SAGE Es2-1 | 63 | 2 | 31502 |
| SAGE Perito-13 | 18 | 1 | 53728 |
| SAGE Meso-12 | 28 | 1 | 35032 |
| SAGE Duke H1126 | 35 | 1 | 27820 |
| SAGE LNCaP | 44 | 1 | 22637 |
| SAGE OVT-6 | 23 | 1 | 42336 |
| SAGE MDA453 | 105 | 2 | 18924 |
| SAGE Duke HMVEC | 38 | 2 | 52532 |
| SAGE HMVEC-VEGF | 17 | 1 | 57928 |
| SAGE DCIS | 194 | 8 | 41230 |
| SAGE Normal cerebeilum | 19 | 1 | 51135 |
| SAGE OVCT-8 | 89 | 3 | 33575 |
| SAGE Duke 96-349 | 179 | 1 | 5560 |
| SAGE A2780-9 | 44 | 1 | 22256 |
| SAGE ML10-10 | 87 | 5 | 56943 |
| SAGE Duke H247 normal | 115 | 7 | 60543 |
| SAGE Duke H247 Hypoxia | 27 | 2 | 71937 |
| SAGE Duke post crisis fibroblasts | 180 | 4 | 22207 |
| SAGE Duke precrisis fibroblasts | 113 | 1 | 8797 |
| SAGE DCIS 2 | 34 | 1 | 28888 |
| SAGE Br N | 53 | 2 | 37558 |
| SAGE A+ | 98 | 3 | 30551 |
| SAGE Jose29-11 | 82 | 4 | 48498 |
| SAGE Duke H1043 | 26 | 2 | 76673 |

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 29, and/or 31;
(b) a variant of a mature form of an amino acid sequence selected from the group consisting of SEQ ID NOS: 2, 4, 6, 8, 10, 12, 14, 16, 18, 20, 22, 24, 26, 29, and/or 31, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, 26, 29, and/or 31; and
(d) a variant of an amino acid sequence selected from the group consisting of SEQ ID NOS: 2, 4, 6, 8, 10, 12, 14, 16, 18, 20, 22, 24, 26, 29, and/or 31 wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting of SEQ ID NOS: 2, 4, 6, 8, 10, 12, 14, 16, 18, 20, 22, 24, 26, 29, and/or 31.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS:1, 3, 5, 7, 9, 11, 13, 15, 17, 19, 21, 23, 25, 27, 28, and/or 30.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24, 26, 29, and/or 31;
(b) a variant of a mature form of an amino acid sequence selected from the group consisting of SEQ ID NOS: 2, 4, 6, 8, 10, 12, 14, 16, 18, 20, 22, 24, 26, 29, and/or 31, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24, 26, 29, and/or 31;
(d) a variant of an amino acid sequence selected from the group consisting of SEQ ID NOS: 2, 4, 6, 8, 10, 12, 14, 16, 18, 20, 22, 24, 26, 29, and/or 31, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS: 2, 4, 6, 8, 10, 12, 14, 16, 18, 20, 22, 24, 26, 29, and/or 31, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 17, 19, 21, 23, 25, 27, 28, and/or 30.

9. The nucleic acid molecule of claim 5, wherein said nucleic acid molecule comprises a nucleotide sequence selected from the group consisting of
(a) a nucleotide sequence selected from the group consisting of SEQ ID NOS: 1, 3, 5, 7, 9, 11, 13, 15, 17, 19, 21, 23, 25, 27, 28, and/or 30;
(b) a nucleotide sequence differing by one or more nucleotides from a nucleotide sequence selected from the group consisting of SEQ ID NOS: 1, 3, 5, 7, 9, 11, 13, 15, 17, 19, 21, 23, 25, 27, 28, and/or 30, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NOS: 1, 3, 5, 7, 9, 11, 13, 15, 17, 19, 21, 23, 25, 27, 28, and/or 30, or a complement of said nucleotide sequence.

11. The nucleic acid molecule of claim 5, wherein the nucleic acid molecule comprises a nucleotide sequence selected from the group consisting of
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that immunospecifically-binds to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. A method of identifying an agent that binds to a polypeptide of claim 1, the method comprising:
(a) contacting said polypeptide with said agent; and
(b) determining whether said agent binds to said polypeptide.

21. A method for identifying an agent that modulates the expression or activity of the polypeptide of claim 1, the method comprising:
(a) providing a cell expressing said polypeptide;
(b) contacting the cell with said agent; and
(c) determining whether the agent modulates expression or activity of said polypeptide,
whereby an alteration in expression or activity of said peptide indicates said agent modulates expression or activity of said polypeptide.

22. A method for modulating the activity of the polypeptide of claim 1, the method comprising contacting a cell sample expressing the polypeptide of said claim with a compound that binds to said polypeptide in an amount sufficient to modulate the activity of the polypeptide.

23. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

24. The method of claim 23, wherein said subject is a human.

25. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

26. The method of claim 25, wherein said subject is a human.

27. A method of treating or preventing a NOVX-associated disorder, said method comprising administering to a subject in which such treatment or prevention is desired the antibody of claim 15 in an amount sufficient to treat or prevent said NOVX-associated disorder in said subject.

28. The method of claim 27, wherein the subject is a human.

29. A pharmaceutical composition comprising the polypeptide of claim 1 and a pharmaceutically-acceptable carrier.

30. A pharmaceutical composition comprising the nucleic acid molecule of claim 5 and a pharmaceutically-acceptable carrier.

31. A pharmaceutical composition comprising the antibody of claim 15 and a pharmaceutically-acceptable carrier.

32. A kit comprising in one or more containers, the pharmaceutical composition of claim 29.

33. A kit comprising in one or more containers, the pharmaceutical composition of claim 30.

34. A kit comprising in one or more containers, the pharmaceutical composition of claim 31.

35. The use of a therapeutic in the manufacture of a medicament for treating a syndrome associated with a human disease, the disease selected from a NOVX-associated disorder, wherein said therapeutic is selected from the group consisting of a NOVX polypeptide, a NOVX nucleic acid, and a NOVX antibody.

36. A method for screening for a modulator of activity or of latency or predisposition to a NOVX-associated disorder, said method comprising:
(a) administering a test compound to a test animal at increased risk for a NOVX-associated disorder, wherein said test animal recombinantly expresses the polypeptide of claim 1;
(b) measuring the activity of said polypeptide in said test animal after administering the compound of step (a);
(c) comparing the activity of said protein in said test animal with the activity of said polypeptide in a control animal not administered said polypeptide, wherein a change in the activity of said polypeptide in said test animal relative to said control animal indicates the test compound is a modulator of latency of or predisposition to a NOVX-associated disorder.

37. The method of claim 36, wherein said test animal is a recombinant test animal that expresses a test protein transgene or expresses said transgene under the control of a promoter at an increased level relative to a wild-type test animal, and wherein said promoter is not the native gene promoter of said transgene.

38. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease,
wherein an alteration in the expression level of the polypeptide in the first subject as compared to the control sample indicates the presence of or predisposition to said disease.

39. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

40. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18, 20, 22, 24, 26, 29, and/or 31, or a biologically active fragment thereof.

41. A method of treating a pathological state in a mammal, the method comprising administering to the mammal the antibody of claim 15 in an amount sufficient to alleviate the pathological state.
